(19) United States
(12) Patent Application Publication
(10) Pub. No.: US 2026/0120797 A1
(43) Pub. Date: Apr. 30, 2026

(54) CELL-FREE DNA BLOOD-BASED TEST FOR CANCER SCREENING

(71) Applicant: GUARDANT HEALTH, INC., Palo Alto, CA (US)

(72) Inventors: Yupeng HE, Alhambra, CA (US); William Walter YOUNG GREENWALD, San Diego, CA (US); Ariel JAIMOVICH, Redwood City, CA (US); William J. GREENLEAF, Menlo Park, CA (US)

(21) Appl. No.: 19/371,641

(22) Filed: Oct. 28, 2025

Related U.S. Application Data
(63) Continuation of application No. PCT/US2024/028061, filed on May 6, 2024.
(60) Provisional application No. 63/500,480, filed on May 5, 2023.
(60) Provisional application No. 63/614,350, filed on Dec. 22, 2023.

(51) Int. Cl. G16B 20/20; G06N 20/00; G16B 20/10; G16B 30/00; G16B 40/20

(52) U.S. Cl. CPC G16B 20/20; G06N 20/00; G16B 20/10; G16B 30/00; G16B 40/20

(57) ABSTRACT

A disease classification method includes determining, using a predictive model, whether cell-free nucleic acid samples are tumor-derived or non-tumor derived based on at least one of the cell-free nucleic acid score or a tumor fraction regression (TFR) score satisfying a respective threshold. The TFR score is determined based on a quantification of an observed tumor-associated aberrant methylation of each of a plurality of cell-free nucleic acid samples using a TFR model. The TFR score includes a fraction of molecules of the plurality of cell-free nucleic acid samples that indicate a tumor. The cell-free nucleic acid score is indicative of the presence of a tumor, and is based on at least one of epigenetic factors or genomic alterations of the cell-free nucleic acid samples.

### CROSS-REFERENCE TO RELATED APPLICATIONS

The present application is Continuation of International Patent Application No. PCT/US2024/028061, filed May 6, 2024, which claims the benefit of U.S. Provisional Patent Application No. 63/500,480, filed May 5, 2023, and U.S. Provisional Patent Application No. 63/614,350, filed Dec. 22, 2023, each of which is incorporated by reference herein in its entirety for all purposes.

### BACKGROUND

Colorectal cancer (CRC) is the third most diagnosed cancer and second leading cause of cancer-related death in men and women in the United States (US). The lifetime risk of CRC in the US is approximately 4% with 52,500 people expected to die from the disease in 2023. Earlier CRC detection impacts overall survival; 5-year relative survival is 91% in those with localized disease compared to 14% in those with metastatic disease. Asymptomatic CRC screening reduces CRC incidence and mortality and is uniformly recommended in clinical guidelines published by leading professional societies, including the US Preventive Services Taskforce (USPSTF), the US Multi-Society Taskforce, and the American Cancer Society (ACS). Numerous screening options are available, including direct visualization and stool-based tests. Despite the broadly recognized benefit of CRC screening, currently available options have significant barriers leading to approximately 59% of eligible individuals age 45 years and older being adherent. This is well below the target of 80% set forth by the National Colorectal Cancer Roundtable (NCCRT), which was established by the Centers for Disease Control and Prevention (CDC) and ACS. Additionally, 76% of CRC-related deaths occur in individuals who are not up-to-date with screening. Therefore, there is a pressing need for CRC screening tests that are easier to administer and increase screening adherence.

Factors contributing to low CRC screening adherence include time required to perform screening, challenges related to scheduling colonoscopy, concern over test invasiveness and pain, fear of the test, discomfort or embarrassment associated with endoscopic exams, lack of insurance coverage, distance from the test provider, and lack of physician recommendation for screening. Incorporating a blood-based test, drawn and completed as part of a routine health care encounter, to the existing screening model would provide an additional screening option that is relatively simple to complete.

### BRIEF SUMMARY

Disclosed herein is a cell-free DNA (cfDNA) blood-based CRC screening test. The CRC screening test includes determining, using a predictive model, whether cell-free nucleic acid samples are tumor-derived or non-tumor derived based on at least one of the cell-free nucleic acid score or a tumor fraction regression (TFR) score satisfying a respective threshold. The TFR score may be determined based on a quantification of an observed tumor-associated aberrant methylation of each of a plurality of cell-free nucleic acid samples using a tumor fraction regression (TFR) model. The TFR score may include a fraction of molecules of the plurality of cell-free nucleic acid samples that indicate a tumor. The cell-free nucleic acid score may be indicative of presence of a tumor, and may be based on at least one of epigenetic factors or genomic alterations of the cell-free nucleic acid samples. In some examples, the epigenetic factors may include fragmentomics data and logistical regression methylation data. The cell-free nucleic acid score may also be based on the TFR score, in some examples.

Additionally or alternatively, the method may include determining the quantification of the observed tumor-associated aberrant methylation of each of the plurality of cell-free nucleic acid samples.

Additionally or alternatively, determining the quantification of the observed tumor-associated aberrant methylation of each of the plurality of cell-free nucleic acid samples includes quantifying a number of unique methylated molecules mapping to each of a plurality of genomic regions.

Additionally or alternatively, the method may include determining the epigenetic factors of each of the plurality of cell-free nucleic acid samples.

Additionally or alternatively, the method may include determining the epigenetic factors of each of the plurality of cell-free nucleic acid samples based on a methylation logistic regression (LR) model cancer or non-cancer classification.

Additionally or alternatively, the method may include determining, using a LR model, the methylation LR model cancer or non-cancer classification.

Additionally or alternatively, the method may include determining the epigenetic factors of each of the plurality of cell-free nucleic acid samples based on a cancer signal from cell-free nucleic acid fragmentation patterns associated with a plurality of sequence fragments from the plurality of cell-free nucleic acid samples.

Additionally or alternatively, the method may include determining, using a fragmentomics model, the cancer signal from the cell-free nucleic acid fragmentation patterns associated with the plurality of sequence fragments from the plurality of cell-free nucleic acid samples.

Additionally or alternatively, the method may include determining the epigenetic factors of each of the plurality of cell-free nucleic acid samples based on a methylation logistic regression (LR) model cancer or non-cancer classification and based on a cancer signal from cell-free nucleic acid fragmentation patterns associated with a plurality of sequence fragments from the plurality of cell-free nucleic acid samples.

Additionally or alternatively, determining the cell-free nucleic acid score is further based on the TFR score.

Additionally or alternatively, the method may include determining the genomic alterations of each of the plurality of cell-free nucleic acid samples.

Additionally or alternatively, determining the genomic alterations of each of the plurality of cell-free nucleic acid samples includes determining somatic variants observed in molecules from each of a plurality of sequence fragments from the plurality of cell-free nucleic acid samples.

Additionally or alternatively, the plurality of cell-free nucleic acid samples may be from a plurality of genomic regions. The plurality of genomic regions may include at least one of: a genomic region known to be associated with a cancer type, a genomic region associated with a known methylation status, a genomic region known to be associated with hypomethylation, or a genomic region known to be associated with therapy response.

Additionally or alternatively, the plurality of genomic regions includes at least one genomic region known to be associated with colorectal cancer.

Additionally or alternatively, determining the cell-free nucleic acid score is further based on the TFR score.

Additionally or alternatively, the plurality of cell-free nucleic acid samples includes cell-free deoxyribonucleic (cfDNA) samples.

Additionally or alternatively, the plurality of cell-free nucleic acid samples includes ribonucleic acid (RNA) samples.

Additionally or alternatively, the plurality of cell-free nucleic acid samples includes cell-free ribonucleic acid (cfRNA) samples.

Additionally or alternatively, the plurality of cell-free nucleic acid samples includes mitochondrial deoxyribonucleic (mtDNA) samples.

Additionally or alternatively, the plurality of cell-free nucleic acid samples includes mitochondrial ribonucleic (mtRNA) samples.

Additionally or alternatively, the plurality of cell-free nucleic acid samples includes extracellular vesicle-bound deoxyribonucleic (evDNA) samples.

Another example method may include determining, based on a quantification of an observed tumor-associated aberrant methylation of each of the plurality of cell-free nucleic acid samples, using a tumor fraction regression (TFR) model, a TFR score. The TFR score may include a fraction of molecules of the plurality of cell-free nucleic acid samples that indicate a tumor. The method may further include determining, based on the TFR score satisfying a respective threshold, using a predictive model, that the plurality of cell-free nucleic acid samples is tumor-derived or non-tumor derived.

Additionally or alternatively, the method may include determining the quantification of the observed tumor-associated aberrant methylation of each of the plurality of cell-free nucleic acid samples.

Additionally or alternatively, determining the quantification of the observed tumor-associated aberrant methylation of each of the plurality of cell-free nucleic acid samples includes quantifying a number of unique methylated molecules mapping to each of a plurality of genomic regions.

Additionally or alternatively, determining that the plurality of cell-free nucleic acid samples is tumor-derived or non-tumor derived is further based on a cell-free nucleic acid score indicative of presence of a tumor.

Additionally or alternatively, the method may include determining, based on at least one of epigenetic factors or genomic alterations of each of the plurality of cell-free nucleic acid samples, the cell-free nucleic acid score indicative of presence of a tumor.

Additionally or alternatively, the method may include determining the epigenetic factors of each of the plurality of cell-free nucleic acid samples.

Additionally or alternatively, the method may include determining the epigenetic factors of each of the plurality of cell-free nucleic acid samples based on a methylation logistic regression (LR) model cancer or non-cancer classification.

Additionally or alternatively, the method may include determining, using a LR model, the methylation LR model cancer or non-cancer classification.

Additionally or alternatively, the method may include determining the epigenetic factors of each of the plurality of cell-free nucleic acid samples based on a cancer signal from cell-free nucleic acid fragmentation patterns associated with a plurality of sequence fragments from the plurality of cell-free nucleic acid samples.

Additionally or alternatively, the method may include determining, using a fragmentomics model, the cancer signal from the cell-free nucleic acid fragmentation patterns associated with the plurality of sequence fragments from the plurality of cell-free nucleic acid samples.

Additionally or alternatively, the method may include determining the genomic alterations of each of the plurality of cell-free nucleic acid samples.

Additionally or alternatively, determining the genomic alterations of each of the plurality of cell-free nucleic acid samples includes determining somatic variants observed in molecules from each of a plurality of sequence fragments from the plurality of cell-free nucleic acid samples.

Additionally or alternatively, the method may include determining the epigenetic factors of each of the plurality of cell-free nucleic acid samples based on a methylation logistic regression (LR) model cancer or non-cancer classification and based on a cancer signal from cell-free nucleic acid fragmentation patterns associated with a plurality of sequence fragments from the plurality of cell-free nucleic acid samples.

Additionally or alternatively, determining the cell-free nucleic acid score is further based on the TFR score.

Additionally or alternatively, the plurality of genomic regions may include at least one of a genomic region known to be associated with a cancer type, a genomic region associated with a known methylation status, a genomic region known to be associated with hypomethylation, or a genomic region known to be associated with therapy response.

Additionally or alternatively, the plurality of genomic regions includes at least one genomic region known to be associated with colorectal cancer.

Additionally or alternatively, the plurality of cell-free nucleic acid samples includes cell-free deoxyribonucleic (cfDNA) samples.

Additionally or alternatively, the plurality of cell-free nucleic acid samples includes ribonucleic acid (RNA) samples.

Additionally or alternatively, the plurality of cell-free nucleic acid samples includes cell-free ribonucleic acid (cfRNA) samples.

Additionally or alternatively, the plurality of cell-free nucleic acid samples includes mitochondrial deoxyribonucleic (mtDNA) samples.

Additionally or alternatively, the plurality of cell-free nucleic acid samples includes mitochondrial ribonucleic (mtRNA) samples.

Additionally or alternatively, the plurality of cell-free nucleic acid samples includes extracellular vesicle-bound deoxyribonucleic (evDNA) samples.

Another example may include determining, based on at least one of epigenetic factors or genomic alterations of each of the plurality of cell-free nucleic acid samples, a cell-free nucleic acid score indicative of presence of a tumor, and determining, based on the cell-free nucleic acid score satisfying a respective threshold, using a predictive model, that the plurality of cell-free nucleic acid samples is tumor-derived or non-tumor derived.

Additionally or alternatively, determining that the plurality of cell-free nucleic acid samples is tumor-derived or non-tumor derived is further based on a tumor fraction regression (TFR) score satisfying a threshold. The TFR score may be indicative of a fraction of molecules of the plurality of cell-free nucleic acid samples that indicate a tumor.

Additionally or alternatively, the method may include determining, based on a quantification of an observed tumor-associated aberrant methylation of each of the plurality of cell-free nucleic acid samples, using a TFR model, the TFR score.

Additionally or alternatively, the method may include determining the quantification of the observed tumor-associated aberrant methylation of each of the plurality of cell-free nucleic acid samples.

Additionally or alternatively, determining the quantification of the observed tumor-associated aberrant methylation of each of the plurality of cell-free nucleic acid samples includes quantifying a number of unique methylated molecules mapping to each of a plurality of genomic regions.

Additionally or alternatively, determining the cell-free nucleic acid score is further based on the TFR score.

Additionally or alternatively, the method may include determining the epigenetic factors of each of the plurality of cell-free nucleic acid samples.

Additionally or alternatively, the method may include determining the epigenetic factors of each of the plurality of cell-free nucleic acid samples based on a methylation logistic regression (LR) model cancer or non-cancer classification.

Additionally or alternatively, the method may include determining, using a LR model, the methylation LR model cancer or non-cancer classification.

Additionally or alternatively, the method may include determining the epigenetic factors of each of the plurality of cell-free nucleic acid samples based on a cancer signal from cell-free nucleic acid fragmentation patterns associated with a plurality of sequence fragments from the plurality of cell-free nucleic acid samples.

Additionally or alternatively, the method may include determining, using a fragmentomics model, the cancer signal from the cell-free nucleic acid fragmentation patterns associated with the plurality of sequence fragments from the plurality of cell-free nucleic acid samples.

Additionally or alternatively, the method may include determining the epigenetic factors of each of the plurality of cell-free nucleic acid samples based on a methylation logistic regression (LR) model cancer or non-cancer classification and based on a cancer signal from cell-free nucleic acid fragmentation patterns associated with a plurality of sequence fragments from the plurality of cell-free nucleic acid samples.

Additionally or alternatively, the method may include determining the cell-free nucleic acid score is further based on a tumor fraction regression (TFR) score. The TFR score may be indicative of a fraction of molecules of the plurality of cell-free nucleic acid samples that indicate a tumor.

Additionally or alternatively, the method may include determining the genomic alterations of each of the plurality of cell-free nucleic acid samples.

Additionally or alternatively, determining the genomic alterations of each of the plurality of cell-free nucleic acid samples includes determining somatic variants observed in molecules from each of a plurality of sequence fragments from the plurality of cell-free nucleic acid samples.

Additionally or alternatively, the plurality of cell-free nucleic acid samples may be from a plurality of genomic regions. The plurality of genomic regions may include at least one of: a genomic region known to be associated with a cancer type, a genomic region associated with a known methylation status, a genomic region known to be associated with hypomethylation, or a genomic region known to be associated with therapy response.

Additionally or alternatively, the plurality of genomic regions may include at least one genomic region known to be associated with colorectal cancer.

Additionally or alternatively, determining that the plurality of cell-free nucleic acid samples is tumor-derived or non-tumor derived is further based on a methylation value satisfying a threshold. The methylation score may be indicative of a quantity of molecules of the plurality of cell-free nucleic acid samples that indicate a tumor.

Additionally or alternatively, the plurality of cell-free nucleic acid samples includes cell-free deoxyribonucleic (cfDNA) samples.

Additionally or alternatively, the plurality of cell-free nucleic acid samples includes ribonucleic acid (RNA) samples.

Additionally or alternatively, the plurality of cell-free nucleic acid samples includes cell-free ribonucleic acid (cfRNA) samples.

Additionally or alternatively, the plurality of cell-free nucleic acid samples includes mitochondrial deoxyribonucleic (mtDNA) samples.

Additionally or alternatively, the plurality of cell-free nucleic acid samples includes mitochondrial ribonucleic (mtRNA) samples.

Additionally or alternatively, the plurality of cell-free nucleic acid samples includes extracellular vesicle-bound deoxyribonucleic (evDNA) samples.

The predictive model may be trained using labeled cell-free nucleic acid sample data. A tumor prediction may be determined based on based on at least one of the cell-free nucleic acid score or the TFR score satisfying a respective threshold. The tumor prediction, along with the tumor-derived label or the non-tumor-derived label of the cell-free nucleic acid data, may be used to train the predictive model. Using the trained predictive model to detect CRC using cfDNA samples may be less invasive than traditional testing or screening used to detect CRC.

Additionally or alternatively, the method may include determining the quantification of the observed tumor-associated aberrant methylation of each of the plurality of cell-free nucleic acid samples.

Additionally or alternatively, determining the quantification of the observed tumor-associated aberrant methylation of each of the plurality of cell-free nucleic acid samples includes quantifying a number of unique methylated molecules mapping to each of a plurality of genomic regions.

Additionally or alternatively, the method may include determining the epigenetic factors of each of the plurality of cell-free nucleic acid samples.

Additionally or alternatively, the method may include determining the epigenetic factors of each of the plurality of cell-free nucleic acid samples based on a methylation logistic regression (LR) model cancer or non-cancer classification.

Additionally or alternatively, the method may include determining, using a LR model, the methylation LR model cancer or non-cancer classification.

Additionally or alternatively, determining the epigenetic factors of each of the plurality of cell-free nucleic acid samples is based on a cancer signal from cell-free nucleic acid fragmentation patterns associated with a plurality of sequence fragments from the plurality of cell-free nucleic acid samples.

Additionally or alternatively, the method may include determining, using a fragmentomics model, the cancer signal from the cell-free nucleic acid fragmentation patterns associated with the plurality of sequence fragments from the plurality of cell-free nucleic acid samples.

Additionally or alternatively, the method may include determining the epigenetic factors of each of the plurality of cell-free nucleic acid samples based on a methylation logistic regression (LR) model cancer or non-cancer classification and based on a cancer signal from cell-free nucleic acid fragmentation patterns associated with a plurality of sequence fragments from the plurality of cell-free nucleic acid samples.

Additionally or alternatively, determining the cell-free nucleic acid score is further based on the TFR score.

Additionally or alternatively, the method may include determining the genomic alterations of each of the plurality of cell-free nucleic acid samples.

Additionally or alternatively, determining the genomic alterations of each of the plurality of cell-free nucleic acid samples includes determining somatic variants observed in molecules from the plurality of cell-free nucleic acid samples.

Additionally or alternatively, the plurality of cell-free nucleic acid samples are from a plurality of genomic regions. The plurality of genomic regions may include at least one of: a genomic region known to be associated with a cancer type, a genomic region associated with a known methylation status, a genomic region known to be associated with hypomethylation, or a genomic region known to be associated with therapy response.

Additionally or alternatively, the plurality of genomic regions includes at least one genomic region known to be associated with colorectal cancer.

Additionally or alternatively, determining the cell-free nucleic acid score is further based on the TFR score.

Additionally or alternatively, the plurality of cell-free nucleic acid samples includes cell-free deoxyribonucleic (cfDNA) samples.

Additionally or alternatively, the plurality of cell-free nucleic acid samples includes ribonucleic acid (RNA) samples.

Additionally or alternatively, the plurality of cell-free nucleic acid samples includes cell-free ribonucleic acid (cfRNA) samples.

Additionally or alternatively, the plurality of cell-free nucleic acid samples includes mitochondrial deoxyribonucleic (mtDNA) samples.

Additionally or alternatively, the plurality of cell-free nucleic acid samples includes mitochondrial ribonucleic (mtRNA) samples.

Additionally or alternatively, the plurality of cell-free nucleic acid samples includes extracellular vesicle-bound deoxyribonucleic (evDNA) samples.

Another example method may include determining, based on a quantification of an observed tumor-associated aberrant methylation of each of a plurality of cell-free nucleic acid samples, using a tumor fraction regression (TFR) model, a TFR score. The TFR score may include, a fraction of molecules of the plurality of cell-free nucleic acid samples that indicate a tumor. Each of the plurality of cell-free nucleic acid samples may be labeled with a tumor-derived label or a non-tumor-derived label. The example method may further include determining, based on the TFR score satisfying a respective threshold, a tumor prediction for each of the plurality of cell-free nucleic acid sample. The example method may further include generating, based on the tumor-derived label or the non-tumor-derived label and the tumor prediction of the plurality of cell-free nucleic acid samples, a predictive model to predict a tumor in the plurality of cell-free nucleic acid samples, and outputting the predictive model.

Additionally or alternatively, the method may include determining the quantification of the observed tumor-associated aberrant methylation of each of the plurality of cell-free nucleic acid samples.

Additionally or alternatively, determining the quantification of the observed tumor-associated aberrant methylation of each of the plurality of cell-free nucleic acid samples includes quantifying a number of unique methylated molecules mapping to each of a plurality of genomic regions.

Additionally or alternatively, determining the tumor prediction for each of the plurality of cell-free nucleic acid samples is further based on a cell-free nucleic acid score indicative of presence of a tumor.

Additionally or alternatively, the method may include based on at least one of epigenetic factors or genomic alterations of each of the plurality of cell-free nucleic acid samples, the cell-free nucleic acid score indicative of presence of a tumor.

Additionally or alternatively, the method may include determining the epigenetic factors of each of the plurality of cell-free nucleic acid samples.

Additionally or alternatively, the method may include determining the epigenetic factors of each of the plurality of cell-free nucleic acid samples based on a methylation logistic regression (LR) model cancer or non-cancer classification.

Additionally or alternatively, the method may include determining, using a LR model, the methylation LR model cancer or non-cancer classification.

Additionally or alternatively, the method may include determining the epigenetic factors of each of the plurality of cell-free nucleic acid samples based on a cancer signal from cell-free nucleic acid fragmentation patterns associated with a plurality of sequence fragments from the plurality of cell-free nucleic acid samples.

Additionally or alternatively, the method may include determining, using a fragmentomics model, the cancer signal from the cell-free nucleic acid fragmentation patterns associated with a plurality of sequence fragments from the plurality of cell-free nucleic acid samples.

Additionally or alternatively, the method may include determining the genomic alterations of each of the plurality of cell-free nucleic acid samples.

Additionally or alternatively, determining the genomic alterations of each of the plurality of cell-free nucleic acid samples includes determining somatic variants observed in molecules from each of a plurality of sequence fragments from the plurality of cell-free nucleic acid samples.

Additionally or alternatively, the method may include determining the epigenetic factors of each of the plurality of cell-free nucleic acid samples based on a methylation logistic regression (LR) model cancer or non-cancer classification and based on a cancer signal from cell-free nucleic acid fragmentation patterns associated with a plurality of sequence fragments from the plurality of cell-free nucleic acid samples.

Additionally or alternatively, determining the cell-free nucleic acid score is further based on the TFR score.

Additionally or alternatively, the plurality of cell-free nucleic acid samples may be from a plurality of genomic regions. The plurality of genomic regions may include at least one of: a genomic region known to be associated with a cancer type, a genomic region associated with a known methylation status, a genomic region known to be associated with hypomethylation, or a genomic region known to be associated with therapy response.

Additionally or alternatively, the plurality of genomic regions includes at least one genomic region known to be associated with colorectal cancer.

Additionally or alternatively, the plurality of cell-free nucleic acid samples includes cell-free deoxyribonucleic (cfDNA) samples.

Additionally or alternatively, the plurality of cell-free nucleic acid samples includes ribonucleic acid (RNA) samples.

Additionally or alternatively, the plurality of cell-free nucleic acid samples includes cell-free ribonucleic acid (cfRNA) samples.

Additionally or alternatively, the plurality of cell-free nucleic acid samples includes mitochondrial deoxyribonucleic (mtDNA) samples.

Additionally or alternatively, the plurality of cell-free nucleic acid samples includes mitochondrial ribonucleic (mtRNA) samples.

Additionally or alternatively, the plurality of cell-free nucleic acid samples includes extracellular vesicle-bound deoxyribonucleic (evDNA) samples.

An example method may include determining, based on at least one of epigenetic factors or genomic alterations of each of a plurality of cell-free nucleic acid samples, a cell-free nucleic acid score indicative of presence of a tumor. Each of the plurality of cell-free nucleic acid samples may be labeled with a tumor-derived label or a non-tumor-derived label. The example method may further include determining, based on the cell-free nucleic acid score satisfying a respective threshold, a tumor prediction for each of the plurality of cell-free nucleic acid samples. The example method may further include generating, based on the tumor-derived label or the non-tumor-derived label and the tumor prediction for each of the plurality of cell-free nucleic acid samples, a predictive model to predict a tumor in the plurality of cell-free nucleic acid samples, and outputting the predictive model.

Additionally or alternatively, determining the tumor prediction for each of the plurality of cell-free nucleic acid is further based on a tumor fraction regression (TFR) score satisfying a threshold. The TFR score may be indicative of a fraction of molecules of the plurality of cell-free nucleic acid samples that indicate a tumor.

Additionally or alternatively, the method may include determining, based on a quantification of an observed tumor-associated aberrant methylation of each of the plurality of cell-free nucleic acid samples, using a TFR model, the TFR score.

Additionally or alternatively, the method may include determining the quantification of the observed tumor-associated aberrant methylation of each of the plurality of cell-free nucleic acid samples.

Additionally or alternatively, determining the quantification of the observed tumor-associated aberrant methylation of each of the plurality of cell-free nucleic acid samples includes quantifying a number of unique methylated molecules mapping to each of a plurality of genomic regions.

Additionally or alternatively, determining the cell-free nucleic acid score is further based on the TFR score.

Additionally or alternatively, the method may include determining the epigenetic factors of each of the plurality of cell-free nucleic acid samples.

Additionally or alternatively, the method may include determining the epigenetic factors of each of the plurality of cell-free nucleic acid samples based on a methylation logistic regression (LR) model cancer or non-cancer classification.

Additionally or alternatively, the method may include determining, using a LR model, the methylation LR model cancer or non-cancer classification.

Additionally or alternatively, the method may include determining the epigenetic factors of each of the plurality of cell-free nucleic acid samples based on a cancer signal from cell-free nucleic acid fragmentation patterns associated with a plurality of sequence fragments from the plurality of cell-free nucleic acid samples.

Additionally or alternatively, the method may include determining, using a fragmentomics model, the cancer signal from the cell-free nucleic acid fragmentation patterns associated with the plurality of sequence fragments from the plurality of cell-free nucleic acid samples.

Additionally or alternatively, the method may include determining the epigenetic factors of each of the plurality of cell-free nucleic acid samples based on a methylation logistic regression (LR) model cancer or non-cancer classification and based on a cancer signal from cell-free nucleic acid fragmentation patterns associated with a plurality of sequence fragments from the plurality of cell-free nucleic acid samples.

Additionally or alternatively, determining the cell-free nucleic acid score is further based on a tumor fraction regression (TFR) score. The TFR score may be indicative of a fraction of molecules of the plurality of cell-free nucleic acid samples that indicate a tumor.

Additionally or alternatively, the method may include determining the genomic alterations of each of the plurality of cell-free nucleic acid samples.

Additionally or alternatively, determining the genomic alterations of each of the plurality of cell-free nucleic acid samples includes determining somatic variants observed in molecules from each of a plurality of sequence fragments from the plurality of cell-free nucleic acid samples.

Additionally or alternatively, the plurality of cell-free nucleic acid samples may be from a plurality of genomic regions. The plurality of genomic regions may include at least one of: a genomic region known to be associated with a cancer type, a genomic region associated with a known methylation status, a genomic region known to be associated with hypomethylation, or a genomic region known to be associated with therapy response.

Additionally or alternatively, the plurality of genomic regions includes at least one genomic region known to be associated with colorectal cancer.

Additionally or alternatively, determining that the plurality of cell-free nucleic acid samples is tumor-derived or non-tumor derived is further based on a methylation value satisfying a threshold. The methylation score may be indicative of a quantity of molecules of the plurality of cell-free nucleic acid samples that indicate a tumor.

Additionally or alternatively, the plurality of cell-free nucleic acid samples includes cell-free deoxyribonucleic (cfDNA) samples.

Additionally or alternatively, the plurality of cell-free nucleic acid samples includes ribonucleic acid (RNA) samples.

Additionally or alternatively, the plurality of cell-free nucleic acid samples includes cell-free ribonucleic acid (cfRNA) samples.

Additionally or alternatively, the plurality of cell-free nucleic acid samples includes mitochondrial deoxyribonucleic (mtDNA) samples.

Additionally or alternatively, the plurality of cell-free nucleic acid samples includes mitochondrial ribonucleic (mtRNA) samples.

Additionally or alternatively, the plurality of cell-free nucleic acid samples includes extracellular vesicle-bound deoxyribonucleic (evDNA) samples.

An example method may include detecting one or more biomarkers in a biological sample, and determining, based on a quantification of an observed tumor-associated aberrant methylation of each of a plurality of cell-free nucleic acid samples, using a tumor fraction regression (TFR) model, a TFR score. The TFR score may include a fraction of molecules of the plurality of cell-free nucleic acid samples that indicate a tumor. The example method may further include determining, based on at least one of epigenetic factors or genomic alterations of each of the plurality of cell-free nucleic acid samples, a cell-free nucleic acid score indicative of presence of a tumor, and determining, based on at least one of the detected biomarkers, the cell-free nucleic acid score, or the TFR score satisfying a respective threshold, that the biological sample is tumor-derived or non-tumor derived.

Additionally or alternatively, the method may include determining the quantification of the observed tumor-associated aberrant methylation of each of the plurality of cell-free nucleic acid samples.

Additionally or alternatively, determining the quantification of the observed tumor-associated aberrant methylation of each of the plurality of cell-free nucleic acid samples includes quantifying a number of unique methylated molecules mapping to each of a plurality of genomic regions.

Additionally or alternatively, the method may include determining the epigenetic factors of each of the plurality of cell-free nucleic acid samples.

Additionally or alternatively, the method may include determining the epigenetic factors of each of the plurality of cell-free nucleic acid samples based on a methylation logistic regression (LR) model cancer or non-cancer classification.

Additionally or alternatively, the method may include determining, using a LR model, the methylation LR model cancer or non-cancer classification.

Additionally or alternatively, the method may include determining the epigenetic factors of each of the plurality of cell-free nucleic acid samples based on a cancer signal from cell-free nucleic acid fragmentation patterns associated with a plurality of sequence fragments from the plurality of cell-free nucleic acid samples.

Additionally or alternatively, the method may include determining, using a fragmentomics model, the cancer signal from the cell-free nucleic acid fragmentation patterns associated with the plurality of sequence fragments from the plurality of cell-free nucleic acid samples.

Additionally or alternatively, the method may include determining the epigenetic factors of each of the plurality of cell-free nucleic acid samples based on a methylation logistic regression (LR) model cancer or non-cancer classification and based on a cancer signal from cell-free nucleic acid fragmentation patterns associated with a plurality of sequence fragments from the plurality of cell-free nucleic acid samples.

Additionally or alternatively, determining the cell-free nucleic acid score is further based on the TFR score.

Additionally or alternatively, the method may include determining the genomic alterations of each of the plurality of cell-free nucleic acid samples.

Additionally or alternatively, determining the genomic alterations of each of the plurality of cell-free nucleic acid samples includes determining somatic variants observed in molecules from each of a plurality of sequence fragments from the plurality of cell-free nucleic acid samples.

Additionally or alternatively, the method may include the plurality of cell-free nucleic acid samples may be from a plurality of genomic regions. The plurality of genomic regions may include at least one of: a genomic region known to be associated with a cancer type, a genomic region associated with a known methylation status, a genomic region known to be associated with hypomethylation, or a genomic region known to be associated with therapy response.

Additionally or alternatively, the plurality of genomic regions includes at least one genomic region known to be associated with colorectal cancer.

Additionally or alternatively, determining the cell-free nucleic acid score is further based on the TFR score.

Additionally or alternatively, the plurality of cell-free nucleic acid samples includes cell-free deoxyribonucleic (cfDNA) samples.

Additionally or alternatively, the plurality of cell-free nucleic acid samples includes ribonucleic acid (RNA) samples.

Additionally or alternatively, the plurality of cell-free nucleic acid samples includes cell-free ribonucleic acid (cfRNA) samples.

Additionally or alternatively, the plurality of cell-free nucleic acid samples includes mitochondrial deoxyribonucleic (mtDNA) samples.

Additionally or alternatively, the plurality of cell-free nucleic acid samples includes mitochondrial ribonucleic (mtRNA) samples.

Additionally or alternatively, the plurality of cell-free nucleic acid samples includes extracellular vesicle-bound deoxyribonucleic (evDNA) samples.

Additionally or alternatively, the biomarker is one or more of those selected from: proteins, exosomes, exomeres, microvesicles, apoptotic bodies, neutrophil extracellular traps (NETs), immune cells, tumor-educated platelets (TEPs), microbiome, virome, toll-like receptors (TLRs), and mitochondrial DNA (mtDNA).

Additionally or alternatively, detecting one ore more biomarkers comprises detecting the presence or levels of the one or more biomarkers.

Additionally or alternatively, determining that the biological sample is tumor-derived or non-tumor derived comprises comparing the levels of the one or more biomarkers in the biological sample to a control.

Additionally or alternatively, the control is a reference level or a level present in a healthy, non-cancer subject.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 is a flow chart that schematically depicts an example artificial intelligence (e.g., machine learning) technique for generating a classifier configured for differentiating or classifying tumor and non-tumor origin nucleic acid variants in a cell-free nucleic acid (cfDNA) sample obtained from a test subject.

FIG. 2 illustrates an example of a system for determining whether a sample of a test subject is tumor-derived, according to an embodiment of the present disclosure.

FIG. 3A is an illustration of a method for sequencing a cfDNA molecule to obtain a methylation state vector.

FIG. 3B is a diagrammatic representation of an example environment 307 that identifies nucleic acids that correspond to classification regions of a reference sequence, where the classification regions have at least a threshold number of CpGs, according to one or more implementations.

FIG. 4 shows examples for end motifs according to embodiments of the present disclosure.

FIG. 5 illustrates one example showing how the degree of overhangs of cell-free DNA molecules (i.e., overhang index) can be determined.

FIG. 6 is an illustration of the calculation of methylation levels along a DNA molecule after mapping to the human reference genome.

FIG. 7 shows a method of determining an overhang index.

FIG. 8 is a flowchart illustrating an example method for generating a predictive model.

FIG. 9 is a flowchart illustrating an example training method for generating the ML module of FIG. 8 using the training module of FIG. 8.

FIG. 10 is an illustration of an exemplary process flow for using a machine learning-based classifier to classify a sequence fragment/read and/or variant as tumor origin or non-tumor origin.

FIG. 11 is an illustration of an exemplary process flow of a method to classify nucleic acid samples as tumor origin or non-tumor origin.

FIG. 12 is an illustration of an exemplary process flow of a method to classify nucleic acid samples as tumor origin or non-tumor origin.

FIG. 13 is an illustration of an exemplary process flow of a method to classify nucleic acid samples as tumor origin or non-tumor origin.

FIG. 14 is an illustration of an exemplary process flow of a method to train a predictive model to classify nucleic acid samples as tumor origin or non-tumor origin.

FIG. 15 is an illustration of an exemplary process flow of a method to train a predictive model to classify nucleic acid samples as tumor origin or non-tumor origin.

FIG. 16 is an illustration of an exemplary process flow of a method to train a predictive model to classify nucleic acid samples as tumor origin or non-tumor origin.

FIG. 17 is a chart indicating colorectal cancer sensitivity according to stage of diagnosis.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide is disclosed and discussed and a number of modifications that can be made to a number of molecules including the amino acids are discussed, each and every combination and permutation of the peptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

In order for the present disclosure to be more readily understood, certain terms are first defined below. Additional definitions for the following terms and other terms may be set forth through the specification. If a definition of a term set forth below is inconsistent with a definition in a patent application or issued patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons of ordinary skill in the art upon reading this disclosure and so forth. It will also be appreciated that there is an implied “about” prior to the temperatures, concentrations, times, number of bases or base pairs, coverage, etc. discussed in the present disclosure, such that slight and insubstantial equivalents are within the scope of the present disclosure. In this application, the use of the singular includes the plural unless specifically stated otherwise. Also, the use of “comprise”, “comprises”, “comprising”, “contain”, “contains”, “containing”, “include”, “includes”, and “including” are not intended to be limiting.

It is also to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this disclosure pertains. In describing and claiming the methods, computer readable media, and systems, the following terminology, and grammatical variants thereof, will be used in accordance with the definitions set forth below.

About: As used herein, “about” or “approximately” as applied to one or more values or elements of interest, refers to a value or element that is similar to a stated reference value or element. In certain embodiments, the term “about” or “approximately” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Adapter: As used herein, “adapter” refers to short nucleic acids (e.g., less than about 500, less than about 100 or less than about 50 nucleotides in length) that are typically at least partially double-stranded and used to link to either or both ends of a given sample nucleic acid molecule. Adapters can include nucleic acid primer binding sites to permit amplification of a nucleic acid molecule flanked by adapters at both ends, and/or a sequencing primer binding site, including primer binding sites for sequencing applications, such as various next generation sequencing (NGS) applications. Adapters can also include binding sites for capture probes, such as an oligonucleotide attached to a flow cell support or the like. Adapters can also include a nucleic acid tag as described herein. Nucleic acid tags are typically positioned relative to amplification primer and sequencing primer binding sites, such that a nucleic acid tag is included in amplicons and sequencing reads of a given nucleic acid molecule. Adapters of the same or different sequence can be linked to the respective ends of a nucleic acid molecule. In certain embodiments, the same adapter is linked to the respective ends of the nucleic acid molecule except that the nucleic acid tag differs in its sequence. In some embodiments, the adapter is a Y-shaped adapter in which one end is blunt ended or tailed as described herein, for joining to a nucleic acid molecule, which is also blunt ended or tailed with one or more complementary nucleotides. In still other exemplary embodiments, an adapter is a bell-shaped adapter that includes a blunt or tailed end for joining to a nucleic acid molecule to be analyzed. Other exemplary adapters include T-tailed and C-tailed adapters.

Administer: As used herein, “administer” or “administering” a therapeutic agent (e.g., an immunological therapeutic agent, a DNA damage response (DDR) inhibitor (e.g., a poly (ADP-ribose) polymerase (PARP) inhibitor (PARPi)), etc.) to a subject means to give, apply or bring the composition into contact with the subject. Administration can be accomplished by any of a number of routes, including, for example, topical, oral, subcutaneous, intramuscular, intraperitoneal, intravenous, intrathecal and intradermal.

Align: As used herein, “align,” alignment,” and “aligning” in the context of nucleic acids refers to arranging sequences of DNA or RNA to identify regions of similarity. Similarity may be related to functional, structural, and/or evolutionary relationships between the sequences. Alignment of DNA sequences involves alignment of genomic DNA of one sequence to genomic DNA of at least one other sequence. Such alignment may exclude non-genomic DNA, such as a molecular barcode, padding bases, and the like. For example, genomic DNA of a sequence read may be aligned to genomic DNA of a reference DNA sequence, excluding any molecular tag that may be attached to the sequence read.

Allele: As used herein, “allele” or “allelic variant” refers to a specific genetic variant at defined genomic location or locus. An allelic variant is usually presented at a frequency of 50% (0.5) or 100%, depending on whether the allele is heterozygous or homozygous. For example, germline variants are inherited and usually have a frequency of 0.5 or 1. Somatic variants; however, are acquired variants and usually have a frequency of <0.5. Major and minor alleles of a genetic locus refer to nucleic acids harboring the locus in which the locus is occupied by a nucleotide of a reference sequence, and a variant nucleotide different than the reference sequence respectively. Measurements at a locus can take the form of allelic fractions (AFs), which measure the frequency with which an allele is observed in a sample.

Amplify: As used herein, “amplify” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes.

Barcode: As used herein, “barcode” in the context of nucleic acids refers to a nucleic acid molecule having a sequence that can serve as an identifier of the molecule (molecular barcode) or an identifier of the sample (sample barcode or sample index). For example, individual “barcode” sequences are typically added to DNA fragments during next-generation sequencing (NGS) library preparation so that each read can be identified and sorted before the final data analysis.

Breakpoint: As used herein, “breakpoint” in the context of a nucleic acid fusion molecule or a corresponding sequencing read refers to a terminal nucleotide position at a junction between fused sub-sequences of the nucleic acid fusion or represented in the corresponding sequencing read. For example, a given split sequence read may include a first sub-sequence that is contiguous with, and 5′ to, a second sub-sequence in that split sequence read in which the first sub-sequence maps to a first locus in a reference sequence that is non-contiguous with a second locus in that reference sequence to which the second sub-sequence maps. In this example, the first sub-sequence of the split sequence read includes a breakpoint at its 3′ terminal nucleotide, while the second subsequence of the split sequence read includes a breakpoint at its 5′ terminal nucleotide. In certain applications, breakpoints such as these are referred to as a “breakpoint pair.”

Cancer Type: As used herein, “cancer,” “cancer type” or “tumor type” refers to a type or subtype of cancer defined, e.g., by histopathology. Cancer type can be defined by any conventional criterion, such as on the basis of occurrence in a given tissue (e.g., blood cancers, central nervous system (CNS), brain cancers, lung cancers (small cell and non-small cell), skin cancers, nose cancers, throat cancers, liver cancers, bone cancers, lymphomas, pancreatic cancers, bowel cancers, rectal cancers, thyroid cancers, bladder cancers, kidney cancers, mouth cancers, stomach cancers, breast cancers, prostate cancers, ovarian cancers, lung cancers, intestinal cancers, soft tissue cancers, neuroendocrine cancers, gastroesophageal cancers, head and neck cancers, gynecological cancers, colorectal cancers, urothelial cancers, solid state cancers, heterogeneous cancers, homogenous cancers), unknown primary origin and the like, and/or of the same cell lineage (e.g., carcinoma, sarcoma, lymphoma, cholangiocarcinoma, leukemia, mesothelioma, melanoma, or glioblastoma) and/or cancers exhibiting cancer markers, such as Her2, CA15-3, CA19-9, CA-125, CEA, AFP, PSA, HCG, KRAS, BRAF, NRAS, hormone receptor and NMP-22. Cancers can also be classified by stage (e.g., stage 1, 2, 3, or 4) and whether of primary or secondary origin.

Cell-Free Nucleic Acid: As used herein, “cell-free nucleic acid” refers to nucleic acids not contained within or otherwise bound to a cell. In some embodiments, “cell-free nucleic acid” refers to nucleic acids which are not contained within or otherwise bound to a cell at the point of isolation from the subject. Cell-free nucleic acids can include, for example, all non-encapsulated nucleic acids sourced from a bodily fluid (e.g., blood, plasma, serum, urine, cerebrospinal fluid (CSF), etc.) from a subject. Cell-free nucleic acids include DNA (cfDNA), RNA (cfRNA), and hybrids thereof, including genomic DNA, mitochondrial DNA, circulating DNA, siRNA, mtRNA, circulating RNA (cRNA), tRNA, rRNA, small nucleolar RNA (snoRNA), Piwi-interacting RNA (piRNA), long non-coding RNA (long ncRNA), and/or fragments of any of these. Cell-free nucleic acids can be double-stranded, single-stranded, or a hybrid thereof. A cell-free nucleic acid can be released into bodily fluid through secretion or cell death processes, e.g., cellular necrosis, apoptosis, or the like. Some cell-free nucleic acids are released into bodily fluid from cancer cells, e.g., circulating tumor DNA (ctDNA). Others are released from healthy cells. CtDNA can be non-encapsulated tumor-derived fragmented DNA. Another example of cell-free nucleic acids is fetal DNA circulating freely in the maternal blood stream, also called cell-free fetal DNA (cffDNA). A cell-free nucleic acid can have one or more epigenetic modifications, for example, a cell-free nucleic acid can be acetylated, 5-methylated, ubiquitylated, phosphorylated, sumoylated, ribosylated, and/or citrullinated.

Cellular Origin: As used herein, “cellular origin” in the context of cell-free nucleic acids means the cell type from which a given cell-free nucleic acid molecule derives or otherwise originates (e.g., via a apoptotic process, a necrotic process, or the like). In certain embodiments, for example, a given cell-free nucleic acid molecule may originate from a tumor cell (e.g., a cancerous pulmonary cell, etc.) or a non-tumor or normal cell (e.g., a non-cancerous pulmonary cell, etc.).

Classification Region As used herein, “classification region” refers to a genomic region that may show sequence-independent changes in neoplastic cells (e.g., tumor cells and cancer cells) or that may show sequence-independent changes in cfDNA from subjects having cancer relative to cfDNA from subjects in which cancer is not present. Examples of sequence-independent changes include, but are not limited to, changes in methylation rate (increases or decreases), nucleosome distribution, CTCF binding, transcription start sites, and regulatory protein binding regions. In one or more examples, sequence-independent changes in a classification region can indicate the presence of a single form of cancer in a subject. In one or more additional examples, sequence-independent changes in a classification region can correspond to the presence of multiple forms in a subject. The classification region can be enriched by one or more probes. In addition, the classification region can be defined by a pair of primer binding sites. Further, the classification region can be defined by a predetermined beginning genomic locus and a predetermined ending genomic locus. The classification region can include from about 25 nucleotides to about 250 nucleotides, from about 50 nucleotides to about 200 nucleotides, or from about 75 nucleotides to about 150 nucleotides. For instance, classification region can be a differentially methylated region. “Differentially methylated region” or “DMR” refers to a region of DNA having a detectably different degree of methylation in at least one cell or tissue type relative to the degree of methylation in the same region of DNA from at least one other cell or tissue type; or having a detectably different degree of methylation in at least one cell or tissue type obtained from a subject having a disease or disorder relative to the degree of methylation in the same region of DNA in the same cell or tissue type obtained from a healthy subject. In some embodiments, a differentially methylated region has a detectably higher degree of methylation (e.g., a hypermethylated region/hypermethylated target region) in at least one cell or tissue type relative to the degree of methylation in the same region of DNA from at least one other cell or tissue type that contribute to cfDNA in healthy individuals, or from the same cell or tissue type from a healthy subject. In some embodiments, a differentially methylated region has a detectably lower degree of methylation (e.g., a hypomethylated region/hypomethylated target region) in at least one cell or tissue type relative to the degree of methylation in the same region of DNA from at least one other cell or tissue type, such as other immune cell types and/or cell types that contribute to cfDNA in healthy individuals, or from the same cell or tissue type from a healthy subject. In some embodiments, the classification regions comprise hypermethylated target regions and/or hypomethylated target regions.

Classifier: As used herein, “classifier” generally refers to algorithm computer code that receives, as input, test data and produces, as output, a classification of the input data as belonging to one or another class (e.g., having a DNA damage repair deficiency (DDRD) or not having DDRD, tumor DNA or non-tumor DNA).

Contiguous Sequence: As used herein, “contiguous sequence” or “contig” refers to a set of overlapping nucleic acid segments that together represent a consensus region of a nucleic acid.

Copy Number Variant: As used herein, “copy number variant,” “CNV,” or “copy number variation” refers to a phenomenon in which sections of the genome are repeated and the number of repeats in the genome varies between individuals in the population under consideration.

Coverage: As used herein, the terms “coverage”, “total molecule count” or “total allele count” are used interchangeably. They refer to the total number of DNA molecules at a particular genomic position in a given sample.

Deoxyribonucleic Acid or Ribonucleic Acid: As used herein, “deoxyribonucleic acid” or “DNA” refers a natural or modified nucleotide which has a hydrogen group at the 2′-position of the sugar moiety. DNA typically includes a chain of nucleotides comprising deoxyribonucleosides that comprise one of four types of nucleobases, namely, adenine (A), thymine (T), cytosine (C), and guanine (G). As used herein, “ribonucleic acid” or “RNA” refers to a natural or modified nucleotide which has a hydroxyl group at the 2′-position of the sugar moiety. RNA typically includes a chain of nucleotides comprising ribonucleosides that comprise one of four types of nucleobases, namely, A, uracil (U), G, and C. As used herein, the term “nucleotide” refers to a natural nucleotide or a modified nucleotide. Certain pairs of nucleotides specifically bind to one another in a complementary fashion (called complementary base pairing). In DNA, adenine (A) pairs with thymine (T) and cytosine (C) pairs with guanine (G). In RNA, adenine (A) pairs with uracil (U) and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “sequence information,” “nucleic acid sequence,” “nucleotide sequence”, “genomic sequence,” “genetic sequence,” or “fragment sequence,” or “nucleic acid sequencing read” denotes any information or data that is indicative of the order and identity of the nucleotide bases (e.g., adenine, guanine, cytosine, and thymine or uracil) in a molecule (e.g., a whole genome, whole transcriptome, exome, oligonucleotide, polynucleotide, or fragment) of a nucleic acid such as DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, and electronic signature-based systems.

Detect: As used herein, “detect,” “detecting,” or “detection” refers to an act of determining the existence or presence of one or more target nucleic acids (e.g., nucleic acids having targeted mutations or other markers) in a sample.

Enriched Sample: As used herein, “enriched sample” refers to a sample that has been enriched for specific regions of interest. The sample can be enriched by amplifying regions of interest or by using single-stranded DNA/RNA probes or double stranded DNA probes that can hybridize to nucleic acid molecules of interest (e.g., SureSelect® probes, Agilent Technologies). In some embodiments, an enriched sample refers to a subset or portion of the processed sample that is enriched, where the subset or portion of the processed sample being enriched contains nucleic acid molecules from a sample of cell-free polynucleotides or polynucleotides.

Epigenetic Information: As used herein, “epigenetic information” in the context of a DNA polymer means one or more epigenetic patterns or signatures exhibited in that polymer.

Epigenetic Locus: As used herein, “epigenetic locus” or “epigenetic site” means a fixed position on a chromosome that exhibits different states or statuses that do not involve changes or alterations in nucleotide sequence. For the avoidance of doubt, a given epigenetic locus can coincide with a given nucleotide position or genomic region that also exhibits genetic or sequence variation (e.g., mutations). For example, a given epigenetic locus may or may not be acetylated, methylated (e.g., modified with 5-methylcytosine (5mC), modified with 5-hydroxymethylcytosine (5hmC), and/or the like), ubiquitylated, phosphorylated, sumoylated, ribosylated, citrullinated, have a histone post-translational modification or other histone variation, and/or the like.

Epigenetic Signature: As used herein, “epigenetic signature” means an epigenetic state or status exhibited by one or more epigenetic loci in a given DNA molecule. For example, DNA molecules or cfDNA fragments that comprise a given genomic region or locus (e.g., a CTCF binding region, etc.) may also exhibit epigenetic patterns in which some of those DNA molecules include a certain number of epigenetic loci that are methylated, whereas in other instances corresponding epigenetic loci in other DNA molecules or cfDNA fragments that comprise the same genomic region are unmethylated. “Methylation signature” means an epigenetic signature associated with a methylation state or status exhibited by one or more epigenetic loci in a given DNA molecule.

Fusion Event: As used herein, “fusion event” or “fusion” refers to a fusion between at least two separate genes at a particular location. Example causes of a fusion event include a translocation, interstitial deletion, or chromosomal inversion event.

Gene: As used herein, “gene” refers to any segment of DNA associated with a biological function. Thus, genes include coding sequences and optionally, the regulatory sequences required for their expression. Genes also optionally include non-expressed DNA segments that, for example, form recognition sequences for other proteins.

Genomic Region: As used herein, “genomic region” means a fixed position on, or section of, a chromosome, such as the position of a gene or a genomic marker. Exemplary genomic markers include transcriptional factor binding regions (e.g., CTCF binding regions, etc.), distal regulatory elements (DREs), repetitive elements (e.g., microsatellites, etc.), intron-exon or exon-intron junctions, transcriptional start sites (TSSs), and the like.

Germline Mutation: As used herein, “germline mutation” means a mutation in a germ cell and accordingly, that can be passed on to progeny.

Indel: As used herein, “indel” refers to mutation that involves the insertion or deletion of nucleotide positions in the genome of a subject.

Machine Learning Algorithm: As used herein, “machine learning algorithm” generally refers to an algorithm, executed by computer, that automates analytical model building, e.g., for clustering, classification or pattern recognition. Machine learning algorithms may be supervised or unsupervised. Learning algorithms include, for example, artificial neural networks (e.g., back propagation networks), discriminant analyses (e.g., Bayesian classifier or Fischer analysis), support vector machines, decision trees (e.g., recursive partitioning processes such as CART-classification and regression trees, or random forests), linear classifiers (e.g., multiple linear regression (MLR), partial least squares (PLS) regression, and principal components regression), hierarchical clustering, and cluster analysis. A dataset on which a machine learning algorithm learns can be referred to as “training data.”

Match: As used herein, “match” means that at least a first value or element is at least approximately equal to at least a second value or element. In certain embodiments, for example, the cellular origin of at least the subset of the DNA molecules from a cfDNA sample is determined when there is at least a substantial or approximate match between a test sample distribution of cfDNA fragment properties and a reference sample distribution of cfDNA fragment properties.

Minor Allele Frequency: As used herein, “minor allele frequency” refers to the frequency at which minor alleles (e.g., not the most common allele) occurs in a given population of nucleic acids, such as a sample obtained from a subject. Genetic variants at a low minor allele frequency typically have a relatively low frequency of presence in a sample.

Mutant Allele Fraction: As used herein, “mutant allele fraction,” or “MAF” refers to the fraction of nucleic acid molecules harboring an allelic alteration or mutation with respect to a reference at a given genomic position in a given sample. MAF is generally expressed as a fraction or percentage. For example, MAF is typically less than about 0.5, 0.1, 0.05, or 0.01 (i.e., less than about 50%, 10%, 5%, or 1%) of all somatic variants or alleles present at a given locus.

Maximum Mutant Allele Fraction: As used herein, “maximum mutant allele fraction,” “maximum MAE,” or “MAX MAE” refers to the maximum or largest MAF of all somatic variants present or observed in a given sample.

Mutation: As used herein, “mutation,” “nucleic acid variant,” “variant,” or “genetic aberration” refers to a variation from a known reference sequence and includes mutations such as, for example, single nucleotide variants (SNVs), copy number variants or variations (CNVs)/aberrations, insertions or deletions (indels), truncation, gene fusions, transversions, translocations, frame shifts, duplications, repeat expansions, and epigenetic variants. A mutation can be a germline or somatic mutation. In some embodiments, a reference sequence for purposes of comparison is a wildtype genomic sequence of the species of the subject providing a test sample, typically the human genome. In certain cases, a mutation or variant is a “tumor-related genetic variant” that causes or at least contributes to oncogenesis.

Negative Control Region: As used herein, “negative control region”, refers to a genomic region that is expected to be unmethylated or hypomethylated in essentially all samples, regardless of whether the DNA is derived from a cancer cell or a normal cell.

Next Generation Sequencing: As used herein, “next generation sequencing” or “NGS” refers to sequencing technologies having increased throughput as compared to traditional Sanger- and capillary electrophoresis-based approaches, for example, with the ability to generate hundreds of thousands of relatively small sequence reads at a time. Some examples of next generation sequencing techniques include, but are not limited to, sequencing by synthesis, sequencing by ligation, and sequencing by hybridization.

Nucleic Acid Tag: As used herein, “nucleic acid tag” refers to a short nucleic acid (e.g., less than about 500, about 100, about 50 or about 10 nucleotides in length), used to label nucleic acid molecules to distinguish nucleic acids from different samples (e.g., representing a sample index), or different nucleic acid molecules in the same sample (e.g., representing a molecular tag), of different types, or which have undergone different processing. Nucleic acid tags can be single stranded, double stranded or at least partially double stranded. Nucleic acid tags optionally have the same length or varied lengths. Nucleic acid tags can also include double-stranded molecules having one or more blunt-ends, include 5′ or 3′ single-stranded regions (e.g., an overhang), and/or include one or more other single-stranded regions at other locations within a given molecule. Nucleic acid tags can be attached to one end or both ends of the other nucleic acids (e.g., sample nucleic acids to be amplified and/or sequenced). Nucleic acid tags can be decoded to reveal information such as the sample of origin, form or processing of a given nucleic acid. Nucleic acid tags can also be used to enable pooling and/or parallel processing of multiple samples comprising nucleic acids bearing different nucleic acid tags and/or sample indexes in which the nucleic acids are subsequently being deconvoluted by reading the nucleic acid tags. Nucleic acid tags can also be referred to as molecular identifiers or tags, sample identifiers, index tags, and/or barcodes. Additionally or alternatively, nucleic acid tags can be used to distinguish different molecules in the same sample. This includes, for example, uniquely tagging different nucleic acid molecules in a given sample, or non-uniquely tagging such molecules. In the case of non-unique tagging applications, tags with a limited number of different sequences may be used to tag nucleic acid molecules such that different molecules can be distinguished based on, for example, start and/or stop positions where they map to a selected reference genome in combination with at least one nucleic acid tag. Typically, a sufficient number of different nucleic acid tags are used such that there is a low probability (e.g., less than about a 10%, less than about a 5%, less than about a 1%, or less than about a 0.1% chance) that any two molecules will have the same start/stop positions and also have the same nucleic acid tag. Some nucleic acid tags include multiple molecular identifiers to label samples, forms of nucleic acid molecules within a sample, and nucleic acid molecules within a form having the same start and stop positions. Such nucleic acid tags can be referenced using the exemplary form “Ali” in which the uppercase letter indicates a sample type, the Arabic numeral indicates a form of molecule within a sample, and the lowercase Roman numeral indicates a molecule within a form.

Polynucleotide: As used herein, “polynucleotide”, “nucleic acid”, “nucleic acid molecule”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Oligonucleotides often range in size from a few monomeric units, e.g. 3-4, to hundreds of monomeric units. Whenever a polynucleotide is represented by a sequence of letters, such as “ATGCCTG,” it will be understood that the nucleotides are in 5′→3′ order from left to right and that in the case of DNA, “A” denotes deoxyadenosine, “C” denotes deoxycytidine, “G” denotes deoxyguanosine, and “T” denotes deoxythymidine, unless otherwise noted. The letters A, C, G, and T may be used to refer to the bases themselves, to nucleosides, or to nucleotides comprising the bases, as is standard in the art.

Positive Control Region. As used herein, As used herein, “positive control region”, refers to a genomic region that is expected to be methylated or hypermethylated in essentially all samples, regardless of whether the DNA is derived from a cancer cell or a normal cell.

Prevalence: As used herein, “prevalence” in the context of nucleic acid variants refers to the degree, pervasiveness, or frequency with which a given nucleic acid variant is or was observed in a given sample (e.g., a given bodily fluid sample, a given non-bodily fluid sample, etc.) or other population (e.g., a given population of bodily fluid samples, a given population of non-bodily fluid samples, etc.).

Reference Sample: As used herein, “reference sample” or “reference cfDNA sample” refers a sample of known composition and/or having or known to have or lack specific properties (e.g., known nucleic acid variant(s), known cellular origin, known tumor fraction, known coverage, and/or the like) that is analyzed along with or compared to test samples in order to evaluate the accuracy of an analytical procedure. A reference sample dataset typically includes from at least about 25 to at least about 30,000 or more reference samples. In some embodiments, the reference sample dataset includes about 50, 75, 100, 150, 200, 300, 400, 500, 600, 700, 800, 900, 1,000, 2,500, 5,000, 7,500, 10,000, 15,000, 20,000, 25,000, 50,000, 100,000, 1,000,000, or more reference samples.

Reference Sequence: As used herein, “reference sequence” or “reference genome” refers to a known sequence used for purposes of comparison with experimentally determined sequences. For example, a known sequence can be an entire genome, a chromosome, or any segment thereof. A reference sequence typically includes at least about 20, at least about 50, at least about 100, at least about 200, at least about 250, at least about 300, at least about 350, at least about 400, at least about 450, at least about 500, at least about 1000, at least about 10,000, at least about 100,000, at least about 1,000,000, at least about 10,000,000, at least about 100,000,000, at least about 1,000,000,000, or more nucleotides. A reference sequence can align with a single contiguous sequence of a genome or chromosome or can include non-contiguous segments that align with different regions of a genome or chromosome. Exemplary reference sequences, include, for example, human genomes, such as, hG19 and hG38.

Sample: As used herein, “sample” means any biological sample capable of being analyzed by the methods and/or systems disclosed herein. In certain aspects of the present disclosure, samples are bodily fluid samples, for example, whole blood or fractions thereof, lymphatic fluid, urine, and/or cerebrospinal fluid, among other bodily fluid types from which cell-free (circulating, not contained within or otherwise bound to a cell) nucleic acids are sourced. In certain implementations, bodily fluid samples are plasma samples, which are the fluid portions of whole blood exclusive of cells, such as red and white blood cells. In some implementations, bodily fluid samples are serum samples, that is, plasma lacking fibrinogen. In some aspects of the present disclosure, samples are “non-bodily fluid samples” or “non-plasma samples,” that is, biological samples other than “bodily fluid samples” such as, as cellular and/or tissue samples, from which nucleic acids other than cell-free nucleic acids are sourced.

Sensitivity: As used herein, “sensitivity” in the context of a given assay or method refers to the ability of the assay or method to detect and distinguish between targeted (e.g., nucleic acid variants) and non-targeted analytes.

Sequence fragment: As used herein, “sequence fragment” refers to a piece of a nucleic acid molecule that can vary in length and can carry the sequence information (or sequence data) of the nucleic acid molecule. The sequence information can be derived from sequencing reads obtained from sequencing the sequence fragments.

Sequence read: As used herein, “sequence read” refers to the sequence of base pairs corresponding to all or a part of a sequence fragment.

Sequencing: As used herein, “sequencing” refers to any of a number of technologies used to determine the sequence (e.g., the identity and order of monomer units) of a biomolecule, e.g., a nucleic acid such as DNA or RNA. Exemplary sequencing methods include, but are not limited to, targeted sequencing, single molecule real-time sequencing, exon or exome sequencing, intron sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, and a combination thereof. In some embodiments, sequencing can be performer by a gene analyzer such as, for example, gene analyzers commercially available from Illumina, Inc., Pacific Biosciences, Inc., or Applied Biosystems/Thermo Fisher Scientific, among many others.

Sequence Information: As used herein, “sequence information” in the context of a nucleic acid polymer means the order and/or identity of monomer units (e.g., nucleotides, etc.) in that polymer.

Sequence Motif: As used herein, “sequence motif” may refer to a short, recurring pattern of bases in DNA fragments (e.g., cell-free DNA fragments). A sequence motif can occur at an end of a fragment, and thus be part of or include an ending sequence. An “end motif” can refer to a sequence motif for an ending sequence that preferentially occurs at ends of DNA fragments, potentially for a particular type of tissue. An end motif may also occur just before or just after ends of a fragment, thereby still corresponding to an ending sequence. A nuclease can have a specific cutting preference for a particular end motif, as well as a second most preferred cutting preference for a second end motif.

Single Nucleotide Variant: As used herein, “single nucleotide variant” or “SNV” means a mutation or variation in a single nucleotide that occurs at a specific position in the genome.

Somatic Mutation: As used herein, “somatic mutation” means a mutation in a given genome that occurs after conception. Somatic mutations can occur in any cell of the body except germ cells and accordingly, are not passed on to progeny.

Specificity: As used herein, “specificity” in the context of a diagnostic analysis or assay refers to the extent to which the analysis or assay detects an intended target analyte to the exclusion of other components of a given sample.

Status: As used herein, “status” in the context of subjects refers to one or more states of a given subject, such as whether or not the subject has cancer.

Subject: As used herein, “subject” or “test subject” refers to an animal, such as a mammalian species (e.g., human) or avian (e.g., bird) species, or other organism, such as a plant. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual that is in need of therapy or suspected of needing therapy. The terms “individual” or “patient” are intended to be interchangeable with “subject.” In some embodiments, the subject is a human who has, or is suspected of having cancer. For example, a subject can be an individual who has been diagnosed with having a cancer, is going to receive a cancer therapy, and/or has received at least one cancer therapy. The subject can be in remission of a cancer. As another example, the subject can be an individual who is diagnosed of having an autoimmune disease. As another example, the subject can be a female individual who is pregnant or who is planning on getting pregnant, who may have been diagnosed with or suspected of having a disease, e.g., a cancer, an auto-immune disease. A “reference subject” refers to a subject known to have or lack specific properties (e.g., known cancer or disease status, known nucleic acid variant(s), known cellular origin, known tumor fraction, known coverage, and/or the like).

Threshold: As used herein, “threshold” refers to a separately determined value used to characterize or classify experimentally determined values. In certain embodiments, for example, “threshold value” refers to a selected value to which a quantitative value is compared in order to determine that a given target nucleic acid variant is absent at a given genetic locus.

Tumor Fraction: As used herein, “tumor fraction” refers to the estimate of the fraction of nucleic acid molecules derived from tumor in a given sample. For example, the tumor fraction of a sample can be a measure derived from the maximum mutant allele frequency (MAX MAF) of the sample or coverage of the sample, or length, epigenetic state, or other properties of the cfDNA fragments in the sample or any other selected feature of the sample. The term “MAX MAF” refers to the maximum or largest MAF of all somatic variants present in a given sample. In some embodiments, the tumor fraction of a sample is equal to the MAX MAF of the sample.

Value: As used herein, “value” or “score” generally refers to an entry in a dataset can be anything that characterizes the feature to which the value refers. This includes, without limitation, numbers, words or phrases, symbols (e.g., + or −) or degrees.

### B. Introduction

Provided herein are methods and systems for differentiating or classifying tumor and non-tumor origin nucleic acid variants in a nucleic acid sample obtained from a test subject. In some aspects, the methods and systems couple genomic alteration data (e.g., somatic genomic data) with epigenetic data (e.g., methylation data, fragmentomic data). In some aspects, the nucleic acid sample can be, but is not limited to, cell-free nucleic acid (cfNA), genomic DNA, or RNA.

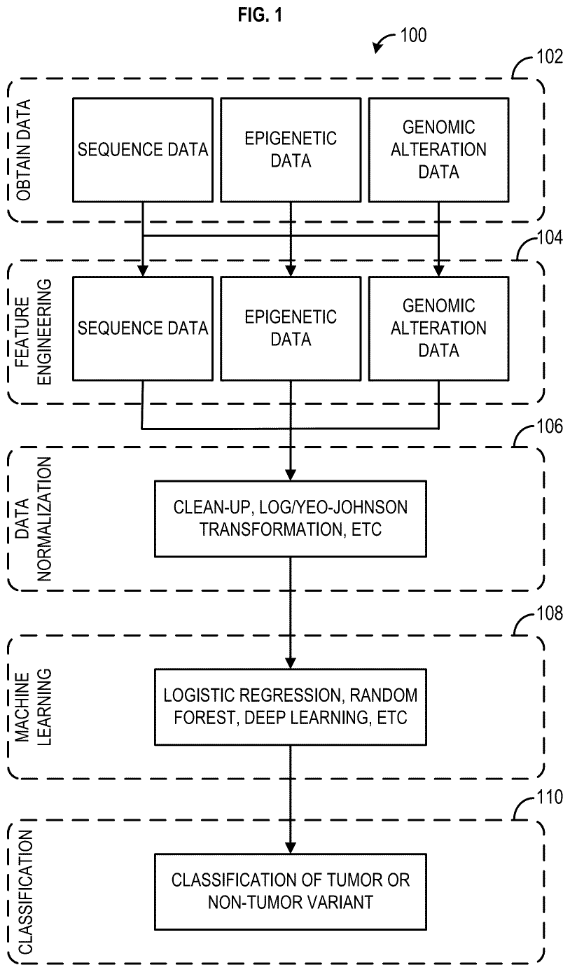

FIG. 1 is a flow chart that schematically depicts an example artificial intelligence (e.g., machine learning) technique for generating a classifier configured for differentiating or classifying tumor and non-tumor origin nucleic acid variants in a cell-free nucleic acid (cfDNA) sample obtained from a test subject. As shown, a method 100, at step 102, may comprise obtaining data, for example, in the form of cancer (e.g., tumor) origin and non-cancer origin sequence data from cell-free nucleic acid (cfDNA) samples of a plurality of subjects. The method 100 may also comprise obtaining epigenetic data and/or genomic alteration data associated with, or otherwise derived from, the sequence data. Epigenetic data and genomic alteration data can all be determined from genomic regions within the cfDNA samples. Epigenetic data may include, for example, information regarding DNA methylation, histone states or modifications, inflammation-mediated cytosine damage products, protein binding, fragmentomic data (information regarding fragment size, nucleotide motifs at fragment ends, single-stranded jagged ends, and/or genomic locations of fragmentation endpoints), or other molecular states reflected in the nucleic acid fragment analyzed that are not ascertained solely from the nucleotide base sequence, e.g., the methylation status of given base or set of bases. In an embodiment, epigenetic data and genomic alteration data of nucleic acid sequences known to be tumor derived may be labeled as tumor derived and epigenetic data and genomic alteration data of nucleic acid sequences known to be non-tumor derived may be labeled as non-tumor derived. Moreover, further labels may be assigned, for example, cancer type, tissue type, and the like.

In some embodiments, the methods, and related systems and computer readable media implementations, disclosed herein include identifying sets of DNA molecules or cfDNA fragments from cfDNA samples in which each member cfDNA fragment of a given set comprises a genomic region in common with one another. Essentially any genomic region can be used as long as cfDNA fragments comprising a given genomic region exhibit different properties (e.g., cfDNA fragment lengths, offsets of cfDNA fragment midpoints relative to midpoints of genomic regions comprised by the cfDNA fragment, epigenetic states, and/or the like) between at least two cell or tissue types. In certain embodiments, for example, genomic regions include regions of differential chromatin organization between at least two cell or tissue types. More specifically, fragmentation patterns of DNA molecules in cfDNA samples carries information about the chromatin organization of the cells or tissues from which the cfDNA fragments originate. In particular, DNA fragments released to the bloodstream is often fragmented or cleaved around nucleosomes and/or other DNA bound proteins in the cells or tissues of origin. Further, nucleosome positioning and the location of DNA binding proteins is highly tissue specific and thus is used herein to amplify signal coming from the cells or tissues from which the cfDNA fragments originate (e.g., tumor cells as well as cells in the tumor microenvironment and cells involved in the immune response). In certain embodiments, genomic regions comprise transcriptional factor binding regions, distal regulatory elements (DREs), repetitive elements, intron-exon or exon-intron junctions (splice junctions), transcriptional start sites (TSSs), and/or the like.

In some embodiments, the methods, and related system and computer readable media implementations, disclosed herein include determining the cellular origin of DNA molecules from cfDNA samples using properties of those DNA molecules, such as epigenetic patterns exhibited by those molecules or fragments. As described herein, epigenetic changes in genomic sections are often accompanied by changes in chromatin organization and nucleosome positioning within those genomic sections. Accordingly, the methods and related aspects of this disclosure combine these sources of signal to increase the ability to detect the presence of targeted cells (e.g., diseased cells, such as tumor cells or the like), fetal cells, transplant donor cells, and the like) in cfDNA samples.

Any epigenetic site or locus that exhibits differential modifications (e.g., a post-replication modification or the like) between at least two cell or tissue types can be used to perform the methods and related aspects of the present disclosure. Examples of such sites, include methylation sites, acetylation sites, ubiquitylation sites, phosphorylation sites, sumoylation sites, ribosylation sites, citrullination sites, histone post-translational modification sites, histone variant sites, and/or the like. Examples of post-replication modifications, include 5-methyl-cytosine, 5-hydroxymethyl-cytosine, 5-carboxyl-cytosine, and 5-formyl-cytosine, among many others. Additional details regarding epigenetic sites or loci are described in, for example, Jin et al., “DNA Methylation: Superior or Subordinate in the Epigenetic Hierarchy?,” Genes Cancer, 2(6):607-617 (2011), Javaid et al., “Acetylation-and Methylation-Related Epigenetic Proteins in the Context of Their Target,” Genes (Basel), 8(8):196 (2017), Cao et al., “Histone Ubiquitination and Deubiquitination in Transcription, DNA Damage Response, and Cancer,” Front Oncol, 2:26 (2012), Rossetto et al., “Histone phosphorylation: A chromatin modification involved in diverse nuclear event,” Epigenetics, 7(10):1098-1108 (2012), Vranych et al., “SUMOylation and deimination of proteins: two epigenetic modifications involved in Giardia encystation,” Biochim Biophys Acta, 1843(9):1805-17 (2014), Sadakierska-Chudy et al., “A Comprehensive View of the Epigenetic Landscape. Part II: Histone Post-translational Modification, Nucleosome Level, and Chromatin Regulation by ncRNAs,” Neurotox Res, 27:172-197 (2015), Fuhrmann et al., “Protein Arginine Methylation and Citrullination in Epigenetic Regulation,” ACS Chem Biol, 11(3):654-668 (2016), Fan et al., “Metabolic regulation of histone post-translational modifications,” ACS Chem Biol, 10(1):95-108 (2015), and Henikoff et al., “Histone Variants and Epigenetics,” Cold Spring Harb Perspect Biol, 7(1) (2015), which are each incorporated by reference.

Epigenetic information can be obtained from cfDNA fragments using any technique known to those of ordinary skill in the art. In some embodiments, for example, DNA molecules from a given cfDNA sample are physically fractionated (e.g., fractionating with methyl-binding domain protein (“MBD”)-beads to stratify the cfDNA fragments into various degrees of methylation or the like) to generate partitions. In these embodiments, differential molecular tags and NGS-enabling adapters are applied to each of the two or more partitions to generate molecular tagged partitions. In addition, these embodiments also include assaying the molecular tagged partitions on an NGS instrument to generate sequence data for deconvoluting the sample into molecules that were differentially partitioned to generate the epigenetic information. In some embodiments, bisulfite sequencing techniques are also used to generate epigenetic information from cfDNA samples. Additional details regarding the analysis of epigenetic modifications that are optionally adapted for use in performing the methods disclosed herein are described in, for example, WO 2018/119452, filed Dec. 22, 2017, which is incorporated by reference.

In some embodiments, the methods, and related system and computer readable media implementations, disclosed herein include determining the cellular origin of DNA molecules from nucleic acid samples, for example, cfDNA samples, using properties of the sequences (e.g., sequence fragments/reads) that are ascertained via a sequencing process, using another form of epigenetic data, such as fragmentomic patterns exhibited by those molecules or fragments. Human plasma DNA comprises a mixture of DNA fragments of different sizes, accordingly size of sequence fragments may form part of a fragmentomic signature. The modal size is approximately 166 base pairs (bp) and may be related to nucleosomal structure. Cell-free tumor-derived DNA in plasma of cancer patients has shorter modal sizes of approximately 143 bp. The size profiles of ctDNA may have a shorter median length and may be more variable in subjects with cancer than in subjects without cancer. Additionally, a pattern of cell-free DNA size peaks may be used to distinguish between tumor and non-tumor sequence fragments.

Cell-free tumor-derived DNA may exhibit different ends when compared to cell-free non-tumor-derived DNA, accordingly end motifs may form part of a fragmentomic signature. The ending sequences reveal overrepresentation of certain motifs that could be characterized by a range of nucleotides, such as 2-nucleotide oligomer (2-mer) or 4-mer motifs. Many human cancers exhibit down-regulation of the expression of DNASE1L3 which results in a reduced plasma DNA with DNASE1L3-associated end motifs. Plasma DNA end motifs demonstrate an advantage in that their maximal diagnostic power may be achieved with a relatively small number of DNA molecules analyzed. For example, on the basis of computer simulation, at a tumor DNA fraction of 10%, it would only require 50,000 plasma DNA molecules (DNA content of each cell is fragmented into about 20 million cell-free DNA molecules) to differentiate patients with and without hepatocellular carcinoma, whereas at least 7.5 million DNA molecules would be needed to detect a 1-megabase (Mb) copy number aberration. The detection of tumor-derived single-nucleotide variants in plasma DNA has been shown to need much higher sequencing depth (for example, >200 times haploid human genome coverage).

Double-stranded cell-free DNA may have blunt ends or jagged ends, accordingly presence and/or extent of a jagged end may form part of a fragmentomic signature. Different nucleases have different preferences for the generation of cleaved double-stranded DNA with blunt versus protruding or jagged ends. Jagged ends may be repaired with either methylated or unmethylated cytosines, and then the abundance of jagged ends may be measured by a change in methylation level from that of the genome. The frequencies of jagged ends have been found to be increased in ctDNA in cancer patients. The frequencies of jagged ends may be related to the relative activities between DNASE1 and DNASE1L3, with the former increasing and the latter decreasing the frequencies of jagged ends.

Plasma DNA fragmentation is a nonrandom process in which certain genomic regions are more prone to be cleaved and to be found at an end of a plasma DNA fragment, called “preferred end sites,” accordingly such sites may form part of a fragmentomic signature. These sites may differ for DNA molecules with different tissue sources. When cell-free DNA is aligned to the human genome, their ends tend to cluster at genomic locations (preferred end sites), which can be variable between DNA molecules that originate from different tissues. A window protection score, which may be calculated as the number of complete fragments minus the number of fragment endpoints within a given window size, may convey information about DNA protection from digestion, which can be used to infer nucleosome positioning. The genomic coverage and directional information of the cell-free DNA ending locations—namely upstream end or downstream end—are reflective of the chromatin structure of the tissue of origin (e.g., TF, transcription factor).

The predominant local positions of nucleosomes across the human genome in tissue(s) contributing to cfDNA may be inferred by comparing the distribution of aligned fragment endpoints, or a mathematical transformation thereof, to one or more reference maps. An example of values that can be used for fragmentomic analysis is a Windowed Protection Score (“WPS”) as described in PCT application WO2016/015058, which was developed to reflect such positioning, accordingly a WPS may form part of a fragmentomic signature. Specifically, it is expected that cfDNA fragment endpoints should cluster adjacent to nucleosome boundaries, while also being depleted on the nucleosome itself. The value of the WPS correlates with the locations of nucleosomes within strongly positioned arrays, as mapped by other groups with in vitro methods or ancient DNA. At other sites, the WPS correlates with genomic features such as DNase I hypersensitive (DHS) sites (e.g., consistent with the repositioning of nucleosomes flanking a distal regulatory element). Fragmentomic analysis typically involves determining a value (or values) based on the number of fragment endpoints that map to a specific genomic location (one base or more) as normalized for the amount of sequence data at or near the genomic location so as to fragmentomic values that can be input into models for comparing healthy and afflicted individuals in order determine the possible presence or absence of disease in the test subject. For example, if 10000 paired end reads have an end that map within 500 bp genomic region and 100 ends map to a single base location within that 500 bp region, then a value of 100/1000 could be a fragmentomic value for that single base locations. While not being bound by theory, fragmentomic values appear to be indicative of the presence or absence of proteins, e.g. histones or transcription factors, bound to the interrogated genomic regions. The presence or absence or such bound proteins is believed to affect the accessibility of nuclease to the DNA protected by the bound proteins.

In an embodiment, in a feature engineering step 104, input features for a machine learning step may be created by, for example, analyzing the sequence data, the epigenetic data, the genomic alteration data, combinations thereof, and the like. Additional or other data types may optionally be used for the feature engineering step. The method 100 may also comprise one or more transformation and/or clean-up processes at a data normalization step 106, such as, clean-up for sample prevalences (e.g., adjust for samples with a low number of a given nucleic acid variant, low number of samples, etc.), perform log transformations (e.g., Log (x+1) or Np.log 1p), and perform normalization (e.g., Yeo-Johnson normalization, min-max normalization, z-score normalization, and/or the like) (step 108).

The method 100 may comprise a machine learning step 108 that generates a machine learning model (e.g., classifier) according to a training dataset generated from the data obtained at step 102 (e.g., through creation of a training data set) and the input features from step 104. The machine learning model may be configured provide classify, predict, or otherwise determine one or more probabilities that the origin of a given nucleic acid variant present in a test sample is tumor or non-tumor. The machine learning step 108 may use any machine learning technique, for example, logistic regression or a deep learning technique. Exemplary models that can be used for training and classification, may include without limitations, one or more of: logistic regression, probit regression, decision trees, random forests, gradient boosting, support vector machines, k-nearest neighbors, neural networks, or an ensemble of more than one of these methods. Ensemble methods are meta-algorithms that combine several machine learning techniques into one predictive model in order to decrease variance (bagging), bias (boosting), or improve predictions (stacking). Most ensemble methods use a single base learning algorithm to produce homogeneous base learners, that is, learners of the same type, leading to homogeneous ensembles. There are also some methods that use heterogeneous learners, that is, learners of different types, leading to heterogeneous ensembles. In order for ensemble methods to be more accurate than any of its individual members, the base learners have to be as accurate as possible and as diverse as possible.

The method 100 may, at step 110, output a machine learning model/classifier that is configured to classify or otherwise predict the origin of a sample when provided with epigenetic data and/or genomic alteration data associated with the sample.

The machine learning model/classifier may be used to determine an origin of a newly presented sequence fragment in a test sample. The origin may be tumor derived or may be non-tumor derived. A sequence fragment classified as tumor derived by the machine learning model/classifier may be used to direct treatment of a subject. It may have been previously unknown whether the subject has a disease or it may be known that the subject has a disease. The disease may be cancer. The methods may comprise administering one or more therapies to the subject to treat the disease. The therapies may comprise administering chemotherapy, administering radiation therapy, or performing surgery to resect all or a portion of the tumor. The methods may comprise assisting in a communication of determination of the origin as being tumor derived to a subject associated with the test sample.

### C. Example Systems and Methods

The systems and methods described herein are directed to a cfDNA blood-based assay for the detection of CRC. The methods interrogate epigenetic factors (aberrant methylation status and fragmentomic patterns) and cfDNA genomic alterations. Results are integrated into a binary “abnormal signal detected” (“positive”) or “normal signal detected” (“negative”). Below is the description of the cancer screening assay and each of the components.

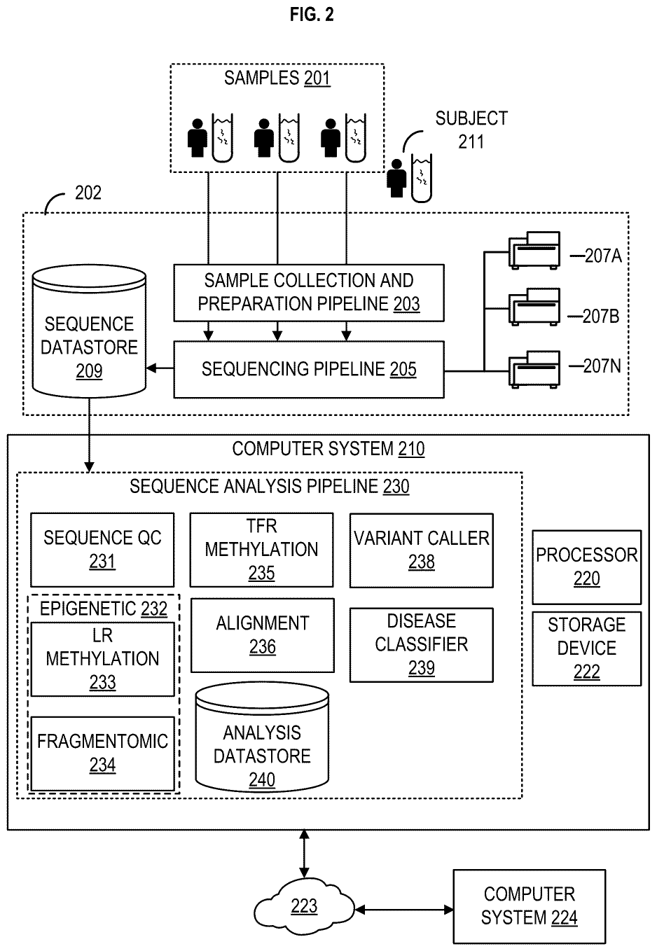

FIG. 2 illustrates an example of a system 200 for determining whether a sample of a test subject 211 is tumor-derived, according to an embodiment of the present disclosure. The system 200 may process one or more samples 201 from the test subject 211 to generate sequence reads. The system 200 may include a laboratory system 202, a computer system 210, and/or other components. It should be noted that the laboratory system 202 and the computer system 210 may be remote from one another, and connected to one another through a computer network (not illustrated). The laboratory system 202 may include a sample collection and preparation pipeline 203, a sequencing pipeline 205, a sequence read datastore 209, and/or other components. The sequencing pipeline 205 may include one or more sequencing devices 207 (illustrated in FIG. 2 as sequencing devices 207a . . . n).

The methods of this disclosure may have a wide variety of uses in the manipulation, preparation, identification, quantification, and/or analysis of cell-free nucleic acids. As shown in FIG. 2, the sample collection and preparation pipeline 203 may include obtaining cfDNA reference samples 201 from one or more reference subjects and a cfDNA test sample 211 from a test subject. As described herein, a polynucleotide can comprise any type of nucleic acid, such as DNA and/or RNA. For example, if a polynucleotide is DNA, it can be genomic DNA, complementary DNA (cDNA), or any other deoxyribonucleic acid. A polynucleotide can also be a cell-free nucleic acid such as cell-free DNA (cfDNA). For example, the polynucleotide can be circulating cfDNA. Circulating cfDNA may comprise DNA shed from bodily cells via apoptosis or necrosis. cfDNA shed via apoptosis or necrosis may originate from normal (e.g., healthy) bodily cells. Where there is abnormal tissue growth, such as for cancer, tumor DNA may be shed. The circulating cfDNA can comprise circulating tumor DNA (ctDNA).

### 1. Samples

Isolation and extraction of cell free polynucleotides may be performed through collection of samples using a variety of techniques. A sample can be any biological sample isolated from a subject. Samples can include body tissues, whole blood, platelets, serum, plasma, stool, red blood cells, white blood cells or leucocytes, endothelial cells, tissue biopsies (e.g., biopsies from known or suspected solid tumors), cerebrospinal fluid, synovial fluid, lymphatic fluid, ascites fluid, interstitial or extracellular fluid (e.g., fluid from intercellular spaces), gingival fluid, crevicular fluid, bone marrow, pleural effusions, cerebrospinal fluid, saliva, mucous, sputum, semen, sweat, urine. Samples are preferably body fluids, particularly blood and fractions thereof, and urine. Such samples include nucleic acids shed from tumors. The nucleic acids can include DNA and RNA and can be in double and single-stranded forms. A sample can be in the form originally isolated from a subject or can have been subjected to further processing to remove or add components, such as cells, enrich for one component relative to another, or convert one form of nucleic acid to another, such as RNA to DNA or single-stranded nucleic acids to double-stranded. Thus, for example, a body fluid sample for analysis is plasma or serum containing cell-free nucleic acids, e.g., cell-free DNA (cfDNA).

In some embodiments, the sample volume of body fluid taken from a subject depends on the desired read depth for sequenced regions. Exemplary volumes are about 0.4-40 ml, about 5-20 ml, about 10-20 ml. For example, the volume can be about 0.5 ml, about 1 ml, about 5 ml, about 10 ml, about 20 ml, about 30 ml, about 40 ml, or more milliliters. A volume of sampled plasma is typically between about 5 ml to about 20 ml.

The sample can comprise various amounts of nucleic acid. Typically, the amount of nucleic acid in a given sample is equated with multiple genome equivalents. For example, a sample of about 30 ng DNA can contain about 10,000 (104) haploid human genome equivalents and, in the case of cfDNA, about 200 billion (2×10¹¹) individual polynucleotide molecules. Similarly, a sample of about 100 ng of DNA can contain about 30,000 haploid human genome equivalents and, in the case of cfDNA, about 600 billion individual molecules.

In some embodiments, a sample comprises nucleic acids from different sources, e.g., from cells and from cell-free sources (e.g., blood samples, etc.). Typically, a sample includes nucleic acids carrying mutations. For example, a sample optionally comprises DNA carrying germline mutations and/or somatic mutations. Typically, a sample comprises DNA carrying cancer-associated mutations (e.g., cancer-associated somatic mutations). In some embodiments of the present disclosure, cell free nucleic acids in a subject may derive from a tumor. For example cell-free DNA isolated from a subject can comprise ctDNA.

Exemplary amounts of cell-free nucleic acids in a sample before amplification typically range from about 1 femtogram (fg) to about 1 microgram (μg), e.g., about 1 picogram (pg) to about 200 nanogram (ng), about 1 ng to about 100 ng, about 10 ng to about 1000 ng. In some embodiments, a sample includes up to about 600 ng, up to about 500 ng, up to about 400 ng, up to about 300 ng, up to about 200 ng, up to about 100 ng, up to about 50 ng, or up to about 20 ng of cell-free nucleic acid molecules. Optionally, the amount is at least about 1 fg, at least about 10 fg, at least about 100 fg, at least about 1 pg, at least about 10 pg, at least about 100 pg, at least about 1 ng, at least about 10 ng, at least about 100 ng, at least about 150 ng, or at least about 200 ng of cell-free nucleic acid molecules. In certain embodiments, the amount is up to about 1 fg, about 10 fg, about 100 fg, about 1 pg, about 10 pg, about 100 pg, about 1 ng, about 10 ng, about 100 ng, about 150 ng, or about 200 ng of cell-free nucleic acid molecules. In some embodiments, methods include obtaining between about 1 fg to about 200 ng cell-free nucleic acid molecules from samples.

Cell-free nucleic acids typically have a size distribution of between about 100 nucleotides in length and about 500 nucleotides in length, with molecules of about 110 nucleotides in length to about 230 nucleotides in length representing about 90% of molecules in the sample, with a mode of about 168 nucleotides length and a second minor peak in a range between about 240 to about 440 nucleotides in length. In certain embodiments, cell-free nucleic acids are from about 160 to about 180 nucleotides in length, or from about 320 to about 360 nucleotides in length, or from about 440 to about 480 nucleotides in length.

In some embodiments, cell-free nucleic acids are isolated from bodily fluids through a partitioning step in which cell-free nucleic acids, as found in solution, are separated from intact cells and other non-soluble components of the bodily fluid. In some of these embodiments, partitioning includes techniques such as centrifugation or filtration. Alternatively, cells in bodily fluids are lysed, and cell-free and cellular nucleic acids processed together. Generally, after addition of buffers and wash steps, cell-free nucleic acids are precipitated with, for example, an alcohol. In certain embodiments, additional clean up steps are used, such as silica-based columns to remove contaminants or salts. Non-specific bulk carrier nucleic acids, for example, are optionally added throughout the reaction to optimize certain aspects of the exemplary procedure, such as yield. After such processing, samples typically include various forms of nucleic acids including double-stranded DNA, single-stranded DNA and/or single-stranded RNA. Optionally, single stranded DNA and/or single stranded RNA are converted to double stranded forms so that they are included in subsequent processing and analysis steps. Additional details regarding cfDNA partitioning and related analysis of epigenetic modifications that are optionally adapted for use in performing the methods disclosed herein are described in, for example, WO 2018/119452, filed Dec. 22, 2017, which is incorporated by reference.

### 2. Sequence Data

Sequence information can be obtained from the cfDNA. The sequence information can be used to further analyze epigenetic factors and genomic alterations. Several components can be involved in obtaining sequence data as described herein.

An example overview of the disclosed workflow is as follows. In some aspects, some of the steps can be performed in a different order, particularly some of the tagging of nucleic acid samples. After obtaining cfDNA samples, the samples can be partitioned based on methylation status. Adapters comprising molecular barcodes can be ligated to the samples. Methylation dependent restriction enzyme (MSRE) treatment can be performed on the hyper methylated partition to remove the incorrectly partitioned molecules. A step of optionally treating the hypomethylated partition with MDRE to remove methylated molecules from the hypo partition can also be performed. After MSRE digestion, the partitions can be pooled and PCR amplification can be performed. Target regions can be enriched using probes (e.g., RNA probes or DNA probes). After enrichment, another PCR amplification can be performed. Nucleic acids can be tagged with sample index via the primers during PCR (can be either the 1st PCR prior to enrichment or post enrichment PCR). Samples can then be pooled and sequenced using an NGS instrument. The sequencing reads generated can then be aligned to the human genome. The molecular barcodes (and optionally along with the alignment position) can be used to group the sequencing reads into families of individual cfDNA molecules, which can in turn be used to estimate the counts of molecules at one or more loci (and at genomic regions). The raw molecule counts can then be normalized using positive control regions and then one of the models—the LR or TFR models—can be applied. The LR and/or TFR models, with or without biomarker analysis, can be used to generate a final score of where there is presence or absence of cancer in a subject that the cfDNA was obtained from (e.g. based on ctDNA). Each of these steps is described in further detail throughout.

i. Partitioning; Analysis of Epigenetic Characteristics

In certain embodiments described herein, a population of different forms of nucleic acids (e.g., hypermethylated and hypomethylated DNA in a sample from the subject, such as tagged DNA or an aliquot thereof) can be physically partitioned based on one or more characteristics of the nucleic acids prior to analysis, e.g., sequencing, or tagging and sequencing. This approach can be used to determine, for example, whether hypermethylation variable epigenetic target regions show hypermethylation characteristic of tumor cells or hypomethylation variable epigenetic target regions show hypomethylation characteristic of tumor cells or otherwise indicative of the presence of disease. Additionally, by partitioning a heterogeneous nucleic acid population, one may increase rare signals, e.g., by enriching rare nucleic acid molecules that are more prevalent in one fraction (or partition) of the population. For example, a genetic variation present in hyper-methylated DNA but less (or not) in hypomethylated DNA can be more easily detected by partitioning a sample into hyper-methylated and hypo-methylated nucleic acid molecules. By analyzing multiple fractions of a sample, a multi-dimensional analysis of a single locus of a genome or species of nucleic acid can be performed and hence, greater sensitivity can be achieved.

In some embodiments, the partitions are differentially tagged and then recombined before dividing the sample into first and second aliquots, followed by subsequent steps of methods described herein. In some embodiments, the sample that is divided into the first and second aliquots is a partition, such as a hypomethylated partition, and the second aliquot is combined with at least one other partition, such as a hypermethylated partition, before undergoing enrichment and/or other steps of the method.

In some instances, a heterogeneous nucleic acid sample is partitioned into two or more partitions (e.g., at least 3, 4, 5, 6 or 7 partitions). In some embodiments, each partition is differentially tagged. Tagged partitions can then be pooled together for collective sample prep and/or sequencing. The partitioning-tagging-pooling steps can occur more than once, with each round of partitioning occurring based on a different characteristics (examples provided herein) and tagged using differential tags that are distinguished from other partitions and partitioning means.

Examples of characteristics that can be used for partitioning include sequence length, methylation level, nucleosome binding, sequence mismatch, immunoprecipitation, and/or proteins that bind to DNA. Resulting partitions can include one or more of the following nucleic acid forms: single-stranded DNA (ssDNA), double-stranded DNA (dsDNA), shorter DNA fragments and longer DNA fragments. In some embodiments, a heterogeneous population of nucleic acids is partitioned into nucleic acids with one or more epigenetic modifications and without the one or more epigenetic modifications. Examples of epigenetic modifications include presence or absence of methylation; level of methylation; type of methylation (e.g., 5-methylcytosine versus other types of methylation, such as adenine methylation and/or cytosine hydroxymethylation); and association and level of association with one or more proteins, such as histones. Alternatively, or additionally, a heterogeneous population of nucleic acids can be partitioned into nucleic acid molecules associated with nucleosomes and nucleic acid molecules devoid of nucleosomes. Alternatively, or additionally, a heterogeneous population of nucleic acids may be partitioned into single-stranded DNA (ssDNA) and double-stranded DNA (dsDNA). Alternatively, or additionally, a heterogeneous population of nucleic acids may be partitioned based on nucleic acid length (e.g., molecules of up to 160 bp and molecules having a length of greater than 160 bp).

In some instances, each partition (representative of a different nucleic acid form) is differentially labelled, and the partitions are pooled together prior to sequencing. In other instances, the different forms are separately sequenced.

Samples can include nucleic acids varying in modifications including post-replication modifications to nucleotides and binding, usually noncovalently, to one or more proteins.

In an embodiment, the population of nucleic acids is one obtained from a serum, plasma or blood sample from a subject suspected of having neoplasia, a tumor, or cancer or previously diagnosed with neoplasia, a tumor, or cancer. The population of nucleic acids includes nucleic acids having varying levels of methylation. Methylation can occur from any one or more post-replication or transcriptional modifications. Post-replication modifications include modifications of the nucleotide cytosine, particularly at the 5-position of the nucleobase, e.g., 5-methylcytosine, 5-hydroxymethylcytosine, 5-formylcytosine and 5-carboxylcytosine.

In some embodiments, the nucleic acids in the original population can be single-stranded and/or double-stranded. Partitioning based on single v. double stranded-ness of the nucleic acids can be accomplished by, e.g. using labelled capture probes to partition ssDNA and using double stranded adapters to partition dsDNA.

The affinity agents can be antibodies with the desired specificity, natural binding partners or variants thereof (Bock et al., Nat Biotech 28: 1106-1114 (2010); Song et al., Nat Biotech 29: 68-72 (2011)), or artificial peptides selected e.g., by phage display to have specificity to a given target.

Examples of capture moieties contemplated herein include methyl binding domain (MBDs) and methyl binding proteins (MBPs) as described herein.

Likewise, partitioning of different forms of nucleic acids can be performed using histone binding proteins which can separate nucleic acids bound to histones from free or unbound nucleic acids. Examples of histone binding proteins that can be used in the methods disclosed herein include RBBP4 (RbAp48) and SANT domain peptides.

Although for some affinity agents and modifications, binding to the agent may occur in an essentially all or none manner depending on whether a nucleic acid bears a modification, the separation may be one of degree. In such instances, nucleic acids overrepresented in a modification bind to the agent at a greater extent that nucleic acids underrepresented in the modification. Alternatively, nucleic acids having modifications may bind in an all or nothing manner. But then, various levels of modifications may be sequentially eluted from the binding agent.

For example, in some embodiments, partitioning can be binary or based on degree/level of modifications. For example, all methylated fragments can be partitioned from unmethylated fragments using methyl-binding domain proteins (e.g., MethylMiner Methylated DNA Enrichment Kit (Thermo Fisher Scientific). Subsequently, additional partitioning may involve eluting fragments having different levels of methylation by adjusting the salt concentration in a solution with the methyl-binding domain and bound fragments. As salt concentration increases, fragments having greater methylation levels are eluted.

In some instances, the final partitions are representatives of nucleic acids having different extents of modifications (overrepresentative or underrepresentative of modifications). Overrepresentation and underrepresentation can be defined by the number of modifications born by a nucleic acid relative to the median number of modifications per strand in a population. For example, if the median number of 5-methylcytosine residues in nucleic acid in a sample is 2, a nucleic acid including more than two 5-methylcytosine residues is overrepresented in this modification and a nucleic acid with 1 or zero 5-methylcytosine residues is underrepresented. The effect of the affinity separation is to enrich for nucleic acids overrepresented in a modification in a bound phase and for nucleic acids underrepresented in a modification in an unbound phase (i.e. in solution). The nucleic acids in the bound phase can be eluted before subsequent processing.

When using MethylMiner Methylated DNA Enrichment Kit (Thermo Fisher Scientific) various levels of methylation can be partitioned using sequential elutions. For example, a hypomethylated partition (e.g., no methylation) can be separated from a methylated partition by contacting the nucleic acid population with the MBD from the kit, which is attached to magnetic beads. The beads are used to separate out the methylated nucleic acids from the non-methylated nucleic acids. Subsequently, one or more elution steps are performed sequentially to elute nucleic acids having different levels of methylation. For example, a first set of methylated nucleic acids can be eluted at a salt concentration of 160 mM or higher, e.g., at least 200 mM, 300 mM, 400 mM, 500 mM, 600 mM, 700 mM, 800 mM, 900 mM, 1000 mM, or 2000 mM. After such methylated nucleic acids are eluted, magnetic separation is once again used to separate higher level of methylated nucleic acids from those with lower level of methylation. The elution and magnetic separation steps can repeat themselves to create various partitions such as a hypomethylated partition (e.g., representative of no methylation), a methylated partition (representative of low level of methylation), and a hyper methylated partition (representative of high level of methylation).

In some methods, nucleic acids bound to an agent used for affinity separation are subjected to a wash step. The wash step washes off nucleic acids weakly bound to the affinity agent. Such nucleic acids can be enriched in nucleic acids having the modification to an extent close to the mean or median (i.e., intermediate between nucleic acids remaining bound to the solid phase and nucleic acids not binding to the solid phase on initial contacting of the sample with the agent).

The affinity separation results in at least two, and sometimes three or more partitions of nucleic acids with different extents of a modification. While the partitions are still separate, the nucleic acids of at least one partition, and usually two or three (or more) partitions are linked to nucleic acid tags, usually provided as components of adapters, with the nucleic acids in different partitions receiving different tags that distinguish members of one partition from another. The tags linked to nucleic acid molecules of the same partition can be the same or different from one another. But if different from one another, the tags may have part of their code in common so as to identify the molecules to which they are attached as being of a particular partition.

For further details regarding portioning nucleic acid samples based on characteristics such as methylation, see WO2018/119452, which is incorporated herein by reference.

In some embodiments, the nucleic acid molecules can be fractionated into different partitions based on the nucleic acid molecules that are bound to a specific protein or a fragment thereof and those that are not bound to that specific protein or fragment thereof.

Nucleic acid molecules can be fractionated based on DNA-protein binding. Protein-DNA complexes can be fractionated based on a specific property of a protein. Examples of such properties include various epitopes, modifications (e.g., histone methylation or acetylation) or enzymatic activity. Examples of proteins which may bind to DNA and serve as a basis for fractionation may include, but are not limited to, protein A and protein G. Any suitable method can be used to fractionate the nucleic acid molecules based on protein bound regions. Examples of methods used to fractionate nucleic acid molecules based on protein bound regions include, but are not limited to, SDS-PAGE, chromatin-immuno-precipitation (ChIP), heparin chromatography, and asymmetrical field flow fractionation (AF4).

In some embodiments, partitioning of the nucleic acids is performed by contacting the nucleic acids with a methylation binding domain (“MBD”) of a methylation binding protein (“MBP”). MBD binds to 5-methylcytosine (5mC). MBD is coupled to paramagnetic beads, such as Dynabeads® M-280 Streptavidin via a biotin linker. Partitioning into fractions with different extents of methylation can be performed by eluting fractions by increasing the NaCl concentration.

Examples of MBPs contemplated herein include, but are not limited to:
(a) MeCP2 is a protein preferentially binding to 5-methyl-cytosine over unmodified cytosine.
(b) RPL26, PRP8 and the DNA mismatch repair protein MHS6 preferentially bind to 5-hydroxymethyl-cytosine over unmodified cytosine.
(c) FOXK1, FOXK2, FOXP1, FOXP4 and FOXI3 preferably bind to 5-formyl-cytosine over unmodified cytosine (Iurlaro et al., Genome Biol. 14: R119 (2013)).
(d) Antibodies specific to one or more methylated nucleotide bases.

In general, elution is a function of number of methylated sites per molecule, with molecules having more methylation eluting under increased salt concentrations. To elute the DNA into distinct populations based on the extent of methylation, one can use a series of elution buffers of increasing NaCl concentration. Salt concentration can range from about 100 mM to about 2500 mM NaCl. In one embodiment, the process results in three (3) partitions. Molecules are contacted with a solution at a first salt concentration and comprising a molecule comprising a methyl binding domain, which molecule can be attached to a capture moiety, such as streptavidin. At the first salt concentration a population of molecules will bind to the MBD and a population will remain unbound. The unbound population can be separated as a “hypomethylated” population. For example, a first partition representative of the hypomethylated form of DNA is that which remains unbound at a low salt concentration, e.g., 100 mM or 160 mM. A second partition representative of intermediate methylated DNA is eluted using an intermediate salt concentration, e.g., between 100 mM and 2000 mM concentration. This is also separated from the sample. A third partition representative of hypermethylated form of DNA is eluted using a high salt concentration, e.g., at least about 2000 mM.

In some embodiments, e.g., wherein an epigenetic target region set is captured, sample DNA (for e.g., between 1 and 300 ng) is mixed with an appropriate amount of methyl binding domain (MBD) buffer (the amount of MBD buffer depends on the amount of DNA used) and magnetic beads conjugated with MBD proteins and incubated overnight. Methylated DNA (hypermethylated DNA) binds the MBD protein on the magnetic beads during this incubation. Non-methylated (hypomethylated DNA) or less methylated DNA (intermediately methylated) is washed away from the beads with buffers containing increasing concentrations of salt. For example, one, two, or more fractions containing non-methylated, hypomethylated, and/or intermediately methylated DNA may be obtained from such washes. Finally, a high salt buffer is used to elute the heavily methylated DNA (hypermethylated DNA) from the MBD protein. In some embodiments, these washes result in three partitions (hypomethylated partition, intermediately methylated fraction and hypermethylated partition) of DNA having increasing levels of methylation.

In some embodiments, the three partitions of DNA are desalted and concentrated in preparation for the enzymatic steps of library preparation.

In some embodiments, the methylation signature of molecules can be determined by methods such as MeDIP-seq, MBD-seq, BS-seq, Ox-BS-seq, TAP-seq, ACE-seq, hmC-seal, and TAB-seq. See, e.g., Schutsky, E. K. et al. Nondestructive, base-resolution sequencing of 5-hydroxymethylcytosine using a DNA deaminase. Nature Biotech, 2018; doi.10.1038/nbt.4204 (ACE-Seq); Yu, Miao et al. Base-resolution analysis of 5-hydroxymethylcytosine in the Mammalian Genome. Cell, 2012; 149(6):1368-80 (TAB-Seq); Han, D. A highly sensitive and robust method for genome-wide 5hmC profiling of rare cell populations. Mol Cell. 2016; 63(4):711-719 (5hmC-Seal); Shen, S. Y. et al. Sensitive tumour detection and classification using plasma cell-free DNA methylomes. Nature. 2018; 563(7732):579-583 (cfMeDIP); Nair, S S et al. Comparison of methyl-DNA immunoprecipitation (MeDIP) and methyl-CpG binding domain (MBD) protein capture for genome-wide DNA. Epigenetics. 2011; 6(1):34-44. In some embodiments, the methylation signature of molecules can be determined by treating the sample with one or more methylation sensitive restriction enzymes (MSRE) and/or methylation dependent restriction enzymes (MDRE). In some embodiments, any of the above methods can be used either alone or in combination, to determine the methylation signature of the molecules.

ii. Nucleic Acid Tags

In some embodiments, the nucleic acid molecules (from the polynucleotides obtained from the samples) may be tagged with sample indexes and/or molecular barcodes (referred to generally as “tags”). Tags may be incorporated into or otherwise joined to adapters by chemical synthesis, ligation (e.g., blunt-end ligation or sticky-end ligation), or overlap extension polymerase chain reaction (PCR), among other methods. Such adapters may be ultimately joined to the target nucleic acid molecule. In other embodiments, one or more rounds of amplification cycles (e.g., PCR amplification) are generally applied to introduce sample indexes to a nucleic acid molecule using conventional nucleic acid amplification methods. The amplifications may be conducted in one or more reaction mixtures (e.g., a plurality of microwells in an array). Molecular barcodes and/or sample indexes may be introduced simultaneously, or in any sequential order. In some embodiments, molecular barcodes and/or sample indexes are introduced prior to and/or after sequence capturing steps are performed. In some embodiments, only the molecular barcodes are introduced prior to probe capturing and the sample indexes are introduced after sequence capturing steps are performed. In some embodiments, both the molecular barcodes and the sample indexes are introduced prior to performing probe-based capturing steps. In some embodiments, the sample indexes are introduced after sequence capturing steps are performed. In some embodiments, molecular barcodes are incorporated to the nucleic acid molecules (e.g. cfDNA molecules) in a sample through adapters via ligation (e.g., blunt-end ligation or sticky-end ligation). In some embodiments, sample indexes are incorporated to the nucleic acid molecules (e.g. cfDNA molecules) in a sample through overlap extension polymerase chain reaction (PCR). Typically, sequence capturing protocols involve introducing a single-stranded nucleic acid molecule complementary to a targeted nucleic acid sequence, e.g., a coding sequence of a genomic region and mutation of such region is associated with a cancer type.

In some embodiments, the tags may be located at one end or at both ends of the sample nucleic acid molecule. In some embodiments, tags are predetermined or random or semi-random sequence oligonucleotides. In some embodiments, the tags may be less than about 500, 200, 100, 50, 20, 10, 9, 8, 7, 6, 5, 4, 3, 2, or 1 nucleotides in length. The tags may be linked to sample nucleic acids randomly or non-randomly.

In some embodiments, each sample is uniquely tagged with a sample index or a combination of sample indexes. In some embodiments, each nucleic acid molecule of a sample or sub-sample is uniquely tagged with a molecular barcode or a combination of molecular barcodes. In other embodiments, a plurality of molecular barcodes may be used such that molecular barcodes are not necessarily unique to one another in the plurality (e.g., non-unique molecular barcodes). In these embodiments, molecular barcodes are generally attached (e.g., by ligation) to individual molecules such that the combination of the molecular barcode and the sequence it may be attached to creates a unique sequence that may be individually tracked. Detection of non-unique molecular barcodes in combination with endogenous sequence information (e.g., the beginning (start) and/or end (stop) genomic location/position corresponding to the sequence of the original nucleic acid molecule in the sample, start and stop genomic positions corresponding to the sequence of the original nucleic acid molecule in the sample, the beginning (start) and/or end (stop) genomic location/position of the sequence read that is mapped to the reference sequence, start and stop genomic positions of the sequence read that is mapped to the reference sequence, sub-sequences of sequence reads at one or both ends, length of sequence reads, and/or length of the original nucleic acid molecule in the sample) typically allows for the assignment of a unique identity to a particular molecule. In some embodiments, beginning region comprises the first 1, first 2, the first 5, the first 10, the first 15, the first 20, the first 25, the first 30 or at least the first 30 base positions at the 5′ end of the sequencing read that align to the reference sequence. In some embodiments, the end region comprises the last 1, last 2, the last 5, the last 10, the last 15, the last 20, the last 25, the last 30 or at least the last 30 base positions at the 3′ end of the sequencing read that align to the reference sequence. The length, or number of base pairs, of an individual sequence read are also optionally used to assign a unique identity to a given molecule. As described herein, fragments from a single strand of nucleic acid having been assigned a unique identity, may thereby permit subsequent identification of fragments from the parent strand, and/or a complementary strand.

In certain embodiments, the number of different tags used to uniquely identify a number of molecules, z, in a class can be between any of 2*z, 3*z, 4*z, 5*z, 6*z, 7*z, 8*z, 9*z, 10*z, 11*z, 12*z, 13*z, 14*z, 15*z, 16*z, 17*z, 18*z, 19*z, 20*z or 100*z (e.g., lower limit) and any of 100,000*z, 10,000*z, 1000*z or 100*z (e.g., upper limit). In some embodiments, molecular barcodes are introduced at an expected ratio of a set of identifiers (e.g., a combination of unique or non-unique molecular barcodes) to molecules in a sample. One example format uses from about 2 to about 1,000,000 different molecular barcode sequences, or from about 5 to about 150 different molecular barcode sequences, or from about 20 to about 50 different molecular barcode sequences, ligated to both ends of a target molecule. Alternatively, from about 25 to about 1,000,000 different molecular barcode sequences may be used. For example, 20-50×20-50 molecular barcode sequences (i.e., one of the 20-50 different molecular barcode sequences can be attached to each end of the target molecule) can be used. Such numbers of identifiers are typically sufficient for different molecules having the same start and stop points to have a high probability (e.g., at least 94%, 99.5%, 99.99%, or 99.999%) of receiving different combinations of identifiers. In some embodiments, about 80%, about 90%, about 95%, or about 99% of molecules have the same combinations of molecular barcodes.

In some embodiments, the assignment of unique or non-unique molecular barcodes in reactions is performed using methods and systems described in, for example, U.S. Patent Application Nos. 20010053519, 20030152490, and 20110160078, and U.S. Pat. Nos. 6,582,908, 7,537,898, 9,598,731, and 9,902,992, each of which is hereby incorporated by reference in its entirety. Alternatively, in some embodiments, different nucleic acid molecules of a sample may be identified using only endogenous sequence information (e.g., start and/or stop positions, sub-sequences of one or both ends of a sequence, and/or lengths).

In certain embodiments described herein, a population of different forms of nucleic acids (e.g., hypermethylated and hypomethylated DNA in a sample) can be physically partitioned prior to analysis, e.g., sequencing, or tagging and sequencing. This approach can be used to determine, for example, whether hypermethylation variable epigenetic target regions show hypermethylation characteristic of tumor cells or hypomethylation variable epigenetic target regions show hypomethylation characteristic of tumor cells. Additionally, by partitioning a heterogeneous nucleic acid population, one may increase rare signals, e.g., by enriching rare nucleic acid molecules that are more prevalent in one fraction (or partition) of the population. For example, a genetic variation present in hyper-methylated DNA but less (or not) in hypomethylated DNA can be more easily detected by partitioning a sample into hyper-methylated and hypo-methylated nucleic acid molecules. By analyzing multiple fractions of a sample, a multi-dimensional analysis of a single locus of a genome or species of nucleic acid can be performed and hence, greater sensitivity can be achieved.

In some instances, a heterogeneous nucleic acid sample is partitioned into two or more partitions (e.g., at least 3, 4, 5, 6 or 7 partitions). In some embodiments, each partition is differentially tagged—i.e., each partition can have a different set of molecular barcodes. Tagged partitions can then be pooled together for collective sample prep and/or sequencing. The partitioning-tagging-pooling steps can occur more than once, with each round of partitioning occurring based on a different characteristics (examples provided herein) and tagged using differential tags that are distinguished from other partitions and partitioning means.

In some instances, each partition (representative of a different nucleic acid form) is differentially tagged with molecular barcodes, and the partitions are pooled together prior to sequencing. In other instances, the different forms are separately sequenced. In some embodiments, a single tag can be used to label a specific partition. In some embodiments, multiple different tags can be used to label a specific partition. In embodiments employing multiple different tags to label a specific partition, the set of tags used to label one partition can be readily differentiated from the set of tags used to label other partitions. In some embodiments, a tag can be multifunctional—i.e., it can simultaneously act as a molecular identifier (i.e., molecular barcode), partition identifier (i.e., partition tag) and sample identifier (i.e., sample index). For example, if there are four DNA samples and each DNA sample is partitioned into three partitions, then the DNA molecules in each of the twelve partitions (i.e., twelve partitions for the four DNA samples in total) can be tagged with a separate set of tags such that the tag sequence attached to the DNA molecule reveals the identity of the DNA molecule, the partition it belongs to and the sample from which it was originated. In some embodiments, a tag can be used both as a molecular barcode and as a partition tag. For example, if a DNA sample is partitioned into three partitions, then DNA molecule in each partition is tagged with a separated set of tags such that the tag sequence attached to a DNA molecule reveals the identity of the DNA molecule and the partition it belongs to. In some embodiments, a tag can be used both as a molecular barcode and as a sample index. For example, if there are four DNA samples, then DNA molecules in each sample with be tagged with a separate set of tags that can be distinguishable from each sample such that the tag sequence attached to the DNA molecule serves as a molecule identifier and as a sample identifier.

In one embodiment, partition tagging comprises tagging molecules in each partition with a partition tag. After re-combining partitions and sequencing molecules, the partition tags identify the source partition. In another embodiment, different partitions are tagged with different sets of molecular tags, e.g., comprised of a pair of barcodes. In this way, each molecular barcode indicates the source partition as well as being useful to distinguish molecules within a partition. For example, a first set of 35 barcodes can be used to tag molecules in a first partition, while a second set of 35 barcodes can be used tag molecules in a second partition.

In some embodiments, after partitioning and tagging with partition tags, the molecules may be pooled for sequencing in a single run. In some embodiments, a sample tag is added to the molecules, e.g., in a step subsequent to addition of partition tags and pooling. Sample tags can facilitate pooling material generated from multiple samples for sequencing in a single sequencing run.

Alternatively, in some embodiments, partition tags may be correlated to the sample as well as the partition. As a simple example, a first tag can indicate a first partition of a first sample; a second tag can indicate a second partition of the first sample; a third tag can indicate a first partition of a second sample; and a fourth tag can indicate a second partition of the second sample.

While tags may be attached to molecules already partitioned based on one or more epigenetic characteristics, the final tagged molecules in the library may no longer possess that epigenetic characteristic. For example, while single stranded DNA molecules may be partitioned and tagged, the final tagged molecules in the library are likely to be double stranded. Similarly, while DNA may be subject to partition based on different levels of methylation, in the final library, tagged molecules derived from these molecules are likely to be unmethylated. Accordingly, the tag attached to molecule in the library typically indicates the characteristic of the “parent molecule” from which the ultimate tagged molecule is derived, not necessarily to characteristic of the tagged molecule, itself.

As an example, barcodes 1, 2, 3, 4, etc. are used to tag and label molecules in the first partition; barcodes A, B, C, D, etc. are used to tag and label molecules in the second partition; and barcodes a, b, c, d, etc. are used to tag and label molecules in the third partition. Differentially tagged partitions can be pooled prior to sequencing. Differentially tagged partitions can be separately sequenced or sequenced together concurrently, e.g., in the same flow cell of an Illumina sequencer.

In some embodiments, tags are introduced at an expected ratio of identifiers (e.g., a combination of unique and/or non-unique barcodes) to microwells. For example, the identifiers may be loaded so that more than about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers are loaded per genome sample. In some embodiments, the identifiers are loaded so that less than about 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers are loaded per genome sample. In certain embodiments, the average number of identifiers loaded per sample genome is less than, or greater than, about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers per genome sample. The identifiers are generally unique and/or non-unique.

One exemplary format uses from about 2 to about 1,000,000 different tags, or from about 5 to about 150 different tags, or from about 20 to about 50 different tags, ligated to both ends of a target nucleic acid molecule. For 20-50×20-50 tags, a total of 400-2500 tags are created. Such numbers of tags are typically sufficient for different molecules having the same start and stop points to have a high probability (e.g., at least 94%, 99.5%, 99.99%, 99.999%) of receiving different combinations of tags.

After sequencing, analysis of reads to detect genetic variants can be performed on a partition-by-partition level, as well as a whole nucleic acid population level. Tags are used to sort reads from different partitions. Analysis can include in silico analysis to determine genetic and epigenetic variation (one or more of methylation, chromatin structure, etc.) using sequence information, genomic coordinates length, coverage and/or copy number. In some embodiments, higher coverage can correlate with higher nucleosome occupancy in genomic region while lower coverage can correlate with lower nucleosome occupancy or a nucleosome depleted region (NDR).

iii. Conversion Procedure

The use of quality control nucleosides in the adapters, as described in the methods disclosed herein, can advantageously be used with enzymatic conversion procedures which convert the base pairing specificity of modified nucleosides (e.g., DM-seq conversion comprising adding a protective group (such as a carboxymethyl group) to unmodified cytosines, and deaminating 5mC, such as using an APOBEC enzyme) or enzymatic conversion procedures which convert the base pairing specificity of unmodified nucleosides. For example, in some embodiments, when a molecule comprising adapters containing two or more quality control nucleosides is exposed to a conversion procedure selected to change the base pairing specificity of quality control nucleosides, the base-pairing specificity of a first portion (e.g., at least one) of the quality control nucleosides is changed but the base-pairing specificity of a second portion (e.g., at least one) of the quality control nucleosides in the adapter is unaffected, which can indicate suboptimal conversion. The use of quality control nucleosides in the adapters, as described in the methods disclosed herein, can advantageously be used to predict/infer/indicate false negative detection and/or identification of modified nucleosides in the DNA sample (i.e., incorrectly identifying a base as being unmodified) and/or false positive detection and/or identification of modified nucleosides in the DNA sample (i.e., incorrectly identifying a base as being modified). Quality control nucleosides as described herein for use to detect the occurrence of false positive detection of modified nucleosides may be referred to as “false positive quality control nucleosides”. Quality control nucleosides as described herein for use to detect the occurrence of false negative detection of modified nucleosides may be referred to as “false negative quality control nucleosides”. A nucleoside having a modification status that means that its base pairing specificity is not changed when exposed to a particular conversion procedure may in some cases be referred to as a “protected” nucleoside or as having a “protected modification status” or similar.

In the case of detecting false negatives using conversion procedures which convert the base pairing specificity of modified nucleosides, the quality control nucleosides in the adapters may comprise modified nucleosides such that the conversion efficiency of the conversion procedure/sub-optimal conversion can measured, and thus the frequency of false negatives predicted. Sub-optimal conversion refers to conversion of fewer than all nucleosides of the type that the reagent used in a conversion procedure normally converts; for example, a sub-optimal conversion by a deaminase as in DM-seq results in conversion of some but not all 5mCs to thymine. The terms sub-optimal and suboptimal have equivalent meanings. Sub-optimal conversion may also be referred to as incomplete conversion in the sense that some nucleosides (modified or unmodified) that should have been converted by the conversion procedure in a complete reaction were not actually converted.

In the case of detecting false positives using conversion procedures which convert the base pairing specificity of unmodified nucleosides, the quality control nucleosides in the adapters may comprise modified nucleosides such that the erroneous conversion frequency of modified nucleosides can measured, and thus the frequency of false positives predicted. Erroneous conversion refers to conversion of a nucleoside other than the nucleosides that are typically converted by a conversion procedure. Conversion of a methylated cytosine by a conversion method that typically converts only unmodified cytosines is an example of erroneous conversion.

In the case of detecting false positives using conversion procedures which convert the base pairing specificity of modified nucleosides, the quality control nucleosides in the adapters may comprise unmodified nucleosides such that the erroneous conversion frequency of unmodified nucleosides can measured, and thus the frequency of false positives predicted.

In the case of detecting false positives using conversion procedures which convert the base pairing specificity of unmodified nucleosides, the quality control nucleosides in the adapters may comprise unmodified nucleosides such that the conversion efficiency of the conversion procedure/sub-optimal conversion can measured, and thus the frequency of false positives predicted.

There are various methods of detecting and/or identifying modified nucleosides that rely on a conversion procedure that changes the base-pairing specificity of a nucleoside, based on the modification status of the nucleosides. These changes of base-pairing specificity can then be detected, and thus the modification status of the nucleoside inferred, by sequencing.

In some cases, the conversion procedure used in the methods of the disclosure is one that changes the base pairing specificity of a modified nucleoside (e.g., methylated cytosine), but does not change the base pairing specificity of the corresponding unmodified nucleoside (e.g. cytosine) or does not change the base pairing specificity of any un-modified nucleoside (e.g. cytosine, adenosine, guanosine and thymidine (or uracil)). Advantages of methods that do not convert the base-pairing specificity of unmodified nucleosides include reduced loss of sequence complexity, higher sequencing efficiency and reduced alignment losses. Additionally, methods such as DM-seq may in some cases be preferred over methods such as bisulfite sequencing and EM-seq because they are less destructive (especially important for low yield samples such as cfDNA) and do not require denaturation, meaning that non-conversion errors are theoretically more likely to be random. In methods that require denaturation for conversion, failure to denature a DNA molecule will result in non-conversion of all bases in the DNA molecule. As biological changes in methylation are predominantly concerted to a localized region of interest, these non-random (localized) conversion can appear as false negatives (non-methylated regions). Random non-conversion methods can maximally affect a low percent of bases within a region, and thus the specificity of methylation change detection can be maximized (reduce false positives) by placing a threshold on % of bases within a region that are methylated/non-methylated. Hence, in some cases, a conversion procedure that does not involve denaturation is preferred.

In some embodiments, an adapter comprises a first quality control nucleoside with a first modification status (e.g., modified, such as methylated) and a second quality control nucleoside with a second modification status (e.g., unmodified). Such adapters can be used to detect both suboptimal conversion and erroneous conversion.

FIG. 1 shows an embodiment of a quality control method for monitoring false negative and/or false positive detection of DNA subjected to a DM-seq base conversion procedure, with optional protection (e.g., by glucosylation) of 5hmC. Adapters containing unmethylated C (e.g., in a molecular barcode) are ligated to DNA and then subjected to a DM-seq conversion procedure, changing base-pairing of the methylated cytosines (sequence read as “T”) and not the non-methylated cytosines (still read as “C”). Each strand is sequenced. Molecules that underwent sub-optimal conversion are identified and can be filtered out at least for purposes of determining methylation. In such examples, a quality control base in an adapter at the 5′ end of a strand of the dsDNA molecule, a quality control base in an adapter at the 3′ end of a strand of the dsDNA molecule, or both, can be assessed to determine whether methylated cytosines in the molecule were successfully deaminated. In some embodiments, molecules that underwent sub-optimal conversion include ssDNA molecules in which methylated Cs are not deaminated and converted to Ts or ssDNA molecules in which 0/2 barcode 5mCs are converted to Ts. In such examples, a quality control base in an adapter at the 5′ end of the ssDNA molecule, a quality control base in an adapter at the 3′ end of the ssDNA molecule, or both, can be assessed to determine whether methylated cytosines in the molecule were successfully deaminated. A sample conversion rate can be calculated by dividing all converted barcode 5mCs by the total of barcode 5mCs.

In other cases, the conversion procedure used in the methods of the disclosure is one that changes the base pairing specificity of an unmodified nucleoside (e.g., cytosine), but does not change the base pairing specificity of the corresponding modified nucleoside (e.g., methylated cytosine).

The skilled person can select a suitable method according to their needs, including which nucleoside modifications are to be detected and/or identified.

In some embodiments, the conversion procedure converts modified nucleosides. In some embodiments, the conversion procedure which converts modified nucleosides comprises enzymatic conversion, such as DM-seq, for example, as described in WO2023/288222A1. In DM-seq, unmodified cytosines in the DNA are enzymatically protected from a subsequent deamination step wherein 5mC in 5mCpG is converted to T. The enzymatically protected unmodified (e.g., unmethylated) cytosines are not converted and are read as “C” during sequencing. Cytosines that are read as thymines (in a CpG context) are identified as methylated cytosines in the DNA.

Thus, when this type of conversion is used, the first nucleobase comprises unmodified (such as unmethylated) cytosine, and the second nucleobase comprises modified (such as methylated) cytosine. Sequencing of the converted DNA identifies positions that are read as cytosine as being unmodified C positions. Meanwhile, positions that are read as T are identified as being T or 5mC. Performing DM-seq conversion thus facilitates identifying positions containing 5mC using the sequence reads obtained. Hence, in these embodiments, the quality control nucleosides in the adapters used in the method comprise unmodified (unmethylated) cytosines.

Exemplary cytosine deaminases for use herein include APOBEC enzymes, for example, APOBEC3A. Generally, AID/APOBEC family DNA deaminase enzymes such as APOBEC3A (A3A) are used to deaminate (unprotected) unmodified cytosine and 5mC. For an exemplary description of APOBEC conversion, see, e.g., Schutsky et al., Nature Biotechnology 2018; 36: 1083-1090.

The enzymatic protection of unmodified cytosines in the DNA comprises addition of a protective group to the unmodified cytosines. Such protective groups can comprise an alkyl group, an alkyne group, a carboxyl group, a carboxyalkyl group, an amino group, a hydroxymethyl group, a glucosyl group, a glucosylhydroxymethyl group, an isopropyl group, or a dye. For example, DNA can be treated with a methyltransferase, such as a CpG-specific methyltransferase, which adds the protective group to unmodified cytosines. The term methyltransferase is used broadly herein to refer to enzymes capable of transferring a methyl or substituted methyl (e.g., carboxymethyl) to a substrate (e.g., a cytosine in a nucleic acid). In some embodiments, the DNA is contacted with a CpG-specific DNA methyltransferase (MTase), such as a CpG-specific carboxymethyltransferase (CxMTase), and a substituted methyl donor, such as a carboxymethyl donor (e.g., carboxymethyl-S-adenosyl-L-methionine). See, e.g., WO2021/236778A2. In particular embodiments, the CxMTase can facilitate the addition of a protective carboxymethyl group to an unmethylated cytosine. In some embodiments, the unmethylated cytosine is unmodified cytosine. The carboxymethyl group can prevent deamination of the cytosine during a deamination step (such as a deamination step using an APOBEC enzyme, such as A3A). Substituted methyl or carboxymethyl donors useful in the disclosed methods include but are not limited to, S-adenosyl-L-methionine (SAM) analogs, optionally wherein the SAM analog is carboxy-S-adenosyl-L-methionine (CxSAM). SAM analogs are described, for example, in WO2022/197593A1. The MTase may be, for example, a CpG methyltransferase from Spiroplasma sp. strain MQ1 (M.SssI), DNA-methyltransferase 1 (DNMT1), DNA-methyltransferase 3 alpha (DNMT3A), DNA-methyltransferase 3 beta (DNMT3B), or DNA adenine methyltransferase (Dam). The CxMTase may be a CpG methyltransferase from Mycoplasma penetrans (M.MpeI). In a particular embodiment, the methyltransferase enzyme is a variant of M.MpeI, wherein the amino acid corresponding to position 374 is R or K, or a sequence at least 90%, at least 92%, at least 94%, at least 96%, at least 97%, at least 98%, or at least 99% identical thereto, optionally wherein the amino acid corresponding to position 374 is R or K.

In one embodiment, the methyltransferase enzyme is a variant of M.MpeI having an N374R substitution or an N374K substitution. The methyltransferase having an N374R substitution or an N374K substitution can further comprise one or more amino acid substitutions selected from a) substitution of one or both residues T300 and E305 with S, A, G, Q, D, or N; b) substitution of one or more residues A323, N306, and Y299 with a positively charged amino acid selected from K, R or H; and/or c) substitution of S323 with A, G, K, R or H, which may enhance the activity of the enzyme.

Optionally, the conversion procedure further includes enzymatic protection of 5hmCs, such as by glucosylation of the 5hmCs (e.g., using PGT) or by carbamoylation of the 5hmCs (e.g., using 5-hydroxymethylcytosine carbamoyltransferase), in the DNA prior to the deamination of unprotected modified cytosines. In this method, 5hmC can be protected from conversion, for example through glucosylation using β-glucosyl transferase (OGT), forming (5-glucosylhydroxymethylcytosine) 5ghmC, or through carbamoylation using 5-hydroxymethylcytosine carbamoyltransferase, forming 5cmC. Examples thereof are described, for example, in Yu et al., Cell 2012; 149: 1368-80, and in Yang et al., Bio-protocol, 2023; 12(17): e4496. Glucosylation or carbamoylation of 5hmC can reduce or eliminate deamination of 5hmC by a deaminase such as APOBEC3A. Treatment with an MTase or CxMTase then adds a protecting group to unmodified (unmethylated) cytosines in the DNA. 5mC (but not protected, unmodified cytosine and not 5ghmC or 5cmC) is then deaminated (converted to T in the case of 5mC) by treatment with a deaminase, for example, an APOBEC enzyme (such as APOBEC3A). Sequencing of the converted DNA identifies positions that are read as cytosine as being either 5hmC or unmodified C positions. Meanwhile, positions that are read as T are identified as being T or 5mC. Performing DM-seq conversion with glucosylation of 5hmC on a sample as described herein thus facilitates distinguishing positions containing unmodified C or 5hmC on the one hand from positions containing 5mC using the sequence reads obtained. Hence, in these embodiments, the quality control nucleosides in the adapters used in the method may comprise both unmodified cytosine and 5hmC. This allows the efficiency of each of the two steps to be determined separately. For example, if sequencing of the adapter indicates that both the 5mC and the 5hmC nucleoside(s) have converted base-pairing specificity, this indicates that the 5hmC-protecting step was ineffective. If the 5mC nucleoside(s) do not have converted base-pairing specificity, this indicates that (at least) the DM-seq process was ineffective. If base-pairing of the 5mC nucleosides, but not the 5hmC nucleosides, in the adapter have converted base-pairing specificity, then both steps were effective.

In addition to controlling for sub-optimal conversion of modified nucleosides, quality control nucleosides in the adapters can also be used to predict false positives (i.e., nucleosides erroneously classified as being modified). In this case, the quality control nucleosides in the adapters comprise, for appropriate conversion procedures, unmodified C. If sequencing of the adapter indicates that quality control nucleoside(s) have converted the base-pairing specificity, this indicates that the unmodified base (e.g., unmodified C) has been erroneously converted. This information can then be used to predict false positive detection of modified nucleosides (e.g., modified C) in the DNA sample.

In particular embodiments, methods of the present disclosure have utility in providing a quality control method for the identification of methylated cytosines which are not present in any sequence context (i.e., CpG and CpH cytosines). Methylated CpH or non-CpG cytosines are infrequent and thus require high levels of sensitivity to reliably detect. Additionally methylated CpGs that co-locate with methylated non-CpGs cannot be detected by methods that use methylation status of non-CpG cytosines as indicator of sub-optimal molecular conversion. The methods of the present disclosure achieve this by providing quality control nucleosides which are known to have a particular modification status, and thus provide a reliable measure of the frequency of erroneous conversion and/or sub-optimal conversion.

In some embodiments, methods of the present disclosure comprise analysis of sequence variations and/or fragmentation patterns, and do not exclude adapted DNA with sub-optimal or erroneous conversion of quality control nucleosides from analysis of sequence variations and/or fragmentation patterns. For example, the methods can comprise detecting the presence or absence of sequence variations and/or determining fragmentation patterns, wherein adapted DNA comprising quality control nucleosides indicative of sub-optimal or erroneous conversion of quality control nucleosides is included in detecting the presence or absence of sequence variations and/or determining fragmentation patterns. In this way, the present methods can reduce the likelihood of false negatives and/or false positives in detecting modified nucleosides (e.g., 5mC) by excluding adapted DNA unsuitable for that purpose due to sub-optimal or erroneous conversion, while retaining such adapted DNA for analyses of sequence variations and/or fragmentation patterns (which are not impacted by suboptimal or erroneous conversion) and therefore avoiding impacting sensitivity.

Some embodiments of the disclosed quality control methods comprise:
(a) ligating the DNA to oligonucleotide adapters, wherein the adapters comprise quality control nucleosides, wherein the quality control nucleosides have the same nucleoside identity and the same or a different modification status to modified nucleosides to be detected in the DNA, and wherein the modification status of the quality control nucleosides is known;
(b) subjecting the adapted DNA, or a subsample thereof, to a conversion procedure that changes the base pairing specificity of the quality control nucleosides or does not change the base pairing specificity of the quality control nucleosides, depending on the modification status of the nucleosides, wherein the conversion procedure comprises deamination of unmodified cytosines, and wherein the conversion procedure is selected to (i) change the base pairing specificity of adapted DNA nucleosides having the same nucleoside identity and modification status as quality control nucleosides in the adapters, and not change the base pairing specificity of adapted DNA nucleosides having the same nucleosides identity as quality control nucleosides in the adapters but a different modification status; and/or (ii) not change the base pairing specificity of adapted DNA nucleosides having the same nucleoside identity and modification status as quality control nucleosides in the adapters, and change the base pairing specificity of adapted DNA nucleosides having the same pairing identity as quality control nucleosides in the adapters but a different modification status;
(c) sequencing the adapted DNA after conversion step (b);
(d) using the sequence data obtained in step (c) to determine base pairing specificity conversion of the quality control nucleosides in the adapters; and
(e) using the base pairing specificity conversion of the quality control nucleosides in the adapters as a quality control measure for conversion step (b), wherein sub-optimal conversion of adapter quality control nucleosides following a conversion procedure of step (b)(i) and/or erroneous conversion of adapter quality control nucleosides following a conversion procedure of step (b)(ii) predicts false negative and/or false positive detection of modified nucleosides in the DNA sample.

In some embodiments of the disclosed methods, the quality control conversion procedure is selected to change the base pairing specificity of unmodified quality control nucleosides in the adapters, but not the base pairing specificity of DNA sample nucleosides having the same nucleoside identity but a different modification status. In some such embodiments, suboptimal conversion of the unmodified quality control nucleosides predicts false negative detection of DNA sample nucleosides having the same nucleoside identity and modification status as the quality control nucleosides or a different modification status and the same change in base pairing specificity on exposure to the conversion procedure. In some such embodiments, suboptimal conversion of the unmodified quality control nucleosides predicts false positive detection of DNA sample nucleosides having the same nucleoside identity and a different modification status as the quality control nucleosides or a different modification status and the same change in base pairing specificity on exposure to the conversion procedure.

In other embodiments of the disclosed methods, the quality control conversion procedure is selected to not change the base pairing specificity of modified quality control nucleosides in the adapters, and to change the base pairing specificity of DNA sample nucleosides having the same nucleoside identity but no modification. In some such embodiments, erroneous conversion of the modified quality control nucleosides predicts false negative detection of DNA sample nucleosides having the same nucleoside identity and modification status as the quality control nucleosides or a different modification status and the same change in base pairing specificity on exposure to the conversion procedure. In some such embodiments, erroneous conversion of the modified quality control nucleosides predicts false positive detection of DNA sample nucleosides having the same nucleoside identity as the quality control nucleosides but no modification or a different modification status and the same change in base pairing specificity on exposure to the conversion procedure.

In some embodiments, the quality control nucleosides in the adapters comprise unmodified cytosine. In some embodiments, the quality control nucleosides in the adapters comprise modified cytosine. In some such embodiments, the quality control nucleosides in the adapters comprise 5-methylcytosine (5mC) and/or 5-hydroxymethyl-cytosine (5hmC). In some embodiments, the quality control nucleosides in the adapters comprise 5-methylcytosine (5mC). In some embodiments, the quality control nucleosides in the adapters comprise 5-hydroxymethyl-cytosine (5hmC).

Thus, also provided herein are methods wherein the conversion procedure comprises deamination of unmodified nucleosides, such as unmodified cytosines. In some embodiments, the conversion procedure comprises enzymatic conversion of unmodified nucleosides, such as unmodified cytosines using a non-specific, modification-sensitive double-stranded DNA deaminase, e.g., as in SEM-seq. See, e.g., Vaisvila et al. (2023) Discovery of novel DNA cytosine deaminase activities enables a nondestructive single-enzyme methylation sequencing method for base resolution high-coverage methylome mapping of cell-free and ultra-low input DNA. bioRxiv; DOI: 10.1101/2023.06.29.547047, available at https://www.biorxiv.org/content/10.1101/2023.06.29.547047v1. SEM-Seq employs a non-specific, modification-sensitive double-stranded DNA deaminase (MsddA) in a nondestructive single-enzyme 5-methylcytosine sequencing (SEM-seq) method that deaminates unmodified cytosines. Accordingly, SEM-seq does not require the TET2 and T4-PGT or 5-hydroxymethylcytosine carbamoyltransferase protection and denaturing steps that are of use, e.g., in APOEC3A-based protocols. Additionally, MsddA does not deaminate 5-formylated cytosines (5fC) or 5-carboxylated cytosines (5caC). In SEM-seq, unmodified cytosines in the DNA are deaminated to uracil and is read as “T” during sequencing. Modified cytosines (e.g., 5mC) are not converted and are read as “C” during sequencing. Cytosines that are read as thymines are identified as unmodified (e.g., unmethylated) cytosines or as thymines in the DNA. Performing SEM-seq conversion thus facilitates identifying positions containing 5mC using the sequence reads obtained. In some embodiments, the procedure that affects a first nucleobase in the DNA differently from a second nucleobase in the DNA comprises enzymatic conversion of the first nucleobase using MsddA. Optionally, however, in some embodiments of the disclosed methods wherein the conversion procedure deaminates unmodified nucleosides (such as unmodified cytosines), the method further comprises enzymatic protection of at least one type of modified nucleoside (such as modified cytosines, such as 5mC and/or 5hmC) in the DNA prior to deamination of unprotected unmodified nucleosides (such as unprotected unmodified cytosines). In some embodiments, the at least one type of modified nucleoside is 5mC. In some embodiments, enzymatic protection of 5mC comprises converting a 5mC to carboxylcytosine. For example, converting a 5mC to carboxylcytosine can comprise contacting the 5mC with a TET enzyme, such as TET1, TET2, or TET3, or any suitable TET enzyme disclosed herein. In some embodiments, the at least one type of modified nucleoside is 5hmC. In some embodiments, the enzymatic protection of 5hmCs in the DNA prior to the deamination of unmodified cytosines glucosylation of the 5hmCs, such as described herein.

Also provided herein are methods in which alternative base conversion schemes are used. For example, unmethylated cytosines can be left intact (such as through being protected, such as using a method disclosed herein) while methylated cytosines and hydroxymethylcytosines are converted to a base read as a thymine (e.g., uracil, thymine, or dihydrouracil).

In some embodiments, converting a modified (such as methylated or hydroxymethylated) cytosine in at least one first or second strand to a thymine or a base read as thymine comprises oxidizing a hydroxymethyl cytosine, e.g., the hydroxymethyl cytosine is oxidized to formylcytosine. In some embodiments, oxidizing the hydroxymethyl cytosine to formylcytosine comprises contacting the hydroxymethyl cytosine with a ruthenate, such as potassium ruthenate (KRuO4).

In some embodiments, the modified cytosine is converted to thymine, uracil, or dihydrouracil. In any such embodiments, amplification methods may comprise uracil- and/or dihydrouracil-tolerant amplification methods, such as PCR using a uracil- and/or dihydrouracil-tolerant DNA polymerase.

In some embodiments, the method comprises converting a formylcytosine and/or a methylcytosine to carboxylcytosine as part of converting the modified cytosine in at least one first or second strand to a thymine or a base read as thymine. For example, converting the formylcytosine and/or the methylcytosine to carboxylcytosine can comprise contacting the formylcytosine and/or the methylcytosine with a TET enzyme, such as TET1, TET2, or TET3. In some embodiments, the method comprises reducing the carboxylcytosine as part of converting the modified cytosine in at least one first or second strand to a thymine or a base read as thymine, and/or the carboxylcytosine is reduced to dihydrouracil. In some embodiments, reducing the carboxylcytosine comprises contacting the carboxylcytosine with a borane or borohydride reducing agent.

In some embodiments, the borane or borohydride reducing agent comprises pyridine borane, 2-picoline borane, borane, tert-butylamine borane, ammonia borane, sodium borohydride, sodium cyanoborohydride (NaBH3CN), lithium borohydride (LiBH4), ethylenediamine borane, dimethylamine borane, sodium triacetoxyborohydride, morpholine borane, 4-methylmorpholine borane, trimethylamine borane, dicyclohexylamine borane, or a salt thereof. In other embodiments, the reducing agent comprises lithium aluminum hydride, sodium amalgam, amalgam, sulfur dioxide, dithionate, thiosulfate, iodide, hydrogen peroxide, hydrazine, diisobutylaluminum hydride, oxalic acid, carbon monoxide, cyanide, ascorbic acid, formic acid, dithiothreitol, beta-mercaptoethanol, or any combination thereof.

Various TET enzymes may be used in the disclosed methods as appropriate. In some embodiments, the one or more TET enzymes comprise TETv. TETv is described in U.S. Pat. No. 10,260,088 and its sequence is SEQ ID NO: 1 therein. In some embodiments, the one or more TET enzymes comprise TETcd. TETcd is described in U.S. Pat. No. 10,260,088 and its sequence is SEQ ID NO: 3 therein. In some embodiments, the one or more TET enzymes comprise TET1. In some embodiments, the one or more TET enzymes comprise TET2. TET2 may be expressed and used as a fragment comprising TET2 residues 1129-1480 joined to TET2 residues 1844-1936 by a linker as described, e.g., in U.S. Pat. No. 10,961,525. In some embodiments, the one or more TET enzymes comprise TET1 and TET2. In some embodiments, the one or more TET enzymes comprise a V1900 TET mutant, such as a V1900A, V1900C, V1900G, V19001, or V1900P TET mutant. In some embodiments, the one or more TET enzymes comprise a V1900 TET2 mutant, such as a V1900A, V1900C, V1900G, V19001, or V1900P TET2 mutant. It can be beneficial to use a TET enzyme that maximizes formation of 5-carboxylcytosine (5-caC) relative to less oxidized modified cytosines, particularly 5-formylcytosine, because 5-caC is not a substrate for enzymatic deamination, e.g., by APOBEC enzymes such as APOBEC3A. Maximizing formation of 5-caC thus reduces the risk of false calls in which a base is identified as unmethylated because it underwent deamination even though it was methylated (or hydroxymethylated) in the original sample. Accordingly, in some embodiments, the TET enzyme comprises a mutation that increases formation of 5-caC. Exemplary mutations are set forth above. “A mutation that increases formation of 5-caC” means that the TET enzyme having the mutation produces more 5-caC than a TET enzyme that lacks the mutation but is otherwise identical. 5-caC production can be measured as described, e.g., in Liu et al., Nat Chem Biol 13:181-187 (2017) (see Online Methods section, TET reactions in vitro subsection, “driving” conditions). Any variants and/or mutants described in Liu et al. (2017) can be used in the disclosed methods as appropriate.

In some embodiments, the one or more TET enzymes comprise a TET2 enzyme comprising a T1372S mutation, such as TET2-CS-T1372S and TET2-CD-T1372S. A TET2 comprising a T1372S mutation is described in U.S. Pat. No. 10,961,525 and may be expressed and used as a fragment comprising TET2 residues 1129-1480 joined to TET2 residues 1844-1936 by a linker. Position 1372 of TET2 corresponds to position 258 of SEQ ID NO: 21 (wild type TET2 catalytic domain) of U.S. Pat. No. 10,961,525. Thus, the sequence of a T1372S TET2 catalytic domain may be obtained by changing the threonine at position 258 of SEQ ID NO: 21 of U.S. Pat. No. 10,961,525 to serine. TET2 comprising a T1372S mutation is also described in Liu et al., Nat Chem Biol. 2017 February; 13(2): 181-187. As demonstrated in Liu et al., TET2 comprising a T1372S mutation can more efficiently oxidize 5mC to produce 5-carboxylcytosine (5caC) than other versions of TET2 such as TET2 lacking a T1372S mutation.

Provided herein is a method comprising contacting DNA contacting DNA with a TET2 enzyme comprising a T1372S mutation to oxidize 5-methylcytosine (5mC) and/or 5-hydroxymethylcytosine (5hmC) present in the DNA to 5-carboxycytosine (5caC), subsequently contacting at least a portion of the DNA with a substituted borane reducing agent, thereby converting 5-caC in the DNA to dihydrouracil (DHU), thereby producing treated DNA, and sequencing at least a portion of the treated DNA.

iv. Nucleic Acid Amplification

Sample nucleic acids flanked by adapters are typically amplified by PCR and other amplification methods using nucleic acid primers binding to primer binding sites in adapters flanking a DNA molecule to be amplified as part of the sample collection and preparation pipeline 203. In some embodiments, amplification methods involve cycles of extension, denaturation and annealing resulting from thermocycling, or can be isothermal as, for example, in transcription mediated amplification. Other exemplary amplification methods that are optionally utilized, include the ligase chain reaction, strand displacement amplification, nucleic acid sequence-based amplification, and self-sustained sequence-based replication, among other approaches.

One or more rounds of amplification cycles are generally applied to introduce molecular tags and/or sample indexes/tags to a nucleic acid molecule using conventional nucleic acid amplification methods. The amplifications are typically conducted in one or more reaction mixtures. Molecular tags and sample indexes/tags are optionally introduced simultaneously, or in any sequential order. In some embodiments, molecular tags and sample indexes/tags are introduced prior to and/or after sequence capturing steps are performed. In some embodiments, only the molecular tags are introduced prior to probe capturing and the sample indexes/tags are introduced after sequence capturing steps are performed. In certain embodiments, both the molecular tags and the sample indexes/tags are introduced prior to performing probe-based capturing steps. In some embodiments, the sample indexes/tags are introduced after sequence capturing steps are performed. Typically, sequence capturing protocols involve introducing a single-stranded nucleic acid molecule complementary to a targeted nucleic acid sequence, e.g., a coding sequence of a genomic region and mutation of such region associated with a cancer type. Typically, the amplification reactions generate a plurality of non-uniquely or uniquely tagged nucleic acid amplicons with molecular tags and sample indexes/tags at size ranging from about 200 nucleotides (nt) to about 700 nt, from 250 nt to about 350 nt, or from about 320 nt to about 550 nt. In some embodiments, the amplicons have a size of about 300 nt. In some embodiments, the amplicons have a size of about 500 nt.

a. Nucleic Acid Enrichment

In some embodiments, sequences are enriched prior to sequencing the nucleic acids as part of the sample collection and preparation pipeline 203. Enrichment is optionally performed for specific target regions or nonspecifically (“target sequences”). In some embodiments, targeted regions of interest may be enriched with nucleic acid capture probes (“baits”) selected for one or more bait set panels using a differential tiling and capture scheme. A differential tiling and capture scheme generally uses bait sets of different relative concentrations to differentially tile (e.g., at different “resolutions”) across genomic sections associated with the baits, subject to a set of constraints (e.g., sequencer constraints such as sequencing load, utility of each bait, etc.), and capture the targeted nucleic acids at a desired level for downstream sequencing. These targeted genomic sections of interest optionally include natural or synthetic nucleotide sequences of the nucleic acid construct. In some embodiments, biotin-labeled beads with probes to one or more sections of interest can be used to capture target sequences, and optionally followed by amplification of those sections, to enrich for the regions of interest.

Sequence capture typically involves the use of oligonucleotide probes that hybridize to the target nucleic acid sequence. In certain embodiments, a probe set strategy involves tiling the probes across a section of interest. Such probes can be, for example, from about 60 to about 120 nucleotides in length. The set can have a depth of about 2×, 3×, 4×, 5×, 6×, 8×, 9×, 10×, 15×, 20×, 50× or more. The effectiveness of sequence capture generally depends, in part, on the length of the sequence in the target molecule that is complementary (or nearly complementary) to the sequence of the probe.

v. Nucleic Acid Sequencing

As shown in FIG. 2, after extraction and isolation of cfDNA from samples via the sample collection and preparation pipeline 203, the cfDNA may be sequenced via the sequencing pipeline 205 including one or more sequencing devices 207. Sample nucleic acids, optionally flanked by adapters, with or without prior amplification are generally subject to sequencing. Sequencing methods or commercially available formats that are optionally utilized include, for example, Sanger sequencing, high-throughput sequencing, bisulfite sequencing, pyrosequencing, sequencing-by-synthesis, single-molecule sequencing, nanopore-based sequencing, semiconductor sequencing, sequencing-by-ligation, sequencing-by-hybridization, RNA-Seq (Illumina), Digital Gene Expression (Helicos), next generation sequencing (NGS), Single Molecule Sequencing by Synthesis (SMSS) (Helicos), massively-parallel sequencing, Clonal Single Molecule Array (Solexa), shotgun sequencing, Ion Torrent, Oxford Nanopore, Roche Genia, Maxim-Gilbert sequencing, primer walking, sequencing using PacBio, SOLiD, Ion Torrent, or nanopore platforms. Sequencing reactions can be performed in a variety of sample processing units, which may include multiple lanes, multiple channels, multiple wells, or other means of processing multiple sample sets substantially simultaneously. Sample processing units can also include multiple sample chambers to enable the processing of multiple runs simultaneously.

In some embodiments, sequencing comprises detecting and/or distinguishing unmodified and modified nucleobases. For example, long-read sequencing (also referred to herein as third generation sequencing) methods include those that can generate longer sequencing reads, such as reads in excess of 10 kilobases, as compared to short-read sequencing methods, which generally produce reads of up to about 600 bases in length. Compared to short reads, long reads can improve de novo assembly, transcript isoform identification, and detection and/or mapping of structural variants. Furthermore, long-read sequencing of native DNA or RNA molecules reduces amplification bias and preserves base modifications, such as methylation status. Long-read sequencing technologies useful herein can include any suitable long-read sequencing methods, including, but not limited to, Pacific Biosciences (PacBio) single-molecule real-time (SMRT) sequencing, Oxford Nanopore Technologies (ONT) nanopore sequencing, and synthetic long-read sequencing approaches, such as linked reads, proximity ligation strategies, and optical mapping. Synthetic long-read approaches comprise assembly of short reads from the same DNA molecule to generate synthetic long reads, and may be used in conjunction with “true” long-read sequencing technologies, such as SMRT and nanopore sequencing methods.

Single-molecule real-time (SMRT) sequencing can facilitate direct detection of, e.g., 5-methylcytosine and 5-hydroxymethylcytosine as well as unmodified cytosine (Weirather J L, et al., “Comprehensive comparison of Pacific Biosciences and Oxford Nanopore Technologies and their applications to transcriptome analysis,” F1000Research, 6:100, 2017). Whereas next-generation sequencing methods detect augmented signals from a clonal population of amplified DNA fragments, SMRT sequencing captures a single DNA molecule, maintaining base modification during sequencing. The error rate of raw PacBio SMRT sequencing-generated data is about 13-15%, as the signal-to-noise ratio from single DNA molecules not high. To increase accuracy, this platform uses a circular DNA template by ligating hairpin adaptors to both ends of target double-stranded DNA. As the polymerase repeatedly traverses and replicates the circular molecule, the DNA template is sequenced multiple times to generate a continuous long read (CLR). The CLR can be split into multiple reads (“subreads”) by removing adapter sequences, and multiple subreads generate circular consensus sequence (“CCS”) reads with higher accuracy. The average length of a CLR is >10 kb and up to 60 kb, with length depending on the polymerase lifetime. Thus, the length and accuracy of CCS reads depends on the fragment sizes. PacBio sequencing has been utilized for genome (e.g., de novo assembly, detection of structural variants and haplotyping) and transcriptome (e.g., gene isoform reconstruction and novel gene/isoform discovery) studies.

SMRT sequencing relies on sequencing-by-synthesis, where the sequence of a circular DNA template is determined from the succession of fluorescence pulses, each resulting from the addition of one labelled nucleotide by a polymerase fixed to the bottom of a well. Base modifications do not affect the base-called sequence, but they affect the kinetics of the polymerase. By considering the inter-pulse duration (IPD), base modifications can be inferred from the comparison of a modified template to an in silico model or an unmodified template. Such methods can therefore use the pulse width of a signal from sequencing bases, the interpulse duration (IPD) of bases, and the identity of the bases in order to detect a modification in a base or in a neighboring base. (See e.g., Weirather et al., F1000Research, 6:100, 2017.) SMRT sequencing can thus be used to detect base modifications such as 5-caC, 4mC, 5mC, 5hmC, 6 mA, and 8oxoG (Gouil & Keniry Essays in Biochemistry (2019) 63 639-648). Accordingly, in some embodiments, the sequencing comprises SMRT sequencing. In such embodiments, the end repair may be performed using dNTPs, which comprise 5-caC, 4mC, 5mC, 5hmC, 6 mA, and/or 8oxoG.

Some sequencing reactions involve use of an enzyme to control passage of a nucleic acid through a nanopore, and in such cases reaction data can include both kinetics and other behavior of the enzyme and fluctuations in current through the nanopore. For example, ratchet proteins, helicases, or motor proteins can be used to push or pull a nucleic acid molecule through a hole in a biological or synthetic membrane. The kinetics of these proteins can vary depending on the sequence context of a nucleic acid on which they are acting. For example, they may slow down or pause at a modified base, and this behavior, captured as a part of the reaction data, is indicative of the presence of the modified base even where the modified base is not within the sensing portion of the nanopore.

One example of a nanopore-based single molecule sequencing system is that commercialized by Oxford Nanopore Technologies (ONT). (Weirather J L, et al., F1000Research, 6:100, 2017). ONT directly sequences a native single-stranded DNA (ssDNA) molecule by measuring characteristic current changes as the bases are threaded through the nanopore by a molecular motor protein. ONT uses a hairpin library structure similar to the PacBio circular DNA template: the DNA template and its complement are bound by a hairpin adaptor. Therefore, the DNA template passes through the nanopore, followed by a hairpin and finally the complement. The raw read can be split into two “1D” reads (“template” and “complement”) by removing the adaptor. The consensus sequence of two “1D” reads is a “2D” read with a higher accuracy.

Nanopore sequencing can be used to detect base modifications including 5-caC, 5mC, 5hmC, 6 mA, BrdU, FldU, IdU, and EdU (see e.g., Gouil & Keniry Essays in Biochemistry (2019) 63 639-648; Kutyavin, Biochemistry (2008), 47, 51, 13666-1367; Müller et al., Nature Methods (2019), volume 16, pages 429-436; Hennion et al., Genome Biology (2020), volume 21, Article number: 125). Accordingly, in some embodiments, the sequencing comprises nanopore sequencing. In such embodiments, the end repair may be performed using dNTPs, which comprise 5-caC, 4mC, 5mC, 5hmC, 6 mA, BrdU, FldU, IdU, and/or EdU.

5-letter and 6-letter sequencing methods include whole genome sequencing methods capable of sequencing A, C, T, and G in addition to 5mC and 5hmC to provide a 5-letter (A, C, T, G, and either 5mC or 5hmC) or 6-letter (A, C, T, G, 5mC, and 5hmC) digital readout in a single workflow. The processing of the DNA sample is entirely enzymatic and avoids the DNA degradation and genome coverage biases of bisulfite treatment. In an exemplary 5-letter sequencing method developed by Cambridge Epigenetix, the sample DNA is first fragmented via sonication and then ligated to short, synthetic DNA hairpin adaptors at both ends (Füllgrabe, et al. 2022, bioRxiv doi: https://doi.org/10.1101/2022.07.08.499285). The construct is then split to separate the sense and antisense sample strands. For each original sample strand a complementary copy strand is synthesized by DNA polymerase extension of the 3′-end to generate a hairpin construct with the original sample DNA strand connected to its complementary strand, lacking epigenetic modifications, via a synthetic loop. Sequencing adapters are then ligated to the end. Modified cytosines are enzymatically protected. The unprotected Cs are then deaminated to uracil, which is subsequently read as thymine. In any such embodiments, amplification methods may comprise uracil- and/or dihydrouracil-tolerant amplification methods, such as PCR using a uracil- and/or dihydrouracil-tolerant DNA polymerase (i.e., a DNA polymerase that can read and amplify templates comprising uracil and/or dihydrouracil bases). The deaminated constructs are no longer fully complementary and have substantially reduced duplex stability, thus the hairpins can be readily opened and amplified by PCR. The constructs can be sequenced in paired-end format whereby read 1 (P1 primed) is the original stand and read 2 (P2 primed) is the copy stand. The read data is pairwise aligned so read 1 is aligned to its complementary read 2. Cognate residues from both reads are computationally resolved to produce a single genetic or epigenetic letter. Pairings of cognate bases that differ from the permissible five are the result of incomplete fidelity at some stage(s) comprising sample preparation, amplification, or erroneous base calling during sequencing. As these errors occur independently to cognate bases on each strand, substitutions result in a non-permissible pair. Non-permissible pairs are masked (marked as N) within the resolved read and the read itself is retained, leading to minimal information loss and high accuracy at read-level. The resolved read is aligned to the reference genome. Genetic variants and methylation counts are produced by read-counting at base-level.

5hmC has been shown to have value as a marker of biological states and disease which includes early cancer detection from cell-free DNA. In adapting 5-letter to 6-letter sequencing, 5mC is disambiguated from 5hmC without compromising genetic base calling within the same sample fragment. The first three steps of the workflow are identical to 5-letter sequencing described above, to generate the adapter ligated sample fragment with the synthetic copy strand. Methylation at 5mC is enzymatically copied across the CpG unit to the C on the copy strand, whilst 5hmC is enzymatically protected from such a copy. Thus, unmodified C, 5mC and 5hmC in each of the original CpG units are distinguished by unique 2-base combinations. The unmodified cytosines are then deaminated to uracil, which is subsequently read as thymine. The DNA is subjected to PCR amplification and sequencing as described earlier. The reads are pairwise aligned and resolved using a 2-base code. Each of unmodified C, 5mC, and 5hmC can be resolved as the three CpG units are distinct sequencing environments of the 2-base code.

The sequencing reactions can be performed on one more nucleic acid fragment types or sections known to contain markers of cancer or of other diseases. The sequencing reactions can also be performed on any nucleic acid fragment present in the sample. The sequence reactions may provide for sequence coverage of the genome of at least about 5%, 10%, 15%, 20%, 25%, 30%, 40%, 50%, 60%, 70%, 80%, 90%, 95%, 99%, 99.9% or 100% of the genome. In other cases, sequence coverage of the genome may be less than about 5%, 10%, 15%, 20%, 25%, 30%, 40%, 50%, 60%, 70%, 80%, 90%, 95%, 99%, 99.9% or 100% of the genome.

Simultaneous sequencing reactions may be performed using multiplex sequencing techniques. In some embodiments, cell-free polynucleotides are sequenced with at least about 1000, 2000, 3000, 4000, 5000, 6000, 7000, 8000, 9000, 10000, 50000, or 100,000 sequencing reactions. In other embodiments, cell-free polynucleotides are sequenced with less than about 1000, 2000, 3000, 4000, 5000, 6000, 7000, 8000, 9000, 10000, 50000, or 100,000 sequencing reactions. Sequencing reactions are typically performed sequentially or simultaneously. Subsequent data analysis is generally performed on all or part of the sequencing reactions. In some embodiments, data analysis is performed on at least about 1000, 2000, 3000, 4000, 5000, 6000, 7000, 8000, 9000, 10000, 50000, or 100,000 sequencing reactions. In other embodiments, data analysis may be performed on less than about 1000, 2000, 3000, 4000, 5000, 6000, 7000, 8000, 9000, 10000, 50000, or 100,000 sequencing reactions. An exemplary read depth is from about 1000 to about 50000 reads per locus (base position).

In some embodiments, a nucleic acid population is prepared for sequencing by enzymatically forming blunt-ends on double-stranded nucleic acids with single-stranded overhangs at one or both ends. In these embodiments, the population is typically treated with an enzyme having a 5′-3′ DNA polymerase activity and a 3′-5′ exonuclease activity in the presence of the nucleotides (e.g., A, C, G and T or U). Exemplary enzymes or catalytic fragments thereof that are optionally used include Klenow large fragment and T4 polymerase. At 5′ overhangs, the enzyme typically extends the recessed 3′ end on the opposing strand until it is flush with the 5′ end to produce a blunt end. At 3′ overhangs, the enzyme generally digests from the 3′ end up to and sometimes beyond the 5′ end of the opposing strand. If this digestion proceeds beyond the 5′ end of the opposing strand, the gap can be filled in by an enzyme having the same polymerase activity that is used for 5′ overhangs. The formation of blunt-ends on double-stranded nucleic acids facilitates, for example, the attachment of adapters and subsequent amplification.

In some embodiments, nucleic acid populations are subject to additional processing, such as the conversion of single-stranded nucleic acids to double-stranded and/or conversion of RNA to DNA. These forms of nucleic acid are also optionally linked to adapters and amplified.

With or without prior amplification, nucleic acids subject to the process of forming blunt-ends described above, and optionally other nucleic acids in a sample, can be sequenced to produce sequenced nucleic acids. A sequenced nucleic acid can refer either to the sequence of a nucleic acid (i.e., sequence information) or a nucleic acid whose sequence has been determined. Sequencing can be performed so as to provide sequence data of individual nucleic acid molecules in a sample either directly or indirectly from a consensus sequence of amplification products of an individual nucleic acid molecule in the sample.

In some embodiments, double-stranded nucleic acids with single-stranded overhangs in a sample after blunt-end formation are linked at both ends to adapters including barcodes, and the sequencing determines nucleic acid sequences as well as in-line barcodes introduced by the adapters. The blunt-end DNA molecules are optionally ligated to a blunt end of an at least partially double-stranded adapter (e.g., a Y shaped or bell-shaped adapter). Alternatively, blunt ends of sample nucleic acids and adapters can be tailed with complementary nucleotides to facilitate ligation (e.g., sticky end ligation).

The nucleic acid sample is typically contacted with a sufficient number of adapters such that there is a low probability (e.g., <1 or 0.1%) that any two copies of the same nucleic acid receive the same combination of adapter barcodes from the adapters linked at both ends. The use of adapters in this manner permits identification of families of nucleic acid sequences with the same start and stop points on a reference nucleic acid and linked to the same combination of barcodes. Such a family represents sequences of amplification products of a nucleic acid in the sample before amplification. The sequences of family members can be compiled to derive consensus nucleotide(s) or a complete consensus sequence for a nucleic acid molecule in the original sample, as modified by blunt end formation and adapter attachment. In other words, the nucleotide occupying a specified position of a nucleic acid in the sample is determined to be the consensus of nucleotides occupying that corresponding position in family member sequences. Families can include sequences of one or both strands of a double-stranded nucleic acid. If members of a family include sequences of both strands from a double-stranded nucleic acid, sequences of one strand are converted to their complement for purposes of compiling all sequences to derive consensus nucleotide(s) or sequences. Some families include only a single member sequence. In this case, this sequence can be taken as the sequence of a nucleic acid in the sample before amplification. Alternatively, families with only a single member sequence can be eliminated from subsequent analysis.

Additional details regarding nucleic acid sequencing, including the formats and applications described herein are also provided in, for example, Levy et al., Annual Review of Genomics and Human Genetics, 17: 95-115 (2016), Liu et al., J. of Biomedicine and Biotechnology, Volume 2012, Article ID 251364:1-11 (2012), Voelkerding et al., Clinical Chem., 55: 641-658 (2009), MacLean et al., Nature Rev. Microbiol., 7: 287-296 (2009), Astier et al., J Am Chem Soc., 128(5):1705-10 (2006), U.S. Pat. Nos. 6,210,891, 6,258,568, 6,833,246, 7,115,400, 6,969,488, 5,912,148, 6,130,073, 7,169,560, 7,282,337, 7,482,120, 7,501,245, 6,818,395, 6,911,345, 7,501,245, 7,329,492, 7,170,050, 7,302,146, 7,313,308, and 7,476,503, which are each incorporated by reference in their entirety.

a. Sequencing Panel

To improve the likelihood of detecting genomic regions of interest and optionally, tumor indicating mutations, the sections of DNA sequenced may comprise a panel of genes or genomic sections that comprise known genomic regions. Selection of a limited section for sequencing (e.g., a limited panel) can reduce the total sequencing needed (e.g., a total amount of nucleotides sequenced). A sequencing panel can target a plurality of different genes or regions, for example, to detect a single cancer, a set of cancers, or all cancers. Alternatively, DNA may be sequenced by whole genome sequencing (WGS) or other unbiased sequencing method without the use of a sequencing panel. Examples of suitable panel and targets for use in panels can be found in the epigenetic targets described in International Application WO2020160414, filed Jan. 31, 2020, which is incorporated by reference in its entirety.

In some aspects, a panel that targets a plurality of different genes or genomic regions (e.g., CHIP genes, transcriptional factor binding regions, distal regulatory elements (DREs), repetitive elements, intron-exon junctions, transcriptional start sites (TSSs), and/or the like) is selected such that a determined proportion of subjects having a cancer exhibits a genetic variant or tumor marker in one or more different genes in the panel. The panel may be selected to limit a region for sequencing to a fixed number of base pairs. The panel may be selected to sequence a desired amount of DNA. The panel may be further selected to achieve a desired sequence read depth. The panel may be selected to achieve a desired sequence read depth or sequence read coverage for an amount of sequenced base pairs. The panel may be selected to achieve a theoretical sensitivity, a theoretical specificity, and/or a theoretical accuracy for detecting one or more genetic variants in a sample.

Genes included in this panel may comprise one or more of: ATM, ATR, BAP1, BARD1, BRCA1, BRCA2, BRIP1, CDK12, CHEK1, CHEK2, FANCA, FANCL, HDAC2, MRE11, NBN, PALB2, RAD50, RAD51, RAD51B, RAD51C, RAD51D, RAD54L, XRCC2, XRCC3 DNMT3A, TP53, LRP1B, KRAS, MARCH11, TAC1, TCF21, SHOX2, p16, Casp8, CDH13, MGMT, MLH1, MSH2, TSLC1, APC, DKK1, DKK3, LKB1, WIF1, RUNX3, GATA4, GATA5, PAX5, E-Cadherin, H-Cadherin, VIM, SEPT9, CYCD2, TFPI2, GATA4, RARB2, p16INK4a, APC, NDRG4, HLTF, HPP1, hMLH1, RASSF1A, IGFBP3, ITGA4, PIK3CA, ERBB2 (HER2), BRCA1/2, NTRK1/2/3, MSI-High, ESR1, ATM, HRR, FGFR2/3, IDH1, KRAS, NRAS, BRAF, KIT, PDGFRA, EGFR, ALK, ROS1, METL TMB, or RET.

Probes for detecting the panel of regions can include those for detecting genomic regions of interest (hotspot regions) as well as nucleosome-aware probes (e.g., KRAS codons 12 and 13) and may be designed to optimize capture based on analysis of cfDNA coverage and fragment size variation impacted by nucleosome binding patterns and GC sequence composition. Regions used herein can also include non-hotspot regions optimized based on nucleosome positions and GC models. The panel can comprise a plurality of subpanels, including subpanels for identifying tissue of origin (e.g., use of published literature to define 50-100 baits representing genes with most diverse transcription profile across tissues (not necessarily promoters)), whole genome scaffold (e.g., for identifying ultra-conservative genomic content and tiling sparsely across chromosomes with handful of probes for copy number base lining purposes), transcription start site (TSS)/CpG islands (e.g., for capturing differential methylated regions (e.g., Differentially Methylated Regions (DMRs)) in for example in promoters of tumor suppressor genes (e.g., SEPT9/VIM in colorectal cancer)). In some embodiments, markers for a tissue of origin are tissue-specific epigenetic markers.

Some examples of listings of genomic locations of interest may be found in Table 1 and Table 2. In some embodiments, genomic locations used in the methods of the present disclosure comprise at least a portion of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or 97 of the genes of Table 1. In some embodiments, genomic locations used in the methods of the present disclosure comprise at least 5, at least 10, at least 15, at least 20, at least 25, at least 30, at least 35, at least 40, at least 45, at least 50, at least 55, at least 60, at least 65, or 70 of the SNVs of Table 1. In some embodiments, genomic locations used in the methods of the present disclosure comprise at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the CNVs of Table 1. In some embodiments, genomic locations used in the methods of the present disclosure comprise at least 1, at least 2, at least 3, at least 4, at least 5, or 6 of the fusions of Table 1. In some embodiments, genomic locations used in the methods of the present disclosure comprise at least a portion of at least 1, at least 2, or 3 of the indels of Table 1. In some embodiments, genomic locations used in the methods of the present disclosure comprise at least a portion of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, or 115 of the genes of Table 2. In some embodiments, genomic locations used in the methods of the present disclosure comprise at least 5, at least 10, at least 15, at least 20, at least 25, at least 30, at least 35, at least 40, at least 45, at least 50, at least 55, at least 60, at least 65, at least 70, or 73 of the SNVs of Table 2. In some embodiments, genomic locations used in the methods of the present disclosure comprise at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the CNVs of Table 2. In some embodiments, genomic locations used in the methods of the present disclosure comprise at least 1, at least 2, at least 3, at least 4, at least 5, or 6 of the fusions of Table 2. In some embodiments, genomic locations used in the methods of the present disclosure comprise at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the indels of Table 2. Each of these genomic locations of interest may be identified as a backbone region or hot-spot region for a given bait set panel. An example of a listing of hot-spot genomic locations of interest may be found in Table 3. In some embodiments, genomic locations used in the methods of the present disclosure comprise at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 of the genes of Table 3. Each hot-spot genomic location is listed with several characteristics, including the associated gene, chromosome on which it resides, the start and stop position of the genome representing the gene's locus, the length of the gene's locus in base pairs, the exons covered by the gene, and the critical feature (e.g., type of mutation) that a given genomic location of interest may seek to capture.

TABLE 1

| | | | | | | Amplifications | | | |
|---|---|---|---|---|---|---|---|---|---|
| Point Mutations (SNVs) | | | | | | (CNVs) | | Fusions | Indels |
| AKT1 | ALK | APC | AR | ARAF | ARID1A | AR | BRAF | ALK | EGFR |
| ATM | BRAF | BRCA1 | BRCA2 | CCND1 | CCND2 | CCND1 | CCND2 | FGFR2 | (exons |
| CCNE1 | CDH1 | CDK4 | CDK6 | CDKN2A | CDKN2B | CCNE1 | CDK4 | FGFR3 | 19 & 20) |
| CTNNB1 | EGFR | ERBB2 | ESR1 | EZH2 | FBXW7 | CDK6 | EGFR | NTRK1 | ERBB2 |
| FGFR1 | FGFR2 | FGFR3 | GATA3 | GNA11 | GNAQ | ERBB2 | FGFR1 | RET | (exons |
| GNAS | HNF1A | HRAS | IDH1 | IDH2 | JAK2 | FGFR2 | KIT | ROS1 | 19 & 20) |
| JAK3 | KIT | KRAS | MAP2K1 | MAP2K2 | MET | KRAS | MET | | MET |
| MLH1 | MPL | MYC | NF1 | NFE2L2 | NOTCH1 | MYC | PDGFRA | | (exon 14 |
| NPM1 | NRAS | NTRK1 | PDGFRA | PIK3CA | PTEN | PIK3CA | RAF1 | | skipping) |
| PTPN11 | RAF1 | RB1 | RET | RHEB | RHOA | | | | |
| RIT1 | ROS1 | SMAD4 | SMO | SRC | STK11 | | | | |
| TERT | TP53 | TSC1 | VHL | | | | | | |

TABLE 2

| | | | | | | Amplifications | | | |
|---|---|---|---|---|---|---|---|---|---|
| Point Mutations (SNVs) | | | | | | (CNVs) | | Fusions | Indels |
| AKT1 | ALK | APC | AR | ARAF | ARID1A | AR | BRAF | ALK | EGFR |
| ATM | BRAF | BRCA1 | BRCA2 | CCND1 | CCND2 | CCND1 | CCND2 | FGFR2 | (exons |
| CCNE1 | CDH1 | CDK4 | CDK6 | CDKN2A | DDR2 | CCNE1 | CDK4 | FGFR3 | 19 & 20) |
| CTNNB1 | EGFR | ERBB2 | ESR1 | EZH2 | FBXW7 | CDK6 | EGFR | NTRK1 | ERBB2 |
| FGFR1 | FGFR2 | FGFR3 | GATA3 | GNA11 | GNAQ | ERBB2 | FGFR1 | RET | (exons |
| GNAS | HNF1A | HRAS | IDH1 | IDH2 | JAK2 | FGFR2 | KIT | ROS1 | 19 & 20) |
| JAK3 | KIT | KRAS | MAP2K1 | MAP2K2 | MET | KRAS | MET | | MET |
| MLH1 | MPL | MYC | NF1 | NFE2L2 | NOTCH1 | MYC | PDGFRA | | (exon 14 |
| NPM1 | NRAS | NTRK1 | PDGFRA | PIK3CA | PTEN | PIK3CA | RAF1 | | skipping) |
| PTPN11 | RAF1 | RB1 | RET | RHEB | RHOA | | | | ATM |
| RIT1 | ROS1 | SMAD4 | SMO | MAPK1 | STK11 | | | | |
| TERT | TP53 | TSC1 | VHL | MAPK3 | MTOR | | | | |
| NTRK3 | | | | | | | | | APC |
| | | | | | | | | | ARID1A |
| | | | | | | | | | BRCA1 |
| | | | | | | | | | BRCA2 |
| | | | | | | | | | CDH1 |
| | | | | | | | | | CDKN2A |
| | | | | | | | | | GATA3 |
| | | | | | | | | | KIT |
| | | | | | | | | | MLH1 |
| | | | | | | | | | MTOR |
| | | | | | | | | | NF1 |
| | | | | | | | | | PDGFRA |
| | | | | | | | | | PTEN |
| | | | | | | | | | RB1 |
| | | | | | | | | | SMAD4 |
| | | | | | | | | | STK11 |
| | | | | | | | | | TP53 |
| | | | | | | | | | TSC1 |
| | | | | | | | | | VHL |

TABLE 3

| | | Start | Stop | | Exons | Critical |
|---|---|---|---|---|---|---|
| Gene | Chromosome | Position | Position | Length (bp) | Covered | Feature |
| ALK | chr2 | 29446405 | 29446655 | 250 | intron 19 | Fusion |
| ALK | chr2 | 29446062 | 29446197 | 135 | intron 20 | Fusion |
| ALK | chr2 | 29446198 | 29446404 | 206 | 20 | Fusion |
| ALK | chr2 | 29447353 | 29447473 | 120 | intron 19 | Fusion |
| ALK | chr2 | 29447614 | 29448316 | 702 | intron 19 | Fusion |
| ALK | chr2 | 29448317 | 29448441 | 124 | 19 | Fusion |
| ALK | chr2 | 29449366 | 29449777 | 411 | intron 18 | Fusion |
| ALK | chr2 | 29449778 | 29449950 | 172 | 18 | Fusion |
| BRAF | chr7 | 140453064 | 140453203 | 139 | 15 | BRAF V600 |
| CTNNB1 | chr3 | 41266007 | 41266254 | 247 | 3 | S37 |
| EGFR | chr7 | 55240528 | 55240827 | 299 | 18 and 19 | G719 and |
| | | | | | | deletions |
| EGFR | chr7 | 55241603 | 55241746 | 143 | 20 | Insertions/T790M |
| EGFR | chr7 | 55242404 | 55242523 | 119 | 21 | L858R |
| ERBB2 | chr17 | 37880952 | 37881174 | 222 | 20 | Insertions |
| ESR1 | chr6 | 152419857 | 152420111 | 254 | 10 | V534, P535, |
| | | | | | | L536, Y537, |
| | | | | | | D538 |
| FGFR2 | chr10 | 123279482 | 123279693 | 211 | 6 | S252 |
| GATA3 | chr10 | 8111426 | 8111571 | 145 | 5 | SS/Indels |
| GATA3 | chr10 | 8115692 | 8116002 | 310 | 6 | SS/Indels |
| GNAS | chr20 | 57484395 | 57484488 | 93 | 8 | R844 |
| IDH1 | chr2 | 209113083 | 209113394 | 311 | 4 | R132 |
| IDH2 | chr15 | 90631809 | 90631989 | 180 | 4 | R140, R172 |
| KIT | chr4 | 55524171 | 55524258 | 87 | 1 | |
| KIT | chr4 | 55561667 | 55561957 | 290 | 2 | |
| KIT | chr4 | 55564439 | 55564741 | 302 | 3 | |
| KIT | chr4 | 55565785 | 55565942 | 157 | 4 | |
| KIT | chr4 | 55569879 | 55570068 | 189 | 5 | |
| KIT | chr4 | 55573253 | 55573463 | 210 | 6 | |
| KIT | chr4 | 55575579 | 55575719 | 140 | 7 | |
| KIT | chr4 | 55589739 | 55589874 | 135 | 8 | |
| KIT | chr4 | 55592012 | 55592226 | 214 | 9 | |
| KIT | chr4 | 55593373 | 55593718 | 345 | 10 and 11 | 557, 559, 560, |
| | | | | | | 576 |
| KIT | chr4 | 55593978 | 55594297 | 319 | 12 and 13 | V654 |
| KIT | chr4 | 55595490 | 55595661 | 171 | 14 | T670, S709 |
| KIT | chr4 | 55597483 | 55597595 | 112 | 15 | D716 |
| KIT | chr4 | 55598026 | 55598174 | 148 | 16 | L783 |
| KIT | chr4 | 55599225 | 55599368 | 143 | 17 | C809, R815, |
| | | | | | | D816, L818, |
| | | | | | | D820, S821F, |
| | | | | | | N822, Y823 |
| KIT | chr4 | 55602653 | 55602785 | 132 | 18 | A829P |
| KIT | chr4 | 55602876 | 55602996 | 120 | 19 | |
| KIT | chr4 | 55603330 | 55603456 | 126 | 20 | |
| KIT | chr4 | 55604584 | 55604733 | 149 | 21 | |
| KRAS | chr12 | 25378537 | 25378717 | 180 | 4 | A146 |
| KRAS | chr12 | 25380157 | 25380356 | 199 | 3 | Q61 |
| KRAS | chr12 | 25398197 | 25398328 | 131 | 2 | G12/G13 |
| MET | chr7 | 116411535 | 116412255 | 720 | 13, 14, | MET exon 14 |
| | | | | | intron 13, | SS |
| | | | | | intron 14 | |
| NRAS | chr1 | 115256410 | 115256609 | 199 | 3 | Q61 |
| NRAS | chr1 | 115258660 | 115258791 | 131 | 2 | G12/G13 |
| PIK3CA | chr3 | 178935987 | 178936132 | 145 | 10 | E545K |
| PIK3CA | chr3 | 178951871 | 178952162 | 291 | 21 | H1047R |
| PTEN | chr10 | 89692759 | 89693018 | 259 | 5 | R130 |
| SMAD4 | chr18 | 48604616 | 48604849 | LR methylation | 12 | D537 |
| | | | | component 233 | | |
| TERT | chr5 | 1294841 | 1295512 | 671 | promoter | chr5: 1295228 |
| TP53 | chr17 | 7573916 | 7574043 | 127 | 11 | Q331, R337, |
| | | | | | | R342 |
| TP53 | chr17 | 7577008 | 7577165 | 157 | 8 | R273 |
| TP53 | chr17 | 7577488 | 7577618 | 130 | 7 | R248 |
| TP53 | chr17 | 7578127 | 7578299 | 172 | 6 | R213/Y220 |
| TP53 | chr17 | 7578360 | 7578564 | 204 | 5 | R175/ |
| | | | | | | Deletions |
| TP53 | chr17 | 7579301 | 7579600 | 299 | 4 | |
| | | | | 12574 | | |
| | | | | (total target region) | | |
| | | | | 16330 (total probe | | |
| | | | | coverage) | | |

In some embodiments, the one or more regions in the panel comprise one or more loci from one or a plurality of genes for detecting residual cancer after surgery. This detection can be earlier than is possible for existing methods of cancer detection. In some embodiments, the one or more genomic locations in the panel comprise one or more loci from one or a plurality of genes for detecting cancer in a high-risk patient population. For example, smokers have much higher rates of lung cancer than the general population. Moreover, smokers can develop other lung conditions that make cancer detection more difficult, such as the development of irregular nodules in the lungs. In some embodiments, the methods described herein detect cancer in high risk patients earlier than is possible for existing methods of cancer detection.

A genomic location may be selected for inclusion in a sequencing panel based on a number of subjects with a cancer that have a tumor marker in that gene or region. A genomic location may be selected for inclusion in a sequencing panel based on prevalence of subjects with a cancer and a tumor marker present in that gene. Presence of a tumor marker in a region may be indicative of a subject having cancer.

In some instances, the panel may be selected using information from one or more databases. The information regarding a cancer may be derived from cancer tumor biopsies or cfDNA assays. A database may comprise information describing a population of sequenced tumor samples. A database may comprise information about mRNA expression in tumor samples. A databased may comprise information about regulatory elements or genomic regions in tumor samples. The information relating to the sequenced tumor samples may include the frequency various genetic variants and describe the genes or regions in which the genetic variants occur. The genetic variants may be tumor markers. A non-limiting example of such a database is COSMIC. COSMIC is a catalogue of somatic mutations found in various cancers. For a particular cancer, COSMIC ranks genes based on frequency of mutation. A gene may be selected for inclusion in a panel by having a high frequency of mutation within a given gene. For instance, COSMIC indicates that 33% of a population of sequenced breast cancer samples have a mutation in TP53 and 22% of a population of sampled breast cancers have a mutation in KRAS. Other ranked genes, including APC, have mutations found only in about 4% of a population of sequenced breast cancer samples. TP53 and KRAS may be included in a sequencing panel based on having relatively high frequency among sampled breast cancers (compared to APC, for example, which occurs at a frequency of about 4%). COSMIC is provided as a non-limiting example, however, any database or set of information may be used that associates a cancer with tumor marker located in a gene or genetic region. In another example, as provided by COSMIC, of 1156 biliary tract cancer samples, 380 samples (33%) carried mutations in TP53. Several other genes, such as APC, have mutations in 4-8% of all samples. Thus, TP53 may be selected for inclusion in the panel based on a relatively high frequency in a population of biliary tract cancer samples.

A gene or genomic section may be selected for a panel where the frequency of a tumor marker is significantly greater in sampled tumor tissue or circulating tumor DNA than found in a given background population. A combination of genomic locations may be selected for inclusion of a panel such that at least a majority of subjects having a cancer may have a tumor marker or genomic region present in at least one of the genomic location or genes in the panel. The combination of genomic location may be selected based on data indicating that, for a particular cancer or set of cancers, a majority of subjects have one or more tumor markers in one or more of the selected regions. For example, to detect cancer 1, a panel comprising regions A, B, C, and/or D may be selected based on data indicating that 90% of subjects with cancer 1 have a tumor marker in regions A, B, C, and/or D of the panel. Alternately, tumor markers may be shown to occur independently in two or more regions in subjects having a cancer such that, combined, a tumor marker in the two or more regions is present in a majority of a population of subjects having a cancer. For example, to detect cancer 2, a panel comprising regions X, Y, and Z may be selected based on data indicating that 90% of subjects have a tumor marker in one or more regions, and in 30% of such subjects a tumor marker is detected only in region X, while tumor markers are detected only in regions Y and/or Z for the remainder of the subjects for whom a tumor marker was detected. Tumor markers present in one or more genomic locations previously shown to be associated with one or more cancers may be indicative of or predictive of a subject having cancer if a tumor marker is detected in one or more of those regions 50% or more of the time. Computational approaches such as models employing conditional probabilities of detecting cancer given a cancer frequency for a set of tumor markers within one or more regions may be used to predict which regions, alone or in combination, may be predictive of cancer. Other approaches for panel selection involve the use of databases describing information from studies employing comprehensive genomic profiling of tumors with large panels and/or whole genome sequencing (WGS, RNA-seq, Chip-seq, bisulfate sequencing, ATAC-seq, and others). Information gleaned from literature may also describe pathways commonly affected and mutated in certain cancers. Panel selection may be further informed by the use of ontologies describing genetic information.

Genes included in the panel for sequencing can include the fully transcribed region, the promoter region, enhancer regions, regulatory elements, and/or downstream sequence. To further increase the likelihood of detecting tumor indicating mutations only exons may be included in the panel. The panel can comprise all exons of a selected gene, or only one or more of the exons of a selected gene. The panel may comprise of exons from each of a plurality of different genes. The panel may comprise at least one exon from each of the plurality of different genes.

In some aspects, a panel of exons from each of a plurality of different genes is selected such that a determined proportion of subjects having a cancer exhibit a genetic variant in at least one exon in the panel of exons.

At least one full exon from each different gene in a panel of genes may be sequenced. The sequenced panel may comprise exons from a plurality of genes. The panel may comprise exons from 2 to 100 different genes, from 2 to 70 genes, from 2 to 50 genes, from 2 to 30 genes, from 2 to 15 genes, or from 2 to 10 genes.

A selected panel may comprise a varying number of exons. The panel may comprise from 2 to 3000 exons. The panel may comprise from 2 to 1000 exons. The panel may comprise from 2 to 500 exons. The panel may comprise from 2 to 100 exons. The panel may comprise from 2 to 50 exons. The panel may comprise no more than 300 exons. The panel may comprise no more than 200 exons. The panel may comprise no more than 100 exons. The panel may comprise no more than 50 exons. The panel may comprise no more than 40 exons. The panel may comprise no more than 30 exons. The panel may comprise no more than 25 exons. The panel may comprise no more than 20 exons. The panel may comprise no more than 15 exons. The panel may comprise no more than 10 exons. The panel may comprise no more than 9 exons. The panel may comprise no more than 8 exons. The panel may comprise no more than 7 exons.

The panel may comprise one or more exons from a plurality of different genes. The panel may comprise one or more exons from each of a proportion of the plurality of different genes. The panel may comprise at least two exons from each of at least 25%, 50%, 75% or 90% of the different genes. The panel may comprise at least three exons from each of at least 25%, 50%, 75% or 90% of the different genes. The panel may comprise at least four exons from each of at least 25%, 50%, 75% or 90% of the different genes.

The sizes of the sequencing panel may vary. A sequencing panel may be made larger or smaller (in terms of nucleotide size) depending on several factors including, for example, the total amount of nucleotides sequenced or a number of unique molecules sequenced for a particular region in the panel. The sequencing panel can be sized 5 kb to 50 kb. The sequencing panel can be 10 kb to 30 kb in size. The sequencing panel can be 12 kb to 20 kb in size. The sequencing panel can be 12 kb to 60 kb in size. The sequencing panel can be at least 10 kb, 12 kb, 15 kb, 20 kb, 25 kb, 30 kb, 35 kb, 40 kb, 45 kb, 50 kb, 60 kb, 70 kb, 80 kb, 90 kb, 100 kb, 110 kb, 120 kb, 130 kb, 140 kb, or 150 kb in size. The sequencing panel may be less than 100 kb, 90 kb, 80 kb, 70 kb, 60 kb, or 50 kb in size.

The panel selected for sequencing can comprise at least 1, 5, 10, 15, 20, 25, 30, 40, 50, 60, 80, or 100 genomic locations (e.g., that each include genomic regions of interest). In some cases, the genomic locations in the panel are selected that the size of the locations are relatively small. In some cases, the regions in the panel have a size of about 10 kb or less, about 8 kb or less, about 6 kb or less, about 5 kb or less, about 4 kb or less, about 3 kb or less, about 2.5 kb or less, about 2 kb or less, about 1.5 kb or less, or about 1 kb or less or less. In some cases, the genomic locations in the panel have a size from about 0.5 kb to about 10 kb, from about 0.5 kb to about 6 kb, from about 1 kb to about 11 kb, from about 1 kb to about 15 kb, from about 1 kb to about 20 kb, from about 0.1 kb to about 10 kb, or from about 0.2 kb to about 1 kb. For example, the regions in the panel can have a size from about 0.1 kb to about 5 kb.

The panel selected herein can allow for deep sequencing that is sufficient to detect low-frequency genetic variants (e.g., in cell-free nucleic acid molecules obtained from a sample). An amount of genetic variants in a sample may be referred to in terms of the minor allele frequency for a given genetic variant. The minor allele frequency may refer to the frequency at which minor alleles (e.g., not the most common allele) occurs in a given population of nucleic acids, such as a sample. Genetic variants at a low minor allele frequency may have a relatively low frequency of presence in a sample. In some cases, the panel allows for detection of genetic variants at a minor allele frequency of at least 0.0001%, 0.001%, 0.005%, 0.01%, 0.05%, 0.1%, or 0.5%. The panel can allow for detection of genetic variants at a minor allele frequency of 0.001% or greater. The panel can allow for detection of genetic variants at a minor allele frequency of 0.01% or greater. The panel can allow for detection of genetic variant present in a sample at a frequency of as low as 0.0001%, 0.001%, 0.005%, 0.01%, 0.025%, 0.05%, 0.075%, 0.1%, 0.25%, 0.5%, 0.75%, or 1.0%. The panel can allow for detection of tumor markers present in a sample at a frequency of at least 0.0001%, 0.001%, 0.005%, 0.01%, 0.025%, 0.05%, 0.075%, 0.1%, 0.25%, 0.5%, 0.75%, or 1.0%. The panel can allow for detection of tumor markers at a frequency in a sample as low as 1.0%. The panel can allow for detection of tumor markers at a frequency in a sample as low as 0.75%. The panel can allow for detection of tumor markers at a frequency in a sample as low as 0.5%. The panel can allow for detection of tumor markers at a frequency in a sample as low as 0.25%. The panel can allow for detection of tumor markers at a frequency in a sample as low as 0.1%. The panel can allow for detection of tumor markers at a frequency in a sample as low as 0.075%. The panel can allow for detection of tumor markers at a frequency in a sample as low as 0.05%. The panel can allow for detection of tumor markers at a frequency in a sample as low as 0.025%. The panel can allow for detection of tumor markers at a frequency in a sample as low as 0.01%. The panel can allow for detection of tumor markers at a frequency in a sample as low as 0.005%. The panel can allow for detection of tumor markers at a frequency in a sample as low as 0.001%. The panel can allow for detection of tumor markers at a frequency in a sample as low as 0.0001%. The panel can allow for detection of tumor markers in sequenced cfDNA at a frequency in a sample as low as 1.0% to 0.0001%. The panel can allow for detection of tumor markers in sequenced cfDNA at a frequency in a sample as low as 0.01% to 0.0001%.

A genetic variant can be exhibited in a percentage of a population of subjects who have a disease (e.g., cancer). In some cases, at least 1%, 2%, 3%, 5%, 10%, 20%, 30%, 40%, 50%, 60%, 70%, 80%, 90%, 95%, or 99% of a population having the cancer exhibit one or more genetic variants in at least one of the regions in the panel. For example, at least 80% of a population having the cancer may exhibit one or more genetic variants in at least one of the genomic positions in the panel.

The panel can comprise one or more locations comprising genomic regions of interest from each of one or more genes. In some cases, the panel can comprise one or more locations comprising genomic regions of interest from each of at least 1, 2, 3, 4, 5, 6, 7, 8, 9, 10, 15, 20, 25, 30, 40, 50, or 80 genes. In some cases, the panel can comprise one or more locations comprising genomic regions of interest from each of at most 1, 2, 3, 4, 5, 6, 7, 8, 9, 10, 15, 20, 25, 30, 40, 50, or 80 genes. In some cases, the panel can comprise one or more locations comprising genomic regions of interest from each of from about 1 to about 80, from 1 to about 50, from about 3 to about 40, from 5 to about 30, from 10 to about 20 different genes.

The locations comprising genomic regions in the panel can be selected so that one or more epigenetically modified regions are detected. The one or more epigenetically modified regions can be acetylated, methylated, ubiquitylated, phosphorylated, sumoylated, ribosylated, and/or citrullinated. For example, the regions in the panel can be selected so that one or more methylated regions are detected. In some embodiments, a genomic region of the panel may comprise one or more of the following genes: DNMT3A, TP53, LRP1B, KRAS, MARCH11, TAC1, TCF21, SHOX2, p16, Casp8, CDH13, MGMT, MLH1, MSH2, TSLC1, APC, DKK1, DKK3, LKB1, WIF1, RUNX3, GATA4, GATA5, PAX5, E-Cadherin, H-Cadherin, VIM, SEPT9, CYCD2, TFPI2, GATA4, RARB2, p16INK4a, APC, NDRG4, HLTF, HPP1, hMLH1, RASSF1A, IGFBP3, ITGA4, PIK3CA, ERBB2 (HER2), BRCA1/2, NTRK1/2/3, MSI-High, ESR1, ATM, HRR, FGFR2/3, IDH1, KRAS, NRAS, BRAF, KIT, PDGFRA, EGFR, ALK, ROS1, MET, TMB, or RET.

The regions in the panel can be selected so that they comprise sequences differentially transcribed across one or more tissues. In some cases, the locations comprising genomic regions can comprise sequences transcribed in certain tissues at a higher level compared to other tissues. For example, the locations comprising genomic regions can comprise sequences transcribed in certain tissues but not in other tissues.

The genomic locations in the panel can comprise coding and/or non-coding sequences. For example, the genomic locations in the panel can comprise one or more sequences in exons, introns, promoters, 3′ untranslated regions, 5′ untranslated regions, regulatory elements, transcription start sites, and/or splice sites. In some cases, the regions in the panel can comprise other non-coding sequences, including pseudogenes, repeat sequences, transposons, viral elements, and telomeres. In some cases, the genomic locations in the panel can comprise sequences in non-coding RNA, e.g., ribosomal RNA, transfer RNA, Piwi-interacting RNA, orphan-non coding RNA and microRNA.

The genomic locations in the panel can be selected to detect (diagnose) a cancer with a desired level of sensitivity (e.g., through the detection of one or more genetic variants). For example, the regions in the panel can be selected to detect the cancer (e.g., through the detection of one or more genetic variants) with a sensitivity of at least 50%, 55%, 60%, 65%, 70%, 75%, 80%, 85%, 90%, 95%, 96%, 97%, 98%, 99%, 99.5%, or 99.9%. The genomic locations in the panel can be selected to detect the cancer with a sensitivity of 100%.

The genomic locations in the panel can be selected to detect (diagnose) a cancer with a desired level of specificity (e.g., through the detection of one or more genetic variants). For example, the genomic locations in the panel can be selected to detect cancer (e.g., through the detection of one or more genetic variants) with a specificity of at least 50%, 55%, 60%, 65%, 70%, 75%, 80%, 85%, 90%, 95%, 96%, 97%, 98%, 99%, 99.5%, or 99.9%. The genomic locations in the panel can be selected to detect the one or more genetic variant with a specificity of 100%.

The genomic locations in the panel can be selected to detect (diagnose) a cancer with a desired positive predictive value. Positive predictive value can be increased by increasing sensitivity (e.g., chance of an actual positive being detected) and/or specificity (e.g., chance of not mistaking an actual negative for a positive). As a non-limiting example, genomic locations in the panel can be selected to detect the one or more genetic variant with a positive predictive value of at least 50%, 55%, 60%, 65%, 70%, 75%, 80%, 85%, 90%, 95%, 96%, 97%, 98%, 99%, 99.5%, or 99.9%. The regions in the panel can be selected to detect the one or more genetic variant with a positive predictive value of 100%.

The genomic locations in the panel can be selected to detect (diagnose) a cancer with a desired accuracy. As used herein, the term “accuracy” may refer to the ability of a test to discriminate between a disease condition (e.g., cancer) and healthy condition. Accuracy may be can be quantified using measures such as sensitivity and specificity, predictive values, likelihood ratios, the area under the ROC curve, Youden's index and/or diagnostic odds ratio.

Accuracy may presented as a percentage, which refers to a ratio between the number of tests giving a correct result and the total number of tests performed. The regions in the panel can be selected to detect cancer with an accuracy of at least 50%, 55%, 60%, 65%, 70%, 75%, 80%, 85%, 90%, 95%, 96%, 97%, 98%, 99%, 99.5%, or 99.9%. The genomic locations in the panel can be selected to detect cancer with an accuracy of 100%.

A panel may be selected to be highly sensitive and detect low frequency genetic variants. For instance, a panel may be selected such that a genetic variant or tumor marker present in a sample at a frequency as low as 0.01%, 0.05%, or 0.001% may be detected at a sensitivity of at least 50%, 55%, 60%, 65%, 70%, 75%, 80%, 85%, 90%, 95%, 96%, 97%, 98%, 99%, 99.5%, or 99.9%. Genomic locations in a panel may be selected to detect a tumor marker present at a frequency of 1% or less in a sample with a sensitivity of 70% or greater. A panel may be selected to detect a tumor marker at a frequency in a sample as low as 0.1% with a sensitivity of at least 50%, 55%, 60%, 65%, 70%, 75%, 80%, 85%, 90%, 95%, 96%, 97%, 98%, 99%, 99.5%, or 99.9%. A panel may be selected to detect a tumor marker at a frequency in a sample as low as 0.01% with a sensitivity of at least 50%, 55%, 60%, 65%, 70%, 75%, 80%, 85%, 90%, 95%, 96%, 97%, 98%, 99%, 99.5%, or 99.9%. A panel may be selected to detect a tumor marker at a frequency in a sample as low as 0.001% with a sensitivity of at least 50%, 55%, 60%, 65%, 70%, 75%, 80%, 85%, 90%, 95%, 96%, 97%, 98%, 99%, 99.5%, or 99.9%.

A panel may be selected to be highly specific and detect low frequency genetic variants. For instance, a panel may be selected such that a genetic variant or tumor marker present in a sample at a frequency as low as 0.01%, 0.05%, or 0.001% may be detected at a specificity of at least 50%, 55%, 60%, 65%, 70%, 75%, 80%, 85%, 90%, 95%, 96%, 97%, 98%, 99%, 99.5%, or 99.9%. Genomic locations in a panel may be selected to detect a tumor marker present at a frequency of 1% or less in a sample with a specificity of 70% or greater. A panel may be selected to detect a tumor marker at a frequency in a sample as low as 0.1% with a specificity of at least 70%, 75%, 80%, 85%, 90%, 95%, 96%, 97%, 98%, 99%, 99.5%, or 99.9%. A panel may be selected to detect a tumor marker at a frequency in a sample as low as 0.01% with a specificity of at least 70%, 75%, 80%, 85%, 90%, 95%, 96%, 97%, 98%, 99%, 99.5%, or 99.9%. A panel may be selected to detect a tumor marker at a frequency in a sample as low as 0.001% with a specificity of at least 70%, 75%, 80%, 85%, 90%, 95%, 96%, 97%, 98%, 99%, 99.5%, or 99.9%.

A panel may be selected to be highly accurate and detect low frequency genetic variants. A panel may be selected such that a genetic variant or tumor marker present in a sample at a frequency as low as 0.01%, 0.05%, or 0.001% may be detected at an accuracy of at least 70%, 75%, 80%, 85%, 90%, 95%, 96%, 97%, 98%, 99%, 99.5%, or 99.9%. Genomic locations in a panel may be selected to detect a tumor marker present at a frequency of 1% or less in a sample with an accuracy of 70% or greater. A panel may be selected to detect a tumor marker at a frequency in a sample as low as 0.1% with an accuracy of at least 70%, 75%, 80%, 85%, 90%, 95%, 96%, 97%, 98%, 99%, 99.5%, or 99.9%. A panel may be selected to detect a tumor marker at a frequency in a sample as low as 0.01% with an accuracy of at least 70%, 75%, 80%, 85%, 90%, 95%, 96%, 97%, 98%, 99%, 99.5%, or 99.9%. A panel may be selected to detect a tumor marker at a frequency in a sample as low as 0.001% with an accuracy of at least 70%, 75%, 80%, 85%, 90%, 95%, 96%, 97%, 98%, 99%, 99.5%, or 99.9%.

A panel may be selected to be highly predictive and detect low frequency genetic variants. A panel may be selected such that a genetic variant or tumor marker present in a sample at a frequency as low as 0.01%, 0.05%, or 0.001% may have a positive predictive value of at least 70%, 75%, 80%, 85%, 90%, 95%, 96%, 97%, 98%, 99%, 99.5%, or 99.9%.

The concentration of probes or baits used in the panel may be increased (2 to 6 ng/μL) to capture more nucleic acid molecule within a sample. The concentration of probes or baits used in the panel may be at least 2 ng/μL, 3 ng/μL, 4 ng/μL, 5 ng/μL, 6 ng/μL, or greater. The concentration of probes may be about 2 ng/μL to about 3 ng/μL, about 2 ng/μL to about 4 ng/μL, about 2 ng/μL to about 5 ng/μL, about 2 ng/μL to about 6 ng/μL. The concentration of probes or baits used in the panel may be 2 ng/μL or more to 6 ng/μL or less. In some instances this may allow for more molecules within a biological to be analyzed thereby enabling lower frequency alleles to be detected.

In an embodiment, utilizing the sequencing pipeline 205, the panel may be subjected to one or more of: whole-genome bisulfite sequencing (WGBS) interrogating genome-wide methylation patterns, whole-genome sequencing (WGS), and/or targeted sequencing approaches interrogating copy-number variants (CNVs) and single-nucleotide variants (SNVs).

Genetic and/or epigenetic information obtained from DNA of the subject can be combined to provide a determination of whether a subject has a cancer or a likelihood that the subject has a cancer. Detailed descriptions of how to analyze cell free human DNA for both genetic and epigenetic variants associated with cancer can be found in U.S. provisional patent application 62/799,637, which is herein incorporated by reference in its entirety. Additional guidance for analyzing cell free DNA for the detecting cancer can be found in, among other places U.S. Pat. No. 9,834,822, PCT application WO2018064629A1, and PCT application WO2017106768A1.

Various embodiments include the step of sequencing DNA (e.g., cfDNA) for the purpose of detecting genetic variants in genes associated with cancer. Various embodiments also include the step of sequencing DNA (e.g., cfDNA) for the purpose of detecting epigenetic variants in genes associated with cancer, for example, but not limited to, include DNA sequences that are differentially methylated in cancerous and noncancerous cells and nucleosomal fragmentation patterns such as those described in US published patent application US2017/0211143.

In some embodiments, a captured set of nucleic acid, e.g., comprising DNA (such as cfDNA) is provided. With respect to the disclosed methods, the captured set of DNA may be provided, e.g., following capturing, and/or separating steps as described herein. The captured set may comprise DNA corresponding to one or both of a sequence-variable target region set and an epigenetic target region set. In some embodiments, the captured set comprises DNA corresponding to a sequence-variable target region set, and an epigenetic target region set. In all embodiments described herein involving a sequence-variable target region set and an epigenetic target region set, the sequence-variable target region set comprises regions not present in the epigenetic target region set and vice versa, although in some instances a fraction of the regions may overlap (e.g., a fraction of genomic positions may be represented in both target region sets).

### (A) Methylation Target Region Set

In some embodiments, an epigenetic target region set is captured. The epigenetic target region set may comprise one or more types of target regions likely to differentiate DNA from neoplastic (e.g., tumor or cancer) cells and from healthy cells, e.g., non-neoplastic circulating cells. The epigenetic target region set can be analyzed in various ways, including methods that do not depend on a high degree of accuracy in sequence determination of specific nucleotides within a target. Exemplary types of such regions are discussed in detail herein. In some embodiments, methods according to the disclosure comprise determining whether cfDNA molecules corresponding to the epigenetic target region set comprise or indicate cancer-associated epigenetic modifications (e.g., hypermethylation in one or more hypermethylation variable target regions; one or more perturbations of CTCF binding; and/or one or more perturbations of transcription start sites) and/or copy number variations (e.g., focal amplifications). Such analyses can be conducted by sequencing and require less data (e.g., number of sequence reads or depth of sequencing coverage) than determining the presence or absence of a sequence mutation such as a base substitution, insertion, or deletion. The epigenetic target region set may also comprise one or more control regions, e.g., as described herein.

In some embodiments, the epigenetic target region set has a footprint of at least 100 kb, e.g., at least 200 kb, at least 300 kb, or at least 400 kb. In some embodiments, the epigenetic target region set has a footprint in the range of 100-1000 kb, e.g., 100-200 kb, 200-300 kb, 300-400 kb, 400-500 kb, 500-600 kb, 600-700 kb, 700-800 kb, 800-900 kb, and 900-1,000 kb.

### (B) Hypermethylation Variable Target Regions

In some embodiments, the epigenetic target region set comprises one or more hypermethylation variable target regions. In general, hypermethylation variable target regions refer to regions where an increase in the level of observed methylation indicates an increased likelihood that a sample (e.g., of cfDNA) contains DNA produced by neoplastic cells, such as tumor or cancer cells. For example, hypermethylation of promoters of tumor suppressor genes has been observed repeatedly. See, e.g., Kang et al., Genome Biol. 18:53 (2017) and references cited therein.

An extensive discussion of methylation variable target regions in colorectal cancer is provided in Lam et al., Biochim Biophys Acta. 1866:106-20 (2016). These include VIM, SEPT9, ITGA4, OSM4, GATA4 and NDRG4. An exemplary set of hypermethylation variable target regions comprising the genes or portions thereof based on the colorectal cancer (CRC) studies is provided in Table 4. Many of these genes likely have relevance to cancers beyond colorectal cancer; for example, TP53 is widely recognized as a critically important tumor suppressor and hypermethylation-based inactivation of this gene may be a common oncogenic mechanism.

TABLE 4
Exemplary hypermethylation target regions (genes
or portions thereof) based on CRC studies.

| | Additional | |
|---|---|---|
| Gene Name | Gene Name | Chromosome |
| VIM | | chr10 |
| SEPT9 | | chr17 |
| CYCD2 | CCND2 | chr12 |
| TFPI2 | | chr7 |
| GATA4 | | chr8 |
| RARB2 | RARB | chr3 |
| p16INK4a | CDKN2A | chr9 |
| MGMT | MGMT | chr10 |
| APC | | chr5 |
| NDRG4 | | chr16 |
| HLTF | | chr3 |
| HPP1 | TMEFF2 | chr2 |
| hMLH1 | MLH1 | chr3 |
| RASSF1A | RASSF1 | chr3 |
| CDH13 | | chr16 |
| IGFBP3 | | chr7 |
| ITGA4 | | chr2 |

In some embodiments, the hypermethylation variable target regions comprise a plurality of genes or portions thereof listed in Table 4, e.g., at least 10%, 20%, 30%, 40%, 50%, 60%, 70%, 80%, 90%, or 100% of the genes or portions thereof listed in Table 4. For example, for each locus included as a target region, there may be one or more probes with a hybridization site that binds between the transcription start site and the stop codon (the last stop codon for genes that are alternatively spliced) of the gene. In some embodiments, the one or more probes bind within 300 bp upstream and/or downstream of the genes or portions thereof listed in Table 4, e.g., within 200 or 100 bp.

Methylation variable target regions in various types of lung cancer are discussed in detail, e.g., in Ooki et al., Clin. Cancer Res. 23:7141-52 (2017); Belinksy, Annu. Rev. Physiol. 77:453-74 (2015); Hulbert et al., Clin. Cancer Res. 23:1998-2005 (2017); Shi et al., BMC Genomics 18:901 (2017); Schneider et al., BMC Cancer. 11:102 (2011); Lissa et al., Transl Lung Cancer Res 5(5):492-504 (2016); Skvortsova et al., Br. J. Cancer. 94(10):1492-1495 (2006); Kim et al., Cancer Res. 61:3419-3424 (2001); Furonaka et al., Pathology International 55:303-309 (2005); Gomes et al., Rev. Port. Pneumol. 20:20-30 (2014); Kim et al., Oncogene. 20:1765-70 (2001); Hopkins-Donaldson et al., Cell Death Differ. 10:356-64 (2003); Kikuchi et al., Clin. Cancer Res. 11:2954-61 (2005); Heller et al., Oncogene 25:959-968 (2006); Licchesi et al., Carcinogenesis. 29:895-904 (2008); Guo et al., Clin. Cancer Res. 10:7917-24 (2004); Palmisano et al., Cancer Res. 63:4620-4625 (2003); and Toyooka et al., Cancer Res. 61:4556-4560, (2001).

An exemplary set of hypermethylation variable target regions comprising genes or portions thereof based on the lung cancer studies is provided in Table 5. Many of these genes likely have relevance to cancers beyond lung cancer; for example, Casp8 (Caspase 8) is a key enzyme in programmed cell death and hypermethylation-based inactivation of this gene may be a common oncogenic mechanism not limited to lung cancer. Additionally, a number of genes appear in both Tables 4 and 5, indicating generality.

TABLE 5
Exemplary hypermethylation target regions (genes
or portions thereof) based on lung cancer studies

| Gene Name | Chromosome |
|---|---|
| MARCH11 | chr5 |
| TAC1 | chr7 |
| TCF21 | chr6 |
| SHOX2 | chr3 |
| p16 | chr3 |
| Casp8 | chr2 |
| CDH13 | chr16 |
| MGMT | chr10 |
| MLH1 | chr3 |
| MSH2 | chr2 |
| TSLC1 | chr11 |
| APC | chr5 |
| DKK1 | chr10 |
| DKK3 | chr11 |
| LKB1 | chr11 |
| WIF1 | chr12 |
| RUNX3 | chr1 |
| GATA4 | chr8 |
| GATA5 | chr20 |
| PAX5 | chr9 |
| E-Cadherin | chr16 |
| H-Cadherin | chr16 |

Any of the foregoing embodiments concerning target regions identified in Table 2 may be combined with any of the embodiments described above concerning target regions identified in Table 1. In some embodiments, the hypermethylation variable target regions comprise a plurality of genes or portions thereof listed in Table 1 or Table 2, e.g., at least 10%, 20%, 30%, 40%, 50%, 60%, 70%, 80%, 90%, or 100% of the genes or portions thereof listed in Table 1 or Table 2.

Additional hypermethylation target regions may be obtained, e.g., from the Cancer Genome Atlas. Kang et al., Genome Biology 18:53 (2017), describe construction of a probabilistic method called Cancer Locator using hypermethylation target regions from breast, colon, kidney, liver, and lung. In some embodiments, the hypermethylation target regions can be specific to one or more types of cancer. Accordingly, in some embodiments, the hypermethylation target regions include one, two, three, four, or five subsets of hypermethylation target regions that collectively show hypermethylation in one, two, three, four, or five of breast, colon, kidney, liver, and lung cancers.

### Hypomethylation Variable Target Regions

Global hypomethylation is a commonly observed phenomenon in various cancers. See, e.g., Hon et al., Genome Res. 22:246-258 (2012) (breast cancer); Ehrlich, Epigenomics 1:239-259 (2009) (review article noting observations of hypomethylation in colon, ovarian, prostate, leukemia, hepatocellular, and cervical cancers). For example, regions such as repeated elements, e.g., LINE1 elements, Alu elements, centromeric tandem repeats, pericentromeric tandem repeats, and satellite DNA, and intergenic regions that are ordinarily methylated in healthy cells may show reduced methylation in tumor cells. Accordingly, in some embodiments, the epigenetic target region set includes hypomethylation variable target regions, where a decrease in the level of observed methylation indicates an increased likelihood that a sample (e.g., of cfDNA) contains DNA produced by neoplastic cells, such as tumor or cancer cells.

In some embodiments, hypomethylation variable target regions include repeated elements and/or intergenic regions. In some embodiments, repeated elements include one, two, three, four, or five of LINE1 elements, Alu elements, centromeric tandem repeats, pericentromeric tandem repeats, and/or satellite DNA.

Exemplary specific genomic regions that show cancer-associated hypomethylation include nucleotides 8403565-8953708 and 151104701-151106035 of human chromosome 1, e.g., according to the hg19 or hg38 human genome construct. In some embodiments, the hypomethylation variable target regions overlap or comprise one or both of these regions.

### (C) CTCF Binding Regions

CTCF is a DNA-binding protein that contributes to chromatin organization and often colocalizes with cohesin. Perturbation of CTCF binding sites has been reported in a variety of different cancers. See, e.g., Katainen et al., Nature Genetics, doi:10.1038/ng.3335, published online 8 Jun. 2015; Guo et al., Nat. Commun. 9:1520 (2018). CTCF binding results in recognizable patterns in cfDNA that can be detected by sequencing, e.g., through fragment length analysis. For example, details regarding sequencing-based fragment length analysis are provided in Snyder et al., Cell 164:57-68 (2016); WO 2018/009723; and US20170211143A1, each of which are incorporated herein by reference.

Thus, perturbations of CTCF binding result in variation in the fragmentation patterns of cfDNA. As such, CTCF binding sites represent a type of fragmentation variable target regions.

There are many known CTCF binding sites. See, e.g., the CTCFBSDB (CTCF Binding Site Database), available on the Internet at insulatordb.uthsc.edu/; Cuddapah et al., Genome Res. 19:24-32 (2009); Martin et al., Nat. Struct. Mol. Biol. 18:708-14 (2011); Rhee et al., Cell. 147:1408-19 (2011), each of which are incorporated by reference. Exemplary CTCF binding sites are at nucleotides 56014955-56016161 on chromosome 8 and nucleotides 95359169-95360473 on chromosome 13, e.g., according to the hg19 or hg38 human genome construct.

Accordingly, in some embodiments, the epigenetic target region set includes CTCF binding regions. In some embodiments, the CTCF binding regions comprise at least 10, 20, 50, 100, 200, or 500 CTCF binding regions, or 10-20, 20-50, 50-100, 100-200, 200-500, or 500-1000 CTCF binding regions, e.g., such as CTCF binding regions described above or in one or more of CTCFBSDB or the Cuddapah et al., Martin et al., or Rhee et al. articles cited above.

In some embodiments, at least some of the CTCF sites can be methylated or unmethylated, wherein the methylation state is correlated with the whether or not the cell is a cancer cell. In some embodiments, the epigenetic target region set comprises at least 100 bp, at least 200 bp, at least 300 bp, at least 400 bp, at least 500 bp, at least 750 bp, at least 1000 bp upstream and/or downstream regions of the CTCF binding sites.

### (D) Transcription Start Sites

Transcription start sites may also show perturbations in neoplastic cells. For example, nucleosome organization at various transcription start sites in healthy cells of the hematopoietic lineage—which contributes substantially to cfDNA in healthy individuals—may differ from nucleosome organization at those transcription start sites in neoplastic cells. This results in different cfDNA patterns that can be detected by sequencing, for example, as discussed generally in Snyder et al., Cell 164:57-68 (2016); WO 2018/009723; and US20170211143A1.

Thus, perturbations of transcription start sites also result in variation in the fragmentation patterns of cfDNA. As such, transcription start sites also represent a type of fragmentation variable target regions.

Human transcriptional start sites are available from DBTSS (DataBase of Human Transcription Start Sites), available on the Internet at dbtss.hgc.jp and described in Yamashita et al., Nucleic Acids Res. 34(Database issue): D86-D89 (2006), which is incorporated herein by reference.

Accordingly, in some embodiments, the epigenetic target region set includes transcriptional start sites. In some embodiments, the transcriptional start sites comprise at least 10, 20, 50, 100, 200, or 500 transcriptional start sites, or 10-20, 20-50, 50-100, 100-200, 200-500, or 500-1000 transcriptional start sites, e.g., such as transcriptional start sites listed in DBTSS. In some embodiments, at least some of the transcription start sites can be methylated or unmethylated, wherein the methylation state is correlated with the whether or not the cell is a cancer cell. In some embodiments, the epigenetic target region set comprises at least 100 bp, at least 200 bp, at least 300 bp, at least 400 bp, at least 500 bp, at least 750 bp, at least 1000 bp upstream and/or downstream regions of the transcription start sites.

### (E) Methylation Control Regions

It can be useful to include control regions to facilitate data validation. In some embodiments, the epigenetic target region set includes control regions that are expected to be methylated or unmethylated in essentially all samples, regardless of whether the DNA is derived from a cancer cell or a normal cell. In some embodiments, the epigenetic target region set includes control hypomethylated regions that are expected to be hypomethylated in essentially all samples. In some embodiments, the epigenetic target region set includes control hypermethylated regions that are expected to be hypermethylated in essentially all samples.

### (F) Copy Number Variations; Focal Amplifications

Although copy number variations such as focal amplifications are somatic mutations, they can be detected by sequencing based on read frequency in a manner analogous to approaches for detecting certain epigenetic changes such as changes in methylation. As such, regions that may show copy number variations such as focal amplifications in cancer can be included in the epigenetic target region set and may comprise one or more of AR, BRAF, CCND1, CCND2, CCNE1, CDK4, CDK6, EGFR, ERBB2, FGFR1, FGFR2, KIT, KRAS, MET, MYC, PDGFRA, PIK3CA, and RAF1. For example, in some embodiments, the epigenetic target region set comprises at least 2, 3, 4, 5, 6, 7, 8, 9, 10, 11, 12, 13, 14, 15, 16, 17, or 18 of the foregoing targets.

vi. Sequence Analysis Pipeline

In an embodiment, after sequencing, sequence reads and any associated data may be stored in the sequence datastore 209. The sequence reads can be stored in any format. The sequence datastore 209 may be local and/or remote to a location where sequencing is performed. As shown in FIG. 2, the stored reads may be subjected to a sequence analysis pipeline 230.

a. Sequence Alignment

The sequence analysis pipeline 230 may include an alignment component 236 that is configured to align sequence fragments/reads from the laboratory system 102 to arrange the sequences of the sequence datastore 209 in order to identify regions of similarity. Similarity may be related to functional, structural, and/or evolutionary relationships between the sequences. For DNA sequences, the alignment by the alignment component 236 may include alignment of genomic DNA of one sequence to genomic DNA of at least one other sequence. Such alignment may exclude non-genomic DNA, such as a molecular barcode, padding bases, and the like. For example, genomic DNA of a sequence read may be aligned to genomic DNA of a reference DNA sequence, excluding any molecular tag that may be attached to the sequence read.

b. Sequence Quality Control

The sequence analysis pipeline 230 may include a sequence quality control (QC) component 231 that may filter sequence fragments/reads from the laboratory system 102. The sequence QC component 231 may assign a quality score to one or more sequence fragments/reads. A quality score may be a representation of sequence fragments/reads that indicates whether those sequence fragments/reads may be useful in subsequent analysis based on a threshold. In some cases, some sequence fragments/reads are not of sufficient quality or length to perform a subsequent mapping step. Sequence fragments/reads with a quality score at least 90%, 95%, 99%, 99.9%, 99.99% or 99.999% may be filtered out of a data set of sequence fragments/reads. In other cases, sequence fragments/reads assigned a quality scored at least 90%, 95%, 99%, 99.9%, 99.99% or 99.999% may be filtered out of the data set.

Sequence fragments/reads that meet a specified quality score threshold may be mapped to a reference genome by the sequence QC component 231. After mapping alignment, sequence fragments/reads may be assigned a mapping score. A mapping score may be a representation of sequence fragments/reads mapped back to the reference sequence indicating whether each position is or is not uniquely mappable. Sequence fragments/reads with a mapping score at least 90%, 95%, 99%, 99.9%, 99.99% or 99.999% may be filtered out of the data set. In other cases, sequencing fragments/reads assigned a mapping scored less than 90%, 95%, 99%, 99.9%, 99.99% or 99.999% may be filtered out of the data set.

c. Epigenetic Factors

Disclosed throughout are epigenetic factors that can be used in the systems and methods herein.

In an embodiment, an epigenetic component 232 may analyze sequence fragments/reads to determine epigenetic data. Epigenetic data may include, for example, information regarding DNA methylation, histone states or modifications, inflammation-mediated cytosine damage products, protein binding, fragmentomics (fragment size, nucleotide motifs at fragment ends, single-stranded jagged ends, and/or genomic locations of fragmentation endpoints), or other molecular states reflected in the nucleic acid fragment analyzed that are not ascertained solely from the nucleotide base sequence. The epigenetic data may be used as an epigenetic signature. Epigenetic data may be determined by any means known in the art. The epigenetic data may be based on fragmentomics data determined by methylation data determined via a LR methylation component 233 and a methylation data determined by a fragmentomics component 234. In some examples, epigenetic data may also be based on fragmentomics data determined via methylation data from the TFR methylation component 235. The epigenetic data may be stored in the analysis datastore 240.

### (A) Methylation Status

The Methylation status can be determined in the cfDNA and used in the determination of whether a sample is tumor-derived as described herein. In general, cfDNA can be separated into methylated and unmethylated partitions based on the overall methylation state of each molecule. The cfDNA can be partitioned based on the differential binding affinity of the methylated nucleic acid molecules to a binding agent (i.e., a binding agent that binds to methylated nucleotides). In some embodiments, no bisulfite conversion is used. The DNA in each partition can then be tagged with a distinct set of dual barcodes, which uniquely identifies the partition associated with every molecule and aid in identification of unique cfDNA molecules post sequencing. DNA molecules in the methylated partitions can then be treated with restriction enzymes to deplete the samples of partially methylated molecules. All partitions can then be PCR amplified and enriched via hybridization to oligonucleotides representing genomic regions of interest targeting approximately 1 Mb of human genome. Enriched partitions can be pooled and tagged with an index uniquely identifying each sample prior to pooling multiple enriched samples into sequencing pools. Sequencing pools were sequenced on the NovaSeq 6000 instruments. Additionally or alternatively, cfDNA fragments from a sample 201 and/or a subject 211 may be treated in the sample collection and preparation pipeline 203, for example by converting unmethylated cytosines to uracils, and sequenced according to the sequencing pipeline 205.

In accordance with the present description, sequence fragments/reads may be compared by the LR methylation component 233 and/or a tumor fraction regression (TFR) methylation component 235 to a reference genome to identify the methylation states at specific CpG sites within the sequence fragments/reads. Each CpG site may be methylated or unmethylated. Identification of anomalously methylated fragments, in comparison to healthy individuals, may provide insight into a subject's cancer status. DNA methylation anomalies (compared to healthy controls) can cause different effects, which may contribute to cancer. Methylation typically occurs in deoxyribonucleic acid (DNA) when a hydrogen atom on the pyrimidine ring of a cytosine base is converted to a methyl group, forming 5-methylcytosine. In particular, methylation tends to occur at dinucleotides of cytosine and guanine referred to herein as “CpG sites.” Anomalous DNA methylation can be identified as hypermethylation or hypomethylation, both of which may be indicative of cancer status. Throughout this disclosure, hypermethylation and hypomethylation may be characterized for a sequence fragment/read, if the sequence fragment/read comprises more than a threshold number of CpG sites with more than a threshold percentage of those CpG sites being methylated or unmethylated. Example thresholds for numbers of CpG sites include more than 3, 4, 5, 6, 7, 8, 9, 10, etc. Example percentage thresholds of methylation or unmethylation include more than 80%, 85%, 90%, or 95%, or any other percentage within the range of 50%-100%. Those of skill in the art will appreciate that the principles described herein are equally applicable for the detection of methylation in a non-CpG context, including non-cytosine methylation.

In an embodiment, the LR methylation component 233 and/or the TFR methylation component 235 may be configured to determine a location and methylation state for each CpG site based on alignment to a reference genome. The LR methylation component 233 and/or the TFR methylation component 235 may generate a methylation state vector for each fragment specifying a location of the fragment in the reference genome (e.g., as specified by the position of the first CpG site in each fragment, or another similar metric), a number of CpG sites in the fragment, and the methylation state of each CpG site in the fragment whether methylated (e.g., denoted as M), unmethylated (e.g., denoted as U), or indeterminate (e.g., denoted as I). Observed states are states of methylated and unmethylated; whereas, an unobserved state is indeterminate. Indeterminate methylation states may originate from sequencing errors and/or disagreements between methylation states of a DNA fragment's complementary strands. The methylation state vectors may be stored in the analysis datastore 240 for later use and processing. Further, the LR methylation component 233 and/or the TFR methylation component 235 may remove duplicate reads or duplicate methylation state vectors from a single sample. The LR methylation component 233 and/or the TFR methylation component 235 may determine that a certain fragment with one or more CpG sites has an indeterminate methylation status over a threshold number or percentage and may exclude such fragments.

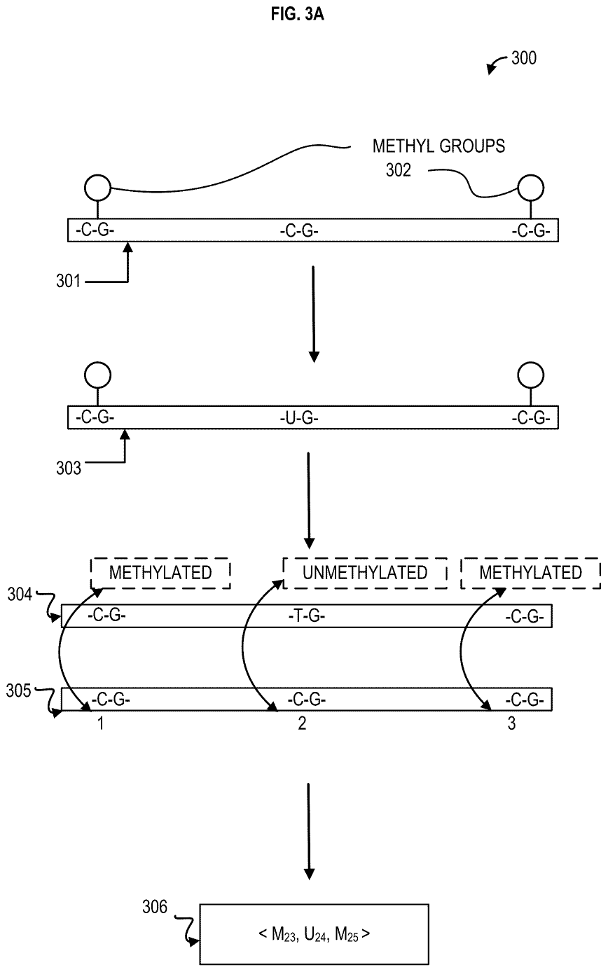

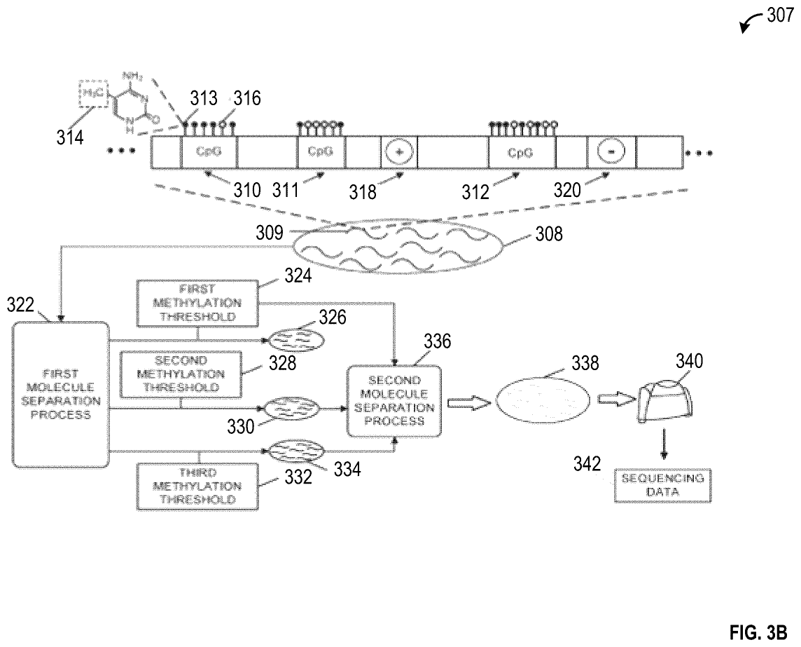

FIG. 3A is an illustration of a method 300 for sequencing a cfDNA molecule to obtain a methylation state vector. The method 300 may include single-site methylation. As an example, the laboratory system 202 receives a cfDNA molecule 301 that, in this example, contains three CpG sites. As shown, the first and third CpG sites of the cfDNA molecule 301 are methylated 302. As part of the sample collection and preparation pipeline 203, the cfDNA molecule 301 is converted to generate a converted cfDNA molecule 303. The second CpG site which was unmethylated has its cytosine converted to uracil but the first and third CpG sites were not converted.

In one or more examples, methylated cytosines can be determined using at least one of sodium bisulfite conversion and sequencing, Tet-assisted bisulfite sequencing (TAB-Seq), differential enzymatic cleavage, treatment with MSRE and/or MDRE, MBD partitioning, ACE-Seq, Ox-BS, Tet-assisted pyridine borane sequencing (TAPS); EM-Seq; SEM-seq, DM-Seq, TrueMethyl oxidative bisulfite sequencing.

After conversion, the sequencing pipeline 205 is used to generating sequence fragments/reads 304. The LR methylation component 233 and/or the TFR methylation component 235 may be configured to align the sequence fragment/read 304 to a reference genome 305. The reference genome 305 provides context as to what position in a human genome the fragment cfDNA originates. In this simplified example, the LR methylation component 233 and/or the TFR methylation component 235 may align the sequence read 304 such that the three CpG sites correlate to CpG sites 1, 2, and 3. Thus, the LR methylation component 233 and/or the TFR methylation component 235 may generate information both on methylation status of all CpG sites on the cfDNA molecule 301 and the position in the human genome to which the CpG sites map. As shown, the CpG sites on sequence read 304 which were methylated are read as cytosines. In this example, the cytosines appear in the sequence read 304 only in the first and third CpG site which allows one to infer that the first and third CpG sites in the original cfDNA molecule were methylated. Whereas, the second CpG site is read as a thymine (U is converted to T during the sequencing process), and thus, one can infer that the second CpG site was unmethylated in the original cfDNA molecule. With these two pieces of information, the methylation status and location, the LR methylation component 233 and/or the TFR methylation component 235 may generate a methylation state vector 306 for the fragment cfDNA 301. In this example, the resulting methylation state vector 306 is <M1, U2, M3>, wherein M corresponds to a methylated CpG site, U corresponds to an unmethylated CpG site, and the subscript number corresponds to a position of each CpG site in the reference genome.

In another embodiment, after sequencing and alignment, the methylation status of an individual CpG site may be inferred from the count of methylated sequence reads “M” (methylated) and the count of unmethylated sequence reads “U” (unmethylated) at the cytosine residue in CpG context. A mean methylated CpG density (also called methylation density m) of specific loci in the plasma can be calculated using the equation: m=M/(M+U) where M is the count of methylated reads and U is the count of unmethylated reads at the CpG sites within the genetic locus. If there is more than one CpG site within a locus, then M and U correspond to the counts across the sites.

Besides sequencing, other techniques can be used to determine information regarding DNA methylation. In one embodiment, methylation profiling can be performed by methylation-specific PCR or methylation-sensitive restriction enzyme digestion followed by PCR or ligase chain reaction followed by PCR. In yet other embodiments, the PCR is a form of single molecule or digital PCR (B. Vogelstein et al. 1999 Proc Natl Acad Sci USA; 96: 9236-9241). In yet further embodiments, the PCR can be a real-time PCR. In other embodiments, the PCR can be multiplex PCR.

Using the methylation status and location, the TFR methylation component 235 may use a TFR model to quantify the fraction of tumor-derived cfDNA (e.g., tumor fraction) in a sample based on the quantification of the observed tumor-associated aberrant methylation of cfDNA molecules. This quantification may be based on the observed number of unique methylated molecules mapping to each of the targeted classification regions. These molecule counts are normalized to the overall number of unique methylated molecules observed in the normalization regions of the panel. After normalization, the dependence of the classification region feature values (normalized molecule counts) on the total number of molecules measured and input cfDNA amount for a sample is minimized. Region level normalized molecule counts may be used as input features into the TFR model. The predicted tumor fraction may be used as a TFR model score for assessment of cancer status of an individual sample.

FIG. 3B is a diagrammatic representation of an example environment 307 that identifies nucleic acids that correspond to classification regions of a reference sequence, where the classification regions have at least a threshold number of CpGs, according to one or more implementations. In one or more examples, the disease under consideration is a type of cancer.

The environment 307 can include a sample 308. The sample 308 can be derived from a biological fluid obtained from a subject. For example, the sample 308 can be derived from blood obtained from a subject. In one or more additional examples, the sample 308 can be derived from tissue of a subject. In various examples, the sample 308 can be derived from multiple sources. To illustrate, the sample 308 can be derived from one or more fluids of a subject and/or from tissue of a subject. In one or more illustrative examples, the subject can be a mammal. In one or more additional illustrative examples, the subject can be a human. In one or more further illustrative examples, the subject can be a non-human mammal.

The sample 308 can include a number of nucleic acids 309. Individual nucleic acids 309 can include a number of regions that have at least a threshold number of cytosine molecules and guanine molecules. In one or more examples, individual nucleic acids 309 can include regions having at least a threshold number of cytosine-guanine dinucleotides. In various examples, at least a portion of the cytosine-guanine pairs included in the regions can be sequentially located in sequences of the nucleic acids 309. In one or more illustrative examples, a region of a nucleic acid having at least a threshold amount of cytosine-guanine pairs can be referred to herein as a “CG region” or a “CpG region.” In one or more examples, a CG region can include at least 200 CpG dinucleotides. In one or more illustrative examples, a CG region can include from 200 CpG dinucleotides to 5000 CpG dinucleotides, from 300 CpG dinucleotides to 3000 CpG dinucleotides, from 200 CpG dinucleotides to 2500 CpG dinucleotides, or from 500 CpG dinucleotides to 1500 CpG dinucleotides. Additionally, a CG region can have a GC percentage of at least 50% and an observed-to-expected CpG ratio of at least 60%. The observed-to-expected CpG ratio can be calculated where the observed CpG is the number of CpGs identified in a given genomic region and the expected CpGs is the number of cytosines multiplied by the number of guanines divided by the number of bases in the genomic region. The expected CpGs can also be calculated by:

((number of cytosines+number of guanines)/2)2/length of genomic region.

For example, a CG region can be determined using the techniques described by Gardiner-Garden M, Frommer M (1987). “CpG islands in vertebrate genomes”. Journal of Molecular Biology. 196 (2): 261-282. and/or Saxonov S, Berg P, Brutlag D L (2006). “A genome-wide analysis of CpG dinucleotides in the human genome distinguishes two distinct classes of promoters”. Proc Natl Acad Sci USA. 103 (5): 1412-1417.

In the illustrative example of FIG. 3B, a portion of a sequence of an example nucleic acid 309 can include a first CG region 310, a second CG region 311, and a third CG region 312. Although the illustrative example of FIG. 3B illustrates a portion of a sequence of a nucleic acid 309 having three CG regions, nucleic acids 309 included in the sample 308 can have a different number of CG regions. For example, individual nucleic acids 309 included in the sample 308 can include at least 1 CG region, at least 5 CG regions, at least 10 CG regions, at least 25 CG regions, at least 50 CG regions, at least 100 CG regions, at least 250 CG regions, at least 500 CG regions, or at least 1000 CG regions.

Individual CG regions can correspond to a number of molecules with one or more methylated cytosines. In the illustrative example of FIG. 3B, the CG region 310 can include a molecule with a methylated cytosine 313. In the illustrative example of FIG. 3B, the molecule with a methylated cytosine 313 is 5-methylcytosine. Individual CG regions can also correspond to a number of molecules with an unmethylated cytosine. For example, the CG region 310 can include a molecule with an unmethylated cytosine 316. In various examples, at least a portion of the CG regions of a nucleic acid 309 can correspond to classification regions of a reference genome. Classification regions can correspond to genomic regions of a reference genome that correspond to non-sequence differences that are consistent with one or more biological conditions, such as one or more types of cancer. In at least some examples, the non-sequence differences can include one or more mutations that are consistent with one or more biological conditions. In one or more examples, a classification region can correspond to a genomic region of the reference sequence for which molecules derived from subjects having at least one form of cancer. In at least some examples, nucleic acid molecules having at least a threshold amount of methylated cytosines in at least one CG region (e.g., hypermethylated molecules) in at least one CG region can be derived from subjects in which cancer is present and correspond to a classification region.

In addition to the classification regions, the CG regions can include one or more positive control regions, such as positive control region 318. The positive control region 311 can be mapped to nucleic acid molecules having at least a threshold number of methylated cytosine molecules in at least one CG region and that are derived from subjects that are free of cancer and are derived from subjects in which cancer is present. In various examples, the positive control region 310 can be hypermethylated in cells derived from subjects that are free of cancer and also in cells derived from subjects in which cancer is present. The CG regions can also include one or more negative control regions, such as negative control region 320. The negative control region 320 can be mapped to nucleic acid molecules having less than a threshold number of methylated cytosine molecules in at least one CG region and that are derived from subjects that are free of cancer and also subjects in which cancer is present. In one or more illustrative examples, the negative control region 320 can be hypomethylated in subjects that are free of cancer and also in subjects in which cancer is present. In various examples, the positive control regions and the negative control regions can be used to perform normalization calculations. The normalization calculations can be performed to generate input data for one or more models that are implemented to determine tumor metrics for a given sample 308.

A first molecule separation process 322 can be performed. The first molecule separation process 322 can separate nucleic acids 309 included in the sample 308 based on an amount of methylated cytosines of the individual nucleic acids 309. In one or more examples, the first molecule separation process can separate nucleic acids 309 included in the sample 308 based on amounts of methylated cytosines included in CG regions of individual nucleic acids 309. In various examples, the first molecule separation process 322 can separate the nucleic acids 309 into a plurality of groups with individual groups corresponding to respective amounts of methylated cytosines of the nucleic acids 309.

In the illustrative example of FIG. 3B, the first molecule separation process 322 can be performed in relation to a first methylation threshold 324. Performing the first molecule separation process 322 with regard to the first methylation threshold 324 can produce a first partition of nucleic acids 326. In one or more examples, the first methylation threshold 324 can indicate a first threshold number of molecules with a methylated cytosine located in CG regions of the nucleic acids 309. The first molecule separation process 322 can identify a number of nucleic acids 309 having fewer molecules with a methylated cytosine in CG regions than the first methylation threshold 324. In various examples, the first methylation threshold 324 can correspond to a first methylation rate.

The first molecule separation process 322 can also be performed with respect to a second methylation threshold 328. The second methylation threshold 328 can indicate an amount of methylated cytosines in one or more genomic regions of the nucleic acids 309 that is greater than the amount of methylated cytosines in the one or more regions corresponding to the first methylation threshold 324. The second methylation threshold 324 can indicate a number of molecules with a methylated cytosine per a number of nucleic acids. In one or more additional examples, the second methylation threshold 324 can correspond to a rate of methylation of nucleic acids that is greater than the rate of methylation that corresponds to the first methylation threshold 324. Performing the first molecule separation process 322 with respect to the second methylation threshold 328 can produce a second partition of nucleic acids 330. In one or more examples, the first molecule separation process 322 can identify nucleic acids 309 having a greater amount of methylated cytosines than the first methylation threshold 324 and having a lower amount of methylated cytosines than the second methylation threshold 328 to produce the second partition of nucleic acids 330.

Additionally, the first molecule separation process 322 can also be performed with respect to a third methylation threshold 332. The third methylation threshold 332 can indicate an amount of methylated cytosines in one or more genomic regions of the nucleic acids 309 that is greater than the amount of methylated cytosines in the one or more regions corresponding to the first methylation threshold 324 and greater than the amount of methylated cytosines in the one or more regions corresponding to the second methylation threshold 328. The third methylation threshold 332 can indicate a number of molecules with a methylated cytosine per a number of nucleic acids. In one or more additional examples, the third methylation threshold 332 can correspond to a rate of methylated cytosines that is greater than the rate of methylation that corresponds to the first methylation threshold 324 and greater than the rate of methylation that corresponds to the second methylation threshold 328. Performing the first molecule separation process 322 with respect to the third methylation threshold 332 can produce a third partition of nucleic acids 334. In one or more examples, the first molecule separation process 322 can identify nucleic acids 309 having a greater amount of methylated cytosines than nucleic acids 309 included in the second partition of nucleic acids 328. In this way, the amount of methylated cytosines of nucleic acids included in the first partition 322, the second partition 326, and the third partition 330 increases from the first partition 322 to the second partition 326 and increases from the second partition 326 to the third partition 330. In one or more illustrative examples, the first partition of nucleic acids 326 can be referred to as a hypomethylation partition, the second partition of nucleic acids 330 can be referred to as an intermediate partition, and the third partition of nucleic acids 334 can be referred to as a hypermethylation partition.

In one or more examples, the amount of methylated cytosines of nucleic acids can correspond to a strength of binding to methyl binding domain (MBD). In these scenarios, the first partition 326, the second partition 330, and the third partition 334 can be produced based on different strengths of binding to MBD for nucleotides having different amounts of methylated cytosines. In one or more examples, the first molecule separation process 322 can include a series of washes where the nucleic acids 309 are contacted with solutions having different concentrations of sodium chloride (NaCl).

Partitioning of the nucleic acids can be performed by contacting the nucleic acids with a modified nucleotide specific binding reagent, such as a MBD of a MBP. A modified nucleotide specific binding reagent can bind to 5-methylcytosine (5mC). The modified nucleotide specific binding reagent, such as a MBD, can be coupled to paramagnetic beads, such as Dynabeads® M-280 Streptavidin via a biotin linker. Partitioning into fractions with different extents of methylation can be performed by increasing the NaCl concentration in a series of washes. The sequences eluted from the modified nucleotide specific binding reagent are partitioned into two or more fractions (e.g., hypo, hyper) depending on which wash (e.g., NaCl concentration) eluted the sequences. Resulting partitions can include one or more of the following nucleic acid forms: double-stranded DNA (dsDNA), shorter DNA fragments and longer DNA fragments.

The binding of the nucleic acids with the modified nucleotide specific binding reagent can be a function of number of methylated (or modified) sites per molecule, with molecules having more methylation eluting under increased salt concentrations. To elute the DNA into distinct populations based on the extent of methylation, one can use a series of elution buffers of increasing NaCl concentration. Salt concentrations can, in one or more implementations, range from about 100 nM to about 2500 mM NaCl. In various implementations, the process results in three (3) partitions. Molecules are contacted with a solution at a first salt concentration and comprising a molecule comprising a methyl binding domain, which molecule can be attached to a capture moiety, such as streptavidin. At the first salt concentration a population of molecules will bind to the MBD and a population will remain unbound. The unbound population can be separated as a “hypomethylated” population (hypo partition). For example, the first partition 326 can be representative of the hypomethylated form of DNA is that which remains unbound at a low salt concentration. In one or more illustrative examples, the concentration of NaCl of the solution used to produce the first partition 326 can be about 100 nM, about 120 nM, about 140 nM, about 160 nM, about 180 nM, about 200 nM. or about 250 nM. The second partition 330 can be referred to as a “residual partition” or an “intermediate partition” and can be representative of intermediate methylated DNA is eluted using an intermediate salt concentration, e.g., between 100 mM and 2000 mM concentration. In one or more additional illustrative examples, the concentration of NaCl of the solution used to produce the second partition 330 can be from about 100 mM to about 500 mM, from about 100 mM to about 1000 mM, from about 100 mM to about 1500 mM, from about 250 mM to about 1000 mM, from about 250 mM to about 1500 mM, from about 500 mM to about 1500 mM, from about 250 mM to about 2000 mM, from about 500 mM to about 2000 mM, or from about 1000 mM to about 2000 mM. This is also separated from the sample. The third partition 334 can be representative of hypermethylated form of DNA (hyper partition) and is eluted using a high salt concentration, e.g., at least about 2000 mM. In one or more further illustrative examples, the concentration of NaCl of the solution used to produce the third partition 334 can be from about 2000 mM to about 5000 mM, from about 2000 mM to about 4000 mM, from about 2000 mM to about 3500 mM, from about 2000 mM to about 3000 mM, or from about 2500 mM to about 4000 mM.

In various examples, the first partition 326 can correspond to a first range of binding strengths of nucleic acids to MBD and to a first range of methylated CG regions and the second partition 330 can correspond to a second range of binding strengths of nucleic acids to MBD and to a second range of methylated CG regions. The first range of binding strengths can be less than the second range of binding strengths. In one or more scenarios, a first solution having a first NaCl concentration can separate a first group of nucleic acids having the first range of binding strengths from MBD and a second solution having a second NaCl concentration can separate a second group of nucleic acids having the second range of binding strengths from MBD with the second NaCl concentration being greater than the first NaCl concentration. Additionally, the third partition 334 can correspond to a third range of binding strengths and a third range of methylated CG regions. The third range of binding strengths can be greater than the first range of binding strengths and the second range of binding strengths. In one or more instances, a third solution having a third NaCl concentration can separate a third group of nucleic acids having the third range of binding strengths from NaCl. The third NaCl concentration can be greater than the first NaCl concentration and the second NaCl concentration.

In one or more illustrative examples, a plurality of nucleic acids derived from at least one of blood or tissue of a subject can be combined with a solution including an amount of MBD to produce a nucleic acid-MBD solution. A first wash of the nucleic acid-MBD solution can be performed with a first solution including a first NaCl concentration to produce a first nucleic acid fraction and a first residual solution. The first nucleic acid fraction can include a first portion of the plurality of nucleic acids and the first residual solution can include a second portion of the plurality of nucleic acids. In one or more examples, the first portion of the plurality of nucleic acids can have a first range of binding strengths to MBD that are less than a second range of binding strengths to MBD of the second portion of the plurality of nucleic acids.

Additionally, a second wash of the first residual solution can be performed with a second solution including a second concentration of NaCl that is greater than the first concentration of NaCl to produce a second nucleic acid fraction and a second residual solution. The second nucleic acid fraction can include a first subset of the second portion of the plurality of nucleic acids and the second residual solution can include a second subset of the second portion of the plurality of nucleic acids. The first subset of the second portion of the plurality of nucleic acids can have a third range of binding strengths to MBD that are less than a fourth range of binding strengths to MBD of the second subset of the second portion of the plurality of nucleic acids. Further, a third wash of the second residual solution can be performed with a third solution including a third concentration of NaCl that is greater than the second concentration of NaCl to produce a third nucleic acid fraction that includes the second subset of the second portion of the plurality of nucleic acids.

Subsequent to the first wash, the second wash, and the third wash a determination can be made that the first portion of the plurality of nucleic acids are associated with the first partition 326. The first portion of the plurality of nucleic acids can be attached with molecular barcodes from a first set of molecular barcodes indicating the first partition 326. In this way, a sequencing read that corresponds to the first partition 326 can be identified based on determining that the sequencing read includes the first molecular barcode. In addition, a determination can be made that the first subset of the second portion of the plurality of nucleic acids is associated with an additional partition of the plurality of partitions. In these situations, a second set of molecular barcodes different from the first set of molecular barcodes can be attached to the second portion of the plurality of nucleic acids with the second molecular barcode indicating the additional partition. As a result, a sequencing read that corresponds to the additional partition can be identified based on determining that the sequencing read includes one or more molecular barcodes from among the second set of molecular barcodes. Further, a determination can be made that the second subset of the second portion of the plurality of nucleic acids is associated with the second partition 330. A third set of molecular barcodes different from the first set of molecular barcodes and the second set of molecular barcodes can then be attached to the second subset of the second portion of the plurality of nucleic acids where the third set of molecular barcodes indicate the second partition 330. In these instances, a sequencing read that corresponds to the second partition 330 can be identified based on determining that the sequencing read includes a third molecular barcode from among the third set of molecular barcodes.

In at least some examples, the first molecule separation process 322 can result in nucleic acids being present in at least one of the first partition 326, the second partition 330, or the third partition 334 having an amount of methylation that is different from the amount of methylation of the other nucleic acids in the respective partition. For example, the first partition 326 can include a number of nucleic acids having amounts of methylation that correspond to the amounts of methylation of nucleic acids included in at least one of the second partition 330 or the third partition 334. Additionally, at least one of the second partition 330 or the third partition 334 can include nucleic acids having amounts of methylation that correspond to the amounts of methylation of nucleic acids included in the first partition 326. The presence of nucleic acids in at least one of the first partition 326, the second partition 330, or the third partition 334 that do not correspond to the amounts of methylation of at least a majority of the other nucleic acids included in the respective partition can cause data noise when performing computational operations with respect to sequence reads produced from nucleic acids included in the first partition 326, the second partition 330, and the third partition 334. The data noise can result in inaccuracies with respect to calculations made based on sequence reads derived from nucleic acids included in the first partition 326, the second partition 330, and the third partition 334.

To reduce or eliminate data noise associated with nucleic acids being present in at least one of the first partition 326, the second partition 330, or the third partition 334 that have amounts of methylation that are not consistent with the amounts of methylation of at least a majority of other molecules included in the respective partitions, a second molecule separation process 336 can be performed after the first molecule separation process 322. The second molecule separation process 336 can be performed with respect to nucleic acids included in the first partition 326, nucleic acids included in the second partition 330, and nucleic acids included in the third partition 334. In one or more examples, the second molecule separation process 336 can include performing digestion of the nucleic acids included in the first partition 326 using methylation dependent restriction enzyme (MDRE) and nucleic acids included in the second partition 330 and the third partition 334 can be digested using methylation sensitive restriction enzyme (MSRE). Digestion of the nucleic acids included in the first partition 326 with MDRE can result in separation of nucleic acids included in the first partition having amounts of methylation corresponding to the second partition 330 and the third partition 334 from nucleic acids having amounts of methylation corresponding to the first partition. Additionally, digestion of nucleic acids included in the second partition 330, and the third partition 334 with MSRE can result in separation of the nucleic acids having amounts of methylation corresponding to the first partition 326 from the nucleic acids of the second partition 330 and the nucleic acids of the third partition 334. By removing nucleic acids from the first partition 326 having amounts of methylation that correspond to the second partition 330 and the third partition 334 and by removing nucleic acids from the second partition 330 and the third partition 334 that have amounts of methylation that correspond to the first partition 326, an additional group of nucleic acids 338 can be produced. The additional group of nucleic acids 338 can include nucleic acids corresponding to methylation amounts of the second partition 330 and the third partition 334 with a minimal amount or no nucleic acids having amounts of methylation corresponding to the first partition 326. For example, less than 50% of the nucleic acids included in the additional group 338 can have amounts of methylation that correspond to the second partition 330 and the third partition 334, at least 50% of the nucleic acids included in the additional group 338 can have amounts of methylation that correspond to the second partition 330 and the third partition 334, at least 60% of the nucleic acids included in the additional group 338 can have amounts of methylation that correspond to the second partition 330 and the third partition 334, at least 70% of the nucleic acids included in the additional group 338 can have amounts of methylation that correspond to the second partition 330 and the third partition 334, at least 90% of the nucleic acids included in the additional group 338 can have amounts of methylation that correspond to the second partition 330 and the third partition 334, at least 95% of the nucleic acids included in the additional group 338 can have amounts of methylation that correspond to the second partition 330 and the third partition 334, at least 97% of the nucleic acids included in the additional group 338 can have amounts of methylation that correspond to the second partition 330 and the third partition 334, at least 99% of the nucleic acids included in the additional group 338 can have amounts of methylation that correspond to the second partition 330 and the third partition 334, at least 99.5% of the nucleic acids included in the additional group 338 can have amounts of methylation that correspond to the second partition 330 and the third partition 334, or at least 99.9% of the nucleic acids included in the additional group 338 can have amounts of methylation that correspond to the second partition 330 and the third partition 334.

The architecture 307 can include a sequencing machine 340. In one or more examples, the sequencing machine 340 can be any of a number of sequencing machines that can perform one or more sequencing operations that amplify nucleic acids present in a sample 309. In various examples, the sequencing machine 340 can perform next generation sequencing operations. In one or more examples, the sample 309 can include an amount of at least one bodily fluid extracted from a subject. In one or more additional examples, the sample 309 can include a tissue sample that is obtained from a subject.

In one or more examples, prior to sequencing, the extracted polynucleotides can be partitioned into two or more partitions based on the binding strength of the of binding strengths of polynucleotides to MBD. A blunt-end ligation can be performed on the partitioned polynucleotides and adapters, as well as tags (e.g., molecular barcodes) can be added to the partitioned polynucleotides. The tagged polynucleotides in the one or more partitions (e.g. hyper and/or intermediate partitions) can be treated with one or more methylation sensitive restriction enzymes (MSREs). In some examples, the hypo partition can be treated with one or more methylated dependent restriction enzymes (MDREs). Post the MSRE and/or MORE treatment, the molecules can also be enriched by causing hybridization between the extracted polynucleotides and probes that correspond to target regions of a reference sequence. The enrichment process can identify thousands, hundreds of thousands, up to millions of polynucleotides that correspond to on-target regions associated with the probes.

Subsequent and/or prior to the enrichment process, the molecules can be amplified according to one or more amplification processes. The one or more amplification processes can produce thousands, up to millions of copies of individual nucleic acid molecules. In one or more examples, a portion of the unenriched polynucleotides can be amplified, in some instances, but not to the extent that the enriched polynucleotides are amplified. The one or more amplification processes can generate an amplification product that undergoes one or more sequencing operations. After performing one or more sequencing operations with respect to the sample 309, the sequencing machine can produce a sequencing data 342.

The sequencing data 342 can include alphanumeric representations of the nucleic acids included in an amplification product. For example, the sequencing data 342 can include, for individual nucleic acids of the amplification product, data that corresponds to a string of letters that represent the respective chains of nucleotides that correspond to the individual nucleic acids.

The sequencing data 342 can be stored in one or more data files. For example, the sequencing data 342 can be stored in a FASTQ file that comprises a textbased sequencing data file format storing raw sequence data and quality scores. In one or more additional examples, the sequencing data 342 can be stored in a data file according to a binary base call (BCL) sequence file format. In one or more further examples, the sequencing data 342 can be stored in a BAM file. In one or more examples, the sequencing data 342 can comprise at least about one gigabyte (GB), at least about 2 GB, at least about 3 GB, at least about 4 GB, at least about 5 GB, at least about 8 GB, or at least about 10 GB. An individual sequence representation included in the sequencing data 310 can be referred to herein as a “read” or a “sequencing read.” In various examples, individual first nucleic acids included in the pool 338 can correspond to multiple sequence representations included in the sequencing data 342 as a result of the amplification of the individual first nucleic acids. In one or more additional examples, individual second nucleic acids included in the pool 338 can correspond to a single sequence representation included in the sequencing data 342 as a result of the absence of amplification of the individual second nucleic acids.

Also using the methylation status and location, the LR methylation component 233 may include a LR model to differentiate the tumor-associated methylation signatures of cfDNA molecules from those observed in subjects without tumors. The methylation LR model may use the same input feature space as the TFR model of the TFR methylation component 235 (e.g., the region level normalized molecule counts). Compared to the TFR model of the TFR methylation component 235, the methylation LR model of the LR methylation component 233 may be trained to predict the binary disease state (e.g., cancer and non-cancer) instead of the quantitative tumor fraction (e.g., of the TFR model of the TFR methylation component 235). The LR model score provided from the LR methylation component 233 may include the binary predicted disease state. In some examples, the LR model of the LR methylation component 233 may be trained on the same set of samples used to train the TFR model of the TFR methylation component 235.

In some embodiments, methods disclosed herein comprise a step of subjecting DNA to a procedure that affects a first nucleobase in the DNA differently from a second nucleobase in the DNA, wherein the first nucleobase is a modified or unmodified nucleobase, the second nucleobase is a modified or unmodified nucleobase different from the first nucleobase, and the first nucleobase and the second nucleobase have the same base pairing specificity. In some embodiments, the procedure chemically converts the first or second nucleobase such that the base pairing specificity of the converted nucleobase is altered. In some embodiments, if the first nucleobase is a modified or unmodified adenine, then the second nucleobase is a modified or unmodified adenine; if the first nucleobase is a modified or unmodified cytosine, then the second nucleobase is a modified or unmodified cytosine; if the first nucleobase is a modified or unmodified guanine, then the second nucleobase is a modified or unmodified guanine; and if the first nucleobase is a modified or unmodified thymine, then the second nucleobase is a modified or unmodified thymine (where modified and unmodified uracil are encompassed within modified thymine for the purpose of this step).

In some embodiments, the first nucleobase is a modified or unmodified cytosine, then the second nucleobase is a modified or unmodified cytosine. For example, first nucleobase may comprise unmodified cytosine (C) and the second nucleobase may comprise one or more of 5-methylcytosine (mC) and 5-hydroxymethylcytosine (hmC). Alternatively, the second nucleobase may comprise C and the first nucleobase may comprise one or more of mC and hmC. Other combinations are also possible, as indicated, e.g., in the Summary above and the following discussion, such as where one of the first and second nucleobases comprises mC and the other comprises hmC.

In some embodiments, the procedure that affects a first nucleobase in the DNA differently from a second nucleobase in the DNA comprises bisulfite conversion. Treatment with bisulfite converts unmodified cytosine and certain modified cytosines (e.g. 5-formyl cytosine (fC) or 5-carboxylcytosine (caC)) to uracil whereas other modified cytosines (e.g., 5-methylcytosine, 5-hydroxylmethylcystosine) are not converted. Performing bisulfite conversion can facilitate identifying positions containing mC or hmC using the sequence reads. For an exemplary description of bisulfite conversion, see, e.g., Moss et al., Nat Commun. 2018; 9: 5068.

In some embodiments, the procedure that affects a first nucleobase in the DNA differently from a second nucleobase in the DNA comprises oxidative bisulfite (Ox-BS) conversion. Performing Ox-BS conversion can facilitate identifying positions containing mC using the sequence reads. For an exemplary description of oxidative bisulfite conversion, see, e.g., Booth et al., Science 2012; 336: 934-937.

In some embodiments, the procedure that affects a first nucleobase in the DNA differently from a second nucleobase in the DNA comprises Tet-assisted bisulfite (TAB) conversion. For example, as described in Yu et al., Cell 2012; 149: 1368-80, b-glucosyl transferase can be used to protect hmC (forming 5-glucosylhydroxymethylcytosine (ghmC)), then a TET protein such as mTetl can be used to convert mC to caC, and then bisulfite treatment can be used to convert C and caC to U while ghmC remains unaffected. Thus, when TAB conversion is used, the first nucleobase comprises one or more of unmodified cytosine, fC, caC, mC, or other cytosine forms affected by bisulfite, and the second nucleobase comprises hmC. Performing TAB conversion can facilitate identifying positions containing hmC using the sequence reads.

In some embodiments, the procedure that affects a first nucleobase in the DNA differently from a second nucleobase in the DNA comprises Tet-assisted conversion with a substituted borane reducing agent, optionally wherein the substituted borane reducing agent is 2-picoline borane, borane pyridine, tert-butylamine borane, or ammonia borane. See, e.g., Liu et al., Nature Biotechnology 2019; 37:424-429 (e.g., at Supplementary FIG. 1 and Supplementary Note 7). Performing TAP conversion can facilitate identifying positions containing unmodified C using the sequence reads. This procedure encompasses Tet-assisted pyridine borane sequencing (TAPS), described in further detail in Liu et al. 2019, supra.

Alternatively, protection of hmC (e.g., using bOT) can be combined with Tet-assisted conversion with a substituted borane reducing agent. Performing such TAPSP conversion can facilitate distinguishing positions containing unmodified C or hmC on the one hand from positions containing mC using the sequence reads. For an exemplary description of this type of conversion, see, e.g., Liu et al., Nature Biotechnology 2019; 37:424-429.

In some embodiments, the procedure that affects a first nucleobase in the DNA differently from a second nucleobase in the DNA comprises APOBEC-coupled epigenetic (ACE) conversion. Performing ACE conversion can facilitate distinguishing positions containing hmC from positions containing mC or unmodified C using the sequence reads. For an exemplary description of ACE conversion, see, e.g., Schutsky et al., Nature Biotechnology 2018; 36: 1083-1090.

In some embodiments, procedure that affects a first nucleobase in the DNA differently from a second nucleobase in the DNA comprises enzymatic conversion of the first nucleobase, e.g., as in EM-Seq. See, e.g., Vaisvila R, et al. (2019) EM-seq: Detection of DNA methylation at single base resolution from picograms of DNA. bioRxiv; DOI [0, available at www.biorxiv.org/content/10.1101/2019.12.20.884692v1.

In some embodiments, procedure that affects a first nucleobase in the DNA differently from a second nucleobase in the DNA comprises the methods involving direct methylation sequencing (DM-Seq) for detecting 5mC. Exemplary methods and compositions related to DM-Seq can be found in, e.g., WO2023/288222 and WO2021/236778, which are hereby incorporated by reference.

In some embodiments, the first nucleobase is a modified or unmodified adenine, and the second nucleobase is a modified or unmodified adenine. In some embodiments, the modified adenine is N6-methyladenine (mA). In some embodiments, the modified adenine is one or more of N6-methyladenine (mA), N6-hydroxymethyladenine (hmA), or N6-formyladenine (fA).

### (B) Fragmentomics

Returning to FIG. 2, in an embodiment, a fragmentomic component fragmentomics component 234 may analyze sequence fragments/reads to determine fragmentomic data. Fragmentomic data may include, for example, information regarding fragment size, nucleotide motifs at fragment ends, single-stranded jagged ends, and/or genomic locations of fragmentation endpoints. The fragmentomic component fragmentomics component 234 may be configured to analyze the sequence fragments/reads to determine one or more of: fragment size, end motif frequency, jagged end length, preferred end coordinates, oriented end density, motif diversity score, a window protection score, cfDNA integrity, nucleosomal footprinting, combinations thereof, and the like. The fragmentomic data may be used as a fragmentomic signature. Fragmentomic data may be determined by any means known in the art. The fragmentomic data may be stored in the analysis datastore 240.

In an embodiment, the fragmentomic component fragmentomics component 234 may be configured to determine an amount of the cell-free DNA fragments that have a particular size. The particular size can be a range. For example, a size range can be greater than or less than a size cutoff, e.g., 100 bp, 150 bp, or 200 bp. As other examples, the size range can be specified by a minimum and a maximum size, e.g., 50-80, 50-100, 50-150, 100-150, 100-200, 150-200, 150-230, 200-300, or 300-400 bases, as well as other ranges. The width of the size range can vary, e.g., to be 50, 100, 150, or 200 bases. As examples, the amount can be a raw count or be normalized, e.g., as a frequency using a total number of sequence reads or DNA fragments analyzed.

In an embodiment, the fragmentomics component 234 may be configured to determine an end motif for a sequence fragment/read and to determine an end motif frequency. An end motif relates to the ending sequence of a cell-free DNA fragment, e.g., the sequence for the K bases at either end of the fragment. The ending sequence can be a k-mer having various numbers of bases, e.g., 1, 2, 3, 4, 5, 6, 7, etc. The end motif (or “sequence motif”) relates to the sequence itself as opposed to a particular position in a reference genome. Thus, a same end motif may occur at numerous positions throughout a reference genome. The end motif may be determined using a reference genome, e.g., to identify bases just before a start position or just after an end position. Such bases will still correspond to ends of cell-free DNA fragments, e.g., as they are identified based on the ending sequences of the fragments.

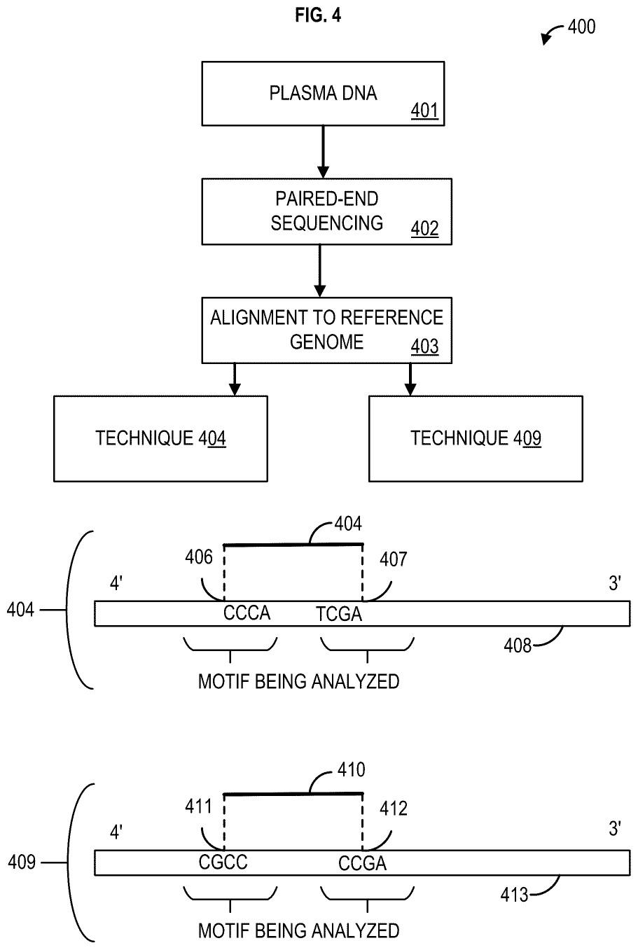

FIG. 4 shows examples for end motifs according to embodiments of the present disclosure. FIG. 4 depicts techniques to define 4-mer end motifs to be analyzed. In technique 404, the 4-mer end motifs are directly constructed from the first 4-bp sequence on each end of a plasma DNA molecule. For example, the first 4 nucleotides or the last 4 nucleotides of a sequenced fragment could be used. In technique 409, the 4-mer end motifs are jointly constructed by making use of the 2-mer sequence from the sequenced ends of fragments and the other 2-mer sequence from the genomic regions adjacent to the ends of that fragment. In other embodiments, other types of motifs can be used, e.g., 1-mer, 2-mer, 3-mer, 5-mer, 6-mer, 7-mer end motifs.

As shown in FIG. 4, a method 400 may begin with obtaining cell-free DNA fragments at step 401 via the laboratory system 202 and the sample collection and preparation pipeline 203 (e.g., using a purification process on a blood sample, such as by centrifuging). Besides plasma DNA fragments, other types of cell-free DNA molecules can be used, e.g., from serum, urine, saliva, and other samples mentioned herein. In one embodiment, the DNA fragments may be blunt-ended.

At step 402, the DNA fragments are subjected to paired-end sequencing via the sequencing pipeline 205. In some embodiments, the paired-end sequencing can produce two sequence reads from the two ends of a DNA fragment, e.g., 30-120 bases per sequence read. These two sequence reads can form a pair of reads for the DNA fragment (molecule), where each sequence read includes an ending sequence of a respective end of the DNA fragment. In other embodiments, the entire DNA fragment can be sequenced, thereby providing a single sequence read, which includes the ending sequences of both ends of the DNA fragment. The two ending sequences at both ends can still be considered paired sequence reads, even if generated together from a single sequencing operation.

At step 403, the fragmentomic component fragmentomics component 234 may align the sequence reads to a reference genome. Such alignment is to illustrate different ways to define a sequence motif, and may not be used in some embodiments. For example, the sequences at the end of a fragment can be used directly without needing to align to a reference genome. However, alignment can be desired to have uniformity of an ending sequence, which does not depend on variations (e.g., SNPs) in the subject. For instance, the ending base could be different from the reference genome due to a variation or a sequencing error, but the base of in the reference may be the one counted. Alternatively, the base on the end of the sequence read can be used, so as to be tailored to the individual. The alignment procedure can be performed using various software packages, such as (but not limited to) BLAST, FASTA, Bowtie, BWA, BFAST, SHRiMP, SSAHA2, NovoAlign, and SOAP.

The method 400 may proceed to utilize technique 404 and/or technique 409 to further assess an end motif. Technique 404 shows a sequence read of a sequence fragment 405, with an alignment to a genome 408. With the 5′ end viewed as the start, a first end motif 406 (CCCA) is at the start of sequence fragment 405. A second end motif 407 (TCGA) is at the tail of the sequence fragment 405. When analyzing the end predominance of cfDNA fragments, this sequence read would contribute to a C-end count for the 5′ end. Such end motifs might, in one embodiment, occur when an enzyme recognizes CCCA and then makes a cut just before the first C. If that is the case, CCCA will preferentially be at the end of the plasma DNA fragment. For TCGA, an enzyme might recognize it, and then make a cut after the A. When a count is determined for the A, this sequence read would contribute to an A-end count.

Technique 409 shows a sequence read of a sequenced fragment 410, with an alignment to a genome 413. With the 5′ end viewed as the start, a first end motif 411 (CGCC) has a first portion (CG) that occurs just before the start of sequence fragment 410 and a second portion (CC) that is part of the ending sequence for the start of sequenced fragment 410. A second end motif 412 (CCGA) has a first portion (GA) that occurs just after the tail of sequenced fragment 410 and a second portion (CC) that is part of the ending sequence for the tail of sequenced fragment 410. Such end motifs might, in one embodiment, occur when an enzyme recognizes CGCC and then makes a cut just before the G and the C. If that is the case, CC will preferentially be at the end of the plasma DNA fragment with CG occurring just before it, thereby providing an end motif of CGCC. As for the second end motif 164 (CCGA), an enzyme can cut between C and G. If that is the case, CC will preferentially be at the end of the plasma DNA fragment. For technique 409, the number of bases from the adjacent genome regions and sequenced plasma DNA fragments can be varied and are not necessarily restricted to a fixed ratio, e.g., instead of 2:2, the ratio can be 2:3, 3:2, 4:4, 2:4, etc.

The higher the number of nucleotides included in the cell-free DNA end signature, the higher the specificity of the motif because the probability of having 6 bases ordered in an exact configuration in the genome is lower than the probability of having 2 bases ordered in an exact configuration in the genome. Thus, the choice of the length of the end motif can be governed by the needed sensitivity and/or specificity of the intended use application.

As the ending sequence is used to align the sequence read to the reference genome, any sequence motif determined from the ending sequence or just before/after is still determined from the ending sequence. Thus, technique 409 makes an association of an ending sequence to other bases, where the reference is used as a mechanism to make that association. A difference between techniques 404 and 409 would be to which two end motifs a particular DNA fragment is assigned, which affects the particular values for the relative frequencies. But, the overall result (e.g., detecting a genetic disorder, determining efficacy of a dosage, monitoring activity of a nuclease, etc.) would not be affected by how the a DNA fragment is assigned to an end motif, as long as a consistent technique is used, e.g., for any training data to determine a reference value, as may occur using a machine learning model.

The counted numbers of DNA fragments having an ending sequence corresponding to a particular end motif (e.g., a particular base) may be counted (e.g., stored in an array in memory) to determine an amount of the particular end motif. The amount can be measured in various ways, such as a raw count or a frequency, where the amount is normalized. The normalization may be done using (e.g., dividing by) a total number of DNA fragments or a number in a specified group of DNA fragments (e.g., from a specified region, having a specified size, or having one or more specified end motifs). Differences in amounts of end motifs have been detected when a genetic disorder exists, as well as when an effective dose of an anticoagulant has been administered, as well as when the activity of a nuclease changes (e.g., increases or decreased).

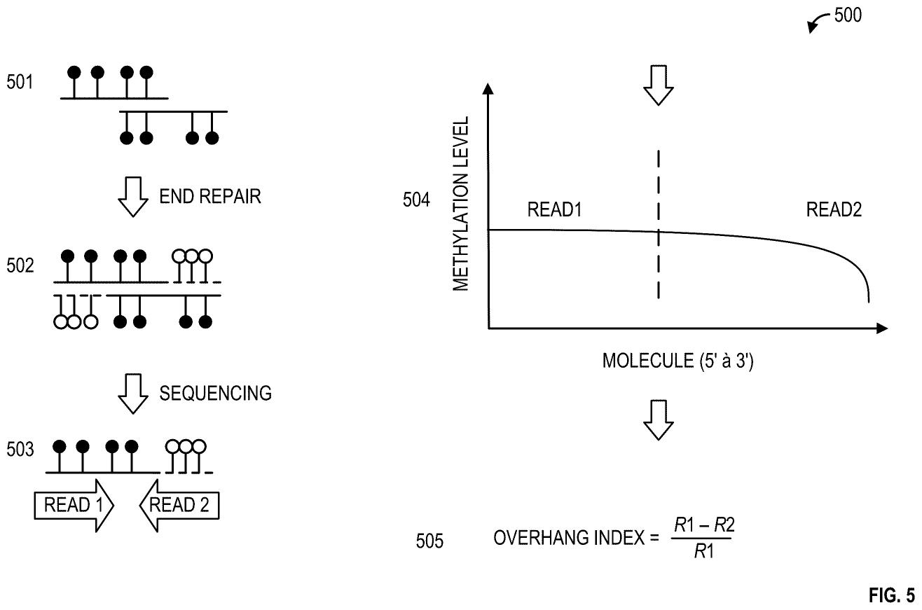

In an embodiment, the fragmentomic component fragmentomics component 234 may be configured to determine a presence of a jagged end (e.g., an overhang) and an associated quantitative value. FIG. 5 illustrates one example showing how the degree of overhangs of cell-free DNA molecules (i.e., overhang index) can be determined. Diagrams 501, 502, and 503 include filled circles that represent methylated CpG sites, and unfilled circles that represent unmethylated CpG sites. Diagrams 502 and 503 include a dashed line that represents newly filled-up nucleotides. Diagram 503 includes an arrow indicative of the first read (read 1) in sequencing results and an arrow indicative of the secondary read (read 2). Graph 504 shows a graph of methylation level in read 1 and read 2 from 5′ to 3′ and an overhang index

$$250 ( \frac{R 1 - R 2}{R 2} )$$

that comprises the following variables: R1 as the methylation level of read 1 and R2 as the methylation level of read 2.

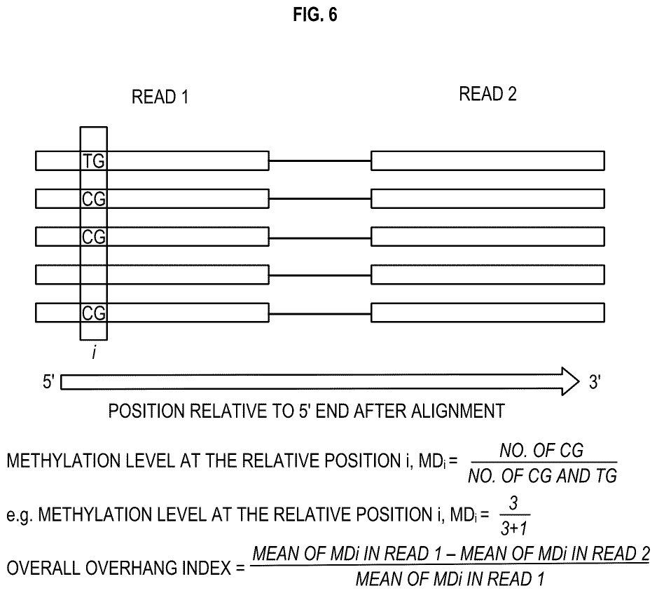

FIG. 6 is an illustration of the calculation of methylation levels along a DNA molecule after mapping to the human reference genome. All DNA molecules from the Watson and Crick strand may be stacked, respectively, according to relative positions and orientations after mapping to the human reference genome. The stacked molecules may be used for calculating an overall overhang index according to the positions relative to 5′ end in the alignment results as shown in FIG. 6.

The methylation level (MD) at a particular position i relative to the closest end (i.e., 5′ end for read 1) may be quantified by the ratio of the number of C's to the total number of C's and T's:

$$M {D}_{i} = \frac{\# of CG}{\# of CG and TG} .$$

The first read (having 5′ end, i.e. read 1) may have a higher averaged methylation level than the second read (having 3′ end, i.e. read 2) because the 3′ gaps in the second read would be filled in by unmethylated C's which would be converted to T's in bisulfite sequencing results. An overall overhang index may be determined according to the following:

$$\frac{MEAN OF M {D}_{i} IN READ 1 - MEAN OF M {D}_{i} IN READ 2}{MEAN OF M {D}_{i} IN READ 1} .$$

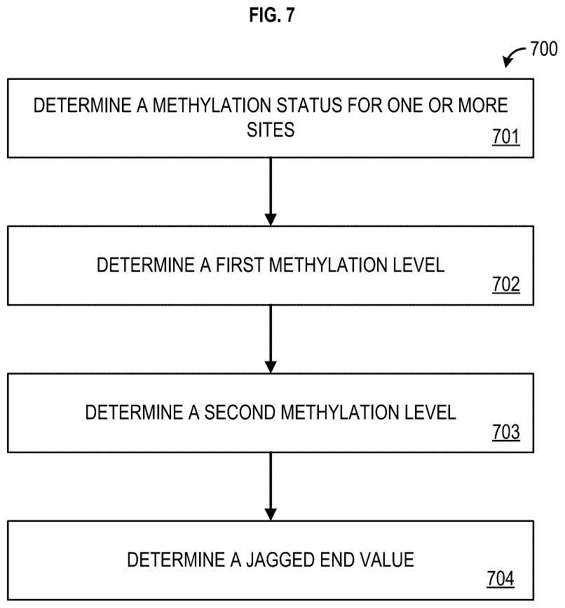

FIG. 7 shows a method 700 of determining an overhang index. A biological sample may include a plurality of nucleic acid molecules. The plurality of nucleic acid molecules may be cell-free. Each nucleic acid molecule of the plurality of nucleic acid molecules may be double-stranded with a first strand having a first portion and a second strand. The first portion of the first strand of at least some of the plurality of nucleic acid molecules may overhang the second strand, may not be hybridized to the second strand, and may be at a first end of the first strand.

At step 701, a methylation status of one or more sites of one or more strands may be determined. A first compound including one or more nucleotides may be hybridized to the first portion of the first strand for each nucleic acid molecule of the plurality of nucleic acid molecules. The first compound may be attached to a first end of the second strand to form an elongated second strand with a first end including the first compound. The first compound may include a first end not contacting the second strand. The one or more nucleotides may be unmethylated. In other implementations, certain nucleotides (e.g., cytosine) are all methylated, with the other nucleotides not being methylated. The first compound may be hybridized to the first portion one nucleotide at a time.

The first strand may be separated from the elongated second strand for each nucleic acid molecule of the plurality of nucleic acid molecules. A first methylation status for each of one or more first sites of the elongated second strand may be determined for each nucleic acid molecule of the plurality of nucleic acid molecules. The one or more first sites may be at the first end of the elongated second strand. A second methylation status for each of one or more second sites of the elongated second strand may optionally be determined for each nucleic acid molecule of the plurality of nucleic acid molecules. The one or more second sites may be at the second end of the elongated second strand. The one or more second sites may include the outermost sites at the second end of the elongated second strand. In some examples, the methylation status for the second sites may not need to be determined and may instead be assumed to be an average methylation status. The average methylation status may be known from a known frequency of methylated CpG sites in a particular region of the genome. In some instances, the average methylation status may be determined from reference samples taken from the same individual from which the biological sample is obtained and/or from other individuals.

At step 702, a first methylation level may be determined using the first methylation statuses for the plurality of elongated second strands at the one or more first sites. The first methylation level may be a mean or median of the first methylation statuses.

At step 703, a second methylation level may optionally be calculated using the second methylation statuses for the plurality of elongated second strands at the one or more second sites. The second methylation level may be a mean or median of the second methylation statuses. In some embodiments, the second methylation level may be assumed to be an average methylation level. The average methylation level may be based on a known frequency of methylated CpG sites in a particular region of the genome. In some instances, the average methylation level may be determined from reference samples taken from the same individual from which the biological sample is obtained and/or from other individuals. For example, the second methylation level may be assumed to be a value from 70% to 80%.

At step 704, an overhang index using the first methylation level and the second methylation level may be determined. A difference between the first methylation level and the second methylation level may be proportional to an average length of the first strands that overhang the second strands. Calculating the overhang index may be by calculating a difference between the first methylation level and the second methylation level and dividing the difference by the first methylation level (e.g., overall overhang index of FIG. 6).

In an embodiment, the fragmentomic component 234 may be configured to determine genomic locations of fragmentation endpoints. The fragmentomic component 234 may determine information about the two physical ends of DNA molecules. Both outer alignment coordinates of paired end data for which both reads aligned to the same chromosome and where reads have opposite orientations may be used as read starts. In cases where paired end data was converted to single read data by adapter trimming, both end coordinates of the single read alignment may be used as read starts. For coverage, all positions between the two (inferred) molecule ends, including these end positions, may be considered. It is expected that cfDNA fragment endpoints should cluster adjacent to nucleosome boundaries, while also being depleted on the nucleosome itself. To quantify this, a windowed protection scores (WPS) of a window size k may be defined as the number of molecules spanning a window minus those starting at any bases encompassed by the window. The determined WPS may be assigned to the center of the window. For molecules in the 35-80 bp range (short fraction), a window size of 16 may be used, for example, and, for molecules in the 120-180 bp (long fraction), a window size of 120 may be used, for example. High WPS values indicate increased protection of DNA from digestion; low values indicate that DNA is unprotected. Peak calls identify contiguous regions of elevated WPS.

Returning to FIG. 2, the results determined by the fragmentomics component 234 may be associated with the sequence fragments and/or variants in the sequence data that were used to generate such results. And, in the instance of the sequence data being derived from known samples 201, the origin of the sequence fragments and/or variants may also be associated with the sequence data, the epigenetic data, and/or the fragmentomic data. For example, sequence data, epigenetic data, and fragmentomic data of sequence fragments and/or variants known to be tumor derived may be labeled as tumor derived and sequence data, epigenetic data, and fragmentomic data of sequence fragments and/or variants known to be non-tumor derived may be labeled as non-tumor derived. Moreover, further labels may be assigned, for example, cancer type, tissue type, and the like.

This summarizes the characteristics of cfDNA fragmentomics and its potential applications, such as fragment length, end motifs, jagged ends, breakpoint coordinates, and motifs, as well as nucleosome footprints, open chromatin region, and gene expression characterized by the distribution pattern of cfDNA fragments across the genome.

d. Genomic Alterations

The cfDNA is also analyzed for the presence of any tumor-derived mutations or other genomic alterations, such as copy number variations. The sequencing data can be used to identify these.

A variant caller 238 may retrieve/receive data from the analysis datastore 240. For example, the variant caller 238 may retrieve/receive data representing a plurality of sequence fragments/reads. The plurality of sequence fragments/reads may be analyzed to determine one or more variants. Variants may include, for example, single nucleotide variants (SNVs), indels, fusions, and copy number variation. Any known technique for variant calling may be used. In an embodiment, nucleotide variations in sequenced nucleic acids can be determined by comparing sequenced nucleic acids with a reference sequence. The reference sequence is often a known sequence, e.g., a known whole or partial genome sequence from a subject (e.g., a whole genome sequence of a human subject). The reference sequence can be, for example, hG19 or hG38. The sequenced nucleic acids can represent sequences determined directly for a nucleic acid in a sample, or a consensus of sequences of amplification products of such a nucleic acid, as described above. A comparison can be performed at one or more designated positions on a reference sequence. A subset of sequenced nucleic acids can be identified including a position corresponding with a designated position of the reference sequence when the respective sequences are maximally aligned. Within such a subset it can be determined which, if any, sequenced nucleic acids include a nucleotide variation at the designated position, the length of a given cfDNA fragment based upon where its endpoints (i.e., it 5′ and 3′ terminal nucleotides) map to the reference sequence, the offset of a midpoint of a given cfDNA fragment from a midpoint of a genomic region in the cfDNA fragment, and optionally which if any, include a reference nucleotide (i.e., same as in the reference sequence). If the number of sequenced nucleic acids in the subset including a nucleotide variant exceeding a selected threshold, then a variant nucleotide can be called at the designated position. The threshold can be a simple number, such as at least 1, 2, 3, 4, 5, 6, 7, 9, or 10 sequenced nucleic acids within the subset including the nucleotide variant or it can be a ratio, such as a least 0.5, 1, 2, 3, 4, 5, 10, 15, or 20 of sequenced nucleic acids within the subset that include the nucleotide variant, among other possibilities. The comparison can be repeated for any designated position of interest in the reference sequence. Sometimes a comparison can be performed for designated positions occupying at least about 20, 100, 200, or 300 contiguous positions on a reference sequence, e.g., about 20-500, or about 50-300 contiguous positions.

The disease classifier 239 may generate a disease test result is based on two scores: a score from the TFR methylation component 235 and an integrated score. The disease classifier 239 may include an integrated score component having a LR model to generate an integrated quantitative score indicating presence of tumor-derived molecules based on the joint assessment of the epigenetic data from the epigenetic component 232 (e.g., the cfDNA methylation status from the LR methylation component 233 and the TFR methylation component 235 and fragmentation patterns from the fragmentomics component 234) and a qualitative mutation detected status (e.g., for somatic mutations based on data from the variant caller 238). Each of these analytes may be first analyzed separately and then the resulting individual quantitative scores of the per-analyte assessments are aggregated by the LR model to produce a single integrated score.

If either the integrated score or the TFR score exceeds their respective pre-defined thresholds, the disease classifier 239 may provide the disease test result indicating a positive result (e.g., abnormal). For example, if the integrated score (e.g., a cell-free nucleic acid score) satisfies a first respective threshold or the TFR score satisfies a second respective threshold, the samples may be determined to be tumor-derived. Otherwise, the disease classifier 239 may provide the disease test result indicating a negative result (e.g., normal). In some examples, the disease classifier 239 may be configured to predict colorectal cancer (CRC). The determination that the samples are tumor-derived or non-tumor derived may be used as a basis for a diagnosis for a particular disease, as well as may inform a treatment plan for the diagnosed disease.

Any data analyzed, determined, and/or output by the sequence analysis pipeline 230 may be stored in the analysis datastore 240. Generally speaking, the processor 220 may implement (be programmed by) various components of the sequence analysis pipeline 230, such as the sequence quality control component 231, the epigenetic component 232, the TFR methylation component 235, the variant caller 238, the disease classifier 239, and/or other components. Alternatively, it should be noted that these components of the sequence analysis pipeline 230 may include a hardware module. Although illustrated separately for convenience, one or more of the various components or instructions, such as the sequence quality control component 231, the epigenetic component 232, the TFR methylation component 235, the variant caller 238, and/or the disease classifier 239 may be integrated with one another.

The computer system 210 may exchange data with a computer system 224 using a network 223. For example, the computer system 224 may retrieve data from the analytics datastore 236. The computer system 224 may be configured for generating a predictive model (e.g., a classifier) and/or for utilizing a predictive model to determine an origin of a sequence fragment and/or variant.

The copy number component (included in 238) may use the sequence fragments/reads to generate a chromosomal region of coverage. The copy number component may divide the chromosomal regions into variable length windows or bins. A window or bin may be at least 5 kb, 10, kb, 25 kb, 30 kb, 35, kb, 40 kb, 50 kb, 60 kb, 75 kb, 100 kb, 150 kb, 200 kb, 500 kb, or 1000 kb. A window or bin may also have bases up to 5 kb, 10, kb, 25 kb, 30 kb, 35, kb, 40 kb, 50 kb, 60 kb, 75 kb, 100 kb, 150 kb, 200 kb, 500 kb, or 1000 kb. A window or bin may also be about 5 kb, 10, kb, 25 kb, 30 kb, 35, kb, 40 kb, 50 kb, 60 kb, 75 kb, 100 kb, 150 kb, 200 kb, 500 kb, or 1000 kb.

The copy number component may normalize coverage by causing the window or bin to contain about the same number of mappable bases. In some cases, each window or bin in a chromosomal region may contain the exact number of mappable bases. In other cases, each window or bin may contain a different number of mappable bases. Additionally, each window or bin may be non-overlapping with an adjacent window or bin. In other cases, a window or bin may overlap with another adjacent window or bin. In some cases a window or bin may overlap by at least 1 bp, 2, bp, 3 bp, 4 bp, 5, bp, 10 bp, 20 bp, 25 bp, 50 bp, 100 bp, 200 bp, 250 bp, 500 bp, or 1000 bp. In other cases, a window or bin may overlap by up to 1 bp, 2, bp, 3 bp, 4 bp, 5, bp, 10 bp, 20 bp, 25 bp, 50 bp, 100 bp, 200 bp, 250 bp, 500. bp, or 1000 bp. In some cases a window or bin may overlap by about 1 bp, 2, bp, 3 bp, 4 bp, 5, bp, 10 bp, 20 bp, 25 bp, 50 bp, 100 bp, 200 bp, 250 bp, 500 bp, or 1000 bp.

In some cases, each of the window regions may be sized so they contain about the same number of uniquely mappable bases. The mappability of each base that comprise a window region is determined and used to generate a mappability file which contains a representation of fragments/reads from the references that are mapped back to the reference for each file. The mappability file contains one row per every position, indicating whether each position is or is not uniquely mappable.

Additionally, predefined windows, known throughout the genome to be hard to sequence, or contain a substantially high GC bias, may be filtered from the data set. For example, regions known to fall near the centromere of chromosomes (i.e., centromeric DNA) are known to contain highly repetitive sequences that may produce false positive results. These regions may be filtered out. Other regions of the genome, such as regions that contain an unusually high concentration of other highly repetitive sequences such as microsatellite DNA, may be filtered from the data set.

The number of windows analyzed may also vary. In some cases, at least 10, 20, 30, 40, 50, 100, 200, 500, 1000, 2000, 5,000, 10,000, 20,000, 50,000 or 100,000 windows are analyzed. In other cases, the number of widows analyzed is up to 10, 20, 30, 40, 50, 100, 200, 500, 1000, 2000, 5,000, 10,000, 20,000, 50,000 or 100,000 windows are analyzed.

The copy number component may determine the read coverage for each window/bin region. This may be performed using either fragments/reads with barcodes, or without barcodes. In cases without barcodes, the previous mapping steps will provide coverage of different base positions. Sequence fragments/reads that have sufficient mapping and quality scores and fall within chromosome windows that are not filtered, may be counted. The number of coverage fragments/reads may be assigned a score per each mappable position.

In an embodiment, a quantitative measure related to sequencing read coverage is a measure indicative of the number of fragments/reads derived from a DNA molecule corresponding to a genetic locus (e.g., a particular position, base, region, gene or chromosome from a reference genome). In order to associate fragments/reads to a genetic locus, the fragments/reads can be mapped or aligned to the reference. Software to perform mapping or aligning (e.g., Bowtie, BWA, mrsFAST, BLAST, BLAT) can associate a sequencing read with a genetic locus. During the mapping process, particular parameters can be optimized. Non-limiting examples of optimization of the mapping processing can include masking repetitive regions; employing mapping quality (e.g., MAPQ) score cut-offs; using different seed lengths to generate alignments; and limiting the edit distance between positions of the genome.

Quantitative measures associated with sequencing read coverage can include counts of fragments/reads associated with a genetic locus. In some cases, the counts are transformed into new metrics to mitigate the effects of differing sequencing depth, library complexity, or size of the genetic locus. Exemplary metrics are Read Per Kilobase per Million (RPKM), Fragments Per Kilobase per Million (FPKM), Trimmed Mean of M values (TMM), variance stabilized raw counts, and log transformed raw counts. Other transformations are also known to those of skill in the art that may be used for particular applications.

Quantitative measures can be determined using numbers of fragment/read families or collapsed fragments/reads, wherein each read family or collapsed read corresponds to an initial template DNA molecule. Methods to collapse and quantify read families are found in PCT/US2013/058061 and PCT/US2014/000048, each of which is herein incorporated by reference in its entirety. In particular, quantifying read families and/or collapsing methods can be employed that use barcodes and sequence information from the sequencing read to sort fragments/reads into families, such that each family shares barcode sequences and at least a portion of the sequencing read sequence and/or the same genomic coordinates when mapped to a reference sequence. Each family is then, for the majority of the families, derived from a single initial template DNA molecule. Counts derived from mapping sequences from families can be referred to as “unique molecular counts” (UMCs). In some cases, determining a quantitative measure related to sequencing read coverage comprises normalizing UMCs by a metric related to library size to provide normalized UMCs (“normalized UMCs”). Exemplary methods are dividing the UMC of a genetic locus by the sum of all UMCs; dividing the UMC of a genetic locus by the sum of all autosomal UMCs. When comparing multiple sequencing read data sets, UMCs can, for example, be normalized by the median UMCs of the genetic loci of the two sequencing read data sets. In some cases, the quantitative measure related to sequencing read coverage can be normalized UMCs that are further normalized as follows: (i) normalized UMCs are determined for corresponding genetic loci from sequencing fragments/reads derived from training samples; (ii) for each genetic locus, normalized UMCs of the sample are normalized by the median of the normalized UMCs of the training samples at the corresponding loci, thereby providing Relative Abundances (RAs) of genetic loci.

Consensus sequences can identified based on their sequences, for example by collapsing sequencing fragments/reads based on identical sequences within the first 5, 10, 15, 20, or 25 bases. In some cases, collapsing allows for 1 difference, 2 differences, 3 differences, 4 differences, or 5 differences in the fragments/reads that are otherwise identical. In some cases, collapsing uses the mapping position of the read, for example the mapping position of the initial base of the sequencing read. In some cases, collapsing uses barcodes, and sequencing fragments/reads that share barcode sequences are collapsed into a consensus sequence. In some cases, collapsing uses both barcodes and the sequence of the initial template molecules. For example, all fragments/reads that share a barcode and map to the same position in the reference genome can be collapsed. In another example, all fragments/reads that share a barcode and a sequence of the initial template molecule (or a percentage identity to a sequence of the initial template molecule) can be collapsed.

In some cases, quantitative measures of sequencing read coverage are determined for specific sub-regions of a genome. Regions can be bins, genes of interest, exons, regions corresponding to sequence probes, regions corresponding to primer amplification products, or regions corresponding to primer binding sites. In some cases, sub-regions of the genome are regions corresponding to sequence capture probes. A read can map to a region corresponding to the sequence capture probe if at least a portion of the read maps at least a portion of the region corresponding to the sequence capture probe. A read can map to a region corresponding to the sequence capture probe if at least a portion of the read maps to the majority of the region corresponding to the sequence capture probe. A read can map to a region corresponding to the sequence capture probe if at least a portion of the read maps across the center point of the region corresponding to the sequence capture probe.

In another embodiment involving barcodes, all sequences with the same barcode, physical properties or combination of the two may be collapsed into one read, as they are all derived from the sample parent molecule to reduce biases which may have been introduced during amplification. For example, if one molecule is amplified 10 times but another is amplified 1000 times, each molecule is only represented once after collapse thereby negating the effect of uneven amplification. Only fragments/reads with unique barcodes may be counted for each mappable position and influence the assigned score.

Consensus sequences can be generated from families of sequence fragments/reads by any method known in the art. Such methods include, for example, linear or non-linear methods of building consensus sequences (such as voting, averaging, statistical, maximum a posteriori or maximum likelihood detection, dynamic programming, Bayesian, hidden Markov or support vector machine methods, etc.) derived from digital communication theory, information theory, or bioinformatics.

After the sequence read coverage has been determined, a stochastic modeling algorithm may be applied to convert the normalized nucleic acid sequence read coverage for each window/bin region to the discrete copy number states. In some cases, this algorithm may comprise one or more of the following: Hidden Markov Model, dynamic programming, support vector machine, Bayesian network, trellis decoding, Viterbi decoding, expectation maximization, Kalman filtering methodologies and neural networks. The discrete copy number states of each window region can be utilized to identify copy number variation in the chromosomal regions. In some cases, all adjacent window/bin regions with the same copy number can be merged into a segment to report the presence or absence of copy number variation state. In some cases, various windows/bins can be filtered before they are merged with other segments. The copy number variation may be stored in the analysis datastore 240 and/or reported as graph, indicating various positions in the genome and a corresponding increase or decrease or maintenance of copy number variation at each respective position. Additionally, copy number variation may be used to report a percentage score indicating how much disease material (or nucleic acids having a copy number variation) exists in the cell free polynucleotide sample.

vii. Predictive Models

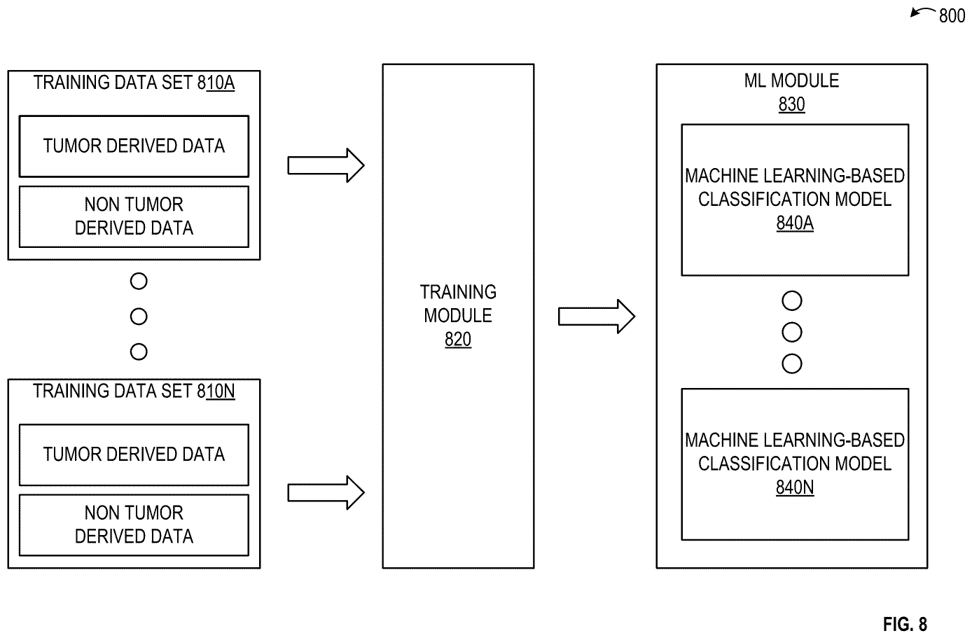

Turning now to FIG. 8, additional methods are described for generating a predictive model (e.g., a classifier). FIG. 8 is a flowchart illustrating an example method 800 for generating a predictive model. The methods described may use machine learning (“ML”) techniques to train, based on an analysis of one or more training data sets 810 by a training module 820, at least one ML module 830 that is configured to classify sequence fragments and/or variants in plasma as tumor origin or non-tumor origin, which can be from clonal hematopoiesis or biological noise.

The training data set 810 may comprise tumor derived and non-tumor derived (e.g., cancer/non-cancer) bodily fluid (e.g., blood, plasma, serum, cerebrospinal fluid, urine) sample data. The sample data may comprise sequence data which may comprise sequence information for one or more sequence fragments/reads and/or variants. The sample data may comprise epigenetic data, including methylation data and fragmentomic data. The epigenetic data may include, for example, information regarding DNA methylation, histone states or modifications, inflammation-mediated cytosine damage products, protein binding, or other molecular states reflected in the nucleic acid fragment analyzed that are not ascertained solely from the nucleotide base sequence, e.g., the methylation status of give base or set bases. The fragmentomic data may include, for example, information regarding fragment mapped starts and stops positions (correlated with nucleosome positions), fragment length and associated nucleosome occupancy. In an embodiment, the origin (tumor derived and non-tumor derived) of the sequence fragments/reads and/or variants in the sequence data may also be associated with the sequence data, the epigenetic data, and/or the fragmentomic data. For example, sequence data, epigenetic data, and fragmentomic data of sequence fragments/reads and/or variants known to be tumor derived may be labeled as tumor derived and sequence data, epigenetic data, and fragmentomic data of sequence fragments and/or variants known to be non-tumor derived may be labeled as non-tumor derived. Moreover, further labels may be assigned, for example, cancer type, tissue type, and the like.

A subset of the tumor derived/non-tumor derived sample data may be randomly assigned to the training data set 810 or to a testing data set. In some implementations, the assignment of data to a training data set or a testing data set may not be completely random. In this case, one or more criteria may be used during the assignment. In general, any suitable method may be used to assign the data to the training or testing data sets, while ensuring that the data distributions are somewhat similar in the training data set and the testing data set.

The training module 820 may train the ML module 830 by extracting a feature set from the tumor derived/non-tumor derived sample data in the training data set 810 according to one or more feature selection techniques. The training module 820 may train the ML module 830 by extracting a feature set from the training data set 810 that includes statistically significant features.

The training module 820 may extract a feature set from the training data set 810 in a variety of ways. The training module 820 may perform feature extraction multiple times, each time using a different feature-extraction technique. In an example, the feature sets generated using the different techniques may each be used to generate different machine learning-based classification models 840. For example, the feature set with the highest quality metrics may be selected for use in training. The training module 820 may use the feature set(s) to build one or more machine learning-based classification models 840A-840N that are configured to classify an origin as tumor or non-tumor for a new variant (e.g., with an unknown origin).

The training data set 810 may be analyzed to determine any dependencies, associations, and/or correlations between features and the experimental parameters in the training data set 810. The identified correlations may have the form of a list of features. The term “feature,” as used herein, may refer to any characteristic of an item of data that may be used to determine whether the item of data falls within one or more specific categories. By way of example, the features described herein may comprise any data and/or calculated values described herein, including: frequency of observance of a genetic variant among samples of particular cancer type, including hematological malignancies; prevalence of variants in plasma, tumor tissue, or white blood cells; methylation state vectors; methylation densities; fragment sizes; fragment size distributions; end motifs; end motif frequencies; jagged end presence; overhang indexes; genomic locations of fragmentation endpoints; windowed protection scores; combinations thereof and the like.

A feature selection technique may comprise one or more feature selection rules. The one or more feature selection rules may comprise a feature occurrence rule. The feature occurrence rule may comprise determining which features in the training data set 810 occur over a threshold number of times and identifying those features that satisfy the threshold as features.

A single feature selection rule may be applied to select features or multiple feature selection rules may be applied to select features. The feature selection rules may be applied in a cascading fashion, with the feature selection rules being applied in a specific order and applied to the results of the previous rule. For example, the feature occurrence rule may be applied to the training data set 810 to generate a first list of features. A final list of features may be analyzed according to additional feature selection techniques to determine one or more feature groups (e.g., groups of features that may be used to classify a sequence fragment/read and/or variant as tumor derived or non-tumor derived). Any suitable computational technique may be used to identify the feature groups using any feature selection technique such as filter, wrapper, and/or embedded methods. One or more feature groups may be selected according to a filter method. Filter methods include, for example, Pearson's correlation, linear discriminant analysis, analysis of variance (ANOVA), chi-square, combinations thereof, and the like. The selection of features according to filter methods are independent of any machine learning algorithms. Instead, features may be selected on the basis of scores in various statistical tests for their correlation with the outcome variable.

As another example, one or more feature groups may be selected according to a wrapper method. A wrapper method may be configured to use a subset of features and train a machine learning model using the subset of features. Based on the inferences that drawn from a previous model, features may be added and/or deleted from the subset. Wrapper methods include, for example, forward feature selection, backward feature elimination, recursive feature elimination, combinations thereof, and the like. As an example, forward feature selection may be used to identify one or more feature groups. Forward feature selection is an iterative method that begins with no feature in the machine learning model. In each iteration, the feature which best improves the model is added until an addition of a new variable does not improve the performance of the machine learning model. As an example, backward elimination may be used to identify one or more feature groups. Backward elimination is an iterative method that begins with all features in the machine learning model. In each iteration, the least significant feature is removed until no improvement is observed on removal of features. Recursive feature elimination may be used to identify one or more feature groups. Recursive feature elimination is a greedy optimization algorithm which aims to find the best performing feature subset. Recursive feature elimination repeatedly creates models and keeps aside the best or the worst performing feature at each iteration. Recursive feature elimination constructs the next model with the features remaining until all the features are exhausted. Recursive feature elimination then ranks the features based on the order of their elimination.

As a further example, one or more feature groups may be selected according to an embedded method. Embedded methods combine the qualities of filter and wrapper methods. Embedded methods include, for example, Least Absolute Shrinkage and Selection Operator (LASSO) and ridge regression which implement penalization functions to reduce overfitting. For example, LASSO regression performs L1 regularization which adds a penalty equivalent to absolute value of the magnitude of coefficients and ridge regression performs L2 regularization which adds a penalty equivalent to square of the magnitude of coefficients.

After the training module 820 has generated a feature set(s), the training module 820 may generate a machine learning-based classification model 840 based on the feature set(s). A machine learning-based classification model may refer to a complex mathematical model for data classification that is generated using machine-learning techniques. In one example, the machine learning-based classification model 840 may include a map of support vectors that represent boundary features. By way of example, boundary features may be selected from, and/or represent the highest-ranked features in, a feature set.

The training module 820 may use the feature sets determined or extracted from the training data set 810 to build a machine learning-based classification model 840A-840N. In some examples, the machine learning-based classification models 840A-840N may be combined into a single machine learning-based classification model 840. Similarly, the ML module 830 may represent a single classifier containing a single or a plurality of machine learning-based classification models 840 and/or multiple classifiers containing a single or a plurality of machine learning-based classification models 840.

The features may be combined in a classification model trained using a machine learning approach such as discriminant analysis; decision tree; a nearest neighbor (NN) algorithm (e.g., k-NN models, replicator NN models, etc.); statistical algorithm (e.g., Bayesian networks, etc.); clustering algorithm (e.g., k-means, mean-shift, etc.); neural networks (e.g., reservoir networks, artificial neural networks, etc.); support vector machines (SVMs); logistic regression algorithms; linear regression algorithms; Markov models or chains; principal component analysis (PCA) (e.g., for linear models); multi-layer perceptron (MLP) ANNs (e.g., for non-linear models); replicating reservoir networks (e.g., for non-linear models, typically for time series); random forest classification; a combination thereof and/or the like. The resulting ML module 830 may comprise a decision rule or a mapping for each feature to determine tumor/non-tumor origin for a variant.

In an embodiment, the training module 820 may train the machine learning-based classification models 840 as a convolutional neural network (CNN). The CNN comprises at least one convolutional feature layer and three fully connected layers leading to a final classification layer (softmax). The final classification layer may finally be applied to combine the outputs of the fully connected layers using softmax functions as is known in the art.

The feature(s) and the ML module 830 may be used to predict the tumor derived or non-tumor derived origin of sequence fragments/reads and/or variants in the testing data set. In one example, the prediction result for each sequence fragment/read and/or variant may include a confidence level that corresponds to a likelihood or a probability that a sequence fragment/read and/or variant in the testing data set is associated with tumor origin or non-tumor origin. The confidence level may be a value between zero and one. In one example, when there are two statuses (e.g., tumor origin and non-tumor origin), the confidence level may correspond to a value p, which refers to a likelihood that a particular variant belongs to the first status (e.g., tumor origin). In this case, the value 1−p may refer to a likelihood that the particular variant belongs to the second status (e.g., non-tumor origin). In general, multiple confidence levels may be provided for each variant in the testing data set and for each feature when there are more than two statuses. A top performing feature may be determined by comparing the result obtained for each test variant with the known tumor/non-tumor origin for each test variant. In general, the top performing feature will have results that closely match the known tumor/non-tumor origin statuses. The top performing feature(s) may be used to predict/classify the tumor/non-tumor origin status of a given variant.

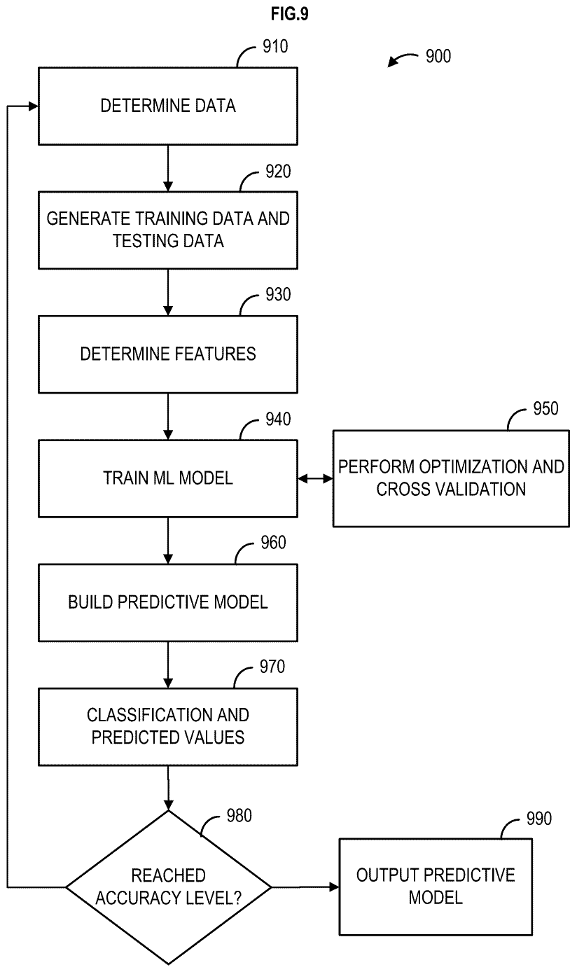

FIG. 9 is a flowchart illustrating an example training method 900 for generating the ML module 830 of FIG. 8 using the training module 820 of FIG. 8. The training module 820 can implement supervised, unsupervised, and/or semi-supervised (e.g., reinforcement based) machine learning-based classification models 840. The method 900 illustrated in FIG. 9 is an example of a supervised learning method; variations of this example of training method are discussed below, however, other training methods can be analogously implemented to train unsupervised and/or semi-supervised machine learning models.

The training method 900 may determine (e.g., access, receive, retrieve, etc.) data at step 910. The data may comprise tumor derived/non-tumor derived bodily fluid sample data. The data may comprise sequence data, epigenetic data, and/or fragmentomic data for one or more sequence fragments reads and/or variants, each sequence fragment/read and/or variant having an assigned tumor derived or non-tumor derived origin status.

The training method 900 may generate, at step 920, a training data set and a testing data set. The training data set and the testing data set may be generated by randomly assigning data to either the training data set or the testing data set. In some implementations, the assignment of computation parameters and associated experimental parameters as training or testing data may not be completely random. As an example, a majority of the computation parameters and associated experimental parameters may be used to generate the training data set. For example, 75% of the computation parameters and associated experimental parameters may be used to generate the training data set and 25% may be used to generate the testing data set. In another example, 80% of the computation parameters and associated experimental parameters may be used to generate the training data set and 20% may be used to generate the testing data set.

The training method 900 may determine (e.g., extract, select, etc.), at step 930, one or more features that can be used by, for example, a classifier to differentiate among different classification of tumor derived vs. non-tumor derived status. As an example, the training method 900 may determine a set of features from the tumor derived/non-tumor derived bodily fluid sample data. In a further example, a set of features may be determined from data that is different than the tumor derived/non-tumor derived bodily fluid sample data in either the training data set or the testing data set. Such other data may be used to determine an initial set of features, which may be further reduced using the training data set.

The training method 900 may train one or more machine learning models using the one or more features at step 940. In one example, the machine learning models may be trained using supervised learning. In another example, other machine learning techniques may be employed, including unsupervised learning and semi-supervised. The machine learning models trained at 940 may be selected based on different criteria depending on the problem to be solved and/or data available in the training data set. For example, machine learning classifiers can suffer from different degrees of bias. Accordingly, more than one machine learning model can be trained at 940, optimized, improved, and cross-validated at step 950.

The training method 900 may select one or more machine learning models to build a predictive model at 960. The predictive model may be evaluated using the testing data set. The predictive model may analyze the testing data set and generate predicted tumor/non-tumor origin statuses at step 970. Predicted tumor/non-tumor origin may be evaluated at step 980 to determine whether such values have achieved a desired accuracy level. Performance of the predictive model may be evaluated in a number of ways based on a number of true positives, false positives, true negatives, and/or false negatives classifications of the plurality of data points indicated by the predictive model.

For example, the false positives of the predictive model may refer to a number of times the predictive model incorrectly classified a sequence fragment/read and/or variant as tumor origin that was in reality non-tumor origin. Conversely, the false negatives of the predictive model may refer to a number of times the machine learning model classified a sequence fragment/read and/or variant as non-tumor origin when, in fact, the sequence fragment/read and/or variant was tumor origin. True negatives and true positives may refer to a number of times the predictive model correctly classified one or more sequence fragment/read and/or variant. Related to these measurements are the concepts of recall and precision. Generally, recall refers to a ratio of true positives to a sum of true positives and false negatives, which quantifies a sensitivity of the predictive model. Similarly, precision refers to a ratio of true positives a sum of true and false positives. When such a desired accuracy level is reached, the training phase ends and the predictive model (e.g., the ML module 830) may be output at step 990; when the desired accuracy level is not reached, however, then a subsequent iteration of the training method 900 may be performed starting at step 910 with variations such as, for example, considering a larger collection of data.

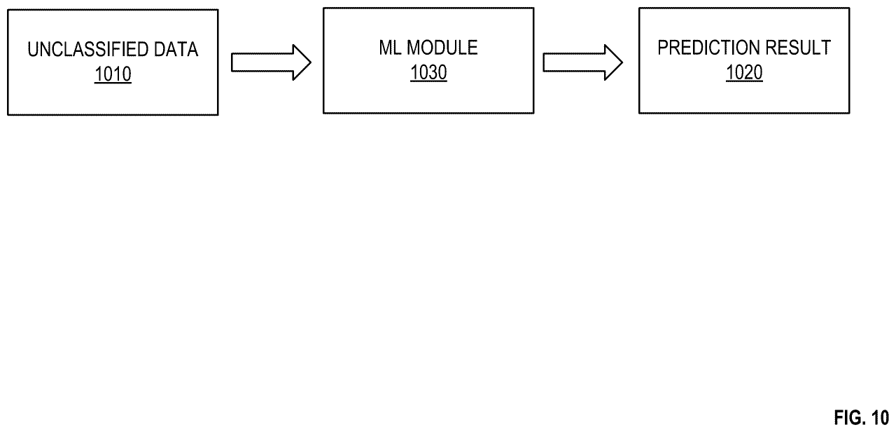

FIG. 10 is an illustration of an exemplary process flow for using a machine learning-based classifier to classify a sequence fragment/read and/or variant as tumor origin or non-tumor origin. As illustrated in FIG. 10, sequence data, epigenetic data, and/or fragmentomic data for an unclassified sequence fragment/read and/or variant 1010 may be provided as input to the ML module 830. The ML module 830 may process the sequence data, epigenetic data, and/or fragmentomic data for the unclassified sequence fragment/read and/or variant 1010 using a machine learning-based classifier(s) to arrive at a prediction result 1020. The prediction result 1020 may identify one or more characteristics of the sequence data, epigenetic data, and/or fragmentomic data for an unclassified sequence fragment/read and/or variant 1010. For example, the classification result 1020 may identify the origin status of the sequence fragment/read and/or variant 1010 (e.g., whether the sequence fragment/read and/or variant is tumor origin or non-tumor origin). Thus, in an embodiment, disclosed is a method implemented using a network-based computer system comprising one or more processors, a network interface, and one or more memories, the method comprising retrieving, by the computer system, sequence data, epigenetic data, and/or fragmentomic data having an indicated tumor derived origin or non-tumor derived origin status; and training, by the one or more processors, a machine-learning model by fitting one or more models to the sequence data, epigenetic data, and/or fragmentomic data, wherein each of the one or more models is configured to receive as input sequence data, epigenetic data, and/or fragmentomic data of an individual, and provide as output a prediction of the individual having or developing a tumor.

viii. Example Methods

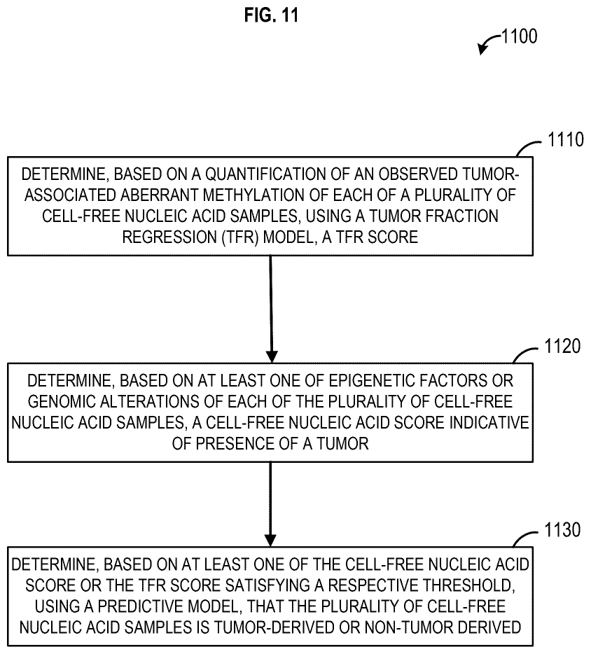

FIG. 11 is an illustration of an exemplary process flow of a method 1100 to classify nucleic acid samples as tumor origin or non-tumor origin. The method 1100 may be performed by the computer system 210 of FIG. 2 in some examples.

The method 1100 may include determining, based on a quantification of an observed tumor-associated aberrant methylation of each of a plurality of cell-free nucleic acid samples, using a TFR model, a TFR score, at 1110. The TFR model may be included in the TFR methylation component 235 of FIG. 2, in some examples. The TFR score may include a fraction of molecules of the plurality of cell-free nucleic acid samples that indicate a tumor. In some examples, the plurality of cell-free nucleic acid samples include cell-free deoxyribonucleic (cfDNA) samples. Additionally or alternatively, the plurality of cell-free nucleic acid samples includes one or more of ribonucleic acid (RNA) samples, cell-free ribonucleic acid (cfRNA) samples, cell-free deoxyribonucleic (cfDNA) samples, mitochondrial deoxyribonucleic (mtDNA) samples, mitochondrial ribonucleic (mtRNA) samples, and extracellular vesicle-bound deoxyribonucleic (evDNA) samples. In some examples, the plurality of cell-free nucleic acid samples are from a plurality of genomic regions. The plurality of genomic regions may include at least one of a genomic region known to be associated with a cancer type, a genomic region associated with a known methylation status, a genomic region known to be associated with hypomethylation, or a genomic region known to be associated with therapy response. In an example, the plurality of genomic regions includes at least one genomic region known to be associated with colorectal cancer.

In some examples, the method 1100 includes determining the quantification of the observed tumor-associated aberrant methylation of each of the plurality of cell-free nucleic acid samples. In some examples, determining the quantification of the observed tumor-associated aberrant methylation of each of the plurality of cell-free nucleic acid samples includes quantifying a number of unique methylated molecules mapping to each of the plurality of genomic regions.

The method 1100 may further include determining, based on at least one of epigenetic factors or genomic alterations of each of the plurality of cell-free nucleic acid samples, a cell-free nucleic acid score indicative of presence of a tumor, at 1120. In some examples, determining the cell-free nucleic acid score is further based on the TFR score. In some examples, the method 1100 includes determining the epigenetic factors of each of the plurality of cell-free nucleic acid samples. In some examples, determining the epigenetic factors of each of the plurality of cell-free nucleic acid samples based on a methylation LR model cancer or non-cancer classification. In some examples, the method 1100 further includes determining, using a LR model, the methylation LR model cancer or non-cancer classification.

In some examples, the method 1100 includes determining the epigenetic factors of each of the plurality of cell-free nucleic acid samples based on a cancer signal from cell-free nucleic acid fragmentation patterns associated with a plurality of sequence fragments from the plurality of cell-free nucleic acid samples. The epigenetic factors may be determined via the epigenetic component 232 of FIG. 2, in some examples. The In some examples, the method 1100 includes determining, using a fragmentomics model, the cancer signal from the cell-free nucleic acid fragmentation patterns associated with the plurality of sequence fragments from the plurality of cell-free nucleic acid samples. In some examples, the method 1100 includes determining the epigenetic factors of each of the plurality of cell-free nucleic acid samples based on a methylation LR model cancer or non-cancer classification and based on a cancer signal from cell-free nucleic acid fragmentation patterns associated with a plurality of sequence fragments from the plurality of cell-free nucleic acid samples.

In some examples, determining the genomic alterations of each of the plurality of cell-free nucleic acid samples includes determining somatic variants observed in molecules from each of a plurality of sequence fragments from the plurality of cell-free nucleic acid samples. The somatic variants may be determined via the variant caller 238 of FIG. 2, in some examples.

The method 1100 may further include determining, based on at least one of the cell-free nucleic acid score or the TFR score satisfying a respective threshold, using a predictive model, that the plurality of cell-free nucleic acid samples is tumor-derived or non-tumor derived, at 1130. In some examples, the predictive model may be implemented in the disease classifier 239 of FIG. 2. For example, if the cell-free nucleic acid score satisfies a first respective threshold or the TFR score satisfies a second respective threshold, the plurality of cell-free nucleic acid samples may be determined to be tumor-derived. Otherwise, the plurality of cell-free nucleic acid samples may be determined to be non-tumor-derived. The determination that the samples are tumor-derived or non-tumor derived may be used as a basis for a diagnosis for a particular disease, as well as may inform a treatment plan for the diagnosed disease.

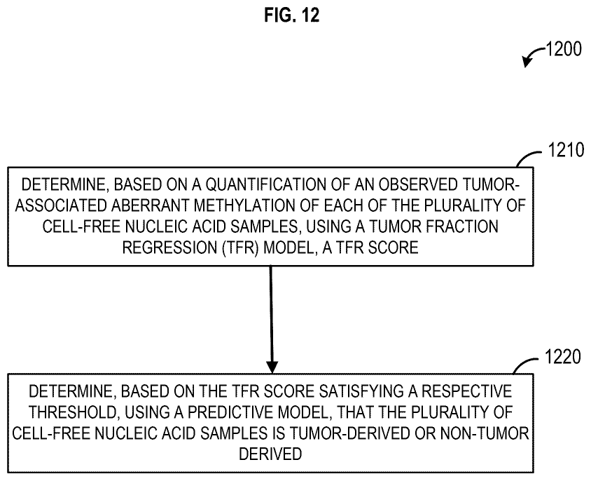

FIG. 12 is an illustration of an exemplary process flow of a method 1200 to classify nucleic acid samples as tumor origin or non-tumor origin. The method 1200 may be performed by the computer system 210 of FIG. 2 in some examples.

The method 1200 may include determining, based on a quantification of an observed tumor-associated aberrant methylation of each of a plurality of cell-free nucleic acid samples, using a TFR model, a TFR score, at 1210. The TFR model may be included in the TFR methylation component 235 of FIG. 2, in some examples. The TFR score may include a fraction of molecules of the plurality of cell-free nucleic acid samples that indicate a tumor. In some examples, the plurality of cell-free nucleic acid samples include cell-free deoxyribonucleic (cfDNA) samples. Additionally or alternatively, the plurality of cell-free nucleic acid samples includes one or more of ribonucleic acid (RNA) samples, cell-free ribonucleic acid (cfRNA) samples, cell-free deoxyribonucleic (cfDNA) samples, mitochondrial deoxyribonucleic (mtDNA) samples, mitochondrial ribonucleic (mtRNA) samples, and extracellular vesicle-bound deoxyribonucleic (evDNA) samples. In some examples, the plurality of cell-free nucleic acid samples are from a plurality of genomic regions. The plurality of genomic regions may include at least one of a genomic region known to be associated with a cancer type, a genomic region associated with a known methylation status, a genomic region known to be associated with hypomethylation, or a genomic region known to be associated with therapy response. In an example, the plurality of genomic regions includes at least one genomic region known to be associated with colorectal cancer.

In some examples, the method 1200 includes determining the quantification of the observed tumor-associated aberrant methylation of each of the plurality of cell-free nucleic acid samples. In some examples, determining the quantification of the observed tumor-associated aberrant methylation of each of the plurality of cell-free nucleic acid samples includes quantifying a number of unique methylated molecules mapping to each of a plurality of genomic regions.

The method 1200 may further include determining, based on the TFR score satisfying a respective threshold, using a predictive model, that the plurality of cell-free nucleic acid samples is tumor-derived or non-tumor derived, at 1220. In some examples, the predictive model may be implemented in the disease classifier 239 of FIG. 2. For example, if the TFR score satisfies the respective threshold, the plurality of cell-free nucleic acid samples may be determined to be tumor-derived. Otherwise, the plurality of cell-free nucleic acid samples may be determined to be non-tumor-derived. The determination that the samples are tumor-derived or non-tumor derived may be used as a basis for a diagnosis for a particular disease, as well as may inform a treatment plan for the diagnosed disease. In some examples, determining that the plurality of cell-free nucleic acid samples is tumor-derived or non-tumor derived is further based on a cell-free nucleic acid score indicative of presence of a tumor. For example, if the cell-free nucleic acid score satisfies a second respective threshold, the plurality of cell-free nucleic acid samples may be determined to be tumor-derived. Otherwise, the plurality of cell-free nucleic acid samples may be determined to be non-tumor-derived.

The method 1200 may further include determining, based on at least one of epigenetic factors or genomic alterations of each of the plurality of cell-free nucleic acid samples, a cell-free nucleic acid score indicative of presence of a tumor. In some examples, determining the cell-free nucleic acid score is further based on the TFR score. In some examples, the method 1200 includes determining the epigenetic factors of each of the plurality of cell-free nucleic acid samples. The epigenetic factors may be determined via the epigenetic component 232 of FIG. 2, in some examples. In some examples, determining the epigenetic factors of each of the plurality of cell-free nucleic acid samples based on a methylation LR model cancer or non-cancer classification. In some examples, the method 1200 further includes determining, using a LR model, the methylation LR model cancer or non-cancer classification.

In some examples, the method 1200 includes determining the epigenetic factors of each of the plurality of cell-free nucleic acid samples based on a cancer signal from cell-free nucleic acid fragmentation patterns associated with a plurality of sequence fragments from the plurality of cell-free nucleic acid samples. In some examples, the method 1200 includes determining, using a fragmentomics model, the cancer signal from the cell-free nucleic acid fragmentation patterns associated with the plurality of sequence fragments from the plurality of cell-free nucleic acid samples. In some examples, the method 1200 includes determining the epigenetic factors of each of the plurality of cell-free nucleic acid samples based on a methylation LR model cancer or non-cancer classification and based on a cancer signal from cell-free nucleic acid fragmentation patterns associated with a plurality of sequence fragments from the plurality of cell-free nucleic acid samples.

In some examples, determining the genomic alterations of each of the plurality of cell-free nucleic acid samples includes determining somatic variants observed in molecules from each of a plurality of sequence fragments from the plurality of cell-free nucleic acid samples. The somatic variants may be determined via the variant caller 238 of FIG. 2, in some examples.

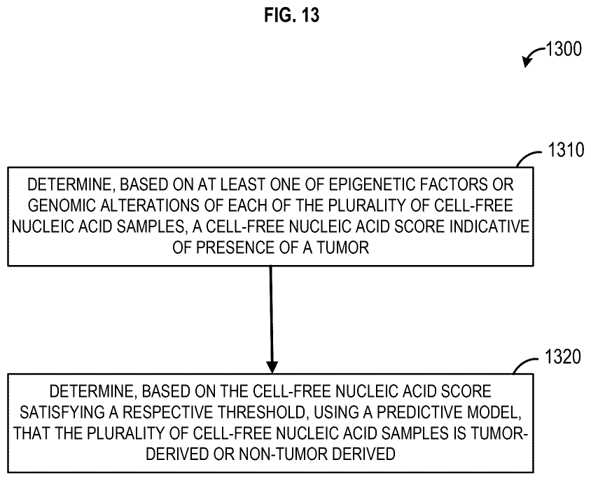

FIG. 13 is an illustration of an exemplary process flow of a method 1300 to classify nucleic acid samples as tumor origin or non-tumor origin. The method 1300 may be performed by the computer system 210 of FIG. 2 in some examples.

The method 1300 may further include determining, based on at least one of epigenetic factors or genomic alterations of each of the plurality of cell-free nucleic acid samples, a cell-free nucleic acid score indicative of presence of a tumor, at 1310. In some examples, the plurality of cell-free nucleic acid samples include cell-free deoxyribonucleic (cfDNA) samples. Additionally or alternatively, the plurality of cell-free nucleic acid samples includes one or more of ribonucleic acid (RNA) samples, cell-free ribonucleic acid (cfRNA) samples, cell-free deoxyribonucleic (cfDNA) samples, mitochondrial deoxyribonucleic (mtDNA) samples, mitochondrial ribonucleic (mtRNA) samples, and extracellular vesicle-bound deoxyribonucleic (evDNA) samples. In some examples, the plurality of cell-free nucleic acid samples are from a plurality of genomic regions. The plurality of genomic regions may include at least one of a genomic region known to be associated with a cancer type, a genomic region associated with a known methylation status, a genomic region known to be associated with hypomethylation, or a genomic region known to be associated with therapy response. In an example, the plurality of genomic regions includes at least one genomic region known to be associated with colorectal cancer. In some examples, the method 1300 includes determining the epigenetic factors of each of the plurality of cell-free nucleic acid samples. The epigenetic factors may be determined via the epigenetic component 232 of FIG. 2, in some examples. In some examples, determining the epigenetic factors of each of the plurality of cell-free nucleic acid samples based on a methylation LR model cancer or non-cancer classification. In some examples, the method 1300 further includes determining, using a LR model, the methylation LR model cancer or non-cancer classification.

In some examples, the method 1300 includes determining the epigenetic factors of each of the plurality of cell-free nucleic acid samples based on a cancer signal from cell-free nucleic acid fragmentation patterns associated with a plurality of sequence fragments from the plurality of cell-free nucleic acid samples. In some examples, the method 1300 includes determining, using a fragmentomics model, the cancer signal from the cell-free nucleic acid fragmentation patterns associated with the plurality of sequence fragments from the plurality of cell-free nucleic acid samples. In some examples, the method 1300 includes determining the epigenetic factors of each of the plurality of cell-free nucleic acid samples based on a methylation LR model cancer or non-cancer classification and based on a cancer signal from cell-free nucleic acid fragmentation patterns associated with a plurality of sequence fragments from the plurality of cell-free nucleic acid samples.

In some examples, determining the genomic alterations of each of the plurality of cell-free nucleic acid samples includes determining somatic variants observed in molecules from each of a plurality of sequence fragments from the plurality of cell-free nucleic acid samples. The somatic variants may be determined via the variant caller 238 of FIG. 2, in some examples.

The method 1300 may further include determining, based on the cell-free nucleic acid score satisfying a respective threshold, using a predictive model, that the plurality of cell-free nucleic acid samples is tumor-derived or non-tumor derived, at 1320. In some examples, the predictive model may be implemented in the disease classifier 239 of FIG. 2. For example, if the cell-free nucleic acid score satisfies the respective threshold, the plurality of cell-free nucleic acid samples may be determined to be tumor-derived. Otherwise, the plurality of cell-free nucleic acid samples may be determined to be non-tumor-derived. The determination that the samples are tumor-derived or non-tumor derived may be used as a basis for a diagnosis for a particular disease, as well as may inform a treatment plan for the diagnosed disease.

In some examples, determining that the plurality of cell-free nucleic acid samples is tumor-derived or non-tumor derived is further based on a TFR score satisfying a threshold. For example, if the TFR score satisfies a second respective threshold, the plurality of cell-free nucleic acid samples may be determined to be tumor-derived. Otherwise, the plurality of cell-free nucleic acid samples may be determined to be non-tumor-derived. In some examples, determining the cell-free nucleic acid score is further based on the TFR score. The method 1300 may include determining, based on a quantification of an observed tumor-associated aberrant methylation of each of a plurality of cell-free nucleic acid samples, using a TFR model, the TFR score. The TFR model may be included in the TFR methylation component 235 of FIG. 2, in some examples. The TFR score may include a fraction of molecules of the plurality of cell-free nucleic acid samples that indicate a tumor. In some examples, the method 1300 includes determining the quantification of the observed tumor-associated aberrant methylation of each of the plurality of cell-free nucleic acid samples. In some examples, determining the quantification of the observed tumor-associated aberrant methylation of each of the plurality of cell-free nucleic acid samples includes quantifying a number of unique methylated molecules mapping to each of the plurality of genomic regions.

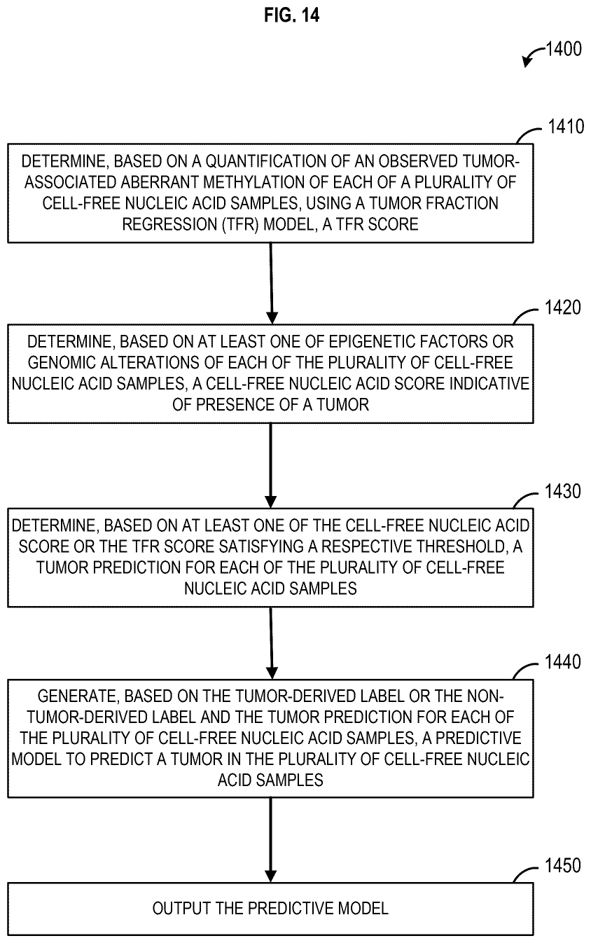

FIG. 14 is an illustration of an exemplary process flow of a method 1400 to train a predictive model to classify nucleic acid samples as tumor origin or non-tumor origin. The method 1400 may be performed by the computer system 210 of FIG. 2 in some examples.

The method 1400 may include determining, based on a quantification of an observed tumor-associated aberrant methylation of each of a plurality of cell-free nucleic acid samples, using a TFR model, a TFR score, at 1410. The TFR model may be included in the TFR methylation component 235 of FIG. 2, in some examples. The TFR score may include a fraction of molecules of the plurality of cell-free nucleic acid samples that indicate a tumor. Each of the plurality of cell-free nucleic acid samples may be labeled with a tumor-derived label or a non-tumor-derived label In some examples, the plurality of cell-free nucleic acid samples include cell-free deoxyribonucleic (cfDNA) samples. Additionally or alternatively, the plurality of cell-free nucleic acid samples includes one or more of ribonucleic acid (RNA) samples, cell-free ribonucleic acid (cfRNA) samples, cell-free deoxyribonucleic (cfDNA) samples, mitochondrial deoxyribonucleic (mtDNA) samples, mitochondrial ribonucleic (mtRNA) samples, and extracellular vesicle-bound deoxyribonucleic (evDNA) samples. In some examples, the plurality of cell-free nucleic acid samples are from a plurality of genomic regions. The plurality of genomic regions may include at least one of a genomic region known to be associated with a cancer type, a genomic region associated with a known methylation status, a genomic region known to be associated with hypomethylation, or a genomic region known to be associated with therapy response. In an example, the plurality of genomic regions includes at least one genomic region known to be associated with colorectal cancer.

In some examples, the method 1400 includes determining the quantification of the observed tumor-associated aberrant methylation of each of the plurality of cell-free nucleic acid samples. In some examples, determining the quantification of the observed tumor-associated aberrant methylation of each of the plurality of cell-free nucleic acid samples includes quantifying a number of unique methylated molecules mapping to each of the plurality of genomic regions.

The method 1400 may further include determining, based on at least one of epigenetic factors or genomic alterations of each of the plurality of cell-free nucleic acid samples, a cell-free nucleic acid score indicative of presence of a tumor, at 1420. In some examples, determining the cell-free nucleic acid score is further based on the TFR score. In some examples, the method 1400 includes determining the epigenetic factors of each of the plurality of cell-free nucleic acid samples. The epigenetic factors may be determined via the epigenetic component 232 of FIG. 2, in some examples. In some examples, determining the epigenetic factors of each of the plurality of cell-free nucleic acid samples based on a methylation LR model cancer or non-cancer classification. In some examples, the method 1400 further includes determining, using a LR model, the methylation LR model cancer or non-cancer classification.

In some examples, the method 1400 includes determining the epigenetic factors of each of the plurality of cell-free nucleic acid samples based on a cancer signal from cell-free nucleic acid fragmentation patterns associated with a plurality of sequence fragments from the plurality of cell-free nucleic acid samples. In some examples, the method 1400 includes determining, using a fragmentomics model, the cancer signal from the cell-free nucleic acid fragmentation patterns associated with the plurality of sequence fragments from the plurality of cell-free nucleic acid samples. In some examples, the method 1400 includes determining the epigenetic factors of each of the plurality of cell-free nucleic acid samples based on a methylation LR model cancer or non-cancer classification and based on a cancer signal from cell-free nucleic acid fragmentation patterns associated with a plurality of sequence fragments from the plurality of cell-free nucleic acid samples.

In some examples, determining the genomic alterations of each of the plurality of cell-free nucleic acid samples includes determining somatic variants observed in molecules from each of a plurality of sequence fragments from the plurality of cell-free nucleic acid samples. The somatic variants may be determined via the variant caller 238 of FIG. 2, in some examples.

The method 1400 may further include determining, based on at least one of the cell-free nucleic acid score or the TFR score satisfying a respective threshold, a tumor prediction for each of the plurality of cell-free nucleic acid samples, at 1430. For example, if the cell-free nucleic acid score satisfies a first respective threshold or the TFR score satisfies a second respective threshold, the plurality of cell-free nucleic acid samples may be determined to be tumor-derived. Otherwise, the plurality of cell-free nucleic acid samples may be determined to be non-tumor-derived.

The method 1400 may further include generating, based on the tumor-derived label or the non-tumor-derived label and the tumor prediction for each of the plurality of cell-free nucleic acid samples, a predictive model to predict a tumor in the plurality of cell-free nucleic acid samples, at 1440. The method 1400 may further include outputting the predictive model, at 1450. In some examples, the predictive model may be implemented in the disease classifier 239 of FIG. 2. The predictive model may be used to predict whether samples are tumor-derived or non-tumor derived, which may be used as a basis for a diagnosis for a particular disease, as well as may inform a treatment plan for the diagnosed disease.

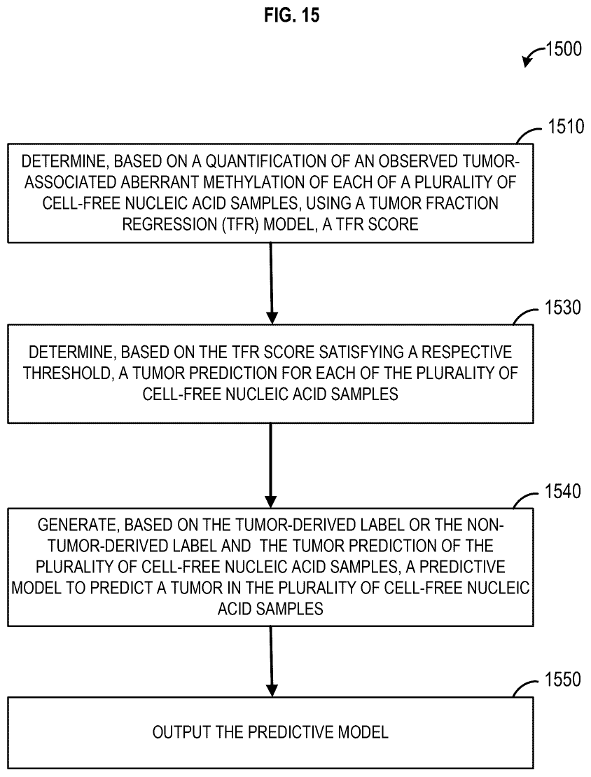

FIG. 15 is an illustration of an exemplary process flow of a method 1500 to train a predictive model to classify nucleic acid samples as tumor origin or non-tumor origin. The method 1500 may be performed by the computer system 210 of FIG. 2 in some examples.

The method 1500 may include determining, based on a quantification of an observed tumor-associated aberrant methylation of each of a plurality of cell-free nucleic acid samples, using a TFR model, a TFR score, at 1510. The TFR model may be included in the TFR methylation component 235 of FIG. 2, in some examples. The TFR score may include a fraction of molecules of the plurality of cell-free nucleic acid samples that indicate a tumor. Each of the plurality of cell-free nucleic acid samples may be labeled with a tumor-derived label or a non-tumor-derived label In some examples, the plurality of cell-free nucleic acid samples include cell-free deoxyribonucleic (cfDNA) samples. Additionally or alternatively, the plurality of cell-free nucleic acid samples includes one or more of ribonucleic acid (RNA) samples, cell-free ribonucleic acid (cfRNA) samples, cell-free deoxyribonucleic (cfDNA) samples, mitochondrial deoxyribonucleic (mtDNA) samples, mitochondrial ribonucleic (mtRNA) samples, and extracellular vesicle-bound deoxyribonucleic (evDNA) samples. In some examples, the plurality of cell-free nucleic acid samples are from a plurality of genomic regions. The plurality of genomic regions may include at least one of a genomic region known to be associated with a cancer type, a genomic region associated with a known methylation status, a genomic region known to be associated with hypomethylation, or a genomic region known to be associated with therapy response. In an example, the plurality of genomic regions includes at least one genomic region known to be associated with colorectal cancer.

In some examples, the method 1500 includes determining the quantification of the observed tumor-associated aberrant methylation of each of the plurality of cell-free nucleic acid samples. In some examples, determining the quantification of the observed tumor-associated aberrant methylation of each of the plurality of cell-free nucleic acid samples includes quantifying a number of unique methylated molecules mapping to each of the plurality of genomic regions.

The method 1500 may further include determining, based on the TFR score satisfying a respective threshold, a tumor prediction for each of the plurality of cell-free nucleic acid samples, at 1530. For example, if the TFR score satisfies the respective threshold, the plurality of cell-free nucleic acid samples may be determined to be tumor-derived. Otherwise, the plurality of cell-free nucleic acid samples may be determined to be non-tumor-derived.

In some examples, determining the tumor prediction for each of the plurality of cell-free nucleic acid samples is further based on a cell-free nucleic acid score indicative of presence of a tumor. For example, if the cell-free nucleic acid score satisfies a second respective threshold, the plurality of cell-free nucleic acid samples may be determined to be tumor-derived. The method 1500 may further include determining, based on at least one of epigenetic factors or genomic alterations of each of the plurality of cell-free nucleic acid samples, a cell-free nucleic acid score indicative of presence of a tumor. In some examples, determining the cell-free nucleic acid score is further based on the TFR score. In some examples, the method 1500 includes determining the epigenetic factors of each of the plurality of cell-free nucleic acid samples. The epigenetic factors may be determined via the epigenetic component 232 of FIG. 2, in some examples. In some examples, determining the epigenetic factors of each of the plurality of cell-free nucleic acid samples based on a methylation LR model cancer or non-cancer classification. In some examples, the method 1500 further includes determining, using a LR model, the methylation LR model cancer or non-cancer classification.

In some examples, the method 1500 includes determining the epigenetic factors of each of the plurality of cell-free nucleic acid samples based on a cancer signal from cell-free nucleic acid fragmentation patterns associated with a plurality of sequence fragments from the plurality of cell-free nucleic acid samples. In some examples, the method 1500 includes determining, using a fragmentomics model, the cancer signal from the cell-free nucleic acid fragmentation patterns associated with the plurality of sequence fragments from the plurality of cell-free nucleic acid samples. In some examples, the method 1500 includes determining the epigenetic factors of each of the plurality of cell-free nucleic acid samples based on a methylation LR model cancer or non-cancer classification and based on a cancer signal from cell-free nucleic acid fragmentation patterns associated with a plurality of sequence fragments from the plurality of cell-free nucleic acid samples.

In some examples, determining the genomic alterations of each of the plurality of cell-free nucleic acid samples includes determining somatic variants observed in molecules from each of a plurality of sequence fragments from the plurality of cell-free nucleic acid samples. The somatic variants may be determined via the variant caller 238 of FIG. 2, in some examples.

The method 1500 may further include generating, based on the tumor-derived label or the non-tumor-derived label and the tumor prediction for each of the plurality of cell-free nucleic acid samples, a predictive model to predict a tumor in the plurality of cell-free nucleic acid samples, at 1540. The method 1500 may further include outputting the predictive model, at 1550. In some examples, the predictive model may be implemented in the disease classifier 239 of FIG. 2. The predictive model may be used to predict whether samples are tumor-derived or non-tumor derived, which may be used as a basis for a diagnosis for a particular disease, as well as may inform a treatment plan for the diagnosed disease.

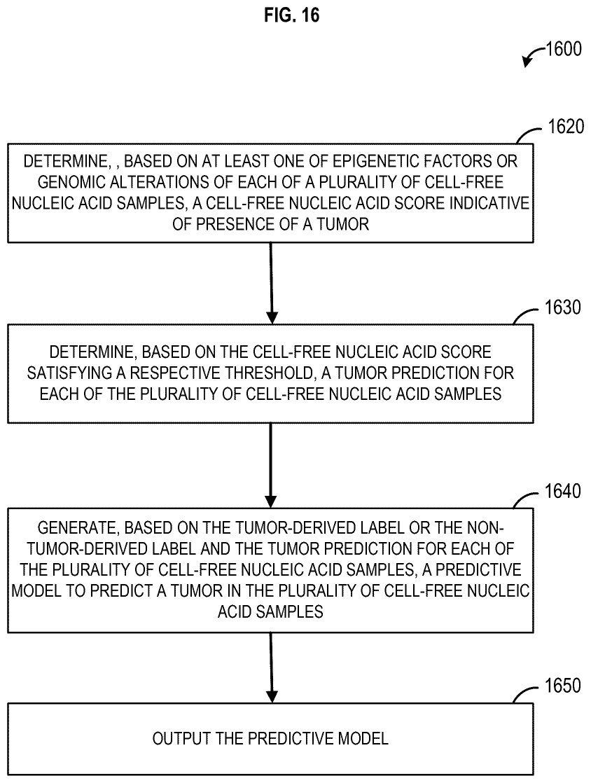

FIG. 16 is an illustration of an exemplary process flow of a method 1600 to train a predictive model to classify nucleic acid samples as tumor origin or non-tumor origin. The method 1600 may be performed by the computer system 210 of FIG. 2 in some examples.

The method 1600 may further include determining, based on at least one of epigenetic factors or genomic alterations of each of the plurality of cell-free nucleic acid samples, a cell-free nucleic acid score indicative of presence of a tumor, at 1620. Each of the plurality of cell-free nucleic acid samples may be labeled with a tumor-derived label or a non-tumor-derived label In some examples, the plurality of cell-free nucleic acid samples include cell-free deoxyribonucleic (cfDNA) samples. Additionally or alternatively, the plurality of cell-free nucleic acid samples includes one or more of ribonucleic acid (RNA) samples, cell-free ribonucleic acid (cfRNA) samples, cell-free deoxyribonucleic (cfDNA) samples, mitochondrial deoxyribonucleic (mtDNA) samples, mitochondrial ribonucleic (mtRNA) samples, and extracellular vesicle-bound deoxyribonucleic (evDNA) samples. In some examples, the plurality of cell-free nucleic acid samples are from a plurality of genomic regions. The plurality of genomic regions may include at least one of a genomic region known to be associated with a cancer type, a genomic region associated with a known methylation status, a genomic region known to be associated with hypomethylation, or a genomic region known to be associated with therapy response. In an example, the plurality of genomic regions includes at least one genomic region known to be associated with colorectal cancer. In some examples, the method 1600 includes determining the epigenetic factors of each of the plurality of cell-free nucleic acid samples. The epigenetic factors may be determined via the epigenetic component 232 of FIG. 2, in some examples. In some examples, determining the epigenetic factors of each of the plurality of cell-free nucleic acid samples based on a methylation LR model cancer or non-cancer classification. In some examples, the method 1600 further includes determining, using a LR model, the methylation LR model cancer or non-cancer classification.

In some examples, the method 1600 includes determining the epigenetic factors of each of the plurality of cell-free nucleic acid samples based on a cancer signal from cell-free nucleic acid fragmentation patterns associated with a plurality of sequence fragments from the plurality of cell-free nucleic acid samples. In some examples, the method 1600 includes determining, using a fragmentomics model, the cancer signal from the cell-free nucleic acid fragmentation patterns associated with the plurality of sequence fragments from the plurality of cell-free nucleic acid samples. In some examples, the method 1600 includes determining the epigenetic factors of each of the plurality of cell-free nucleic acid samples based on a methylation LR model cancer or non-cancer classification and based on a cancer signal from cell-free nucleic acid fragmentation patterns associated with a plurality of sequence fragments from the plurality of cell-free nucleic acid samples.

In some examples, determining the genomic alterations of each of the plurality of cell-free nucleic acid samples includes determining somatic variants observed in molecules from each of a plurality of sequence fragments from the plurality of cell-free nucleic acid samples. The somatic variants may be determined via the variant caller 238 of FIG. 2, in some examples.

The method 1600 may further include determining, based on the cell-free nucleic acid score satisfying a respective threshold, a tumor prediction for each of the plurality of cell-free nucleic acid samples, at 1630. For example, if the cell-free nucleic acid score satisfies the respective threshold, the plurality of cell-free nucleic acid samples may be determined to be tumor-derived. Otherwise, the plurality of cell-free nucleic acid samples may be determined to be non-tumor-derived.

In some examples, determining the tumor prediction for each of the plurality of cell-free nucleic acid is further based on a TFR score satisfying a threshold. For example, if the cell-free nucleic acid score satisfies a second respective threshold, the plurality of cell-free nucleic acid samples may be determined to be tumor-derived. The TFR score is indicative of a fraction of molecules of the plurality of cell-free nucleic acid samples that indicate a tumor. In some examples, determining the cell-free nucleic acid score is further based on the TFR score. The method 1600 may include determining, based on a quantification of an observed tumor-associated aberrant methylation of each of a plurality of cell-free nucleic acid samples, using a TFR model, the TFR score. The TFR model may be included in the TFR methylation component 235 of FIG. 2, in some examples.

In some examples, the method 1600 includes determining the quantification of the observed tumor-associated aberrant methylation of each of the plurality of cell-free nucleic acid samples. In some examples, determining the quantification of the observed tumor-associated aberrant methylation of each of the plurality of cell-free nucleic acid samples includes quantifying a number of unique methylated molecules mapping to each of the plurality of genomic regions.

The method 1600 may further include generating, based on the tumor-derived label or the non-tumor-derived label and the tumor prediction for each of the plurality of cell-free nucleic acid samples, a predictive model to predict a tumor in the plurality of cell-free nucleic acid samples, at 1640. The method 1600 may further include outputting the predictive model, at 1650. In some examples, the predictive model may be implemented in the disease classifier 239 of FIG. 2. The predictive model may be used to predict whether samples are tumor-derived or non-tumor derived, which may be used as a basis for a diagnosis for a particular disease, as well as may inform a treatment plan for the diagnosed disease.

### 3. Biomarkers

In some aspects, biomarkers can be analyzed in samples from a subject. In some aspects, the biomarkers can be used in cancer screening (e.g., to detect the presence of cancer or monitor cancer in a subject). In some aspects, a combination of one or more biomarkers and one or more of the algorithms described herein can be used to detect the presence of cancer or monitor cancer in a subject. In some aspects, the biomarkers can be, but are not limited to, proteins, exosomes, exomeres, microvesicles, apoptotic bodies, NETs, immune cells, TEPs, microbiome, virome, TLRs, and mtDNA.

In some aspects, biomarkers can be detected in one or more of the samples described herein. For example, a sample can be obtained from a subject and a portion of the sample can be used to testing for biomarkers while another portion of the sample can be used for epigenetic and genetic information. In some aspects, the sample used for testing biomarkers and epigenetic and genetic information can be the same sample but it can be processed differently. For example, because epigenetic and genetic information can be based on nucleic acid sequence information, the sample can be purified to a nucleic acid sample, particularly a cell free nucleic acid sample, whereas detecting a protein biomarker or cellular biomarker would require the presence of these elements in the processed sample.

In some aspects, the biomarkers described herein can be isolated or obtained from or detected in biological fluids including, without limitation, blood, serum, plasma, ascites, cyst fluid, pleural fluid, peritoneal fluid, cerebrospinal fluid, tears, urine, saliva, sputum, nipple aspirates, lymph fluid, fluid of the respiratory, intestinal, and genitourinary trances, breast milk, infra-organ system fluid, conditioned media from tissue explant culture, or combinations thereof.

As used herein, the term “protein” refers to one or more polypeptides complexed to execute biological functions within mammalian cells. Polypeptides are comprised of amino acid sequences wherein the sequence is determined by one or more messenger RNA molecules. Protein expression can be determined by a wide variety of regulators including but not limited to small noncoding RNAs (sncRNAs), post-translational modifications (PTMs), and regulatory mRNA binding proteins. Methods to evaluate protein abundance or expression include ELISAs, immunohistochemistry, immunoblotting, flow cytometry, cytometric bead assays, and fluorescence microscopy.

Many onco-proteins and tumor suppressor proteins have been linked to a wide variety of cancers and cancer progression. For example, cancer diagnostics can detect serum protein levels from blood samples of patients. Additionally, protein levels from patient biopsies can be used to determine expression of onco-proteins and tumor suppressor proteins. These diagnostics can also delineate cancer type, progression, and potential therapeutics.

As used herein, the term “exosomes” refers to extracellular vesicles comprised of phospholipid bilayer derived from the cellular membrane of mammalian cells. These vesicles can contain nucleic acids, proteins, polypeptides, lipids, and small metabolites. Exosomes shed or bud off of the plasma membrane of mammalian cells, and are generally 30 to 150 nm in size. The exosomes can facilitate cell to cell communication across various distances to enable paracrine, autocrine, and endocrine signaling. Quantification and detection of exosomes can be facilitated by nanoparticle tracking, tunable resistive pulse sensing, ELISA, vesicle flow cytometry, transmission electron microscopy, dynamic light scattering, and surface plasmon resonance.

As used herein, the term “exomeres” refers to small, non-membranous extracellular nanoparticles lacking lipid bilayer membranes released by cells. Exomeres are often abundantly enriched in the cellular microenvironment, and are generally less than 50 nm in size. Exomeres can comprise metabolic enzymes, signaling proteins, nucleic acids, and/or lipids. Detection of exomeres can be performed similarly to exosomal detection and evaluation, as listed previously. Exomeres can be isolated using asymmetric-flow field-flow fractionation as well as ultracentrifugation. Exomeres can be detected for higher or lower levels relative to a standard for subjects not having cancer, or for the presence or absence of one or more proteins, lipids, N-glycans, or nucleic acids contained in the exomeres. Exomeres, as well as exosomes, often carry surface molecules such as antigens from their donor cells, thus, surface molecules may be used to identify, isolate or enrich for exomeres or exosomes from a specific donor cell type. For example, tumor (malignant and non-malignant) exosomes carry tumor-associated surface antigens and these exomeres or exosomes can be isolated or enriched via these specific tumor-associated surface antigens. In one example, the tumor-associated surface antigen is epithelial-cell-adhesion-molecule (EpCAM), which is specific to exosomes from carcinomas of lung, colorectal, breast, prostate, head and neck, and hepatic origin, but not of hematological cell origin. Alternatively, tumor specific exosomes may be characterized by the lack of surface markers, such as the lack of CD80 and CD86 expression.

As used herein, the term “microvesicles” refers to membrane-enclosed vesicles released from cells via outward budding and pinching of the lipid bilayer plasma membrane of mammalian cells. In contrast to exosomes and exomeres, microvesicles are between 100 to 1000 nm in size, and often contain signaling proteins, receptors, lipids, carbohydrates, and genetic material such as sncRNAs and mRNAs. These vesicles also facilitate cell-to-cell communication. Similarly to exosomes and exomeres, microvesicles can be detected and quantified using the methods listed above.

Microvesicles, exosomes, and exomeres are extracellular vesicles that can be harvested from blood plasma and isolated via ultracentrifugation or other methods. Cancer derived vesicles contain protein or genetic biomarkers indicative of cancer disease pathogenesis. Increased expression of cancer biomarkers within microvesicles, exosomes, and exomeres can also be evaluated using techniques to determine protein expression.

As used herein, the term “apoptotic bodies” (ApoBDs) refers to membrane bound vesicles generated by cells undergoing apoptosis. These extracellular vesicles are formed from the remnants of apopotic cell disassembly, and often range from 500 nm to 2 μm in size. Apoptotic cell disassembly is tightly regulated by distinct morphological steps including membrane blebbing, apoptotic membrane protrusion formation, and fragmentation. Apoptotic bodies can contain various cellular debris and components, including but not limited to degraded or intact proteins, lipids, DNA fragments, mRNA, mtRNA, rRNA, chromatin, cytosolic material, or degraded or intact organelles. Many assays have been used to detect cellular apoptosis events. Such assays include: annexin V detection assay via immunofluorescence or flow cytometry; alterations in mitochondria via immunofluorescence, flow cytometry, and live cell imaging; caspase detection immunofluorescence, flow cytometry, and colorimetric assay; DNA fragmentation via TUNEL assay.

Apoptotic bodies are often increased near tumors due to decreased immune cell infiltration and phagocyte-mediated clearance of ApoBDs. Therefore, increased apopotic body detection can be used for nearby tumor detection. Specifically, cancer derived ApoBDs are often markers of tumor progression due to the ability to modulate cell proliferation, tumor growth, angiogenesis, and drug resistance. Due to derivative nature, the presence and detection of cancer-derived ApoBDs can be utilized as biomarkers.

As used herein, the term “neutrophil extracellular traps” (NETs) refers to a complex network of extracellular protein-coated nucleic acids that entrap pathogenic microorganisms. Though neutrophils primarily create NETs, other leukocytes (including but not limited to) monocytes, macrophages, basophils, eosinophils, and mast cells also generate extracellular traps. Some methods to detect and measure NET formation include ELISA, immunofluorescence microscopy, electron microscopy, live imaging, flow cytometry, multispectral image flow cytometer, and immunoblotting.

NETs are present in the cancer microenvironment and tumor progression. NETs can act as scaffolds for cancer cells to facilitate cell-to-cell communication, allowing delivery of pro-tumor, onco-proteins to nearby cells. Secretion of onco-proteins and other growth molecules induces accelerated growth and enhance cell mobility. Therefore, NET detection can identify cancer progression and determine outcomes for a wide range of cancers. Additionally, monitoring the level of NETs released in the blood is useful as a noninvasive biomarker for early diagnosis and monitoring disease progression of lung, gastroesophageal and endometrial adenocarcinomas. Elevated levels of neutrophil associated proteins and circulating NET-DNA complexes also are used as biomarkers for breast, gastric, and pancreatic cancers.

As used herein, the term “immune cells” refers to cellular component of the immune system that circulate throughout an organism to ensure proper function and protection from innate (cancer) and foreign disruptions (pathogens). Immune cells include granulocytes (basophils, eosinophils, and neutrophils), mast cells, monocytes, dendritic cells, natural killer cells, B cells, and T cells. Immune cells can reside in most tissue types, but often reside on or in skin, bone marrow, blood stream, lymphatic system, spleen, and mucosal tissue. Immune cells originate within the bone marrow, mature in the bone marrow or thymus, and are activated by a wide variety of extracellular signals such as cytokines, chemokines, and growth factors. Mature, naïve immune cells become activated by extracellular signals and contact with foreign antigens or aberrant innate antigens. Once activated, immune cells present antigens, perform phagocytosis, degranulate, differentiate, proliferate, kill pathogens and target cells, release cytokines to activate and recruit other immune cells, and secrete immune-related proteins such as antibodies, histamines, etc. Immune cells can be measured and detected using various methods, such as flow cytometry, immunofluorescence, microscopy, blood tests to measure total immune cell counts.

Generally, the immune system detects and destroys abnormal cells, preventing the growth and progression of tumors. As a result, tumor microenvironment often includes immune cells. In a biopsy, an increased abundance of tumor infiltrating immune cells indicates immune cell activation; thus, an abnormal increase in surrounding and infiltrating immune cells than normal would be a biomarker of cancer progression. Similarly, abnormally high or low levels of circulating immune cells can indicate leukemia or other types of cancer.

As used herein, the term “tumor-educated platelets” (TEPs) are platelets with an altered mRNA expression profile due to cancer cell interactions. The interactions lead to platelet alterations in adhesion molecules, glycoproteins, nucleic acids, proteins and various receptors. Subsequently, TEPs circulate throughout the body to enable tumor growth and dissemination at distant areas from the original tumor site. Methods to detect and quantify TEPs include but are not limited to liquid biopsies, blood tests, and RNA-based blood tests. Subsequent testing such as high throughput sequencing improve accuracy and sensitivity for TEP detection.

RNA-sequencing analysis has shown promising identification of various cancer biomarkers, including TEPs. For example, mRNA sequencing of TEP blood platelets can distinguish cancer patients from healthy people with 96% accuracy. TEP RNA-based blood tests can also detect 18 cancer types. Additionally, another study found that the thromboSeq PSO-algorithm enabled the selection of an RNA biomarker panel and the validation of two blood tests. The high sensitivity test detected 95% of non-small cell lung cancer, while the high specificity test detected 94% of controls.

As used herein, the term “microbiome” refers the consortia of microorganisms, such as bacteria, fungi, viruses, protozoans, archaea, and their respective genes within a particular environment. Sometimes, these environments include other organisms. In humans, the microbiome consists of trillions of symbiotic microbes that reside within and on various organs including the gut and skin. Many bioinformatics tools have been formulated to detect and analyze the microbiome, including marker gene analysis, shotgun metagenomics, metatranscriptomics, metabolomics, and metaproteomics.

Though the microbiome has been shown to work synergistically with the host organism, microbes can also be the etiological agents of various diseases, including cancer. Microbes can: impact pro-tumorigenic metabolite mediated interactions, directly interact with cancer cells and tumor microenvironment to influence cell cycle and proliferation, can activate inflammatory pathways, and disrupt vascular barriers to promote metastasis. Using the methods above to evaluate microbial abundance, substantial increase or decrease in various microbes within the total flora can be a predictive indicator of cancer pathogenesis.

As used herein, the term “virome” refers to the collection of viruses and genetic material within a particular environment. The mammalian virome consists of mammalian-infecting viruses, bacteriophages, and other virus-derived elements. In humans specifically, the virome is vast and complex, consisting of approximately 10¹³ particles per human individual, with great heterogeneity. Methods to measure and evaluate the virome include plaque assays, focus-forming assays, real-time qPCR, immunoblotting, ELISA, electron microscopy, and flow cytometry.

Accumulating evidence shows that various viruses including human papillomaviruses (HPV), hepatitis B virus (HBV), hepatitis C virus (HCV), Epstein-Barr virus (EBV), Kaposi's sarcoma-associated herpesvirus (KSHV) (also called human herpesvirus 8), human T-cell lymphotropic virus (HTLV-1), and Merkel cell polyomavirus (MCPyV) are caustative agents of various cancers. The presence and increased expression of viral onco-proteins such as E6 and E7 of HPVs, LMP1 of EBV, Tax of HTLV-1, and T antigen of MCPyV are used as cancer biomarkers. For example, the HPV E6/E7 mRNA and HPV E6/E7 tests are highly specific and sensitive, thus viral proteins such as HPV E6/E7 are used as diagnostic cancer biomarkers.

As used herein, the term “toll like receptors” (TLRs) refers to evolutionarily conserved plasma and organelle membrane bound receptors belonging the pattern recognition receptors family (PRRs), which play a vital role in immune responses especially pathogen recognition. TLRs hold a key position in the first line of defense against pathogens because of their ability to recognize the conserved pathogen-associated molecular patterns (PAMPs), conserved structures of the pathogens, or the damage caused by the pathogens within the host. TLRs generally consist of three domains: an N-terminal domain (NTD) located outside the membrane, a middle single helix transmembrane domain traversing the membrane, and a C-terminal domain (CTD) located towards the cytoplasm. TLRs can be detected similarly to protein detection methods listed previously.

TLRs can regulate the immune response to tumors by inducing pro and anti-tumor responses. TLRs expressed on tumor cells contribute to pathogenesis and disease progression via enhancing of proliferation, invasion and metastasis, dampening immune suppression factors, and increasing activation immune regulatory cells, such as T_{regs}. Tissue biopsies and elevated gene and protein expression of TLR2, TLR3, TLR5, and TLR9 (along with other morphological, phenotypic, and genotypic alterations) are strong indicators of cancer progression. Additionally, various combinations of TLR expression denote presence of different cancer types. For example, TLR3 and TLR4 are elevated in the early stages of kidney renal clear cell carcinoma.

As used herein, the term “mitochondrial DNA” (mtDNA) refers to a circular, double stranded DNA molecule about 16.6 kb in size that resides the mitochondria of mammalian cells. MtDNA encodes 22 transfer RNAs, 2 ribosomal RNAs, and 13 structural polypeptide components required for oxidative phosphorylation. Maternal inheritance of mtDNA is observed in sexually reproducing species. The mitochondria organelle is responsible for cellular energy production, metabolism, apoptosis, and oxidative stress control. Methods to detect mtDNA include but are not limited microarray, real time qPCR, DNA sequencing, and immunoblotting.

The mitochondrial genome (mtDNA) encodes essential machinery for oxidative phosphorylation and metabolic homeostasis. Tumor mtDNA is among the most somatically mutated regions of the cancer genome. MtDNA variations such as mutations, deletions, or single nucleotide polymorphisms (SNPs) are strong indicators of genetic propensity to develop cancer and other diseases. The presence of specific SNPs in mtDNA have been confirmed to be correlated to cancer progression and disease severity. Mutations in tRNA encoding genes have also been linked to cancer progression. MtDNA within circulating extracellular vesicles can be tested for mutations and used as early, reliable cancer biomarkers. In some aspects, CpG methylation of one or more regions in mtDNA can be a strong indicator of genetic propensity to develop cancer and other diseases.

In some aspects, the presence or levels of one or more biomarkers can be detected. In some aspects, the presence of a biomarker can be indicative of cancer. In some aspects, detecting the level of a biomarker further comprises comparing the detected level of the biomarker to a reference level of the biomarker. For example, for many cancer biomarkers there are established or known amounts considered to be “normal” or standard levels. Thus, the methods can further comprise identifying the presence of cancer in the subject from which the sample was obtained when the presence of the biomarkers is detected and the detected level of the biomarker is higher than a reference level of the biomarkers. In some aspects, the detection of a biomarker at least 1×, 2×, 3×, 4×, or 5× the reference level would be considered a sample from a subject having cancer. In some aspects, any amount of biomarker present in a sample above the reference level can be considered a sample from a subject having cancer. In some aspects, instead of a reference level, the detected biomarker can be compared to an amount of the biomarker present in a healthy, or non-cancer subject. In some aspects, a reference level or the amount in a healthy, non-cancer subject can be considered a control. Thus, the presence or level of one or more biomarkers in a biological sample of a subject compared to a control can determine whether the biological sample is tumor-derived or non-tumor derived.

In some aspects, the reference level is a known or set reference level for each biomarker. In some aspects, the amount of biomarker present in a healthy, or non-cancer subject can be determined in parallel with the sample being tested or can be an amount previously determined in healthy, or non-cancer subjects.

### 4. Algorithms

The classification of a sample relies upon the multiple biomarkers derived from cfDNA and known to be distinct between normal and cancer-derived tissues. The cfDNA cancer screening test is an assay which interrogates thousands of individual features that characterize three types of cfDNA signals or patterns: epigenetic changes resulting in the aberrant methylation state, epigenetic changes resulting in the aberrant cfDNA molecule fragmentation patterns, and genomic changes resulting in somatic mutations.

The cfDNA cancer screening test result described herein can be determined based on two scores: the score from a methylation-based TFR model and the cfDNA integrated score. If either the cfDNA integrated score or the TFR score exceeds their respective pre-defined thresholds, the cfDNA cancer screening test result is positive (abnormal). Otherwise, the cfDNA cancer screening test result is negative (normal).

In an embodiment, the algorithms can be trained using hundreds or thousands of development samples representing diverse cohorts of healthy donor samples, colonoscopy-screened CRC negative donors, as well as CRC patients. Parameters of the cfDNA sample processing, QC, and cfDNA regression models and weights can be locked in a software algorithm prior to the initiation of the testing.

i. Methylation-Based Tumor Fraction Regression (TFR) Model Score

In an embodiment, the TFR model can quantify the fraction of tumor-derived cfDNA (tumor fraction) in a sample based on the quantification of the observed tumor-associated aberrant methylation of cfDNA molecules. This quantification can be based on the observed number of unique methylated molecules mapping to each of the targeted classification regions. These molecule counts can be normalized to the overall number of unique methylated molecules observed in the normalization regions of the panel. After normalization, the dependence of the classification region feature values (normalized molecule counts) on the total number of molecules measured and input cfDNA amount for a sample is minimized. Region level normalized molecule counts are used as input features into the TFR model. The model can be trained on over 4,000 development samples to predict their tumor fraction. The predicted tumor fraction is used as a score for assessment of cancer status of an individual sample.

ii. cfDNA Integrated Model Score

In an embodiment, the cfDNA integrated model developed is a logistic regression model to generate a quantitative score indicating presence of tumor-derived molecules based on the joint assessment of the epigenetic signals (cfDNA methylation status and fragmentation patterns) and a qualitative mutation detected status (for somatic mutations). Each of these analytes are first analyzed separately and then the resulting individual quantitative scores of the per-analyte assessments are aggregated by the cfDNA integrated model to produce a single cfDNA integrated score. Thus, in an embodiment, the cfDNA integrated score comprises four components: methylation TFR model, methylation logistic regression (LR) model, fragmentomics, and genetic alterations.

The details about these individual scores are described below:

a. Methylation Models

In an embodiment, the scores of both the TFR model, described above, and a methylation logistic regression (LR) model are used as input to the cfDNA integrated model. A methylation LR model was developed to differentiate the tumor-associated methylation signatures of cfDNA molecules from those observed in subjects without tumors. The methylation LR model uses the same input feature space as the TFR model, namely the region level normalized molecule counts described above. Compared to the TFR model, the methylation LR model can be trained to predict the binary disease state (cancer and non-cancer) instead of the quantitative tumor fraction. The methylation LR model can be trained on the same set of samples used to train the TFR model.

b. Fragmentomics Model

In an embodiment, a fragmentomics model captures the cancer signal from tumor-associated cfDNA fragmentation patterns. To derive quantitative scores associated with the fragmentation patterns, a mixture model of molecule endpoint densities within each of the fragmentomics relevant classification regions can be trained to estimate endpoint densities across normal and CRC samples. A molecule endpoint density is defined for each genomic region/sample as follows. For each genomic position, the number of molecule endpoints present at that position is aggregated and normalized by the total endpoint count for that region and sample. In an embodiment for predicting one type of disease (e.g., colorectal cancer), only molecules between 120 and 240 bp in the unmethylated partition mapping to the set of regions identified as informative for fragmentomics signal differences may be used. In other embodiments, other ranges of molecules in the unmethylated partition mapping to the set of regions identified as informative for fragmentomics signal differences may be used. In yet other embodiment, molecules in the unmethylated partition mapping and/or methylated partition mapping to the set of regions identified as informative for fragmentomics signal differences may be used. The pattern in an individual sample can then be fit as a mixture of the CRC and normal endpoint densities and a posterior expected value of the mixing proportion between normal and CRC densities is derived for each region. Finally, a logistic regression model can be trained to combine the mixture scores from all classification regions within the fragmentomics subpanel into a single quantitative score.

c. Somatic Caller (Variant Caller)

In an embodiment, the cfDNA integrated model can also be informed by whether any tumor-derived mutations are identified. Somatic caller leverages the somatic variants observed in the molecules from all partitions and its output is dichotomous: one or more tumor-derived mutations detected or none detected. Somatic caller can be trained to minimize false positive rates associated with non-tumor derived variants commonly found in cfDNA samples of healthy individuals at low allelic frequencies. Only somatic nonsense SNVs (single nucleotide variants), splice variants, and indels with variant allele fraction (VAF) >0.1% in APC or KRAS are considered when generating a positive somatic call, and these variants can be further filtered based on the variant frequency and clonality observed in the large internal reference database of cancer samples.

In an embodiment, four components, the three quantitative scores from the two methylation models (TFR and LR models) and the fragmentomic model and the qualitative assessment of somatic mutation detection (encoded as 0 for absence of mutations and 1 for positive detection of somatic mutations), can be standardized and integrated using a logistic regression model (cfDNA integrated model) to produce a single integrated score per sample. The cfDNA integrated model can be trained to predict CRC status using an independent set of samples that were not used to train either the methylation models or the fragmentomics model.

### D. Exemplary Applications

The methods presented herein may be used as part of any method that benefits from obtaining an accurate modified nucleoside profile of DNA in any sample.

One exemplary application of the methods of the disclosure is using the modified nucleoside profile in diagnosing and prognosing cancer or other genetic diseases or conditions.

Hence, in some embodiments, a method described herein comprises identifying or predicting the presence or absence of DNA produced by a tumor (or neoplastic cells, or cancer cells), determining the probability that a test subject has a tumor or cancer, and/or characterizing a tumor, neoplastic cells or cancer as described herein.

### 1. Cancer and Other Diseases; Cell Type Quantification

The present methods can be used to diagnose presence of a condition, e.g., cancer or precancer, in a subject, to characterize a condition (such as to determine a cancer stage or heterogeneity of a cancer), to monitor a subject's response to receiving a treatment for a condition (such as a response to a chemotherapeutic or immunotherapeutic), assess prognosis of a subject (such as to predict a survival outcome in a subject having a cancer), to determine a subject's risk of developing a condition, to predict a subsequent course of a condition in a subject, to determine metastasis or recurrence of a cancer in a subject (or a risk of cancer metastasis or recurrence), and/or to monitor a subject's health as part of a preventative health monitoring program (such as to determine whether and/or when a subject is in need of further diagnostic screening). The present disclosure can also be useful in determining the efficacy of a particular treatment option. Successful treatment options may increase the amount of rare mutations detected in subject's blood if the treatment is successful as more cancers may die and shed DNA. In other examples, this may not occur. In another example, certain treatment options may be correlated with genetic profiles of cancers over time. This correlation may be useful in selecting a therapy. In some embodiments, target regions (e.g., hypermethylation variable epigenetic target regions) are analyzed to determine whether they show methylation (e.g., hypermethylation) characteristics of tumor cells or cells that do not ordinarily contribute significantly to cfDNA and/or target regions (e.g., hypomethylation variable target regions) are analyzed to determine whether they show methylation (e.g., hypomethylation) characteristic of tumor cells or cells that do not ordinarily contribute significantly to cfDNA. In some embodiments, successful treatment options may result in changes in levels of different immune cell types (including rare immune cell types), and/or increases in the amount of target proteins, copy number variation, rare mutations, and/or cancer-related epigenetic signatures (such as hypermethylated regions or hypomethylated regions) detected in, e.g., a sample from a subject, such as detected in a subject's blood (such as in DNA isolated from a buffy coat sample or any other sample comprising cells, such as in a blood sample (e.g., a whole blood sample, a plasma sample, a leukapheresis sample, or a PBMC sample) from the subject) if the treatment is successful as more cancer cells may die and shed DNA, or, e.g., if a successful treatment results in an increase or decrease in the quantity of a specific protein in the blood and an unsuccessful treatment results in no change.

Additionally, if a cancer is observed to be in remission after treatment, the present methods can be used to monitor the likelihood of residual disease or the likelihood of recurrence of disease.

In some embodiments, the present methods are used for screening for a cancer, such as a metastasis, or in a method for screening cancer, such as in a method of detecting the presence or absence of a metastasis. For example, the sample can be a sample from a subject who has or has not been previously diagnosed with cancer. In some embodiments, one or more, such as 1, 2, 3, 4, 5, 6, 7, 8, 9, 10, or more samples are collected from a subject as described herein, such as before and/or after the subject is diagnosed with a cancer. In some embodiments, the subject may or may not have cancer. In some embodiments, the subject may or may not have an early-stage cancer. In some embodiments, the subject has one or more risk factors for cancer, such as tobacco use (e.g., smoking), being overweight or obese, having a high body mass index (BMI), being of advanced age, poor nutrition, high alcohol consumption, or a family history of cancer.

In some embodiments, the subject has used tobacco, e.g., for at least 1, 5, 10, or 15 years. In some embodiments, the subject has a high BMI, e.g., a BMI of 25 or greater, 26 or greater, 27 or greater, 28 or greater, 29 or greater, or 30 or greater. In some embodiments, the subject is at least 40, 45, 50, 55, 60, 65, 70, 75, or 80 years old. In some embodiments, the subject has poor nutrition, e.g., high consumption of one or more of red meat and/or processed meat, trans fat, saturated fat, and refined sugars, and/or low consumption of fruits and vegetables, complex carbohydrates, and/or unsaturated fats. High and low consumption can be defined, e.g., as exceeding or falling below, respectively, recommendations in Dietary Guidelines for Americans 2020-2025, available at dietaryguidelines.gov/sites/default/files/2021-03/Dietary_Guidelines_for_Americans-2020-2025.pdf. In some embodiments, the subject has high alcohol consumption, e.g., at least three, four, or five drinks per day on average (where a drink is about one ounce or 30 mL of 80-proof hard liquor or the equivalent). In some embodiments, the subject has a family history of cancer, e.g., at least one, two, or three blood relatives were previously diagnosed with cancer. In some embodiments, the relatives are at least third-degree relatives (e.g., great-grandparent, great aunt or uncle, first cousin), at least second-degree relatives (e.g., grandparent, aunt or uncle, or half-sibling), or first-degree relatives (e.g., parent or full sibling).

In some embodiments, the methods and systems disclosed herein may be used to identify customized or targeted therapies to treat a given disease or condition in patients based on the classification of a nucleic acid variant as being of somatic or germline origin.

Typically, the disease under consideration is a type of cancer, such as any referred to herein. The types and number of cancers that may be detected may include blood cancers, brain cancers, lung cancers, skin cancers, nose cancers, throat cancers, liver cancers, bone cancers, lymphomas, pancreatic cancers, skin cancers, bowel cancers, rectal cancers, thyroid cancers, bladder cancers, kidney cancers, mouth cancers, stomach cancers, solid state tumors, heterogeneous tumors, homogenous tumors and the like. Specific examples of such cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic leukemia (CLL), chronic myeloid leukemia (CML), chronic myelomonocytic leukemia (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas. Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma.

In some embodiments, the cancer is a type of cancer that is not a hematological cancer, e.g., a solid tumor cancer such as a carcinoma, adenocarcinoma, or sarcoma. Type and/or stage of cancer can be detected from genetic variations including mutations, rare mutations, indels, rearrangements, copy number variations, transversions, translocations, recombinations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, such as 5mC and/or 5hmC profiles. Hence, the present methods can in some cases be used in combination with methods used to detect other genetic/epigenetic variations, e.g. in a method of detecting or characterizing a cancer or other methods described herein.

In some embodiments, a method described herein comprises identifying the presence of target regions and/or DNA produced by a tumor (or neoplastic cells, or cancer cells) or by precancer cells. In some embodiments, a method described herein comprises determining the level of target regions and/or identifying the presence of DNA produced by a tumor (or neoplastic cells, or cancer cells) or by precancer cells. In some embodiments, determining the level of target regions comprises determining either an increased level or decreased level of target regions, wherein the increased or decreased level of target regions is determined by comparing the level of target regions with a threshold level/value.

Genetic and/or epigenetic data can also be used for characterizing a specific form of cancer. Cancers are often heterogeneous in both composition and staging. Genetic and/or epigenetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The system and methods of this disclosure may be useful in determining disease progression.

Further, the methods of the disclosure may be used to characterize the heterogeneity of an abnormal condition in a subject. Such methods can include, e.g., generating a genetic and/or epigenetic profile of extracellular polynucleotides, such as cfDNA, derived from the subject, wherein the genetic and/or epigenetic profile comprises a plurality of data resulting from copy number variation and rare mutation analyses. In some embodiments, an abnormal condition is cancer, e.g. as described herein. In some embodiments, the abnormal condition may be one resulting in a heterogeneous genomic population. In the example of cancer, some tumors are known to comprise tumor cells in different stages of the cancer. In other examples, heterogeneity may comprise multiple foci of disease such as where one or more foci (such as one or more tumor foci) are the result of metastases that have spread from a primary site of a cancer. The tissue(s) of origin can be useful for identifying organs affected by the cancer, including the primary cancer and/or metastatic tumors.

The present methods can also be used to quantify levels of different cell types, such as immune cell types, including rare immune cell types, such as activated lymphocytes and myeloid cells at particular stages of differentiation. Such quantification can be based on the numbers of molecules corresponding to a given cell type in a sample. Sequence information obtained in the present methods may comprise sequence reads of the nucleic acids generated by a nucleic acid sequencer. In some embodiments, the nucleic acid sequencer performs pyrosequencing, single-molecule sequencing, nanopore sequencing, semiconductor sequencing, sequencing-by-synthesis, 5-letter sequencing, 6-letter sequencing, sequencing-by-ligation or sequencing-by-hybridization on the nucleic acids to generate sequencing reads. In some embodiments, the method further comprises grouping the sequence reads into families of sequence reads, each family comprising sequence reads generated from a nucleic acid in the sample. In some embodiments, the methods comprise determining the likelihood that the subject from which the sample was obtained has cancer or precancer, or has a metastasis, that is related to changes in proportions of types of immune cells.

The present methods can be used to generate or profile, fingerprint or set of data that is a summation of genetic and/or epigenetic information derived from different cells in a heterogeneous disease. This set of data may comprise copy number variation, epigenetic variation, and mutation analyses alone or in combination.

The present methods can be used to diagnose, prognose, monitor or observe cancers, or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules.

Non-limiting examples of other genetic-based diseases, disorders, or conditions that are optionally evaluated using the methods and systems disclosed herein include achondroplasia, alpha-1 antitrypsin deficiency, antiphospholipid syndrome, autism, autosomal dominant polycystic kidney disease, Charcot-Marie-Tooth (CMT), cri du chat, Crohn's disease, cystic fibrosis, Dercum disease, down syndrome, Duane syndrome, Duchenne muscular dystrophy, Factor V Leiden thrombophilia, familial hypercholesterolemia, familial Mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency (SCID), sickle cell disease, spinal muscular atrophy, Tay-Sachs, thalassemia, trimethylaminuria, Turner syndrome, velocardiofacial syndrome, WAGR syndrome, Wilson disease, or the like.

In some embodiments, a method described herein comprises detecting a presence or absence of DNA originating or derived from a tumor cell at a preselected timepoint following a previous cancer treatment of a subject previously diagnosed with cancer using a set of sequence information obtained as described herein. The method may further comprise determining a cancer recurrence score that is indicative of the presence or absence of the DNA originating or derived from the tumor cell for the subject.

Where a cancer recurrence score is determined, it may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

In some embodiments, a cancer recurrence score is compared with a predetermined cancer recurrence threshold, and the subject is classified as a candidate for a subsequent cancer treatment when the cancer recurrence score is above the cancer recurrence threshold or not a candidate for therapy when the cancer recurrence score is below the cancer recurrence threshold. In particular embodiments, a cancer recurrence score equal to the cancer recurrence threshold may result in classification as either a candidate for a subsequent cancer treatment or not a candidate for therapy.

The present methods can also be used to quantify levels of different cell types, such as immune cell types, including rare immune cell types, such as activated lymphocytes and myeloid cells at particular stages of differentiation. Such quantification can be based on the numbers of molecules corresponding to a given cell type in a sample. Sequence information obtained in the present methods may comprise sequence reads of the nucleic acids generated by a nucleic acid sequencer. In some embodiments, the nucleic acid sequencer performs pyrosequencing, single-molecule sequencing, nanopore sequencing, semiconductor sequencing, sequencing-by-synthesis, 5-letter sequencing, 6-letter sequencing, sequencing-by-ligation or sequencing-by-hybridization on the nucleic acids to generate sequencing reads. In some embodiments, the method further comprises grouping the sequence reads into families of sequence reads, each family comprising sequence reads generated from a nucleic acid in the sample. In some embodiments, the methods comprise determining the likelihood that the subject from which the sample was obtained has cancer, precancer, an infection, transplant rejection, or other diseases or disorder that is related to changes in proportions of types of immune cells. Comparisons of immune cell identities and/or immune cell quantities/proportions between two or more samples collected from a subject at two different time points can allow for monitoring of one or more aspects of a condition in the subject over time, such as a response of the subject to a treatment, the severity of the condition (such as a cancer stage) in the subject, a recurrence of the condition (such as a cancer), and/or the subject's risk of developing the condition (such as a cancer).

The methods discussed above may further comprise any compatible feature or features set forth elsewhere herein, including in the section regarding methods of determining a risk of cancer recurrence in a subject and/or classifying a subject as being a candidate for a subsequent cancer treatment.

2. Methods of Determining a Risk of Cancer Recurrence in a Test Subject and/or Classifying a Subject as being a Candidate for a Subsequent Cancer Treatment

In some embodiments, a method provided herein is or comprises a method of determining a risk of cancer recurrence in a subject. In some embodiments, a method provided herein is or comprises a method of detecting the presence of absence of a metastasis in a subject. In some embodiments, a method provided herein is or comprises a method of classifying a subject as being a candidate for a subsequent cancer treatment.

Any of such methods may comprise collecting a sample (such as DNA, such as DNA originating or derived from a tumor cell) from the subject diagnosed with the cancer at one or more preselected timepoints following one or more previous cancer treatments to the subject. The subject may be any of the subjects described herein. The sample may comprise chromatin, cfDNA, or other cell materials. The sample, such as the DNA sample, may be a tissue sample. The DNA may be DNA, such as cfDNA, from a blood sample (e.g., a whole blood sample, a buffy coat sample, a leukapheresis sample, or a PBMC sample). The DNA may comprise DNA obtained from a tissue sample.

Any of such methods may comprise contacting the sample or a subsample thereof with a plurality of primers, generating capture probes, capturing and detecting the presence or level of at least one structural variation according to any of the embodiments as described herein. In some embodiments, the methods may comprise contacting the sample or a subsample thereof with a plurality of capture probes specific for members of an epigenetic target region set according to any of the embodiments as described herein. In some embodiments, the capture probes comprise capture probes generated using a sample obtained from the same subject at an earlier timepoint. Any of such methods may comprise capturing a plurality of sets of target regions from DNA from the subject, wherein the plurality of target region sets comprises a sequence-variable target region set and an epigenetic target region set, whereby a captured set of DNA molecules is produced. The capturing step may be performed according to any of the embodiments described elsewhere herein.

In any of such methods, the previous cancer treatment may comprise surgery, administration of a therapeutic composition, and/or chemotherapy.

Any of such methods may comprise sequencing the captured DNA molecules, whereby a set of sequence information is produced. The captured DNA molecules of the sequence-variable target region set may be sequenced to a greater depth of sequencing than the captured DNA molecules of the epigenetic target region set.

Any of such methods may comprise detecting a presence or absence of DNA originating or derived from a tumor cell at a preselected timepoint using the set of sequence information. The detection of the presence or absence of DNA, such as cfDNA, originating or derived from a tumor cell may be performed according to any of the embodiments thereof described elsewhere herein.

Methods of determining a risk of cancer recurrence in a subject may comprise determining a cancer recurrence score that is indicative of the presence or absence, or amount, of the DNA, such as genomic regions of interest and target regions, originating or derived from the tumor cell for the subject. The cancer recurrence score may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

Methods of detecting the presence or absence of metastasis in a subject may comprise comparing the presence or level of a tissue-specific cell material to the presence or level of the tissue-specific cell material obtained from the subject at a different time, a reference level of the tissue-specific cell material, or to a comparator cell material. Methods herein may comprise additional steps to determine whether a metastasis is present.

Methods of classifying a subject as being a candidate for a subsequent cancer treatment may comprise comparing the cancer recurrence score of the subject with a predetermined cancer recurrence threshold, thereby classifying the subject as a candidate for the subsequent cancer treatment when the cancer recurrence score is above the cancer recurrence threshold or not a candidate for therapy when the cancer recurrence score is below the cancer recurrence threshold. In particular embodiments, a cancer recurrence score equal to the cancer recurrence threshold may result in classification as either a candidate for a subsequent cancer treatment or not a candidate for therapy. In some embodiments, the subsequent cancer treatment comprises chemotherapy or administration of a therapeutic composition.

Any of such methods may comprise determining a disease-free survival (DFS) period for the subject based on the cancer recurrence score; for example, the DFS period may be 1 year, 2 years, 3, years, 4 years, 5 years, or 10 years.

In some embodiments, sequence-variable target region sequences are obtained, and determining the cancer recurrence score may comprise determining at least a first subscore indicative of the amount of the levels of particular immune cell types, SNVs, insertions/deletions, CNVs and/or fusions present in sequence-variable target region sequences.

In some embodiments, a number of mutations in the sequence-variable target regions chosen from 1, 2, 3, 4, or 5 is sufficient for the first subscore to result in a cancer recurrence score classified as positive for cancer recurrence. In some embodiments, the number of mutations is chosen from 1, 2, or 3.

In some embodiments, epigenetic target region sequences are obtained, and determining the cancer recurrence score comprises determining a second subscore indicative of the amount of molecules (obtained from the epigenetic target region sequences) that represent an epigenetic state different from DNA found in a corresponding sample from a healthy subject (e.g., DNA, such as cfDNA, found in a blood sample (e.g., a whole blood sample, a buffy coat sample, a leukapheresis sample, or a PBMC sample) from a healthy subject, or DNA found in a tissue sample from a healthy subject where the tissue sample is of the same type of tissue as was obtained from the subject). These abnormal molecules (i.e., molecules with an epigenetic state different from DNA found in a corresponding sample from a healthy subject) may be consistent with epigenetic changes associated with cancer (such as with a metastasis), e.g., methylation of hypermethylation variable target regions and/or perturbed fragmentation of fragmentation variable target regions, where “perturbed” means different from DNA found in a corresponding sample from a healthy subject.

In some embodiments, a proportion of molecules corresponding to the hypermethylation variable target region set and/or fragmentation variable target region set that indicate hypermethylation in the hypermethylation variable target region set and/or abnormal fragmentation in the fragmentation variable target region set greater than or equal to a value in the range of 0.001%-10% is sufficient for the subscore to be classified as positive for cancer recurrence. The range may be 0.001%-1%, 0.005%-1%, 0.01%-5%, 0.01%-2%, or 0.01%-1%.

In some embodiments, any of such methods may comprise determining a fraction of tumor DNA from the fraction of molecules in the set of sequence information that indicate one or more features indicative of origination from a tumor cell. This may be done for molecules corresponding to some or all of the target regions, e.g., including one or more of hypermethylation variable target regions, hypomethylation variable target regions, and fragmentation variable target regions (hypermethylation of a hypermethylation variable target region and/or abnormal fragmentation of a fragmentation variable target region may be considered indicative of origination from a tumor cell). This may be done for molecules corresponding to sequence-variable target regions, e.g., molecules comprising alterations consistent with cancer, such as SNVs, indels, CNVs, and/or fusions. The fraction of tumor DNA may be determined based on a combination of molecules corresponding to epigenetic target regions and molecules corresponding to sequence-variable target regions.

Determination of a cancer recurrence score may be based at least in part on the fraction of tumor DNA, wherein a fraction of tumor DNA greater than a threshold in the range of 10⁻¹¹ to 1 or 10⁻¹⁰ to 1 is sufficient for the cancer recurrence score to be classified as positive for cancer recurrence. In some embodiments, a fraction of tumor DNA greater than or equal to a threshold in the range of 10⁻¹⁰ to 10⁻⁹, 10⁻⁹ to 10⁻⁸, 10⁻⁸ to 10⁻⁷, 10⁻⁷ to 10⁻⁶, 10⁻⁶ to 10⁻⁵, 10⁻⁵ to 10⁻⁴, 10⁻⁴ to 10⁻³, 10⁻³ to 10⁻², or 10⁻² to 10⁻¹ is sufficient for the cancer recurrence score to be classified as positive for cancer recurrence. In some embodiments, the fraction of tumor DNA greater than a threshold of at least 10⁻⁷ is sufficient for the cancer recurrence score to be classified as positive for cancer recurrence. A determination that a fraction of tumor DNA is greater than a threshold, such as a threshold corresponding to any of the foregoing embodiments, may be made based on a cumulative probability. For example, the sample was considered positive if the cumulative probability that the tumor fraction was greater than a threshold in any of the foregoing ranges exceeds a probability threshold of at least 0.5, 0.75, 0.9, 0.95, 0.98, 0.99, 0.995, or 0.999. In some embodiments, the probability threshold is at least 0.95, such as 0.99.

In some embodiments, the set of sequence information comprises sequence-variable target region sequences and epigenetic target region sequences, and determining the cancer recurrence score comprises determining a subscore indicative of the amount of SNVs, insertions/deletions, CNVs and/or fusions present in sequence-variable target region sequences and a subscore indicative of the amount of abnormal molecules in epigenetic target region sequences, and combining the subscores to provide the cancer recurrence score. Where the subscores are combined, they may be combined by applying a threshold to each subscore independently (e.g., greater than a predetermined number of mutations (e.g., >1) in sequence-variable target regions, and greater than a predetermined fraction of abnormal molecules (i.e., molecules with an epigenetic state different from the DNA found in a corresponding sample from a healthy subject; e.g., tumor) in epigenetic target regions), or training a machine learning classifier to determine status based on a plurality of positive and negative training samples.

In some embodiments, the set of sequence information comprises sequence-variable target region sequences and epigenetic target region sequences, and determining the cancer recurrence score comprises determining a first subscore indicative of the levels of particular immune cell types, a second subscore indicative of the amount of SNVs, insertions/deletions, CNVs and/or fusions present in sequence-variable target region sequences and a third subscore indicative of the amount of abnormal molecules in epigenetic target region sequences, and combining the first, second, and third subscores to provide the cancer recurrence score. Where the subscores are combined, they may be combined by applying a threshold to each subscore independently in sequence-variable target regions, respectively, and greater than a predetermined fraction of abnormal molecules (i.e., molecules with an epigenetic state different from the DNA found in a corresponding sample from a healthy subject; e.g., tumor) in epigenetic target regions), or training a machine learning classifier to determine status based on a plurality of positive and negative training samples.

In some embodiments, a value for the combined score in the range of −4 to 2 or −3 to 1 is sufficient for the cancer recurrence score to be classified as positive for cancer recurrence.

In any embodiment where a cancer recurrence score is classified as positive for cancer recurrence, the cancer recurrence status of the subject may be at risk for cancer recurrence and/or the subject may be classified as a candidate for a subsequent cancer treatment.

In some embodiments, the cancer is any one of the types of cancer described elsewhere herein, e.g., colorectal cancer.

### 3. Methods of Monitoring a Cancer in a Subject Over Time; Sample Collection at Two or More Time Points

In some embodiments, the present methods can be used to monitor one or more aspects of a condition in a subject over time, such as a subject's response to receiving a treatment for a condition (such as a response to a chemotherapeutic or immunotherapeutic), the severity of the condition (such as a cancer stage) in the subject, a recurrence of the condition (such as a cancer), and/or the subject's risk of developing the condition (such as a cancer) and/or to monitor a subject's health as part of a preventative health monitoring program (such as to determine whether and/or when a subject is in need of further diagnostic screening). In some embodiments, monitoring comprises analysis of at least two samples collected from a subject at at least two different time points as described herein.

The methods according to the present disclosure can be useful in predicting a subject's response to a particular treatment option, such as over a period of time. As described elsewhere herein, successful treatment options may increase the amount of cancer associated DNA sequences detected in a subject's blood, such as if the treatment is successful as more cancers may die and shed DNA. In such examples, certain treatment options may be correlated with genetic profiles of cancers over time. This correlation may be useful in selecting a therapy. In some embodiments, successful treatment options may result in an increase or decrease in the levels of different immune cell types (including rare immune cell types), and/or an increase or decrease in the levels of a specific protein or proteins and/or a specific DNA sequence (e.g., of a CDR3), such as in the blood, and an unsuccessful treatment may result in no change. In other examples, this may not occur.

As disclosed herein, in some embodiments, quantities of each of a plurality of cell types, such as immune cell types, are determined based on sequencing and analysis (such as determination of epigenetic and/or genomic signatures) of DNA isolated from at least one sample comprising cells (such as a tissue sample or a blood sample, e.g., a whole blood sample, a buffy coat sample, a leukapheresis sample, or a PBMC sample) from a subject. In some embodiments, differences in levels and/or presence of particular genetic and/or epigenetic signatures in DNA isolated from blood samples from a subject can be used to quantify cell types, such as immune cell types, within the sample. Thus, a comparison of the disclosed genetic and/or epigenetic signatures in DNA isolated from blood samples collected from a subject at two or more time points can be used to monitor changes in cell type quantities in the subject under different conditions (such as prior to and after a treatment), or over time (e.g., as part of a preventative health monitoring program).

The disclosed methods can include evaluating (such as quantifying) and/or interpreting cell types (such as immune cell types) present in one or more samples (such as a tissue sample or a blood sample, e.g., a whole blood sample, a buffy coat sample, a leukapheresis sample, or a PBMC sample) collected from a subject at one or more timepoints in comparison to a selected baseline value or reference standard (or a selected set of baseline values or reference standards). A baseline value or reference standard may be a quantity of cell types measured in one or more samples (such as an average quantity or range of quantities of cell types present in at least two samples) collected from the subject at one or more time points, such as prior to receiving a treatment, prior to diagnosis of a condition (such as a cancer), or as part of a preventative health monitoring program. A baseline value or reference standard may be a quantity of cell types measured in one or more samples (such as an average quantity or range of quantities of cell types present in at least two samples) collected at one or more timepoints from one or more subjects that do not have the condition (such as a healthy subject that does not have a cancer), one or more subjects that responded favorably to the treatment, or one or more subjects that have not received the treatment. In certain embodiments, the baseline value or reference standard utilized is a standard or profile derived from a single reference subject. In other embodiments, the baseline value or reference standard utilized is a standard or profile derived from averaged data from multiple reference subjects. The reference standard, in various embodiments, can be a single value, a mean, an average, a numerical mean or range of numerical means, a numerical pattern, or a graphical pattern created from the cell type quantity data derived from a single reference subject or from multiple reference subjects. Selection of the particular baseline values or reference standards, or selection of the one or more reference subjects, depends upon the use to which the methods described herein are to be put by, for example, a research scientist or a clinician (such as a physician).

In some embodiments, one or more samples (such as a tissue sample or a blood sample, e.g., a whole blood sample, a buffy coat sample, a leukapheresis sample, or a PBMC sample) may be collected from a subject at two or more timepoints, to assess changes in cell types (such as changes in quantities of cell types) between the two or more timepoints. In some embodiments, a sample collected at a first time point is a tissue sample or a blood sample, and a sample collected at a subsequent time point (such as a second time point) is a blood sample. In some embodiments, a sample collected at a first time point is a tissue sample and a sample collected at a subsequent time point (such as a second time point) is a blood sample. By monitoring cell types and identifying differences between cell types in samples collected from a subject at two or more timepoints, the present methods can be used, for example, to determine the presence or absence of a condition (such as a cancer), a response of the subject to a treatment, one or more characteristic of a condition (such as a cancer stage) in the subject, recurrence of a condition (such as a cancer), and/or a subject's risk of developing a condition (such as a cancer). Thus, in some embodiments, methods are provided wherein quantities of cell types present in at least one sample (such as at least one tissue sample and/or at least one blood sample, e.g., a whole blood sample, buffy coat sample, leukapheresis sample, or PBMC sample) collected from a subject at one or more timepoints (such as prior to receiving a treatment) are compared to quantities of cell types present in at least one sample collected from the subject at one or more different time points (such as after receiving the treatment). The disclosed methods can allow for patient-specific monitoring, such that, for example, differences in cell type quantities between samples collected from the subject at different timepoints may indicate changes (such as presence or absence of a condition, response to a treatment, a prognosis, or the like) that are significant with respect to the subject but may yet fall within a normal range of a general healthy population.

As disclosed herein, methods are provided for monitoring one or more aspects of a condition in a subject over time, such as but not limited to, a subject's response to receiving a treatment for a condition (such as a response to a chemotherapeutic or immunotherapeutic). In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points prior to the subject receiving the treatment. In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points after the subject has received the treatment. Sample collection from a subject can be ongoing during and/or after treatment to monitor the subject's response to the treatment.

In some embodiments, samples are not collected from a subject prior to diagnosis of a condition (such as a cancer) or prior to receiving a treatment. In such embodiments, wherein the response of a subject to a treatment, or the course or stage of a condition (such as a cancer) in the subject is being monitored over time, cell types are compared between samples taken at at least 2-10, at least 2-5, at least 3-6, or at least 2, such as at least 3, at least 4, at least 5, at least 6, at least 7, at least 8, at least 9, at least 10, at least 15, or at least 20 time points collected after the subject has been diagnosed and/or after the subject has received the treatment. Sample collection from a subject can be ongoing during and/or after treatment to monitor the subject's response to the treatment.

In some embodiments of the disclosed methods, one or more samples (such as one or more tissue, whole blood, buffy coat, leukapheresis, or PBMC samples) is collected from a subject at least once per year, such as about 1-12 times or about 2-6 times, such as about 1, 2, 3, 4, 5, 6, 7, 8, 9, 10, 11, or 12 times per year. In other embodiments, one or more samples is collected from the subject less than once per year, such as about once every 13, 14, 15, 16, 17, 18, 19, 20, 21, 22, 23, or 24 months. In some embodiments, one or more samples is collected from the subject about once every 1-5 years or about once every 1-2 years, such as about every 1, 1.5, 2, 2.5, 3, 3.5, 4, 4.5, or 5 years.

In other embodiments of the disclosed methods, one or more samples (such as one or more tissue samples or blood samples, e.g., or one or more buffy coat samples, whole blood samples, leukapheresis samples, or PBMC samples) are collected from a subject at least once per week, such as on 1-4 days, 1-2 days, or on 1, 2, 3, 4, 5, 6, or 7 days per week. In certain embodiments, one or more samples is collected from the subject at least once per month, such as 1-15 times, 1-10 times, 2-5 times, or 1, 2, 3, 4, 5, 6, 7, 8, 9, 10, 11, 12, 13, 14, or 15 times per month. In other embodiments, one or more samples is collected from the subject every month, every 2 months, every 3 months, every 4 months, every 5 months, every 6 months, every 7 months, every 8 months, every 9 months, every 10 months, every 11 months, or every 12 months. In some embodiments, one or more samples is collected from the subject at least once per day, such as 1, 2, 3, 4, 5, or 6 times per day. Selection of the one or more sample collection timepoints (e.g., the frequency of sample collection), or of the number of samples to be collected at each timepoint, depends upon the use to which the methods described herein are to be put by, for example, a research scientist or a clinician (such as a physician).

### 4. Therapies and Related Administration

In certain embodiments, the methods disclosed herein relate to identifying and administering customized therapies, such as customized therapies to patients. In some embodiments, determination of the levels of particular immune cell types, including rare immune cell types, facilitates selection of appropriate treatment. In some embodiments, the patient or subject has a given disease, disorder or condition, e.g., any of the cancers or other conditions described elsewhere herein. Essentially any cancer therapy (e.g., surgical therapy, radiation therapy, chemotherapy, immunotherapy, and/or the like) may be included as part of these methods. In certain embodiments, the therapy administered to a subject comprises at least one chemotherapy drug. In some embodiments, the chemotherapy drug may comprise alkylating agents (for example, but not limited to, Chlorambucil, Cyclophosphamide, Cisplatin and Carboplatin), nitrosoureas (for example, but not limited to, Carmustine and Lomustine), anti-metabolites (for example, but not limited to, Fluorauracil, Methotrexate and Fludarabine), plant alkaloids and natural products (for example, but not limited to, Vincristine, Paclitaxel and Topotecan), anti-tumor antibiotics (for example, but not limited to, Bleomycin, Doxorubicin and Mitoxantrone), hormonal agents (for example, but not limited to, Prednisone, Dexamethasone, Tamoxifen and Leuprolide) and biological response modifiers (for example, but not limited to, Herceptin and Avastin, Erbitux and Rituxan). In some embodiments, the chemotherapy administered to a subject may comprise FOLFOX or FOLFIRI. In certain embodiments, a therapy may be administered to a subject that comprises at least one PARP inhibitor. In certain embodiments, the PARP inhibitor may include OLAPARIB, TALAZOPARIB, RUCAPARIB, NIRAPARIB (trade name ZEJULA), among others. Typically, therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In some embodiments, therapy is customized based on the status of a nucleic acid variant as being of somatic or germline origin. In some embodiments, essentially any cancer therapy (e.g., surgical therapy, radiation therapy, chemotherapy, immunotherapy, and/or the like) may be included as part of these methods. Customized therapies can include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In some embodiments, the immunotherapy or immunotherapeutic agent targets an immune checkpoint molecule. Certain tumors are able to evade the immune system by co-opting an immune checkpoint pathway. Thus, targeting immune checkpoints has emerged as an effective approach for countering a tumor's ability to evade the immune system and activating anti-tumor immunity against certain cancers. Pardoll, Nature Reviews Cancer, 2012, 12:252-264.

In certain embodiments, the immune checkpoint molecule is an inhibitory molecule that reduces a signal involved in the T cell response to antigen. For example, CTLA4 is expressed on T cells and plays a role in downregulating T cell activation by binding to CD80 (aka B7.1) or CD86 (aka B7.2) on antigen presenting cells. PD-1 is another inhibitory checkpoint molecule that is expressed on T cells. PD-1 limits the activity of T cells in peripheral tissues during an inflammatory response. In addition, the ligand for PD-1 (PD-L1 or PD-L2) is commonly upregulated on the surface of many different tumors, resulting in the downregulation of anti-tumor immune responses in the tumor microenvironment. In certain embodiments, the inhibitory immune checkpoint molecule is CTLA4 or PD-1. In other embodiments, the inhibitory immune checkpoint molecule is a ligand for PD-1, such as PD-L1 or PD-L2. In other embodiments, the inhibitory immune checkpoint molecule is a ligand for CTLA4, such as CD80 or CD86. In other embodiments, the inhibitory immune checkpoint molecule is lymphocyte activation gene 3 (LAG3), killer cell immunoglobulin like receptor (KIR), T cell membrane protein 3 (TIM3), galectin 9 (GAL9), or adenosine A2a receptor (A2aR).

Antagonists that target these immune checkpoint molecules can be used to enhance antigen-specific T cell responses against certain cancers. Accordingly, in certain embodiments, the immunotherapy or immunotherapeutic agent is an antagonist of an inhibitory immune checkpoint molecule. In certain embodiments, the inhibitory immune checkpoint molecule is PD-1. In certain embodiments, the inhibitory immune checkpoint molecule is PD-L1. In certain embodiments, the antagonist of the inhibitory immune checkpoint molecule is an antibody (e.g., a monoclonal antibody). In certain embodiments, the antibody or monoclonal antibody is an anti-CTLA4, anti-PD-1, anti-PD-L1, or anti-PD-L2 antibody. In certain embodiments, the antibody is a monoclonal anti-PD-1 antibody. In some embodiments, the antibody is a monoclonal anti-PD-L1 antibody. In certain embodiments, the monoclonal antibody is a combination of an anti-CTLA4 antibody and an anti-PD-1 antibody, an anti-CTLA4 antibody and an anti-PD-L1 antibody, or an anti-PD-L1 antibody and an anti-PD-1 antibody. In certain embodiments, the anti-PD-1 antibody is one or more of pembrolizumab (Keytruda®) or nivolumab (Opdivo®). In certain embodiments, the anti-CTLA4 antibody is ipilimumab (Yervoy®). In certain embodiments, the anti-PD-L1 antibody is one or more of atezolizumab (Tecentriq®), avelumab (Bavencio®), or durvalumab (Imfinzi®).

In certain embodiments, the immunotherapy or immunotherapeutic agent is an antagonist (e.g., antibody) against CD80, CD86, LAG3, KIR, TIM3, GAL9, or A2aR. In other embodiments, the antagonist is a soluble version of the inhibitory immune checkpoint molecule, such as a soluble fusion protein comprising the extracellular domain of the inhibitory immune checkpoint molecule and an Fc domain of an antibody. In certain embodiments, the soluble fusion protein comprises the extracellular domain of CTLA4, PD-1, PD-L1, or PD-L2. In some embodiments, the soluble fusion protein comprises the extracellular domain of CD80, CD86, LAG3, KIR, TIM3, GAL9, or A2aR. In one embodiment, the soluble fusion protein comprises the extracellular domain of PD-L2 or LAG3.

In certain embodiments, the immune checkpoint molecule is a co-stimulatory molecule that amplifies a signal involved in a T cell response to an antigen. For example, CD28 is a co-stimulatory receptor expressed on T cells. When a T cell binds to antigen through its T cell receptor, CD28 binds to CD80 (aka B7.1) or CD86 (aka B7.2) on antigen-presenting cells to amplify T cell receptor signaling and promote T cell activation. Because CD28 binds to the same ligands (CD80 and CD86) as CTLA4, CTLA4 is able to counteract or regulate the co-stimulatory signaling mediated by CD28. In certain embodiments, the immune checkpoint molecule is a co-stimulatory molecule selected from CD28, inducible T cell co-stimulator (ICOS), CD137, OX40, or CD27. In other embodiments, the immune checkpoint molecule is a ligand of a co-stimulatory molecule, including, for example, CD80, CD86, B7RP1, B7-H3, B7-H4, CD137L, OX40L, or CD70.

Agonists that target these co-stimulatory checkpoint molecules can be used to enhance antigen-specific T cell responses against certain cancers. Accordingly, in certain embodiments, the immunotherapy or immunotherapeutic agent is an agonist of a co-stimulatory checkpoint molecule. In certain embodiments, the agonist of the co-stimulatory checkpoint molecule is an agonist antibody and preferably is a monoclonal antibody. In certain embodiments, the agonist antibody or monoclonal antibody is an anti-CD28 antibody. In other embodiments, the agonist antibody or monoclonal antibody is an anti-ICOS, anti-CD137, anti-OX40, or anti-CD27 antibody. In other embodiments, the agonist antibody or monoclonal antibody is an anti-CD80, anti-CD86, anti-B7RP1, anti-B7-H3, anti-B7-H4, anti-CD137L, anti-OX40L, or anti-CD70 antibody.

In certain embodiments, the status of a nucleic acid variant from a sample from a subject as being of somatic or germline origin may be compared with a database of comparator results from a reference population to identify customized or targeted therapies for that subject. Typically, the reference population includes patients with the same cancer or disease type as the subject and/or patients who are receiving, or who have received, the same therapy as the subject. A customized or targeted therapy (or therapies) may be identified when the nucleic variant and the comparator results satisfy certain classification criteria (e.g., are a substantial or an approximate match).

In certain embodiments, the customized therapies described herein are typically administered parenterally (e.g., intravenously or subcutaneously). Pharmaceutical compositions containing an immunotherapeutic agent are typically administered intravenously. Certain therapeutic agents are administered orally. However, customized therapies (e.g., immunotherapeutic agents, etc.) may also be administered by any method known in the art, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

In some embodiments, e.g., where genetic variants are detected, therapy is customized based on the status of a nucleic acid variant as being of somatic or germline origin. In some embodiments, determination of the levels of particular cell types, e.g., immune cell types, including rare immune cell types, facilitates selection of appropriate treatment.

The present methods can be used to diagnose the presence of a condition, e.g., cancer or precancer, in a subject, to characterize a condition (such as to determine a cancer stage or heterogeneity of a cancer), to monitor a subject's response to receiving a treatment for a condition (such as a response to a chemotherapeutic or immunotherapeutic), assess prognosis of a subject (such as to predict a survival outcome in a subject having a cancer), to determine a subject's risk of developing a condition, to predict a subsequent course of a condition in a subject, to determine metastasis or recurrence of a cancer in a subject (or a risk of cancer metastasis or recurrence), and/or to monitor a subject's health as part of a preventative health monitoring program (such as to determine whether and/or when a subject is in need of further diagnostic screening). The methods according to the present disclosure can also be useful in predicting a subject's response to a particular treatment option. Successful treatment options may increase the amount of copy number variation, rare mutations, and/or cancer-related epigenetic signatures (such as hypermethylated regions or hypomethylated regions) detected in a subject's blood (such as in DNA isolated from a buffy coat sample or any other sample comprising cells, such as a blood sample (e.g., a whole blood sample, a buffy coat sample, a leukapheresis sample, or a PBMC sample) from the subject) if the treatment is successful as more cancer cells may die and shed DNA, or if a successful treatment results in an increase or decrease in the quantity of a specific immune cell type in the blood and an unsuccessful treatment results in no change. In other examples, this may not occur. In another example, certain treatment options may be correlated with genetic profiles of cancers over time. This correlation may be useful in selecting a therapy for a subject. In some embodiments, determination of the metastasis site facilitates selection of appropriate treatment.

Thus, in some embodiments, quantities of each of one or more of a particular genetic and/or epigenetic signature (e.g., quantities of fusions, indels, SNPs, CNVs, and/or rare mutations, and/or cancer-related epigenetic signatures (such as specific (e.g., DMRs) or global hypermethylated or hypomethylated regions, and/or fragmentation variable regions)) in DNA from a subject's blood (such as in DNA (e.g., cfDNA) isolated from a blood sample (e.g., a whole blood sample) from the subject)) are determined based on sequencing and analysis. In some embodiments, quantities of each of a plurality of cell types, such as immune cell types, are determined based on sequencing and analysis (such as determination of epigenetic and/or genomic signatures) of DNA isolated from at least one sample comprising cells (such as blood sample (e.g., a whole blood sample, a buffy coat sample, a leukapheresis sample, or a PBMC sample) from a subject. The plurality of immune cell types can include, but is not limited to, macrophages (including M1 macrophages and M2 macrophages), activated B cells (including regulatory B cells, memory B cells and plasma cells); T cell subsets, such as central memory T cells, naïve-like T cells, and activated T cells (including cytotoxic T cells, regulatory T cells (Tregs), CD4 effector memory T cells, CD4 central memory T cells, CD8 effector memory T cells, and CD8 central memory T cells); immature myeloid cells (including myeloid-derived suppressor cells (MDSCs), low-density neutrophils, immature neutrophils, and immature granulocytes); and natural killer (NK) cells. As disclosed herein, differences in levels and/or presence of particular genetic and/or epigenetic signatures in DNA isolated from blood samples from a subject can be used to quantify cell types, such as immune cell types, within the sample. Thus, a comparison of one or more genetic and/or epigenetic signatures in DNA isolated from blood samples collected from a subject at two or more time points can be used to monitor changes in the one or more signatures and/or the one or more cell type quantities in the subject under different conditions (such as prior to and after a treatment), or over time (e.g., as part of a preventative health monitoring program).

In some embodiments, therapy is customized based on the status of a detected nucleic acid variant as being of somatic or germline origin. In some embodiments, essentially any cancer therapy (e.g., surgical therapy, radiation therapy, chemotherapy, and/or the like) may be included as part of these methods. Typically, customized therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In certain embodiments, the status of a nucleic acid variant from a sample from a subject as being of somatic or germline origin may be compared with a database of comparator results from a reference population to identify customized or targeted therapies for that subject. Typically, the reference population includes patients with the same cancer or disease type as the subject and/or patients who are receiving, or who have received, the same therapy as the subject. A customized or targeted therapy (or therapies) may be identified when the nucleic variant and the comparator results satisfy certain classification criteria (e.g., are a substantial or an approximate match).

The disclosed methods can include evaluating (such as quantifying) and/or interpreting at least one cell material released from a potential metastasis site (such as at least one cell material in a sample from a subject) and/or cell types that contribute to DNA, such as cfDNA, in one or more samples collected from a subject at one or more timepoints in comparison to a selected baseline value or reference standard (or a selected set of baseline values or reference standards). A baseline value or reference standard may be a presence or level of at least one cell material and/or a quantity of cell types measured in one or more samples (such as an average quantity or range of quantities of cell types present in at least two samples) collected from the subject at one or more time points, such as prior to receiving a treatment, prior to diagnosis of a condition (such as a cancer), or as part of a preventative health monitoring program. A baseline value or reference standard may be a presence or level of at least one cell material and/or a quantity of cell types measured with respect to one or more samples (such as an average quantity or range of quantities of cell types present in at least two samples) collected at one or more timepoints from one or more subjects that do not have the condition (such as a healthy subject that does not have a cancer), one or more subjects that responded favorably to the treatment, or one or more subjects that have not received the treatment. In certain embodiments, the baseline value or reference standard utilized is a standard or profile derived from a single reference subject. In other embodiments, the baseline value or reference standard utilized is a standard or profile derived from averaged data from multiple reference subjects. The reference standard, in various embodiments, can be a single value, a mean, an average, a numerical mean or range of numerical means, a numerical pattern, or a graphical pattern created from the cell type quantity data derived from a single reference subject or from multiple reference subjects. Selection of the particular baseline values or reference standards, or selection of the one or more reference subjects, depends upon the use to which the methods described herein are to be put by, for example, a research scientist or a clinician (such as a physician).

The disclosed methods can include evaluating (such as quantifying) and/or interpreting one or more genetic and/or epigenetic signatures, and/or one or more cell types (such as one or more immune cell types), present in one or more samples (e.g., in DNA, such as cfDNA, from a blood sample (e.g., a whole blood sample, a buffy coat sample, a leukapheresis sample, or a PBMC sample)) collected from a subject at one or more timepoints in comparison to a selected baseline value or reference standard (or a selected set of baseline values or reference standards). A baseline value or reference standard may be a quantity of copy number variation, rare mutations, cancer-related epigenetic signatures (such as hypermethylated regions or hypomethylated regions), and/or cell types measured in one or more samples (such as an average quantity or range of quantities of such signatures present in at least two samples) collected from the subject at one or more time points, such as prior to receiving a treatment, prior to diagnosis of a condition (such as a cancer), or as part of a preventative health monitoring program. A baseline value or reference standard may be a quantity of, e.g., copy number variation, rare mutations, cancer-related epigenetic signatures (such as hypermethylated regions or hypomethylated regions), and/or cell types measured in one or more samples (such as an average quantity or range of quantities of such signatures and/or cell types present in at least two samples) collected at one or more timepoints from one or more subjects that do not have the condition (such as a healthy subject that does not have a cancer), one or more subjects that responded favorably to the treatment, or one or more subjects that have not received the treatment.

In certain embodiments, the baseline value or reference standard utilized is a standard or profile derived from a single reference subject. In other embodiments, the baseline value or reference standard utilized is a standard or profile derived from averaged data from multiple reference subjects. The reference standard, in various embodiments, can be a single value, a mean, an average, a numerical mean or range of numerical means, a numerical pattern, or a graphical pattern created from the genetic and/or epigenetic signature quantity data derived from a single reference subject or from multiple reference subjects. Selection of the particular baseline values or reference standards, or selection of the one or more reference subjects, depends upon the use to which the methods described herein are to be put by, for example, a research scientist or a clinician (such as a physician).

In some embodiments, one or more samples comprising cells (such as a buffy coat sample or any other sample comprising cells, such as a blood sample (e.g., a whole blood sample, a leukapheresis sample, or a PBMC sample) may be collected from a subject at two or more timepoints, to assess changes in cell types (such as changes in quantities of cell types) between the two timepoints. By monitoring cell types and identifying differences between cell types in samples collected from a subject at two or more timepoints, the present methods can be used, for example, to determine the presence or absence of a condition (such as a cancer), a response of the subject to a treatment, one or more characteristic of a condition (such as a cancer stage) in the subject, recurrence of a condition (such as a cancer), and/or a subject's risk of developing a condition (such as a cancer). Thus, in some embodiments, methods are provided wherein quantities of cell types present in at least one sample (such as at least one whole blood sample, buffy coat sample, leukapheresis sample, or PBMC sample) collected from a subject at one or more timepoints (such as prior to receiving a treatment) are compared to quantities of cell types present in at least one sample collected from the subject at one or more different time points (such as after receiving the treatment). The disclosed methods can allow for patient-specific monitoring, such that, for example, differences in cell type quantities between samples collected from the subject at different timepoints may indicate changes (such as presence or absence of a condition, response to a treatment, a prognosis, or the like) that are significant with respect to the subject but may yet fall within a normal range of a general healthy population.

In some embodiments, methods are provided for monitoring a response (such as a change in disease state, such as a presence or absence of a metastasis in a subject, such as measured by assessing a presence or level of at least one cell material released from a potential metastasis site in a sample from the subject) of a subject to a treatment (such as a chemotherapy or an immunotherapy). In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points prior to the subject receiving the treatment. In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points after the subject has received the treatment. Sample collection from a subject can be ongoing during and/or after treatment to monitor the subject's response to the treatment.

In some embodiments, samples are not collected from a subject prior to diagnosis of a condition (such as a cancer) or prior to receiving a treatment. In such embodiments, wherein the response of a subject to a treatment or the course or stage of a condition (such as a cancer) in the subject is being monitored over time, genetic and/or epigenetic signatures, and/or cell types are compared between samples taken at at least 2-10, at least 2-5, at least 3-6, or at least 2, such as at least 3, at least 4, at least 5, at least 6, at least 7, at least 8, at least 9, at least 10, at least 15, or at least 20 time points collected after the subject has been diagnosed and/or after the subject has received the treatment. Sample collection from a subject can be ongoing during and/or after treatment to monitor the subject's response to the treatment.

In some embodiments of the disclosed methods, one or more samples is collected from a subject at least once per year, such as about 1-12 times or about 2-6 times, such as about 1, 2, 3, 4, 5, 6, 7, 8, 9, 10, 11, or 12 times per year. In other embodiments, one or more samples is collected from the subject less than once per year, such as about once every 13, 14, 15, 16, 17, 18, 19, 20, 21, 22, 23, or 24 months. In some embodiments, one or more samples is collected from the subject about once every 1-5 years or about once every 1-2 years, such as about every 1, 1.5, 2, 2.5, 3, 3.5, 4, 4.5, or 5 years.

In other embodiments of the disclosed methods, one or more samples (such as one or more whole blood, buffy coat, leukapheresis, or PBMC samples) are collected from a subject at least once per week, such as on 1-4 days, 1-2 days, or on 1, 2, 3, 4, 5, 6, or 7 days per week. In certain embodiments, one or more samples are collected from the subject at least once per month, such as 1-15 times, 1-10 times, 2-5 times, or 1, 2, 3, 4, 5, 6, 7, 8, 9, 10, 11, 12, 13, 14, or 15 times per month. In other embodiments, one or more samples is collected from the subject every month, every 2 months, every 3 months, every 4 months, every 5 months, every 6 months, every 7 months, every 8 months, every 9 months, every 10 months, every 11 months, or every 12 months. In some embodiments, one or more samples is collected from the subject at least once per day, such as 1, 2, 3, 4, 5, or 6 times per day. Selection of the one or more sample collection timepoints (e.g., the frequency of sample collection), or of the number of samples to be collected at each timepoint, depends upon the use to which the methods described herein are to be put by, for example, a research scientist or a clinician (such as a physician).

In certain embodiments, the customized therapies described herein are typically administered parenterally (e.g., intravenously or subcutaneously). Pharmaceutical compositions containing an immunotherapeutic agent are typically administered intravenously. Certain therapeutic agents are administered orally. However, customized therapies (e.g., immunotherapeutic agents, etc.) may also be administered by methods such as, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

Therapeutic options for treating specific genetic-based diseases, disorders, or conditions, other than cancer, are generally well-known to those of ordinary skill in the art and will be apparent given the particular disease, disorder, or condition under consideration.

### E. Systems and Computer Readable Media

The various processing operations and/or methods depicted in the Figures may be accomplished using some or all of the system components described in detail herein and, in some implementations, various operations may be performed in different sequences and various operations may be omitted. Additional operations may be performed along with some or all of the operations shown in the depicted flow diagrams. One or more operations may be performed simultaneously. Accordingly, the operations as illustrated (and described in greater detail herein) are provided as example and, as such, should not be viewed as limiting.

The present methods can be computer-implemented, such that any or all of the operations described in the specification or appended claims other than wet chemistry steps can be performed in a suitable programmed computer. The computer can be a mainframe, personal computer, tablet, smart phone, cloud, online data storage, remote data storage, or the like. The computer can be operated in one or more locations.

Various operations of the present methods can utilize information and/or programs and generate results that are stored on computer-readable media (e.g., hard drive, auxiliary memory, external memory, server; database, portable memory device (e.g., CD-R, DVD, ZIP disk, flash memory cards), and the like.

The present disclosure also includes an article of manufacture for analyzing a nucleic acid population that includes a machine-readable medium containing one or more programs which when executed implement the steps of the present methods.

The disclosure can be implemented in hardware and/or software. For example, different aspects of the disclosure can be implemented in either client-side logic or server-side logic. The disclosure or components thereof can be embodied in a fixed media program component containing logic instructions and/or data that when loaded into an appropriately configured computing device cause that device to perform according to the disclosure. A fixed media containing logic instructions can be delivered to a viewer on a fixed media for physically loading into a viewer's computer or a fixed media containing logic instructions may reside on a remote server that a viewer accesses through a communication medium to download a program component.

The present disclosure provides computer control systems that are programmed to implement methods of the disclosure. Returning to FIG. 2, the processor 220 may include a single core or multi core processor, or a plurality of processors for parallel processing. The storage device 222 may include random-access memory, read-only memory, flash memory, a hard disk, and/or other type of storage. The computer system 210 may include a communication interface (e.g., network adapter) for communicating with one or more other systems, and peripheral devices, such as cache, other memory, data storage and/or electronic display adapters. The components of the computer system 210 may communicate with one another through an internal communication bus, such as a motherboard. The storage device 222 may be a data storage unit (or data repository) for storing data. The computer system 210 may be operatively coupled to a network 223 (“network”) with the aid of the communication interface. The network 223 may be the Internet, an internet and/or extranet, or an intranet and/or extranet that is in communication with the Internet. The network 223 in some cases is a telecommunication and/or data network. The network 223 may include a local area network. The network 23 may include one or more computer servers, which can enable distributed computing, such as cloud computing. The network 223, in some cases with the aid of the computer system 210, may implement a peer-to-peer network, which may enable devices coupled to the computer system 220 to behave as a client or a server. The computer system 210 may exchange data with a computer system 224 using the network 223. For example, the computer system 224 may retrieve data from the analytics datastore 236.

The processor 220 may execute a sequence of machine-readable instructions, which can be embodied in a program or software. The instructions may be stored in a memory location, such as the storage device 222. The instructions can be directed to the processor 220, which can subsequently program or otherwise configure the processor 220 to implement methods of the present disclosure. Examples of operations performed by the processor 220 may include fetch, decode, execute, and writeback.

The processor 220 may be part of a circuit, such as an integrated circuit. One or more other components of the system 200 may be included in the circuit. In some cases, the circuit may include an application specific integrated circuit (ASIC).

The storage device 222 may store files, such as drivers, libraries and saved programs. The storage device 222 can store user data, e.g., user preferences and user programs. The computer system 210 in some cases may include one or more additional data storage units that are external to the computer system 210, such as located on a remote server that is in communication with the computer system 210 through an intranet or the Internet.

The computer system 210 can communicate with one or more remote computer systems through the network. For instance, the computer system 210 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 110 via the network.

Methods as described herein can be implemented by way of machine (e.g., computer processor) executable code stored on an electronic storage location of the computer system 210, such as, for example, on the storage device 222. The machine executable or machine readable code can be provided in the form of software (e.g., computer readable media). During use, the code can be executed by the processor 220. In some cases, the code can be retrieved from the storage device 222 and stored on the storage device 222 for ready access by the processor 220.

The code may be pre-compiled and configured for use with a machine having a processer adapted to execute the code, or can be compiled during runtime. The code can be supplied in a programming language that can be selected to enable the code to execute in a precompiled or as-compiled fashion.

Aspects of the systems and methods provided herein, such as the computer system 210, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk.

“Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible, storage media, “media” may include other types of (intangible) media.

“Storage” media, terms such as computer or machine “readable medium” refer to any tangible (such as physical), non-transitory, medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 210 can include or be in communication with an electronic display 935 that comprises a user interface (UI) for providing, for example, a report. Examples of UI's include, without limitation, a graphical user interface (GUI) and web-based user interface.

Methods and systems of the present disclosure can be implemented by way of one or more algorithms. An algorithm can be implemented by way of software upon execution by the processor 220.

### EXAMPLES

### A. Example: A Cell-Free DNA Blood-Based Test for Colorectal Cancer Screening

### 1. Introduction

Colorectal cancer is the third most diagnosed cancer and second leading cause of cancer-related death in adults in the United States. The lifetime risk of colorectal cancer in the United States is approximately 4%, with 53,000 persons expected to die from the disease in 2024. Earlier detection of colorectal cancer affects overall survival; 5-year survival is 91% among persons with localized disease as compared with 14% among those with metastatic disease. Asymptomatic screening reduces the incidence of colorectal cancer and related deaths and is uniformly recommended by leading professional societies, including the U.S. Preventive Services Task Force (USPSTF), the U.S. Multi-Society Task Force on Colorectal Cancer, and the American Cancer Society (ACS). Numerous screening options are available, including direct visualization and stool-based tests, but owing to inherent barriers, approximately 59% of eligible persons 45 years of age or older are adherent to screening guidelines, well below the target of 80% set forth by the National Colorectal Cancer Roundtable (established by the Centers for Disease Control and Prevention and the ACS). In addition, 76% of colorectal cancer-related deaths occur in persons who are not up to date with screening. There is a pressing need for screening tests for colorectal cancer that are easier to administer and increase adherence.

Factors contributing to low screening adherence include the time required to perform screening, scheduling challenges, concern over test invasiveness and pain, fear of the test, discomfort or embarrassment associated with endoscopic examinations, lack of insurance coverage, distance from the test provider, and lack of physician recommendation for screening. Incorporating a blood-based test, performed as part of a routine health care encounter, to the existing screening paradigm would provide an additional screening option that is relatively simple to complete, thus improving adherence. The performance of a cell-free DNA (cfDNA) blood-based screening test for colorectal cancer in an average-risk population is described herein.

i. Specimen Collection

The clinical blood specimens were collected into up to 8 provided 10 mL Streck whole blood collection tubes per subject. The blinded samples were then sent overnight at ambient temperature to the central biorepository for plasma isolation and storage. Upon receipt, the blood was fractionated via centrifugation and plasma was isolated and stored at −80 C. Frozen plasma isolated from up to 4, 10 mL blood tubes (one blood collection kit) was then shipped blinded to the central laboratory for testing. All resulting plasma isolated from one blood collection kit for each tested individual was pooled per subject (median total plasma volume: 14.5 mL, mean total volume: 13.4 mL). Plasma was divided into primary and retain aliquots with a minimum volume of 2 mL and a maximum volume of 8 mL in each aliquot. cfDNA (cell-free DNA) was first extracted from primary plasma aliquot. The retain aliquot was extracted to retest failed samples as needed. 64% of extracted tubes contained the full 8 mL of plasma. The remainder contained an average of 5.4 mL of plasma. After extraction, cfDNA was separated into methylated and unmethylated partitions based on the overall methylation state of each molecule. The cfDNA was partitioned based on the differential binding affinity of the methylated nucleic acid molecules to a binding agent (i.e., a binding agent that binds to methylated nucleotides). No bisulfite conversion was used. The DNA in each partition was then tagged with a distinct set of dual barcodes, which uniquely identifies the partition associated with every molecule and aid in identification of unique cfDNA molecules post sequencing. DNA molecules in the methylated partitions were then treated with restriction enzymes to deplete the samples of partially methylated molecules. All partitions were then PCR amplified and enriched via hybridization to oligonucleotides representing genomic regions of interest targeting approximately 1 Mb of human genome. Enriched partitions were pooled and tagged with an index uniquely identifying each sample prior to pooling multiple enriched samples into sequencing pools. Sequencing pools were sequenced on the NovaSeq 6000 instruments.

ii. Sequencing Data Analysis

Following sequencing, reads were demultiplexed and replicate reads of the same molecule were grouped into families representing each of the unique molecules. The methylation partition associated with every molecule was identified by the barcodes added during library preparation to enable differentiation of methylated and unmethylated molecules in the analysis step. Only unique molecules which align to genomic regions within the enrichment panels were leveraged in the downstream algorithms.

iii. Neoplasia Detection Algorithm in the Cancer Screening Test

The classification of a clinical sample relies upon the multiple biomarkers derived from cfDNA and known to be distinct between normal and cancer-derived tissues. The cancer screening test is an in vitro diagnostic multi-index assay (IVD-MIA) which interrogates thousands of individual features that characterize three types of cfDNA signals or patterns: epigenetic changes resulting in the aberrant methylation state, epigenetic changes resulting in the aberrant cfDNA molecule fragmentation patterns, and genomic changes resulting in somatic mutations.

The cancer screening test result is determined based on two scores: the score from a methylation-based tumor fraction regression model (TFR model) and the cfDNA integrated score. If either the cfDNA integrated score or the TFR score exceeds their respective pre-defined thresholds, the cancer screening test result is positive (abnormal). Otherwise, the cancer screening test result is negative (normal).

The algorithms were trained using over 4,000 development samples representing diverse cohorts of healthy donor samples, colonoscopy-screened CRC negative donors, as well as CRC patients. Parameters of the cfDNA sample processing, QC, and cfDNA regression models and weights were locked in a software algorithm prior to the initiation of the clinical testing.

iv. Methylation-Based Tumor Fraction Regression (TFR) Model Score

The TFR model was developed to quantify the fraction of tumor-derived cfDNA (tumor fraction) in a sample based on the quantification of the observed tumor-associated aberrant methylation of cfDNA molecules. This quantification is based on the observed number of unique methylated molecules mapping to each of the targeted classification regions. These molecule counts are normalized to the overall number of unique methylated molecules observed in the normalization regions of the panel. After normalization, the dependence of the classification region feature values (normalized molecule counts) on the total number of molecules measured and input cfDNA amount for a sample is minimized. Region level normalized molecule counts are used as input features into the TFR model. The model was trained on over 4,000 development samples to predict their tumor fraction. The predicted tumor fraction is used as a score for assessment of cancer status of an individual sample.

v. cfDNA Integrated Score

The cfDNA integrated model developed is a logistic regression model to generate a quantitative score indicating presence of tumor-derived molecules based on the joint assessment of the epigenetic signals (cfDNA methylation status and fragmentation patterns) and a qualitative mutation detected status (for somatic mutations). Each of these analytes are first analyzed separately and then the resulting individual quantitative scores of the per-analyte assessments are aggregated by the cfDNA integrated model to produce a single cfDNA integrated score. The details about these individual scores are described below:

a. Methylation Models

In addition to the TFR model described above, a different methylation model, methylation logistic regression (LR) model, was developed to differentiate the tumor-associated methylation signatures of cfDNA molecules from those observed in subjects without tumors. The methylation LR model uses the same input feature space as the TFR model, namely the region level normalized molecule counts described above. Compared to the TFR model, the methylation LR model was trained to predict the binary disease state (cancer and non-cancer) instead of the quantitative tumor fraction. The methylation LR model was trained on the same set of samples used to train the TFR model. The scores of both the TFR model and the methylation LR model are used as input to the cfDNA integrated model.

b. Fragmentomics Model

A fragmentomics model was developed to capture the cancer signal from tumor-associated cfDNA fragmentation patterns. To derive quantitative scores associated with the fragmentation patterns, a mixture model of molecule endpoint densities within each of the fragmentomics relevant classification regions was trained to estimate endpoint densities across normal and CRC samples. A molecule endpoint density is defined for each genomic region/sample as follows. For each genomic position, the number of molecule endpoints present at that position is aggregated and normalized by the total endpoint count for that region and sample. Only molecules between 120 and 240 bp in the unmethylated partition mapping to the set of regions identified as informative for fragmentomics signal differences are used. The pattern in an individual sample is then fit as a mixture of the CRC and normal endpoint densities and a posterior expected value of the mixing proportion between normal and CRC densities is derived for each region. Finally, a logistic regression model was trained to combine the mixture scores from all classification regions within the fragmentomics subpanel into a single quantitative score.

c. Somatic Caller

The cfDNA integrated model is also informed by whether any tumor-derived mutations are identified. Somatic caller leverages the somatic variants observed in the molecules from all partitions and its output is dichotomous: one or more tumor-derived mutations detected or none detected. Somatic caller was trained to minimize false positive rates associated with non-tumor derived variants commonly found in cfDNA samples of healthy individuals at low allelic frequencies. Only somatic nonsense SNVs (single nucleotide variants), splice variants, and indels with variant allele fraction (VAF) >0.1% in APC or KRAS are considered when generating a positive somatic call, and these variants are further filtered based on the variant frequency and clonality observed in the large internal reference database of cancer samples.

The three quantitative scores from the two methylation models and the fragmentomic model and the qualitative assessment of somatic mutation detection (encoded as 0 for absence of mutations and 1 for positive detection of somatic mutations) are standardized and integrated using a logistic regression model (cfDNA integrated model) to produce a single integrated score per sample. The cfDNA integrated model was trained to predict CRC status using an independent set of samples that were not used to train either the methylation models or the fragmentomics model.

A separate device that leverages the above cfDNA workflow in combination with analysis of tumor specific plasma proteins was also evaluated as part of this study. The cfDNA-only device described above outperformed the device that integrated protein.

All samples were received in the central biorepository and central laboratory blinded to clinical findings. Central laboratory remained blinded to clinical attributes of individuals throughout the entire duration of the study. Binary results were reported to the CRO where they were associated with the clinical outcomes for analysis. Patient level results remain blinded to maintain integrity of future data analyses.

vi. Post Hoc Missing Data Multiple Imputation Analysis

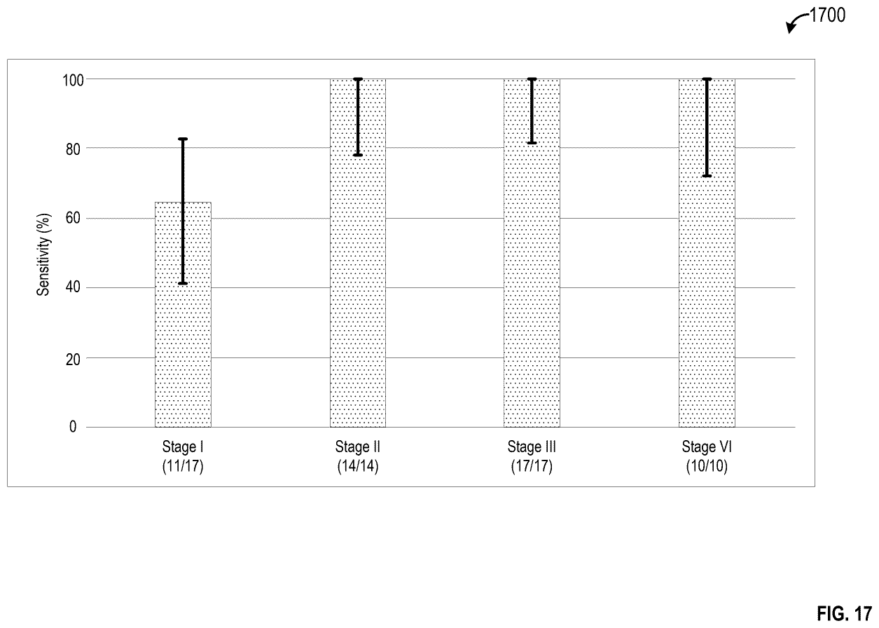

As a post-hoc sensitivity analysis, we performed a missing data multiple imputation analysis in all 10,101 individuals who met eligibility criteria (FIG. 17, which includes a chart 1700 indicating colorectal cancer sensitivity according to stage of diagnosis) to assess for the potential of bias due to missing data. Analysis assumed missing data at random (20 imputations using chained equations with fully conditional specification). Data included in the multiple imputation model were gender, age, body mass index (BMI), race, tobacco use (Table 7), colonoscopy result (Table 9) and cfDNA blood-based test result (positive or negative). No meaningful new significant findings were identified in the univariate or multivariate analyses.

Based on multiple imputation analysis, CRC sensitivity was 80.2% (95% CI: 68.7%-88.2%). Specificity for any advanced neoplasia (APL or CRC) was 89.4% (95% CI: 88.7%-90.1%) Specificity in those individuals without any colonoscopy identified colorectal neoplasia was 89.8% (88.9%-90.6%). Advanced precancerous lesion (APL) sensitivity was 13.5% (95% CI: 11.4%-16.0%).

TABLE 6
Key Terms in Cancer Screening:

| | | Application to Current |
|---|---|---|
| Key Term | Definition | Study |
| Sensitivity | The proportion of people who test | Of the individuals who had CRC |
| | positive for a specific condition among | diagnosis on colonoscopy, what |
| | a group of people who have the | percentage had a positive test |
| | condition | result |
| | | Of the individuals who had an APL |
| | | diagnosis on colonoscopy, what |
| | | percentage had a positive test |
| | | result |
| Overall | The proportion of people who test | Of the individuals whose |
| Specificity | negative for a specific condition | colonoscopy had a non-AN or |
| | among a group of people who do not | negative or non-neoplastic finding, |
| | have the condition | what percentage of had a negative |
| | | test result |
| Specificity for | The proportion of people who test | Of the individuals whose |
| negative/non- | negative for a specific condition | colonoscopy had a negative or |
| neoplastic | among a group of people who do not | non-neoplastic finding, what |
| finding | have the condition | percentage of had a negative test |
| | | result |
| Positive | The likelihood that a person who has a | Of the individuals with a positive |
| Predictive | positive test result does have the | test result, what percentage had a |
| Value | condition. | colonoscopy finding of AN |
| Negative | The likelihood that a person who has a | Of the individuals with a negative |
| Predictive | negative test result does not have the | screening test result, what |
| Value | condition | percentage had a non-CRC |
| | | colonoscopy finding |
| False Negative | A test result that indicates a person | Of those individuals who had a |
| Result | does not have a specific condition | negative test result, what |
| | when the person actually does have the | percentage had a colonoscopy |
| | or condition | finding of CRC |
| False Positive | A result that indicates that a person has | Of those individuals who had a |
| Result | a specific condition when the person | positive test result, what |
| | actually does not have the condition | percentage had a colonoscopy |
| | | finding of non-AN or negative or |
| | | non-neoplastic finding |
| Adherence | The extent to which a person's | Of those individuals for whom |
| | behavior corresponds with agreed | CRC screening is recommended, |
| | recommendations from a health care | what percentage are up to date with |
| | provider | that recommendation |
| Likelihood | Assessment of how a test result | The likelihood ratio in this study is |
| Ratio | modifies the pre-test probability of | calculated by dividing the positive |
| | having the disorder of interest | predictive value of the cfDNA |
| | | blood-based test result by the pre- |
| | | test probability of having the |
| | | disease (population risk) |

TABLE 7
Colonoscopy finding classification

| Category | Clinical Findings | Grouping |
|---|---|---|
| 1 | Colorectal cancer, any stage | CRC |
| 2 | Advanced adenoma | Advanced pre-cancerous lesion |
| 2a | Carcinoma in situ, any size | (APL) |
| 2b | High-grade dysplasia, any size | |
| 2c | Villous growth % (>25%), any size | |
| 2d | Tubular adenoma, ≥10 mm | |
| 2e | Serrated lesion, ≥10 mm (includes | |
| | sessile serrated adenoma/polyp) | |
| 3 | Non-advanced adenoma, >3 | Non-advanced precancerous lesion |
| | adenomas, <10 mm | |
| 4 | Non-advanced adenoma, 1 or 2 | |
| | adenomas, >5 mm, <10 mm | |
| | Non-advanced adenoma, 1 or 2 | |
| 5 | adenomas, ≤5 mm | |
| 6 | Negative, or other findings | Negative/no neoplastic lesion |
| 7 | Not evaluable | Not evaluable/incomplete |

TABLE 8
Cohort Demographics

| | Enrolled Cohort | | Clinical Validation Cohort | | Evaluable Subjects | |
|---|---|---|---|---|---|---|
| Characteristic | (N = 22,877) | | (N = 10,258) | | (N = 7,861) | |
| BMI (kg/m²) at | N | (%) | N | (%) | N | (%) |
| Baseline | | | | | | |
| <30 | 13382 | (58.5) | 5873 | (57.3) | 4610 | (58.6) |
| ≥30 & <35 | 5298 | (23.2) | 2460 | (24.0) | 1873 | (23.8) |
| 35+ | 4148 | (18.1) | 1913 | (18.6) | 1375 | (17.5) |
| Missing | 49 | (0.2) | 12 | (0.1) | 3 | (0.0) |
| Tobacco Use | N | (%) | N | (%) | N | (%) |
| Never | 15902 | (69.5) | 7082 | (69.0) | 5522 | (70.2) |
| Current | 2358 | (10.3) | 1079 | (10.5) | 737 | (9.4) |
| Former | 4574 | (20.0) | 2086 | (20.3) | 1601 | (20.4) |
| Missing | 43 | (0.2) | 11 | (0.1) | 1 | (0.0) |
| Alcohol Use | N | (%) | N | (%) | N | (%) |
| Never | 10580 | (46.2) | 4673 | (45.6) | 3449 | (43.9) |
| Current | 11018 | (48.2) | 4998 | (48.7) | 4004 | (50.9) |
| Former | 1233 | (5.4) | 574 | (5.6) | 406 | (5.2) |
| Missing | 46 | (0.2) | 13 | (0.1) | 2 | (0.0) |
| Illicit Drug Use | N | (%) | N | (%) | N | (%) |
| Never | 21656 | (94.7) | 9721 | (94.8) | 7481 | (95.2) |
| Current | 477 | (2.1) | 202 | (2.0) | 148 | (1.9) |
| Former | 697 | (3.0) | 321 | (3.1) | 229 | (2.9) |
| Missing | 47 | (0.2) | 14 | (0.1) | 3 | (0.0) |

TABLE 9
Demographics of the Clinical Validation Cohort divided by those tested with the
cfDNA blood-based test and those not tested. “Clinical Validation Tested (N = 8,874)” are all
individuals who were tested with the device and included individuals who were subsequently
excluded from analysis as outlined in the methods section of the main manuscript. “Clinical
Validation non-Tested (N = 1,384)” includes those individuals whose blood sample was not
tested for the reasons outlined in the Methodology section of the main paper. “Evaluable
Subjects (N = 7,861)” includes those individuals who were included in the final analysis.

| | Clinical | | Clinical | | Clinical | | | |
|---|---|---|---|---|---|---|---|---|
| | Validation | | Validation | | Validation Not- | | Evaluable | |
| | Cohort | | Tested | | Tested | | Subjects | |
| Characteristic | (N = 10,258) | | (N = 8,874) | | (N = 1,384) | | (N = 7,861) | |
| Age (years) | | | | | | | | |
| n | 10,258 | | 8,874 | | 1,384 | | 7,861 | |
| Mean (SD) | 60.6 | (9.13) | 60.6 | (9.14) | 60.9 | (9.03) | 60.3 | (9.14) |
| Median | 60 | | 60 | | 61 | | 60 | |
| Min, Max | 45, 90 | | 45, 90 | | 45, 84 | | 45, 84 | |
| Age Group (years) | N | (%) | N | (%) | N | (%) | N | (%) |
| 45-49 | 776 | (7.6) | 689 | (7.8) | 87 | (6.3) | 640 | (8.1) |
| 50-59 | 3,877 | (37.8) | 3,337 | (37.6) | 540 | (39.0) | 3,055 | (38.9) |
| 60-69 | 3,284 | (32.0) | 2,835 | (31.9) | 449 | (32.4) | 2,440 | (31.0) |
| 70-79 | 2,226 | (21.7) | 1940 | (21.9) | 286 | (20.7) | 1,670 | (21.2) |
| 80+ | 95 | (0.9) | 73 | (0.8) | 22 | (1.6) | 56 | (0.7) |
| Missing | 0 | | 0 | | 0 | | 0 | |
| Sex | N | (%) | N | (%) | N | (%) | N | (%) |
| Female | 5,493 | (53.5) | 4,742 | (53.4) | 752 | (54.3) | 4,218 | (53.7) |
| Male | 4,765 | (46.5) | 4,133 | (46.6) | 632 | (45.7) | 3,643 | (46.3) |
| Missing | 0 | | 0 | | 0 | | 0 | |
| Race | N | (%) | N | (%) | N | (%) | N | (%) |
| American Indian or | 19 | (0.2) | 18 | (0.2) | 1 | (0.1) | 14 | (0.2) |
| Alaska Native | | | | | | | | |
| Asian | 685 | (6.7) | 644 | (7.3) | 41 | (3.0) | 560 | (7.1) |
| Black or African | 1,353 | (13.2) | 1,096 | (12.4) | 257 | (18.6) | 931 | (11.8) |
| American | | | | | | | | |
| Native Hawaiian or | 24 | (0.2) | 20 | (0.2) | 4 | (0.3) | 19 | (0.2) |
| Other Pacific Islander | | | | | | | | |
| White | 7,939 | (77.4) | 6,895 | (77.7) | 1,044 | (75.4) | 6,167 | (78.5) |
| Other | 189 | (1.8) | 158 | (1.8) | 31 | (2.2) | 137 | (1.7) |
| Multiple | 32 | (0.3) | 29 | (0.3) | 3 | (0.2) | 23 | (0.3) |
| Missing | 17 | (0.2) | 14 | (0.2) | 3 | (0.2) | 10 | (0.1) |
| Ethnicity | N | (%) | N | (%) | N | (%) | N | (%) |
| Hispanic | 1,561 | (15.2) | 1,218 | (13.7) | 343 | (24.8) | 1,044 | (13.3) |
| Not Hispanic or Latino | 8,643 | (84.3) | 7,618 | (85.8) | 1,030 | (74.4) | 6,779 | (86.2) |
| Missing | 54 | (0.5) | 43 | (0.5) | 11 | (0.8) | 38 | (0.5) |
| BMI (kg/m²) at Baseline | N | (%) | N | (%) | N | (%) | N | (%) |
| <30 | 5873 | (57.3) | 5,169 | (58.2) | 704 | (50.9) | 4610 | (58.6) |
| ≥30 & <35 | 2460 | (24.0) | 2,109 | (23.8) | 351 | (25.4) | 1873 | (23.8) |
| 35+ | 1913 | (18.6) | 1,592 | (17.9) | 321 | (23.2) | 1375 | (17.5) |
| Missing | 12 | (0.1) | 4 | (0.0) | 8 | (0.6) | 3 | (0.0) |
| Tobacco Use | N | (%) | N | (%) | N | (%) | N | (%) |
| Never | 7082 | (69.0) | 6,185 | (69.7) | 897 | (64.8) | 5522 | (70.2) |
| Current | 1079 | (10.5) | 875 | (9.9) | 204 | (14.7) | 737 | (9.4) |
| Former | 2086 | (20.3) | 1,812 | (20.4) | 274 | (19.8) | 1601 | (20.4) |
| Missing | 11 | (0.1) | 2 | (0.0) | 9 | (0.7) | 1 | (0.0) |
| Alcohol Use | N | (%) | N | (%) | N | (%) | N | (%) |
| Never | 4673 | (45.6) | 3,982 | (44.9) | 691 | (49.9) | 3449 | (43.9) |
| Current | 4998 | (48.7) | 4,419 | (49.8) | 579 | (41.8) | 4004 | (50.9) |
| Former | 574 | (5.6) | 470 | (5.3) | 104 | (7.5) | 406 | (5.2) |
| Missing | 13 | (0.1) | 3 | (0.0) | 10 | (0.7) | 2 | (0.0) |
| Illicit Drug Use | N | (%) | N | (%) | N | (%) | N | (%) |
| Never | 9721 | (94.8) | 8,440 | (95.1) | 1,281 | (92.6) | 7481 | (95.2) |
| Current | 202 | (2.0) | 169 | (1.9) | 33 | (2.4) | 148 | (1.9) |
| Former | 321 | (3.1) | 261 | (2.9) | 60 | (4.3) | 229 | (2.9) |
| Missing | 14 | (0.1) | 4 | (0.0) | 10 | (0.7) | 3 | (0.0) |

TABLE 10
Clinical Demographics Data Collection

| Question | Answer Format |
|---|---|
| Date of Birth | ddMMMyyyy |
| Age | Numerical |
| Gender | Female |
| | Male |
| Ethnicity | Hispanic |
| | Not Hispanic/Latino |
| Race (select all that apply) | American Indian/Alaska Native |
| | Asian |
| | Black/African-American |
| | Native Hawaiian or Other Pacific |
| | Islander |
| | White |
| | Other (free text to specify) |

TABLE 11
CRC and APL Sensitivity based on Key Clinical Features

| | | | Advanced | |
|---|---|---|---|---|
| | | | Precancerous Lesion | |
| | Colorectal Cancer Sensitivity | | (APL) Sensitivity | |
| | (N = 65) | | (N = 1116) | |
| Tumor Location % (n/N) | | | | |
| (95% CI) | | | | |
| Proximal Colon | 88.9 | (8/9) | 14.5 | (92/634) |
| | (56.5-98.0) | | (12.0-17.5) | |
| Distal Colon | 84.4 | (27/32) | 10.5 | (40/380) |
| | (68.3-93.1) | | (7.8-14.0) | |
| Rectum | 79.2 | (19/24) | 14.1 | (14/99) |
| | (59.5-90.8) | | (8.6-22.3) | |
| Missing | — | | 33.3 | (1/3) |
| | | | (6.2-79.2) | |
| Most Significant Lesion Size % (n/N) | | | | |
| (95% CI) | | | | |
| ≤9 mm | 0 | (0/6) | 17.3 | (9/52) |
| | (0.0-39.0) | | (9.4-29.7) | |
| 10-19 mm | 85.7 | (6/7) | 11.9 | (102/859) |
| | (48.7-97.4) | | (9.9-14.2) | |
| ≥20 mm | 92.2 | (47/51) | 17.2 | (35/204) |
| | (81.5-96.9) | | (12.6-22.9) | |
| Missing | 100 | (1/1) | 100 | (1/1) |
| | (20.7-100) | | (20.7-100.0) | |
| CRC Tumor Grade % (n/N) | | | | |
| (95% CI) | | | | |
| Grade 1 | 80.0 | (4/5) | | |
| | (37.6-96.4) | | | |
| Grade 2 | 80.0 | (36/45) | | |
| | (66.2-89.1) | | | |
| Grade 3 | 100.0 | (6/6) | | |
| | (61.0-100.0) | | | |
| Missing | 88.9 | (8/9) | | |

| | (56.5-98.0) | | |
|---|---|---|---|
| APL Histopathology Diagnosis | | | |
| Subcategories % (n/N) | | | |
| (95% CI)* | | | |
| Advanced Adenoma, Carcinoma in | | 0.0 | (0/1) |
| situ, any size | | (0.0-79.4) | |
| Advanced Adenoma, with High- | | 22.6 | (7/31) |
| grade dysplasia(HGD), any size | | (11.4-39.8) | |
| Advanced Adenoma with villous | | 17.9 | (37/207) |
| component (>=25%), any size | | (13.3-23.7) | |
| Tubular Adenoma >=10 mm in size | | 12.0 | (82/685) |
| | | (9.8-14.6) | |
| Serrated lesion >=10 mm in size | | 11.0 | (21/191) |

(7.3-16.2)
*Histopathology on one APL missing

TABLE 12
Sensitivity and Specificity of the cfDNA blood-based test by key demographic features

| | | | | | | | Negative | |
|---|---|---|---|---|---|---|---|---|
| | | | | | | | Colonoscopy | |
| | CRC Sensitivity | | APL Sensitivity | | AN Specificity | | Specificity | |
| | (N = 65) | | (N = 1116) | | (N = 6680) | | (N = 4514) | |
| | % (n/N) | | % (n/N) | | % (n/N) | | % (n/N) | |
| | (95% CI) | | (95% CI) | | (95% CI) | | (95% CI) | |
| Gender | | | | | | | | |
| Female | 86.7 | (26/30) | 13.3 | (68/511) | 90.1 | (3314/3677) | 90.6 | (2413/2664) |
| | (70.3, 94.7) | | (10.6, 16.5) | | (89.1, 91.1) | | (89.4, 91.6) | |
| Male | 80.0 | (28/35) | 13.1 | (79/605) | 88.8 | (2668/3003) | 88.9 | (1644/1850) |
| | (64.1, 90.0) | | (10.6, 16.0) | | (87.7, 89.9) | | (87.4, 90.2) | |
| Age (in years) | | | | | | | | |
| 45-59 | 76.5 | (13/17) | 7.9 | (35/441) | 93.4 | (3024/3237) | 93.4 | (2161/2314) |
| | (52.7, 90.4) | | (5.8, 10.8) | | (92.5, 94.2) | | (92.3, 94.3) | |
| 60-69 | 88.2 | (30/34) | 15.1 | (63/417) | 89.7 | (1785/1989) | 89.6 | (1159/1293) |
| | (73.4, 95.3) | | (12.0, 18.9) | | (88.3, 91.0) | | (87.9, 91.2) | |
| 70+ | 78.6 | (11/14) | 19.0 | (49/258) | 80.7 | (1173/1454) | 81.3 | (737/907) |
| | (52.4, 92.4) | | (14.7, 24.2) | | (78.6, 82.6) | | (78.6, 83.7) | |
| Race | | | | | | | | |
| American Indian | (0/0) | | 0.0 | (0/2) | 83.3 | (10/12) | 83.3 | (5/6) |
| or Alaska Native | | | (0.0, 65.8) | | (55.2, 95.3) | | (43.7, 97.0) | |
| Asian | 75.0 | (3/4) | 17.9 | (10/56) | 84.4 | (422/500) | 86.1 | (327/380) |
| | (30.1, 95.4) | | (10.0, 29.8) | | (81.0, 87.3) | | (82.2, 89.2) | |
| Black or African | 90.0 | (9/10) | 13.2 | (16/121) | 92.1 | (737/800) | 92.6 | (538/581) |
| American | (59.6, 98.2) | | (8.3, 20.4) | | (90.1, 93.8) | | (90.2, 94.5) | |
| Native Hawaiian | (0/0) | | 0.0 | (0/2) | 94.1 | (16/17) | 100.0 | (13/13) |
| or Other Pacific | | | (0.0, 65.8) | | (73.0, 99.0) | | (77.2, 100.0) | |
| Islander | | | | | | | | |
| White | 81.6 | (40/49) | 13.0 | (119/917) | 89.8 | (4672/5201) | 90.0 | (3080/3422) |
| | (68.6, 90.0) | | (11.0, 15.3) | | (89.0, 90.6) | | (89.0, 91.0) | |
| Other | 100.0 | (1/1) | 6.3 | (1/16) | 84.2 | (101/120) | 84.9 | (79/93) |
| | (20.7, 100.0) | | (1.1, 28.3) | | (76.6, 89.6) | | (76.3, 90.8) | |
| Multiple | 100.0 | (1/1) | 50.0 | (1/2) | 80.0 | (16/20) | 71.4 | (10/14) |
| | (20.7, 100.0) | | (9.5, 90.6) | | (58.4, 91.9) | | (45.4, 88.9) | |
| Missing | (0/0) | | (0/0) | | 80.0 | (8/10) | 100.0 | (5/5) |
| | | | | | (49.0, 94.3) | | (56.6, 100.0) | |
| Ethnicity | | | | | | | | |
| Hispanic | 90.9 | (10/11) | 18.9 | (24/127) | 87.3 | (791/906) | 87.2 | (564/647) |
| | (62.3, 98.4) | | (13.0, 26.6) | | (84.98, 89.32) | | (84.4, 89.5) | |
| Not Hispanic or | 81.5 | (44/54) | 12.5 | (123/984) | 89.9 | (5162/5741) | 90.3 | (3474/3846) |
| Latino | (69.2, 89.6) | | (10.6, 14.7) | | (89.11, 90.67) | | (89.4, 91.2) | |
| Missing | (0/0) | | 0.0 | (0/5) | 87.9 | (29/33) | 90.5 | (19/21) |
| | | | (0.0, 43.5) | | (72.67, 95.18) | | (71.1, 97.4) | |
| Tobacco Use | | | | | | | | |
| Never | 82.9 | (34/41) | 13.4 | (97/711) | 89.5 | (4269/4770) | 89.8 | (2978/3316) |
| | (68.7, 91.5) | | (11.1, 16.1) | | (88.59, 90.34) | | (88.7, 90.8) | |
| Former | 93.3 | (14/15) | 13.8 | (34/247) | 90.2 | (1208/1339) | 90.7 | (764/842) |
| | (70.2, 98.8) | | (10.0, 18.6) | | (88.51, 91.69) | | (88.6, 92.5) | |
| Current | 66.7 | (6/9) | 11.4 | (18/158) | 88.4 | (504/570) | 88.5 | (314/355) |

| | (35.4, 87.9) | (7.3, 17.3) | (85.53, 90.79) | | (84.7, 91.4) | |
|---|---|---|---|---|---|---|
| Missing | (0/0) | (0/0) | 100 | (1/1) | 100.0 | (1/1) |

| | |
|---|---|
| (20.65, 100.0) | (20.7, 100.0) |

TABLE 13
Clinical details for the five “malignant polyps”

| | | | Size of the | | |
|---|---|---|---|---|---|
| | | | most | | |
| | | | significant | | |
| Case | Age | | lesion (in | Location of most | Pathology of the most |
| Number | (years) | Gender | mm) | significant lesion | significant lesion |
| 1 | 76 | Male | 15-18 | rectum | invasive adenocarcinoma, |
| | | | | | moderately differentiated, |
| | | | | | arising in tubular adenoma |
| | | | | | invasive well-differentiated |
| 2 | 50 | Male | 25 | rectum | adenocarcinoma arising in a |
| | | | | | tubulovillous adenoma with |
| | | | | | high grade dysplasia with focal |
| | | | | | features of a traditional |
| | | | | | serrated adenoma |
| 3 | 49 | Male | 22 | sigmoid colon | adenocarcinoma, moderately |
| | | | | | differentiated |
| 4 | 60 | Female | 50 mm × | rectum | adenocarcinoma arising in a |
| | | | 30 mm × | | tubulovillous adenoma |
| | | | 30 mm | | |
| 5 | 56 | Male | 20 | sigmoid colon | intramucosal and invasive |
| | | | | | adenocarcinoma arising in |
| | | | | | tubulovillous adenoma |

TABLE 14
Study Adverse Events:
There were no unanticipated adverse device events
observed across the 22,877 enrolled subjects
in the clinical study. Of the 43 reported adverse
events, 30 (70%) were minor related to
phlebotomy and 13 (30%) were unrelated to the study
intervention, including the two reported
serious adverse events.

| | | Related to | | Unique |
|---|---|---|---|---|
| | | Study | | Occurrences |
| Term | Serious? | Procedure? | Outcome | in the Study |
| NON-ST ELEVATED | Yes | Not Related | Recovered/ | 1 |
| MYOCARDIAL INFARCTION | | | Resolved | |
| CIRCULATORY COLLAPSE | Yes | Not Related | Recovered/ | 1 |
| | | | Resolved with | |
| | | | Sequelae | |
| BRUISE ON TOP OF RIGHT | No | Related | Recovered/ | 1 |
| HAND. | | | Resolved | |
| BRUISING AND SWELLING | No | Related | Recovered/ | 1 |
| AT BLOOD DRAW SITE | | | Resolved | |
| DIZZINESS | No | Possibly | Recovered/ | 1 |
| | | Related | Resolved | |
| DIZZINESS | No | Not Related | Recovered/ | 1 |
| | | | Resolved | |
| DURING THE BLOOD DRAW | No | Related | Recovered/ | 1 |
| PATIENT SAID HE WAS DONE | | | Resolved | |
| ANDLEANED BACK ON THE | | | | |
| CHAIR, HE EXPERIENCED | | | | |
| SYNCOPE. | | | | |
| EMESIS, DURING SYNCOPE | No | Related | Recovered/ | 1 |
| PATIENT LEANED FORWARD | | | Resolved | |
| AND VOMITED. | | | | |
| FAINTED DURING BLOOD | No | Related | Recovered/ | 3 |
| DRAW | | | Resolved | |
| HEMATOMA LEFT ARM | No | Related | Recovered/ | 1 |
| | | | Resolved | |
| LIGHT HEADED | No | Related | Recovered/ | 1 |
| | | | Resolved | |
| LIGHT HEADED, SWEATY, | No | Unlikely | Recovered/ | 1 |
| NAUSEATED, SAID WAS | | Related | Resolved | |
| GOING TO PASS OUT | | | | |
| LIGHTHEADED, CLAMMY | No | Related | Recovered/ | 1 |
| SKIN, AND SWEATY DURING | | | Resolved | |
| BLOOD SPECIMEN | | | | |
| COLLECTION THAT | | | | |
| RESOLVED WITHIN 10 | | | | |
| MINUTE | | | | |
| LIGHTHEADEDNESS DURING | No | Not Related | Recovered/ | 1 |
| BLOOD DRAW | | | Resolved | |
| NAUSEA | No | Not Related | Recovered/ | 1 |
| | | | Resolved | |
| NAUSEA AND DIZZINESS | No | Related | Recovered/ | 1 |
| | | | Resolved | |
| NEAR SYNCOPAL EPISODE | No | Possibly | Recovered/ | 1 |
| | | Related | Resolved | |
| NEAR SYNCOPY | No | Related | Recovered/ | 1 |
| | | | Resolved | |
| PAIN, BRUISING AND | No | Related | Not | 1 |
| SWOLLEN LEFT CUBITAL | | | Recovered/Not | |
| AREA & SIDE OF THE UPPER | | | Resolved | |
| ARM | | | | |
| PT FAINTED FOR LESS THAN | No | Related | Recovered/ | 1 |
| ONE MINUTE. | | | Resolved | |
| SUBJECT REPORTED FEELING | No | Not Related | Recovered/ | 1 |
| DIZZY | | | Resolved | |
| SUBJECT WAS FEELING | No | Related | Recovered/ | 1 |
| DISCOMFORT IN ARM AND | | | Resolved with | |
| ASKED TO HAVE LABS | | | Sequelae | |
| STOPPED AFTER 4 TUBES | | | | |
| SYNCOPAL EPISODE | No | Related | Recovered/ | 2 |
| | | | Resolved | |
| SYNCOPAL EPISODE | No | Not Related | Recovered/ | 1 |
| | | | Resolved | |
| SYNCOPE | No | Not Related | Recovered/ | 4 |
| | | | Resolved | |
| SYNCOPE | No | Related | Recovered/ | 2 |
| | | | Resolved | |
| SYNCOPE POST BLOOD | No | Possibly | Recovered/ | 1 |
| DRAW | | Related | Resolved | |
| VASOVAGAL EPISODE | No | Related | Recovered/ | 1 |
| | | | Resolved | |
| VASOVAGAL REACTION TO | No | Related | Recovered/ | 1 |
| VENIPUNCTURE | | | Resolved | |
| VASOVAGAL RESPONSE | No | Not Related | Recovered/ | 1 |
| | | | Resolved | |
| VASOVAGAL SYNCOPE | No | Related | Recovered/ | 4 |
| | | | Resolved | |
| VASOVAGAL SYNCOPE WITH | No | Related | Recovered/ | 1 |
| BLOOD DRAW. SUBJECT | | | Resolved | |
| STARTED TO FEEL | | | | |
| NAUSEOUS DURING BLOOD | | | | |
| DRAW. | | | | |
| VASOVAGAL SYNCOPE X 2 | No | Not Related | Recovered/ | 1 |
| | | | Resolved | |

TABLE 15
Supplementary Table on the Representativeness of Study Participants.

| | |
|---|---|
| Disease, problem, or | Average risk colorectal cancer screening |
| condition under | for all individuals age 45 to 84 years |
| investigation | |
| Special considerations | |
| related to: | |
| Sex and gender | CRC incidence in higher in men versus women with similar |
| | mortality rates |
| Age | CRC prevalence increases with age, with 94% of CRC diagnosed |
| | at age 45 years or older. |
| Race or ethnic group | CRC incidence and mortality rates are highest in individuals who |
| | are American Indian or Alaskan Native and individuals who are |
| | non-Hispanic Black |
| Geography | CRC incidence and mortality rates are lowest in the Western |
| | United Stages and highest in Appalachia and parts of the South |
| | and Midwest |
| Other considerations | Only 59% of individuals aged 45 years and older were up to date |
| | on CRC screening in 2021, ranging from 50% of Asian individuals |
| | to 61% of White and Black individuals; well below the target of |
| | 80%. |
| | The racial and ethnic diversity of the participants within the |
| | clinical study trial was reflective of the demographics of the |
| | United States Population, specifically for those identifying as |
| | Black/African-American, Asian, and who reported Hispanic |
| Overall representativeness | ethnicity. Clinical study sites covered 76% (38/50) of the United |
| of this study | States and included community-based clinics in addition to |
| | academic centers. In fact, more than 90% of study sites were |
| | outside of the academic hospital system, ensuring recruited |
| | participants were reflective of how the intended use population |
| | receives their care. |

Refer to FIG. 17, which includes a chart 1700 indicating colorectal cancer sensitivity according to stage of diagnosis. In an average-risk screening population, this cfDNA blood-based test showed performance metrics of 83% sensitivity for the detection of colorectal cancer, 90% specificity for advanced neoplasia, and 13% sensitivity for advanced pre-cancerous lesions.

Example 1 is a method comprising: determining, based on a quantification of an observed tumor-associated aberrant methylation of each of a plurality of cell-free nucleic acid samples, using a tumor fraction regression (TFR) model, a TFR score, wherein the TFR score includes, a fraction of molecules of the plurality of cell-free nucleic acid samples that indicate a tumor; determining, based on at least one of epigenetic factors or genomic alterations of each of the plurality of cell-free nucleic acid samples, a cell-free nucleic acid score indicative of presence of a tumor; and determining, based on at least one of the cell-free nucleic acid score or the TFR score satisfying a respective threshold, using a predictive model, that the plurality of cell-free nucleic acid samples is tumor-derived or non-tumor derived.

In Example 2, the subject matter of Example 1 includes, determining the quantification of the observed tumor-associated aberrant methylation of each of the plurality of cell-free nucleic acid samples.

In Example 3, the subject matter of Example 2 includes, wherein determining the quantification of the observed tumor-associated aberrant methylation of each of the plurality of cell-free nucleic acid samples includes quantifying a number of unique methylated molecules mapping to each of a plurality of genomic regions.

In Example 4, the subject matter of Examples 1-3 includes, determining the epigenetic factors of each of the plurality of cell-free nucleic acid samples.

In Example 5, the subject matter of Example 4 includes, determining the epigenetic factors of each of the plurality of cell-free nucleic acid samples based on a methylation logistic regression (LR) model cancer or non-cancer classification.

In Example 6, the subject matter of Example 5 includes, determining, using a LR model, the methylation LR model cancer or non-cancer classification.

In Example 7, the subject matter of Examples 4-6 includes, determining the epigenetic factors of each of the plurality of cell-free nucleic acid samples based on a cancer signal from cell-free nucleic acid fragmentation patterns associated with a plurality of sequence fragments from the plurality of cell-free nucleic acid samples.

In Example 8, the subject matter of Example 7 includes, determining, using a fragmentomics model, the cancer signal from the cell-free nucleic acid fragmentation patterns associated with the plurality of sequence fragments from the plurality of cell-free nucleic acid samples.

In Example 9, the subject matter of Examples 4-8 includes, determining the epigenetic factors of each of the plurality of cell-free nucleic acid samples based on a methylation logistic regression (LR) model cancer or non-cancer classification and based on a cancer signal from cell-free nucleic acid fragmentation patterns associated with a plurality of sequence fragments from the plurality of cell-free nucleic acid samples.

In Example 10, the subject matter of Example 9 includes, wherein determining the cell-free nucleic acid score is further based on the TFR score.

In Example 11, the subject matter of Examples 1-10 includes, determining the genomic alterations of each of the plurality of cell-free nucleic acid samples.

In Example 12, the subject matter of Example 11 includes, wherein determining the genomic alterations of each of the plurality of cell-free nucleic acid samples includes determining somatic variants observed in molecules from each of a plurality of sequence fragments from the plurality of cell-free nucleic acid samples.

In Example 13, the subject matter of Examples 1-12 includes, wherein the plurality of cell-free nucleic acid samples are from a plurality of genomic regions, wherein the plurality of genomic regions includes at least one of: a genomic region known to be associated with a cancer type, a genomic region associated with a known methylation status, a genomic region known to be associated with hypomethylation, or a genomic region known to be associated with therapy response.

In Example 14, the subject matter of Examples 1-13 includes, wherein the plurality of cell-free nucleic acid samples are from a plurality of genomic regions, wherein the plurality of genomic regions includes at least one genomic region known to be associated with colorectal cancer.

In Example 15, the subject matter of Examples 1-14 includes, wherein determining the cell-free nucleic acid score is further based on the TFR score.

In Example 16, the subject matter of Examples 1-15 includes, wherein the plurality of cell-free nucleic acid samples includes cell-free deoxyribonucleic (cfDNA) samples.

In Example 17, the subject matter of Examples 1-16 includes, wherein the plurality of cell-free nucleic acid samples includes ribonucleic acid (RNA) samples.

In Example 18, the subject matter of Examples 1-17 includes, wherein the plurality of cell-free nucleic acid samples includes cell-free ribonucleic acid (cfRNA) samples.

In Example 19, the subject matter of Examples 1-18 includes, wherein the plurality of cell-free nucleic acid samples includes mitochondrial deoxyribonucleic (mtDNA) samples.

In Example 20, the subject matter of Examples 1-19 includes, wherein the plurality of cell-free nucleic acid samples includes mitochondrial ribonucleic (mtRNA) samples.

In Example 21, the subject matter of Examples 1-20 includes, wherein the plurality of cell-free nucleic acid samples includes extracellular vesicle-bound deoxyribonucleic (evDNA) samples.

Example 22 is a method comprising: determining, based on a quantification of an observed tumor-associated aberrant methylation of each of the plurality of cell-free nucleic acid samples, using a tumor fraction regression (TFR) model, a TFR score, wherein the TFR score includes, a fraction of molecules of the plurality of cell-free nucleic acid samples that indicate a tumor; and determining, based on the TFR score satisfying a respective threshold, using a predictive model, that the plurality of cell-free nucleic acid samples is tumor-derived or non-tumor derived.

In Example 23, the subject matter of Example 22 includes, determining the quantification of the observed tumor-associated aberrant methylation of each of the plurality of cell-free nucleic acid samples.

In Example 24, the subject matter of Example 23 includes, wherein determining the quantification of the observed tumor-associated aberrant methylation of each of the plurality of cell-free nucleic acid samples includes quantifying a number of unique methylated molecules mapping to each of a plurality of genomic regions.

In Example 25, the subject matter of Examples 22-24 includes, wherein determining that the plurality of cell-free nucleic acid samples is tumor-derived or non-tumor derived is further based on a cell-free nucleic acid score indicative of presence of a tumor.

In Example 26, the subject matter of Example 25 includes, determining, based on at least one of epigenetic factors or genomic alterations of each of the plurality of cell-free nucleic acid samples, the cell-free nucleic acid score indicative of presence of a tumor.

In Example 27, the subject matter of Example 26 includes, determining the epigenetic factors of each of the plurality of cell-free nucleic acid samples.

In Example 28, the subject matter of Example 27 includes, determining the epigenetic factors of each of the plurality of cell-free nucleic acid samples based on a methylation logistic regression (LR) model cancer or non-cancer classification.

In Example 29, the subject matter of Example 28 includes, determining, using a LR model, the methylation LR model cancer or non-cancer classification.

In Example 30, the subject matter of Examples 27-29 includes, determining the epigenetic factors of each of the plurality of cell-free nucleic acid samples based on a cancer signal from cell-free nucleic acid fragmentation patterns associated with a plurality of sequence fragments from the plurality of cell-free nucleic acid samples.

In Example 31, the subject matter of Example 30 includes, determining, using a fragmentomics model, the cancer signal from the cell-free nucleic acid fragmentation patterns associated with the plurality of sequence fragments from the plurality of cell-free nucleic acid samples.

In Example 32, the subject matter of Examples 26-31 includes, determining the genomic alterations of each of the plurality of cell-free nucleic acid samples.

In Example 33, the subject matter of Example 32 includes, wherein determining the genomic alterations of each of the plurality of cell-free nucleic acid samples includes determining somatic variants observed in molecules from each of a plurality of sequence fragments from the plurality of cell-free nucleic acid samples.

In Example 34, the subject matter of Examples 26-33 includes, determining the epigenetic factors of each of the plurality of cell-free nucleic acid samples based on a methylation logistic regression (LR) model cancer or non-cancer classification and based on a cancer signal from cell-free nucleic acid fragmentation patterns associated with a plurality of sequence fragments from the plurality of cell-free nucleic acid samples.

In Example 35, the subject matter of Example 34 includes, wherein determining the cell-free nucleic acid score is further based on the TFR score.

In Example 36, the subject matter of Examples 22-35 includes, wherein the plurality of genomic regions includes at least one of: a genomic region known to be associated with a cancer type, a genomic region associated with a known methylation status, a genomic region known to be associated with hypomethylation, or a genomic region known to be associated with therapy response.

In Example 37, the subject matter of Examples 22-36 includes, wherein the plurality of genomic regions includes at least one genomic region known to be associated with colorectal cancer.

In Example 38, the subject matter of Examples 22-37 includes, wherein the plurality of cell-free nucleic acid samples includes cell-free deoxyribonucleic (cfDNA) samples.

In Example 39, the subject matter of Examples 22-38 includes, wherein the plurality of cell-free nucleic acid samples includes ribonucleic acid (RNA) samples.

In Example 40, the subject matter of Examples 22-39 includes, wherein the plurality of cell-free nucleic acid samples includes cell-free ribonucleic acid (cfRNA) samples.

In Example 41, the subject matter of Examples 22-40 includes, wherein the plurality of cell-free nucleic acid samples includes mitochondrial deoxyribonucleic (mtDNA) samples.

In Example 42, the subject matter of Examples 22-41 includes, wherein the plurality of cell-free nucleic acid samples includes mitochondrial ribonucleic (mtRNA) samples.

In Example 43, the subject matter of Examples 22-42 includes, wherein the plurality of cell-free nucleic acid samples includes extracellular vesicle-bound deoxyribonucleic (evDNA) samples.

Example 44 is a method comprising: determining, based on at least one of epigenetic factors or genomic alterations of each of the plurality of cell-free nucleic acid samples, a cell-free nucleic acid score indicative of presence of a tumor; and determining, based on the cell-free nucleic acid score satisfying a respective threshold, using a predictive model, that the plurality of cell-free nucleic acid samples is tumor-derived or non-tumor derived.

In Example 45, the subject matter of Example 44 includes, wherein determining that the plurality of cell-free nucleic acid samples is tumor-derived or non-tumor derived is further based on a tumor fraction regression (TFR) score satisfying a threshold, wherein the TFR score is indicative of a fraction of molecules of the plurality of cell-free nucleic acid samples that indicate a tumor.

In Example 46, the subject matter of Example 45 includes, determining, based on a quantification of an observed tumor-associated aberrant methylation of each of the plurality of cell-free nucleic acid samples, using a TFR model, the TFR score.

In Example 47, the subject matter of Example 46 includes, determining the quantification of the observed tumor-associated aberrant methylation of each of the plurality of cell-free nucleic acid samples.

In Example 48, the subject matter of Example 47 includes, wherein determining the quantification of the observed tumor-associated aberrant methylation of each of the plurality of cell-free nucleic acid samples includes quantifying a number of unique methylated molecules mapping to each of a plurality of genomic regions.

In Example 49, the subject matter of Examples 45-48 includes, wherein determining the cell-free nucleic acid score is further based on the TFR score.

In Example 50, the subject matter of Examples 44-49 includes, determining the epigenetic factors of each of the plurality of cell-free nucleic acid samples.

In Example 51, the subject matter of Example 50 includes, determining the epigenetic factors of each of the plurality of cell-free nucleic acid samples based on a methylation logistic regression (LR) model cancer or non-cancer classification.

In Example 52, the subject matter of Example 51 includes, determining, using a LR model, the methylation LR model cancer or non-cancer classification.

In Example 53, the subject matter of Examples 50-52 includes, determining the epigenetic factors of each of the plurality of cell-free nucleic acid samples based on a cancer signal from cell-free nucleic acid fragmentation patterns associated with a plurality of sequence fragments from the plurality of cell-free nucleic acid samples.

In Example 54, the subject matter of Example 53 includes, determining, using a fragmentomics model, the cancer signal from the cell-free nucleic acid fragmentation patterns associated with the plurality of sequence fragments from the plurality of cell-free nucleic acid samples.

In Example 55, the subject matter of Examples 50-54 includes, determining the epigenetic factors of each of the plurality of cell-free nucleic acid samples based on a methylation logistic regression (LR) model cancer or non-cancer classification and based on a cancer signal from cell-free nucleic acid fragmentation patterns associated with a plurality of sequence fragments from the plurality of cell-free nucleic acid samples.

In Example 56, the subject matter of Example 55 includes, wherein determining the cell-free nucleic acid score is further based on a tumor fraction regression (TFR) score, wherein the TFR score is indicative of a fraction of molecules of the plurality of cell-free nucleic acid samples that indicate a tumor.

In Example 57, the subject matter of Examples 44-56 includes, determining the genomic alterations of each of the plurality of cell-free nucleic acid samples.

In Example 58, the subject matter of Example 57 includes, wherein determining the genomic alterations of each of the plurality of cell-free nucleic acid samples includes determining somatic variants observed in molecules from each of a plurality of sequence fragments from the plurality of cell-free nucleic acid samples.

In Example 59, the subject matter of Examples 44-58 includes, wherein the plurality of cell-free nucleic acid samples are from a plurality of genomic regions, wherein the plurality of genomic regions includes at least one of: a genomic region known to be associated with a cancer type, a genomic region associated with a known methylation status, a genomic region known to be associated with hypomethylation, or a genomic region known to be associated with therapy response.

In Example 60, the subject matter of Examples 44-59 includes, wherein the plurality of cell-free nucleic acid samples are from a plurality of genomic regions, wherein the plurality of genomic regions includes at least one genomic region known to be associated with colorectal cancer.

In Example 61, the subject matter of Examples 44-60 includes, wherein determining that the plurality of cell-free nucleic acid samples is tumor-derived or non-tumor derived is further based on a methylation value satisfying a threshold, wherein the methylation score is indicative of a quantity of molecules of the plurality of cell-free nucleic acid samples that indicate a tumor.

In Example 62, the subject matter of Examples 44-61 includes, wherein the plurality of cell-free nucleic acid samples includes cell-free deoxyribonucleic (cfDNA) samples.

In Example 63, the subject matter of Examples 44-62 includes, wherein the plurality of cell-free nucleic acid samples includes ribonucleic acid (RNA) samples.

In Example 64, the subject matter of Examples 44-63 includes, wherein the plurality of cell-free nucleic acid samples includes cell-free ribonucleic acid (cfRNA) samples.

In Example 65, the subject matter of Examples 44-64 includes, wherein the plurality of cell-free nucleic acid samples includes mitochondrial deoxyribonucleic (mtDNA) samples.

In Example 66, the subject matter of Examples 44-65 includes, wherein the plurality of cell-free nucleic acid samples includes mitochondrial ribonucleic (mtRNA) samples.

In Example 67, the subject matter of Examples 44-66 includes, wherein the plurality of cell-free nucleic acid samples includes extracellular vesicle-bound deoxyribonucleic (evDNA) samples.

Example 68 is a method comprising: determining, based on a quantification of an observed tumor-associated aberrant methylation of each of a plurality of cell-free nucleic acid samples, using a tumor fraction regression (TFR) model, a TFR score, wherein the TFR score includes, a fraction of molecules of the plurality of cell-free nucleic acid samples that indicate a tumor, wherein each of the plurality of cell-free nucleic acid samples is labeled with a tumor-derived label or a non-tumor-derived label; determining, based on at least one of epigenetic factors or genomic alterations of each of the plurality of cell-free nucleic acid samples, a cell-free nucleic acid score indicative of presence of a tumor; determining, based on at least one of the cell-free nucleic acid score or the TFR score satisfying a respective threshold, a tumor prediction for each of the plurality of cell-free nucleic acid samples; generating, based on the tumor-derived label or the non-tumor-derived label and the tumor prediction for each of the plurality of cell-free nucleic acid samples, a predictive model to predict a tumor in the plurality of cell-free nucleic acid samples; and outputting the predictive model.

In Example 69, the subject matter of Example 68 includes, determining the quantification of the observed tumor-associated aberrant methylation of each of the plurality of cell-free nucleic acid samples.

In Example 70, the subject matter of Example 69 includes, wherein determining the quantification of the observed tumor-associated aberrant methylation of each of the plurality of cell-free nucleic acid samples includes quantifying a number of unique methylated molecules mapping to each of a plurality of genomic regions.

In Example 71, the subject matter of Examples 68-70 includes, determining the epigenetic factors of each of the plurality of cell-free nucleic acid samples.

In Example 72, the subject matter of Example 71 includes, determining the epigenetic factors of each of the plurality of cell-free nucleic acid samples based on a methylation logistic regression (LR) model cancer or non-cancer classification.

In Example 73, the subject matter of Example 72 includes, determining, using a LR model, the methylation LR model cancer or non-cancer classification.

In Example 74, the subject matter of Examples 71-73 includes, wherein determining the epigenetic factors of each of the plurality of cell-free nucleic acid samples is based on a cancer signal from cell-free nucleic acid fragmentation patterns associated with a plurality of sequence fragments from the plurality of cell-free nucleic acid samples.

In Example 75, the subject matter of Example 74 includes, determining, using a fragmentomics model, the cancer signal from the cell-free nucleic acid fragmentation patterns associated with the plurality of sequence fragments from the plurality of cell-free nucleic acid samples.

In Example 76, the subject matter of Examples 71-75 includes, determining the epigenetic factors of each of the plurality of cell-free nucleic acid samples based on a methylation logistic regression (LR) model cancer or non-cancer classification and based on a cancer signal from cell-free nucleic acid fragmentation patterns associated with a plurality of sequence fragments from the plurality of cell-free nucleic acid samples.

In Example 77, the subject matter of Example 76 includes, wherein determining the cell-free nucleic acid score is further based on the TFR score.

In Example 78, the subject matter of Examples 68-77 includes, determining the genomic alterations of each of the plurality of cell-free nucleic acid samples.

In Example 79, the subject matter of Example 78 includes, wherein determining the genomic alterations of each of the plurality of cell-free nucleic acid samples includes determining somatic variants observed in molecules from the plurality of cell-free nucleic acid samples.

In Example 80, the subject matter of Examples 68-79 includes, wherein the plurality of cell-free nucleic acid samples are from a plurality of genomic regions, wherein the plurality of genomic regions includes at least one of: a genomic region known to be associated with a cancer type, a genomic region associated with a known methylation status, a genomic region known to be associated with hypomethylation, or a genomic region known to be associated with therapy response.

In Example 81, the subject matter of Examples 68-80 includes, wherein the plurality of cell-free nucleic acid samples are from a plurality of genomic regions, wherein the plurality of genomic regions includes at least one genomic region known to be associated with colorectal cancer.

In Example 82, the subject matter of Examples 68-81 includes, wherein determining the cell-free nucleic acid score is further based on the TFR score.

In Example 83, the subject matter of Examples 68-82 includes, wherein the plurality of cell-free nucleic acid samples includes cell-free deoxyribonucleic (cfDNA) samples.

In Example 84, the subject matter of Examples 68-83 includes, wherein the plurality of cell-free nucleic acid samples includes ribonucleic acid (RNA) samples.

In Example 85, the subject matter of Examples 68-84 includes, wherein the plurality of cell-free nucleic acid samples includes cell-free ribonucleic acid (cfRNA) samples.

In Example 86, the subject matter of Examples 68-85 includes, wherein the plurality of cell-free nucleic acid samples includes mitochondrial deoxyribonucleic (mtDNA) samples.

In Example 87, the subject matter of Examples 68-86 includes, wherein the plurality of cell-free nucleic acid samples includes mitochondrial ribonucleic (mtRNA) samples.

In Example 88, the subject matter of Examples 68-87 includes, wherein the plurality of cell-free nucleic acid samples includes extracellular vesicle-bound deoxyribonucleic (evDNA) samples.

Example 89 is a method comprising: determining, based on a quantification of an observed tumor-associated aberrant methylation of each of a plurality of cell-free nucleic acid samples, using a tumor fraction regression (TFR) model, a TFR score, wherein the TFR score includes, a fraction of molecules of the plurality of cell-free nucleic acid samples that indicate a tumor, wherein each of the plurality of cell-free nucleic acid samples is labeled with a tumor-derived label or a non-tumor-derived label; determining, based on the TFR score satisfying a respective threshold, a tumor prediction for each of the plurality of cell-free nucleic acid samples; generating, based on the tumor-derived label or the non-tumor-derived label and the tumor prediction of the plurality of cell-free nucleic acid samples, a predictive model to predict a tumor in the plurality of cell-free nucleic acid samples; and outputting the predictive model.

In Example 90, the subject matter of Example 89 includes, determining the quantification of the observed tumor-associated aberrant methylation of each of the plurality of cell-free nucleic acid samples.

In Example 91, the subject matter of Example 90 includes, wherein determining the quantification of the observed tumor-associated aberrant methylation of each of the plurality of cell-free nucleic acid samples includes quantifying a number of unique methylated molecules mapping to each of a plurality of genomic regions.

In Example 92, the subject matter of Examples 89-91 includes, wherein determining the tumor prediction for each of the plurality of cell-free nucleic acid samples is further based on a cell-free nucleic acid score indicative of presence of a tumor.

In Example 93, the subject matter of Example 92 includes, determining, based on at least one of epigenetic factors or genomic alterations of each of the plurality of cell-free nucleic acid samples, the cell-free nucleic acid score indicative of presence of a tumor.

In Example 94, the subject matter of Example 93 includes, determining the epigenetic factors of each of the plurality of cell-free nucleic acid samples.

In Example 95, the subject matter of Example 94 includes, determining the epigenetic factors of each of the plurality of cell-free nucleic acid samples based on a methylation logistic regression (LR) model cancer or non-cancer classification.

In Example 96, the subject matter of Example 95 includes, determining, using a LR model, the methylation LR model cancer or non-cancer classification.

In Example 97, the subject matter of Examples 94-96 includes, determining the epigenetic factors of each of the plurality of cell-free nucleic acid samples based on a cancer signal from cell-free nucleic acid fragmentation patterns associated with a plurality of sequence fragments from the plurality of cell-free nucleic acid samples.

In Example 98, the subject matter of Example 97 includes, determining, using a fragmentomics model, the cancer signal from the cell-free nucleic acid fragmentation patterns associated with a plurality of sequence fragments from the plurality of cell-free nucleic acid samples.

In Example 99, the subject matter of Examples 93-98 includes, determining the genomic alterations of each of the plurality of cell-free nucleic acid samples.

In Example 100, the subject matter of Example 99 includes, wherein determining the genomic alterations of each of the plurality of cell-free nucleic acid samples includes determining somatic variants observed in molecules from each of a plurality of sequence fragments from the plurality of cell-free nucleic acid samples.

In Example 101, the subject matter of Examples 93-100 includes, determining the epigenetic factors of each of the plurality of cell-free nucleic acid samples based on a methylation logistic regression (LR) model cancer or non-cancer classification and based on a cancer signal from cell-free nucleic acid fragmentation patterns associated with a plurality of sequence fragments from the plurality of cell-free nucleic acid samples.

In Example 102, the subject matter of Example 101 includes, wherein determining the cell-free nucleic acid score is further based on the TFR score.

In Example 103, the subject matter of Examples 89-102 includes, wherein the plurality of cell-free nucleic acid samples are from a plurality of genomic regions, wherein the plurality of genomic regions includes at least one of: a genomic region known to be associated with a cancer type, a genomic region associated with a known methylation status, a genomic region known to be associated with hypomethylation, or a genomic region known to be associated with therapy response.

In Example 104, the subject matter of Examples 89-103 includes, wherein the plurality of cell-free nucleic acid samples are from a plurality of genomic regions, wherein the plurality of genomic regions includes at least one genomic region known to be associated with colorectal cancer.

In Example 105, the subject matter of Examples 89-104 includes, wherein the plurality of cell-free nucleic acid samples includes cell-free deoxyribonucleic (cfDNA) samples.

In Example 106, the subject matter of Examples 88-105 includes, wherein the plurality of cell-free nucleic acid samples includes ribonucleic acid (RNA) samples.

In Example 107, the subject matter of Examples 89-106 includes, wherein the plurality of cell-free nucleic acid samples includes cell-free ribonucleic acid (cfRNA) samples.

In Example 108, the subject matter of Examples 89-107 includes, wherein the plurality of cell-free nucleic acid samples includes mitochondrial deoxyribonucleic (mtDNA) samples.

In Example 109, the subject matter of Examples 89-108 includes, wherein the plurality of cell-free nucleic acid samples includes mitochondrial ribonucleic (mtRNA) samples.

In Example 110, the subject matter of Examples 89-109 includes, wherein the plurality of cell-free nucleic acid samples includes extracellular vesicle-bound deoxyribonucleic (evDNA) samples.

Example 111 is a method comprising: determining, based on at least one of epigenetic factors or genomic alterations of each of a plurality of cell-free nucleic acid samples, a cell-free nucleic acid score indicative of presence of a tumor, wherein each of the plurality of cell-free nucleic acid samples is labeled with a tumor-derived label or a non-tumor-derived label; determining, based on the cell-free nucleic acid score satisfying a respective threshold, a tumor prediction for each of the plurality of cell-free nucleic acid samples; generating, based on the tumor-derived label or the non-tumor-derived label and the tumor prediction for each of the plurality of cell-free nucleic acid samples, a predictive model to predict a tumor in the plurality of cell-free nucleic acid samples; and outputting the predictive model.

In Example 112, the subject matter of Example 111 includes, wherein determining the tumor prediction for each of the plurality of cell-free nucleic acid is further based on a tumor fraction regression (TFR) score satisfying a threshold, wherein the TFR score is indicative of a fraction of molecules of the plurality of cell-free nucleic acid samples that indicate a tumor.

In Example 113, the subject matter of Example 112 includes, determining, based on a quantification of an observed tumor-associated aberrant methylation of each of the plurality of cell-free nucleic acid samples, using a TFR model, the TFR score.

In Example 114, the subject matter of Example 113 includes, determining the quantification of the observed tumor-associated aberrant methylation of each of the plurality of cell-free nucleic acid samples.

In Example 115, the subject matter of Example 114 includes, wherein determining the quantification of the observed tumor-associated aberrant methylation of each of the plurality of cell-free nucleic acid samples includes quantifying a number of unique methylated molecules mapping to each of a plurality of genomic regions.

In Example 116, the subject matter of Examples 112-115 includes, wherein determining the cell-free nucleic acid score is further based on the TFR score.

In Example 117, the subject matter of Examples 111-116 includes, determining the epigenetic factors of each of the plurality of cell-free nucleic acid samples.

In Example 118, the subject matter of Example 117 includes, determining the epigenetic factors of each of the plurality of cell-free nucleic acid samples based on a methylation logistic regression (LR) model cancer or non-cancer classification.

In Example 119, the subject matter of Example 118 includes, determining, using a LR model, the methylation LR model cancer or non-cancer classification.

In Example 120, the subject matter of Examples 117-119 includes, determining the epigenetic factors of each of the plurality of cell-free nucleic acid samples based on a cancer signal from cell-free nucleic acid fragmentation patterns associated with a plurality of sequence fragments from the plurality of cell-free nucleic acid samples.

In Example 121, the subject matter of Example 120 includes, determining, using a fragmentomics model, the cancer signal from the cell-free nucleic acid fragmentation patterns associated with the plurality of sequence fragments from the plurality of cell-free nucleic acid samples.

In Example 122, the subject matter of Examples 117-121 includes, determining the epigenetic factors of each of the plurality of cell-free nucleic acid samples based on a methylation logistic regression (LR) model cancer or non-cancer classification and based on a cancer signal from cell-free nucleic acid fragmentation patterns associated with a plurality of sequence fragments from the plurality of cell-free nucleic acid samples.

In Example 123, the subject matter of Example 122 includes, wherein determining the cell-free nucleic acid score is further based on a tumor fraction regression (TFR) score, wherein the TFR score is indicative of a fraction of molecules of the plurality of cell-free nucleic acid samples that indicate a tumor.

In Example 124, the subject matter of Examples 111-123 includes, determining the genomic alterations of each of the plurality of cell-free nucleic acid samples.

In Example 125, the subject matter of Example 124 includes, wherein determining the genomic alterations of each of the plurality of cell-free nucleic acid samples includes determining somatic variants observed in molecules from each of a plurality of sequence fragments from the plurality of cell-free nucleic acid samples.

In Example 126, the subject matter of Examples 111-125 includes, wherein the plurality of cell-free nucleic acid samples are from a plurality of genomic regions, wherein the plurality of genomic regions includes at least one of: a genomic region known to be associated with a cancer type, a genomic region associated with a known methylation status, a genomic region known to be associated with hypomethylation, or a genomic region known to be associated with therapy response.

In Example 127, the subject matter of Examples 111-126 includes, wherein the plurality of cell-free nucleic acid samples are from a plurality of genomic regions, wherein the plurality of genomic regions includes at least one genomic region known to be associated with colorectal cancer.

In Example 128, the subject matter of Examples 111-127 includes, wherein determining that the plurality of cell-free nucleic acid samples is tumor-derived or non-tumor derived is further based on a methylation value satisfying a threshold, wherein the methylation score is indicative of a quantity of molecules of the plurality of cell-free nucleic acid samples that indicate a tumor.

In Example 129, the subject matter of Examples 111-128 includes, wherein the plurality of cell-free nucleic acid samples includes cell-free deoxyribonucleic (cfDNA) samples.

In Example 130, the subject matter of Examples 111-129 includes, wherein the plurality of cell-free nucleic acid samples includes ribonucleic acid (RNA) samples.

In Example 131, the subject matter of Examples 111-130 includes, wherein the plurality of cell-free nucleic acid samples includes cell-free ribonucleic acid (cfRNA) samples.

In Example 132, the subject matter of Examples 111-131 includes, wherein the plurality of cell-free nucleic acid samples includes mitochondrial deoxyribonucleic (mtDNA) samples.

In Example 133, the subject matter of Examples 111-132 includes, wherein the plurality of cell-free nucleic acid samples includes mitochondrial ribonucleic (mtRNA) samples.

In Example 134, the subject matter of Examples 111-133 includes, wherein the plurality of cell-free nucleic acid samples includes extracellular vesicle-bound deoxyribonucleic (evDNA) samples.

Example 135 is a method comprising: detecting one or more biomarkers in a biological sample; determining, based on a quantification of an observed tumor-associated aberrant methylation of each of a plurality of cell-free nucleic acid samples, using a tumor fraction regression (TFR) model, a TFR score, wherein the TFR score includes, a fraction of molecules of the plurality of cell-free nucleic acid samples that indicate a tumor; determining, based on at least one of epigenetic factors or genomic alterations of each of the plurality of cell-free nucleic acid samples, a cell-free nucleic acid score indicative of presence of a tumor; and determining, based on at least one of the detected biomarkers, the cell-free nucleic acid score, or the TFR score satisfying a respective threshold, that the biological sample is tumor-derived or non-tumor derived.

In Example 136, the subject matter of Example 135 includes, determining the quantification of the observed tumor-associated aberrant methylation of each of the plurality of cell-free nucleic acid samples.

In Example 137, the subject matter of Example 136 includes, wherein determining the quantification of the observed tumor-associated aberrant methylation of each of the plurality of cell-free nucleic acid samples includes quantifying a number of unique methylated molecules mapping to each of a plurality of genomic regions.

In Example 138, the subject matter of Examples 135-137 includes, determining the epigenetic factors of each of the plurality of cell-free nucleic acid samples.

In Example 139, the subject matter of Example 138 includes, determining the epigenetic factors of each of the plurality of cell-free nucleic acid samples based on a methylation logistic regression (LR) model cancer or non-cancer classification.

In Example 140, the subject matter of Example 139 includes, determining, using a LR model, the methylation LR model cancer or non-cancer classification.

In Example 141, the subject matter of Examples 138-140 includes, determining the epigenetic factors of each of the plurality of cell-free nucleic acid samples based on a cancer signal from cell-free nucleic acid fragmentation patterns associated with a plurality of sequence fragments from the plurality of cell-free nucleic acid samples.

In Example 142, the subject matter of Example 141 includes, determining, using a fragmentomics model, the cancer signal from the cell-free nucleic acid fragmentation patterns associated with the plurality of sequence fragments from the plurality of cell-free nucleic acid samples.

In Example 143, the subject matter of Examples 138-142 includes, determining the epigenetic factors of each of the plurality of cell-free nucleic acid samples based on a methylation logistic regression (LR) model cancer or non-cancer classification and based on a cancer signal from cell-free nucleic acid fragmentation patterns associated with a plurality of sequence fragments from the plurality of cell-free nucleic acid samples.

In Example 144, the subject matter of Example 143 includes, wherein determining the cell-free nucleic acid score is further based on the TFR score.

In Example 145, the subject matter of Examples 135-144 includes, determining the genomic alterations of each of the plurality of cell-free nucleic acid samples.

In Example 146, the subject matter of Example 145 includes, wherein determining the genomic alterations of each of the plurality of cell-free nucleic acid samples includes determining somatic variants observed in molecules from each of a plurality of sequence fragments from the plurality of cell-free nucleic acid samples.

In Example 147, the subject matter of Examples 135-146 includes, wherein the plurality of cell-free nucleic acid samples are from a plurality of genomic regions, wherein the plurality of genomic regions includes at least one of: a genomic region known to be associated with a cancer type, a genomic region associated with a known methylation status, a genomic region known to be associated with hypomethylation, or a genomic region known to be associated with therapy response.

In Example 148, the subject matter of Examples 135-147 includes, wherein the plurality of cell-free nucleic acid samples are from a plurality of genomic regions, wherein the plurality of genomic regions includes at least one genomic region known to be associated with colorectal cancer.

In Example 149, the subject matter of Examples 135-148 includes, wherein determining the cell-free nucleic acid score is further based on the TFR score.

In Example 150, the subject matter of Examples 135-149 includes, wherein the plurality of cell-free nucleic acid samples includes cell-free deoxyribonucleic (cfDNA) samples.

In Example 151, the subject matter of Examples 135-150 includes, wherein the plurality of cell-free nucleic acid samples includes ribonucleic acid (RNA) samples.

In Example 152, the subject matter of Examples 135-151 includes, wherein the plurality of cell-free nucleic acid samples includes cell-free ribonucleic acid (cfRNA) samples.

In Example 153, the subject matter of Examples 135-152 includes, wherein the plurality of cell-free nucleic acid samples includes mitochondrial deoxyribonucleic (mtDNA) samples.

In Example 154, the subject matter of Examples 135-153 includes, wherein the plurality of cell-free nucleic acid samples includes mitochondrial ribonucleic (mtRNA) samples.

In Example 155, the subject matter of Examples 135-154 includes, wherein the plurality of cell-free nucleic acid samples includes extracellular vesicle-bound deoxyribonucleic (evDNA) samples.

In Example 156, the subject matter of Examples 135-155 includes, wherein the biomarker is one or more of those selected from: proteins, exosomes, exomeres, microvesicles, apoptotic bodies, neutrophil extracellular traps (NETs), immune cells, tumor-educated platelets (TEPs), microbiome, virome, toll-like receptors (TLRs), and mitochondrial DNA (mtDNA).

In Example 157, the subject matter of Examples 135-156 includes, wherein detecting one ore more biomarkers comprises detecting the presence or levels of the one or more biomarkers.

In Example 158, the subject matter of Examples 135-157 includes, wherein determining that the biological sample is tumor-derived or non-tumor derived comprises comparing the levels of the one or more biomarkers in the biological sample to a control.

In Example 159, the subject matter of Example 158 includes, wherein the control is a reference level or a level present in a healthy, non-cancer subject.

Example 160 is a computing system comprising: one or more hardware processors; and one or more non-transitory computer-readable storage media including computer-readable instructions that, when executed by the one or more hardware processors, cause the one or more hardware processors to perform operations comprising: determining, based on a quantification of an observed tumor-associated aberrant methylation of each of a plurality of cell-free nucleic acid samples, using a tumor fraction regression (TFR) model, a TFR score, wherein the TFR score includes, a fraction of molecules of the plurality of cell-free nucleic acid samples that indicate a tumor; determining, based on at least one of epigenetic factors or genomic alterations of each of the plurality of cell-free nucleic acid samples, a cell-free nucleic acid score indicative of presence of a tumor; and determining, based on at least one of the cell-free nucleic acid score or the TFR score satisfying a respective threshold, using a predictive model, that the plurality of cell-free nucleic acid samples is tumor-derived or non-tumor derived.

In Example 161, the subject matter of Example 160 includes, wherein the one or more non-transitory computer-readable storage media store additional computer-readable instructions that, when executed by the one or more hardware processors, cause the one or more hardware processors to perform additional operations comprising determining the quantification of the observed tumor-associated aberrant methylation of each of the plurality of cell-free nucleic acid samples.

In Example 162, the subject matter of Example 161 includes, wherein the computer-readable instructions that cause the one or more hardware processors to perform operations comprising determining the quantification of the observed tumor-associated aberrant methylation of each of the plurality of cell-free nucleic acid samples includes computer-readable instructions that cause the one or more hardware processors to perform operations comprising quantifying a number of unique methylated molecules mapping to each of a plurality of genomic regions.

In Example 163, the subject matter of Examples 160-162 includes, wherein the one or more non-transitory computer-readable storage media store additional computer-readable instructions that, when executed by the one or more hardware processors, cause the one or more hardware processors to perform additional operations comprising determining the epigenetic factors of each of the plurality of cell-free nucleic acid samples.

In Example 164, the subject matter of Example 163 includes, wherein the one or more non-transitory computer-readable storage media store additional computer-readable instructions that, when executed by the one or more hardware processors, cause the one or more hardware processors to perform additional operations comprising determining the epigenetic factors of each of the plurality of cell-free nucleic acid samples based on a methylation logistic regression (LR) model cancer or non-cancer classification.

In Example 165, the subject matter of Example 164 includes, wherein the one or more non-transitory computer-readable storage media store additional computer-readable instructions that, when executed by the one or more hardware processors, cause the one or more hardware processors to perform additional operations comprising determining, using a LR model, the methylation LR model cancer or non-cancer classification.

In Example 166, the subject matter of Examples 163-165 includes, wherein the one or more non-transitory computer-readable storage media store additional computer-readable instructions that, when executed by the one or more hardware processors, cause the one or more hardware processors to perform additional operations comprising determining the epigenetic factors of each of the plurality of cell-free nucleic acid samples based on a cancer signal from cell-free nucleic acid fragmentation patterns associated with a plurality of sequence fragments from the plurality of cell-free nucleic acid samples.

In Example 167, the subject matter of Example 166 includes, wherein the one or more non-transitory computer-readable storage media store additional computer-readable instructions that, when executed by the one or more hardware processors, cause the one or more hardware processors to perform additional operations comprising determining, using a fragmentomics model, the cancer signal from the cell-free nucleic acid fragmentation patterns associated with the plurality of sequence fragments from the plurality of cell-free nucleic acid samples.

In Example 168, the subject matter of Examples 163-167 includes, wherein the one or more non-transitory computer-readable storage media store additional computer-readable instructions that, when executed by the one or more hardware processors, cause the one or more hardware processors to perform additional operations comprising determining the epigenetic factors of each of the plurality of cell-free nucleic acid samples based on a methylation logistic regression (LR) model cancer or non-cancer classification and based on a cancer signal from cell-free nucleic acid fragmentation patterns associated with a plurality of sequence fragments from the plurality of cell-free nucleic acid samples.

In Example 169, the subject matter of Example 168 includes, wherein determining the cell-free nucleic acid score is further based on the TFR score.

In Example 170, the subject matter of Examples 160-169 includes, wherein the one or more non-transitory computer-readable storage media store additional computer-readable instructions that, when executed by the one or more hardware processors, cause the one or more hardware processors to perform additional operations comprising determining the genomic alterations of each of the plurality of cell-free nucleic acid samples.

In Example 171, the subject matter of Example 170 includes, wherein the computer-readable instructions that cause the one or more hardware processors to perform operations comprising determining the genomic alterations of each of the plurality of cell-free nucleic acid samples includes computer-readable instructions that cause the one or more hardware processors to perform operations comprising determining somatic variants observed in molecules from each of a plurality of sequence fragments from the plurality of cell-free nucleic acid samples.

In Example 172, the subject matter of Examples 160-171 includes, wherein the plurality of cell-free nucleic acid samples are from a plurality of genomic regions, wherein the plurality of genomic regions includes at least one of: a genomic region known to be associated with a cancer type, a genomic region associated with a known methylation status, a genomic region known to be associated with hypomethylation, or a genomic region known to be associated with therapy response.

In Example 173, the subject matter of Examples 160-172 includes, wherein the plurality of cell-free nucleic acid samples are from a plurality of genomic regions, wherein the plurality of genomic regions includes at least one genomic region known to be associated with colorectal cancer.

In Example 174, the subject matter of Examples 160-173 includes, wherein determining the cell-free nucleic acid score is further based on the TFR score.

In Example 175, the subject matter of Examples 160-174 includes, wherein the plurality of cell-free nucleic acid samples includes cell-free deoxyribonucleic (cfDNA) samples.

In Example 176, the subject matter of Examples 160-175 includes, wherein the plurality of cell-free nucleic acid samples includes ribonucleic acid (RNA) samples.

In Example 177, the subject matter of Examples 160-176 includes, wherein the plurality of cell-free nucleic acid samples includes cell-free ribonucleic acid (cfRNA) samples.

In Example 178, the subject matter of Examples 160-177 includes, wherein the plurality of cell-free nucleic acid samples includes mitochondrial deoxyribonucleic (mtDNA) samples.

In Example 179, the subject matter of Examples 160-178 includes, wherein the plurality of cell-free nucleic acid samples includes mitochondrial ribonucleic (mtRNA) samples.

In Example 180, the subject matter of Examples 160-179 includes, wherein the plurality of cell-free nucleic acid samples includes extracellular vesicle-bound deoxyribonucleic (evDNA) samples.

Example 181 is a computing system comprising: one or more hardware processors; and one or more non-transitory computer-readable storage media including computer-readable instructions that, when executed by the one or more hardware processors, cause the one or more hardware processors to perform operations comprising: determining, based on a quantification of an observed tumor-associated aberrant methylation of each of the plurality of cell-free nucleic acid samples, using a tumor fraction regression (TFR) model, a TFR score, wherein the TFR score includes, a fraction of molecules of the plurality of cell-free nucleic acid samples that indicate a tumor; and determining, based on the TFR score satisfying a respective threshold, using a predictive model, that the plurality of cell-free nucleic acid samples is tumor-derived or non-tumor derived.

In Example 182, the subject matter of Example 181 includes, wherein the one or more non-transitory computer-readable storage media store additional computer-readable instructions that, when executed by the one or more hardware processors, cause the one or more hardware processors to perform additional operations comprising determining the quantification of the observed tumor-associated aberrant methylation of each of the plurality of cell-free nucleic acid samples.

In Example 183, the subject matter of Example 182 includes, wherein the computer-readable instructions that cause the one or more hardware processors to perform operations comprising determining the quantification of the observed tumor-associated aberrant methylation of each of the plurality of cell-free nucleic acid samples includes computer-readable instructions that cause the one or more hardware processors to perform operations comprising quantifying a number of unique methylated molecules mapping to each of a plurality of genomic regions.

In Example 184, the subject matter of Examples 181-183 includes, wherein determining that the plurality of cell-free nucleic acid samples is tumor-derived or non-tumor derived is further based on a cell-free nucleic acid score indicative of presence of a tumor.

In Example 185, the subject matter of Example 184 includes, wherein the one or more non-transitory computer-readable storage media store additional computer-readable instructions that, when executed by the one or more hardware processors, cause the one or more hardware processors to perform additional operations comprising determining, based on at least one of epigenetic factors or genomic alterations of each of the plurality of cell-free nucleic acid samples, the cell-free nucleic acid score indicative of presence of a tumor.

In Example 186, the subject matter of Example 185 includes, wherein the one or more non-transitory computer-readable storage media store additional computer-readable instructions that, when executed by the one or more hardware processors, cause the one or more hardware processors to perform additional operations comprising determining the epigenetic factors of each of the plurality of cell-free nucleic acid samples.

In Example 187, the subject matter of Example 186 includes, wherein the one or more non-transitory computer-readable storage media store additional computer-readable instructions that, when executed by the one or more hardware processors, cause the one or more hardware processors to perform additional operations comprising determining the epigenetic factors of each of the plurality of cell-free nucleic acid samples based on a methylation logistic regression (LR) model cancer or non-cancer classification.

In Example 188, the subject matter of Example 187 includes, wherein the one or more non-transitory computer-readable storage media store additional computer-readable instructions that, when executed by the one or more hardware processors, cause the one or more hardware processors to perform additional operations comprising determining, using a LR model, the methylation LR model cancer or non-cancer classification.

In Example 189, the subject matter of Examples 186-188 includes, wherein the one or more non-transitory computer-readable storage media store additional computer-readable instructions that, when executed by the one or more hardware processors, cause the one or more hardware processors to perform additional operations comprising determining the epigenetic factors of each of the plurality of cell-free nucleic acid samples based on a cancer signal from cell-free nucleic acid fragmentation patterns associated with a plurality of sequence fragments from the plurality of cell-free nucleic acid samples.

In Example 190, the subject matter of Example 189 includes, wherein the one or more non-transitory computer-readable storage media store additional computer-readable instructions that, when executed by the one or more hardware processors, cause the one or more hardware processors to perform additional operations comprising determining, using a fragmentomics model, the cancer signal from the cell-free nucleic acid fragmentation patterns associated with the plurality of sequence fragments from the plurality of cell-free nucleic acid samples.

In Example 191, the subject matter of Examples 185-190 includes, wherein the one or more non-transitory computer-readable storage media store additional computer-readable instructions that, when executed by the one or more hardware processors, cause the one or more hardware processors to perform additional operations comprising determining the genomic alterations of each of the plurality of cell-free nucleic acid samples.

In Example 192, the subject matter of Example 191 includes, wherein the computer-readable instructions that cause the one or more hardware processors to perform operations comprising determining the genomic alterations of each of the plurality of cell-free nucleic acid samples includes computer-readable instructions that cause the one or more hardware processors to perform operations comprising determining somatic variants observed in molecules from each of a plurality of sequence fragments from the plurality of cell-free nucleic acid samples.

In Example 193, the subject matter of Examples 185-192 includes, wherein the one or more non-transitory computer-readable storage media store additional computer-readable instructions that, when executed by the one or more hardware processors, cause the one or more hardware processors to perform additional operations comprising determining the epigenetic factors of each of the plurality of cell-free nucleic acid samples based on a methylation logistic regression (LR) model cancer or non-cancer classification and based on a cancer signal from cell-free nucleic acid fragmentation patterns associated with a plurality of sequence fragments from the plurality of cell-free nucleic acid samples.

In Example 194, the subject matter of Example 193 includes, wherein determining the cell-free nucleic acid score is further based on the TFR score.

In Example 195, the subject matter of Examples 181-194 includes, wherein the plurality of genomic regions includes at least one of: a genomic region known to be associated with a cancer type, a genomic region associated with a known methylation status, a genomic region known to be associated with hypomethylation, or a genomic region known to be associated with therapy response.

In Example 196, the subject matter of Examples 181-195 includes, wherein the plurality of genomic regions includes at least one genomic region known to be associated with colorectal cancer.

In Example 197, the subject matter of Examples 181-196 includes, wherein the plurality of cell-free nucleic acid samples includes cell-free deoxyribonucleic (cfDNA) samples.

In Example 198, the subject matter of Examples 181-197 includes, wherein the plurality of cell-free nucleic acid samples includes ribonucleic acid (RNA) samples.

In Example 199, the subject matter of Examples 181-198 includes, wherein the plurality of cell-free nucleic acid samples includes cell-free ribonucleic acid (cfRNA) samples.

In Example 200, the subject matter of Examples 181-199 includes, wherein the plurality of cell-free nucleic acid samples includes mitochondrial deoxyribonucleic (mtDNA) samples.

In Example 201, the subject matter of Examples 181-200 includes, wherein the plurality of cell-free nucleic acid samples includes mitochondrial ribonucleic (mtRNA) samples.

In Example 202, the subject matter of Examples 181-201 includes, wherein the plurality of cell-free nucleic acid samples includes extracellular vesicle-bound deoxyribonucleic (evDNA) samples.

Example 203 is a computing system comprising: one or more hardware processors; and one or more non-transitory computer-readable storage media including computer-readable instructions that, when executed by the one or more hardware processors, cause the one or more hardware processors to perform operations comprising: determining, based on at least one of epigenetic factors or genomic alterations of each of the plurality of cell-free nucleic acid samples, a cell-free nucleic acid score indicative of presence of a tumor; and determining, based on the cell-free nucleic acid score satisfying a respective threshold, using a predictive model, that the plurality of cell-free nucleic acid samples is tumor-derived or non-tumor derived.

In Example 204, the subject matter of Example 203 includes, wherein determining that the plurality of cell-free nucleic acid samples is tumor-derived or non-tumor derived is further based on a tumor fraction regression (TFR) score satisfying a threshold, wherein the TFR score is indicative of a fraction of molecules of the plurality of cell-free nucleic acid samples that indicate a tumor.

In Example 205, the subject matter of Example 204 includes, wherein the one or more non-transitory computer-readable storage media store additional computer-readable instructions that, when executed by the one or more hardware processors, cause the one or more hardware processors to perform additional operations comprising determining, based on a quantification of an observed tumor-associated aberrant methylation of each of the plurality of cell-free nucleic acid samples, using a TFR model, the TFR score.

In Example 206, the subject matter of Example 205 includes, wherein the one or more non-transitory computer-readable storage media store additional computer-readable instructions that, when executed by the one or more hardware processors, cause the one or more hardware processors to perform additional operations comprising determining the quantification of the observed tumor-associated aberrant methylation of each of the plurality of cell-free nucleic acid samples.

In Example 207, the subject matter of Example 206 includes, wherein the computer-readable instructions that cause the one or more hardware processors to perform operations comprising determining the quantification of the observed tumor-associated aberrant methylation of each of the plurality of cell-free nucleic acid samples includes computer-readable instructions that cause the one or more hardware processors to perform operations comprising quantifying a number of unique methylated molecules mapping to each of a plurality of genomic regions.

In Example 208, the subject matter of Examples 204-207 includes, wherein determining the cell-free nucleic acid score is further based on the TFR score.

In Example 209, the subject matter of Examples 203-208 includes, wherein the one or more non-transitory computer-readable storage media store additional computer-readable instructions that, when executed by the one or more hardware processors, cause the one or more hardware processors to perform additional operations comprising determining the epigenetic factors of each of the plurality of cell-free nucleic acid samples.

In Example 210, the subject matter of Example 209 includes, wherein the one or more non-transitory computer-readable storage media store additional computer-readable instructions that, when executed by the one or more hardware processors, cause the one or more hardware processors to perform additional operations comprising determining the epigenetic factors of each of the plurality of cell-free nucleic acid samples based on a methylation logistic regression (LR) model cancer or non-cancer classification.

In Example 211, the subject matter of Example 210 includes, wherein the one or more non-transitory computer-readable storage media store additional computer-readable instructions that, when executed by the one or more hardware processors, cause the one or more hardware processors to perform additional operations comprising determining, using a LR model, the methylation LR model cancer or non-cancer classification.

In Example 212, the subject matter of Examples 209-211 includes, wherein the one or more non-transitory computer-readable storage media store additional computer-readable instructions that, when executed by the one or more hardware processors, cause the one or more hardware processors to perform additional operations comprising determining the epigenetic factors of each of the plurality of cell-free nucleic acid samples based on a cancer signal from cell-free nucleic acid fragmentation patterns associated with a plurality of sequence fragments from the plurality of cell-free nucleic acid samples.

In Example 213, the subject matter of Example 212 includes, wherein the one or more non-transitory computer-readable storage media store additional computer-readable instructions that, when executed by the one or more hardware processors, cause the one or more hardware processors to perform additional operations comprising determining, using a fragmentomics model, the cancer signal from the cell-free nucleic acid fragmentation patterns associated with the plurality of sequence fragments from the plurality of cell-free nucleic acid samples.

In Example 214, the subject matter of Examples 209-213 includes, wherein the one or more non-transitory computer-readable storage media store additional computer-readable instructions that, when executed by the one or more hardware processors, cause the one or more hardware processors to perform additional operations comprising determining the epigenetic factors of each of the plurality of cell-free nucleic acid samples based on a methylation logistic regression (LR) model cancer or non-cancer classification and based on a cancer signal from cell-free nucleic acid fragmentation patterns associated with a plurality of sequence fragments from the plurality of cell-free nucleic acid samples.

In Example 215, the subject matter of Example 214 includes, wherein determining the cell-free nucleic acid score is further based on a tumor fraction regression (TFR) score, wherein the TFR score is indicative of a fraction of molecules of the plurality of cell-free nucleic acid samples that indicate a tumor.

In Example 216, the subject matter of Examples 203-215 includes, wherein the one or more non-transitory computer-readable storage media store additional computer-readable instructions that, when executed by the one or more hardware processors, cause the one or more hardware processors to perform additional operations comprising determining the genomic alterations of each of the plurality of cell-free nucleic acid samples.

In Example 217, the subject matter of Example 216 includes, wherein the computer-readable instructions that cause the one or more hardware processors to perform operations comprising determining the genomic alterations of each of the plurality of cell-free nucleic acid samples includes computer-readable instructions that cause the one or more hardware processors to perform operations comprising determining somatic variants observed in molecules from each of a plurality of sequence fragments from the plurality of cell-free nucleic acid samples.

In Example 218, the subject matter of Examples 203-217 includes, wherein the plurality of cell-free nucleic acid samples are from a plurality of genomic regions, wherein the plurality of genomic regions includes at least one of: a genomic region known to be associated with a cancer type, a genomic region associated with a known methylation status, a genomic region known to be associated with hypomethylation, or a genomic region known to be associated with therapy response.

In Example 219, the subject matter of Examples 203-218 includes, wherein the plurality of cell-free nucleic acid samples are from a plurality of genomic regions, wherein the plurality of genomic regions includes at least one genomic region known to be associated with colorectal cancer.

In Example 220, the subject matter of Examples 203-219 includes, wherein determining that the plurality of cell-free nucleic acid samples is tumor-derived or non-tumor derived is further based on a methylation value satisfying a threshold, wherein the methylation score is indicative of a quantity of molecules of the plurality of cell-free nucleic acid samples that indicate a tumor.

In Example 221, the subject matter of Examples 203-220 includes, wherein the plurality of cell-free nucleic acid samples includes cell-free deoxyribonucleic (cfDNA) samples.

In Example 222, the subject matter of Examples 203-221 includes, wherein the plurality of cell-free nucleic acid samples includes ribonucleic acid (RNA) samples.

In Example 223, the subject matter of Examples 203-222 includes, wherein the plurality of cell-free nucleic acid samples includes cell-free ribonucleic acid (cfRNA) samples.

In Example 224, the subject matter of Examples 203-223 includes, wherein the plurality of cell-free nucleic acid samples includes mitochondrial deoxyribonucleic (mtDNA) samples.

In Example 225, the subject matter of Examples 203-224 includes, wherein the plurality of cell-free nucleic acid samples includes mitochondrial ribonucleic (mtRNA) samples.

In Example 226, the subject matter of Examples 203-225 includes, wherein the plurality of cell-free nucleic acid samples includes extracellular vesicle-bound deoxyribonucleic (evDNA) samples.

Example 227 is a computing system comprising: one or more hardware processors; and one or more non-transitory computer-readable storage media including computer-readable instructions that, when executed by the one or more hardware processors, cause the one or more hardware processors to perform operations comprising: determining, based on a quantification of an observed tumor-associated aberrant methylation of each of a plurality of cell-free nucleic acid samples, using a tumor fraction regression (TFR) model, a TFR score, wherein the TFR score includes, a fraction of molecules of the plurality of cell-free nucleic acid samples that indicate a tumor, wherein each of the plurality of cell-free nucleic acid samples is labeled with a tumor-derived label or a non-tumor-derived label; determining, based on at least one of epigenetic factors or genomic alterations of each of the plurality of cell-free nucleic acid samples, a cell-free nucleic acid score indicative of presence of a tumor; determining, based on at least one of the cell-free nucleic acid score or the TFR score satisfying a respective threshold, a tumor prediction for each of the plurality of cell-free nucleic acid samples; generating, based on the tumor-derived label or the non-tumor-derived label and the tumor prediction for each of the plurality of cell-free nucleic acid samples, a predictive model to predict a tumor in the plurality of cell-free nucleic acid samples; and outputting the predictive model.

In Example 228, the subject matter of Example 227 includes, wherein the one or more non-transitory computer-readable storage media store additional computer-readable instructions that, when executed by the one or more hardware processors, cause the one or more hardware processors to perform additional operations comprising determining the quantification of the observed tumor-associated aberrant methylation of each of the plurality of cell-free nucleic acid samples.

In Example 229, the subject matter of Example 228 includes, wherein the computer-readable instructions that cause the one or more hardware processors to perform operations comprising determining the quantification of the observed tumor-associated aberrant methylation of each of the plurality of cell-free nucleic acid samples includes computer-readable instructions that cause the one or more hardware processors to perform operations comprising quantifying a number of unique methylated molecules mapping to each of a plurality of genomic regions.

In Example 230, the subject matter of Examples 227-229 includes, wherein the one or more non-transitory computer-readable storage media store additional computer-readable instructions that, when executed by the one or more hardware processors, cause the one or more hardware processors to perform additional operations comprising determining the epigenetic factors of each of the plurality of cell-free nucleic acid samples.

In Example 231, the subject matter of Example 230 includes, wherein the one or more non-transitory computer-readable storage media store additional computer-readable instructions that, when executed by the one or more hardware processors, cause the one or more hardware processors to perform additional operations comprising determining the epigenetic factors of each of the plurality of cell-free nucleic acid samples based on a methylation logistic regression (LR) model cancer or non-cancer classification.

In Example 232, the subject matter of Example 231 includes, wherein the one or more non-transitory computer-readable storage media store additional computer-readable instructions that, when executed by the one or more hardware processors, cause the one or more hardware processors to perform additional operations comprising determining, using a LR model, the methylation LR model cancer or non-cancer classification.

In Example 233, the subject matter of Examples 230-232 includes, wherein determining the epigenetic factors of each of the plurality of cell-free nucleic acid samples is based on a cancer signal from cell-free nucleic acid fragmentation patterns associated with a plurality of sequence fragments from the plurality of cell-free nucleic acid samples.

In Example 234, the subject matter of Example 233 includes, wherein the one or more non-transitory computer-readable storage media store additional computer-readable instructions that, when executed by the one or more hardware processors, cause the one or more hardware processors to perform additional operations comprising determining, using a fragmentomics model, the cancer signal from the cell-free nucleic acid fragmentation patterns associated with the plurality of sequence fragments from the plurality of cell-free nucleic acid samples.

In Example 235, the subject matter of Examples 230-234 includes, wherein the one or more non-transitory computer-readable storage media store additional computer-readable instructions that, when executed by the one or more hardware processors, cause the one or more hardware processors to perform additional operations comprising determining the epigenetic factors of each of the plurality of cell-free nucleic acid samples based on a methylation logistic regression (LR) model cancer or non-cancer classification and based on a cancer signal from cell-free nucleic acid fragmentation patterns associated with a plurality of sequence fragments from the plurality of cell-free nucleic acid samples.

In Example 236, the subject matter of Example 235 includes, wherein determining the cell-free nucleic acid score is further based on the TFR score.

In Example 237, the subject matter of Examples 227-236 includes, wherein the one or more non-transitory computer-readable storage media store additional computer-readable instructions that, when executed by the one or more hardware processors, cause the one or more hardware processors to perform additional operations comprising determining the genomic alterations of each of the plurality of cell-free nucleic acid samples.

In Example 238, the subject matter of Example 237 includes, wherein the computer-readable instructions that cause the one or more hardware processors to perform operations comprising determining the genomic alterations of each of the plurality of cell-free nucleic acid samples includes computer-readable instructions that cause the one or more hardware processors to perform operations comprising determining somatic variants observed in molecules from the plurality of cell-free nucleic acid samples.

In Example 239, the subject matter of Examples 227-238 includes, wherein the plurality of cell-free nucleic acid samples are from a plurality of genomic regions, wherein the plurality of genomic regions includes at least one of: a genomic region known to be associated with a cancer type, a genomic region associated with a known methylation status, a genomic region known to be associated with hypomethylation, or a genomic region known to be associated with therapy response.

In Example 240, the subject matter of Examples 227-239 includes, wherein the plurality of cell-free nucleic acid samples are from a plurality of genomic regions, wherein the plurality of genomic regions includes at least one genomic region known to be associated with colorectal cancer.

In Example 241, the subject matter of Examples 227-240 includes, wherein determining the cell-free nucleic acid score is further based on the TFR score.

In Example 242, the subject matter of Examples 227-241 includes, wherein the plurality of cell-free nucleic acid samples includes cell-free deoxyribonucleic (cfDNA) samples.

In Example 243, the subject matter of Examples 227-242 includes, wherein the plurality of cell-free nucleic acid samples includes ribonucleic acid (RNA) samples.

In Example 244, the subject matter of Examples 227-243 includes, wherein the plurality of cell-free nucleic acid samples includes cell-free ribonucleic acid (cfRNA) samples.

In Example 245, the subject matter of Examples 227-244 includes, wherein the plurality of cell-free nucleic acid samples includes mitochondrial deoxyribonucleic (mtDNA) samples.

In Example 246, the subject matter of Examples 227-245 includes, wherein the plurality of cell-free nucleic acid samples includes mitochondrial ribonucleic (mtRNA) samples.

In Example 247, the subject matter of Examples 227-246 includes, wherein the plurality of cell-free nucleic acid samples includes extracellular vesicle-bound deoxyribonucleic (evDNA) samples.

Example 248 is a computing system comprising: one or more hardware processors; and one or more non-transitory computer-readable storage media including computer-readable instructions that, when executed by the one or more hardware processors, cause the one or more hardware processors to perform operations comprising: determining, based on a quantification of an observed tumor-associated aberrant methylation of each of a plurality of cell-free nucleic acid samples, using a tumor fraction regression (TFR) model, a TFR score, wherein the TFR score includes, a fraction of molecules of the plurality of cell-free nucleic acid samples that indicate a tumor, wherein each of the plurality of cell-free nucleic acid samples is labeled with a tumor-derived label or a non-tumor-derived label; determining, based on the TFR score satisfying a respective threshold, a tumor prediction for each of the plurality of cell-free nucleic acid samples; generating, based on the tumor-derived label or the non-tumor-derived label and the tumor prediction of the plurality of cell-free nucleic acid samples, a predictive model to predict a tumor in the plurality of cell-free nucleic acid samples; and outputting the predictive model.

In Example 249, the subject matter of Example 248 includes, wherein the one or more non-transitory computer-readable storage media store additional computer-readable instructions that, when executed by the one or more hardware processors, cause the one or more hardware processors to perform additional operations comprising determining the quantification of the observed tumor-associated aberrant methylation of each of the plurality of cell-free nucleic acid samples.

In Example 250, the subject matter of Example 249 includes, wherein the computer-readable instructions that cause the one or more hardware processors to perform operations comprising determining the quantification of the observed tumor-associated aberrant methylation of each of the plurality of cell-free nucleic acid samples includes computer-readable instructions that cause the one or more hardware processors to perform operations comprising quantifying a number of unique methylated molecules mapping to each of a plurality of genomic regions.

In Example 251, the subject matter of Examples 248-250 includes, wherein determining the tumor prediction for each of the plurality of cell-free nucleic acid samples is further based on a cell-free nucleic acid score indicative of presence of a tumor.

In Example 252, the subject matter of Example 251 includes, wherein the one or more non-transitory computer-readable storage media store additional computer-readable instructions that, when executed by the one or more hardware processors, cause the one or more hardware processors to perform additional operations comprising determining, based on at least one of epigenetic factors or genomic alterations of each of the plurality of cell-free nucleic acid samples, the cell-free nucleic acid score indicative of presence of a tumor.

In Example 253, the subject matter of Example 252 includes, wherein the one or more non-transitory computer-readable storage media store additional computer-readable instructions that, when executed by the one or more hardware processors, cause the one or more hardware processors to perform additional operations comprising determining the epigenetic factors of each of the plurality of cell-free nucleic acid samples.

In Example 254, the subject matter of Example 253 includes, wherein the one or more non-transitory computer-readable storage media store additional computer-readable instructions that, when executed by the one or more hardware processors, cause the one or more hardware processors to perform additional operations comprising determining the epigenetic factors of each of the plurality of cell-free nucleic acid samples based on a methylation logistic regression (LR) model cancer or non-cancer classification.

In Example 255, the subject matter of Example 254 includes, wherein the one or more non-transitory computer-readable storage media store additional computer-readable instructions that, when executed by the one or more hardware processors, cause the one or more hardware processors to perform additional operations comprising determining, using a LR model, the methylation LR model cancer or non-cancer classification.

In Example 256, the subject matter of Examples 253-255 includes, wherein the one or more non-transitory computer-readable storage media store additional computer-readable instructions that, when executed by the one or more hardware processors, cause the one or more hardware processors to perform additional operations comprising determining the epigenetic factors of each of the plurality of cell-free nucleic acid samples based on a cancer signal from cell-free nucleic acid fragmentation patterns associated with a plurality of sequence fragments from the plurality of cell-free nucleic acid samples.

In Example 257, the subject matter of Example 256 includes, wherein the one or more non-transitory computer-readable storage media store additional computer-readable instructions that, when executed by the one or more hardware processors, cause the one or more hardware processors to perform additional operations comprising determining, using a fragmentomics model, the cancer signal from the cell-free nucleic acid fragmentation patterns associated with a plurality of sequence fragments from the plurality of cell-free nucleic acid samples.

In Example 258, the subject matter of Examples 252-257 includes, wherein the one or more non-transitory computer-readable storage media store additional computer-readable instructions that, when executed by the one or more hardware processors, cause the one or more hardware processors to perform additional operations comprising determining the genomic alterations of each of the plurality of cell-free nucleic acid samples.

In Example 259, the subject matter of Example 258 includes, wherein the computer-readable instructions that cause the one or more hardware processors to perform operations comprising determining the genomic alterations of each of the plurality of cell-free nucleic acid samples includes computer-readable instructions that cause the one or more hardware processors to perform operations comprising determining somatic variants observed in molecules from each of a plurality of sequence fragments from the plurality of cell-free nucleic acid samples.

In Example 260, the subject matter of Examples 252-259 includes, wherein the one or more non-transitory computer-readable storage media store additional computer-readable instructions that, when executed by the one or more hardware processors, cause the one or more hardware processors to perform additional operations comprising determining the epigenetic factors of each of the plurality of cell-free nucleic acid samples based on a methylation logistic regression (LR) model cancer or non-cancer classification and based on a cancer signal from cell-free nucleic acid fragmentation patterns associated with a plurality of sequence fragments from the plurality of cell-free nucleic acid samples.

In Example 261, the subject matter of Example 260 includes, wherein determining the cell-free nucleic acid score is further based on the TFR score.

In Example 262, the subject matter of Examples 248-261 includes, wherein the plurality of cell-free nucleic acid samples are from a plurality of genomic regions, wherein the plurality of genomic regions includes at least one of: a genomic region known to be associated with a cancer type, a genomic region associated with a known methylation status, a genomic region known to be associated with hypomethylation, or a genomic region known to be associated with therapy response.

In Example 263, the subject matter of Examples 248-262 includes, wherein the plurality of cell-free nucleic acid samples are from a plurality of genomic regions, wherein the plurality of genomic regions includes at least one genomic region known to be associated with colorectal cancer.

In Example 264, the subject matter of Examples 248-263 includes, wherein the plurality of cell-free nucleic acid samples includes cell-free deoxyribonucleic (cfDNA) samples.

In Example 265, the subject matter of Examples 247-264 includes, wherein the plurality of cell-free nucleic acid samples includes ribonucleic acid (RNA) samples.

In Example 266, the subject matter of Examples 248-265 includes, wherein the plurality of cell-free nucleic acid samples includes cell-free ribonucleic acid (cfRNA) samples.

In Example 267, the subject matter of Examples 248-266 includes, wherein the plurality of cell-free nucleic acid samples includes mitochondrial deoxyribonucleic (mtDNA) samples.

In Example 268, the subject matter of Examples 248-267 includes, wherein the plurality of cell-free nucleic acid samples includes mitochondrial ribonucleic (mtRNA) samples.

In Example 269, the subject matter of Examples 248-268 includes, wherein the plurality of cell-free nucleic acid samples includes extracellular vesicle-bound deoxyribonucleic (evDNA) samples.

Example 270 is a computing system comprising: one or more hardware processors; and one or more non-transitory computer-readable storage media including computer-readable instructions that, when executed by the one or more hardware processors, cause the one or more hardware processors to perform operations comprising: determining, based on at least one of epigenetic factors or genomic alterations of each of a plurality of cell-free nucleic acid samples, a cell-free nucleic acid score indicative of presence of a tumor, wherein each of the plurality of cell-free nucleic acid samples is labeled with a tumor-derived label or a non-tumor-derived label; determining, based on the cell-free nucleic acid score satisfying a respective threshold, a tumor prediction for each of the plurality of cell-free nucleic acid samples; generating, based on the tumor-derived label or the non-tumor-derived label and the tumor prediction for each of the plurality of cell-free nucleic acid samples, a predictive model to predict a tumor in the plurality of cell-free nucleic acid samples; and outputting the predictive model.

In Example 271, the subject matter of Example 270 includes, wherein determining the tumor prediction for each of the plurality of cell-free nucleic acid is further based on a tumor fraction regression (TFR) score satisfying a threshold, wherein the TFR score is indicative of a fraction of molecules of the plurality of cell-free nucleic acid samples that indicate a tumor.

In Example 272, the subject matter of Example 271 includes, wherein the one or more non-transitory computer-readable storage media store additional computer-readable instructions that, when executed by the one or more hardware processors, cause the one or more hardware processors to perform additional operations comprising determining, based on a quantification of an observed tumor-associated aberrant methylation of each of the plurality of cell-free nucleic acid samples, using a TFR model, the TFR score.

In Example 273, the subject matter of Example 272 includes, wherein the one or more non-transitory computer-readable storage media store additional computer-readable instructions that, when executed by the one or more hardware processors, cause the one or more hardware processors to perform additional operations comprising determining the quantification of the observed tumor-associated aberrant methylation of each of the plurality of cell-free nucleic acid samples.

In Example 274, the subject matter of Example 273 includes, wherein the computer-readable instructions that cause the one or more hardware processors to perform operations comprising determining the quantification of the observed tumor-associated aberrant methylation of each of the plurality of cell-free nucleic acid samples includes computer-readable instructions that cause the one or more hardware processors to perform operations comprising quantifying a number of unique methylated molecules mapping to each of a plurality of genomic regions.

In Example 275, the subject matter of Examples 271-274 includes, wherein determining the cell-free nucleic acid score is further based on the TFR score.

In Example 276, the subject matter of Examples 270-275 includes, wherein the one or more non-transitory computer-readable storage media store additional computer-readable instructions that, when executed by the one or more hardware processors, cause the one or more hardware processors to perform additional operations comprising determining the epigenetic factors of each of the plurality of cell-free nucleic acid samples.

In Example 277, the subject matter of Example 276 includes, wherein the one or more non-transitory computer-readable storage media store additional computer-readable instructions that, when executed by the one or more hardware processors, cause the one or more hardware processors to perform additional operations comprising determining the epigenetic factors of each of the plurality of cell-free nucleic acid samples based on a methylation logistic regression (LR) model cancer or non-cancer classification.

In Example 278, the subject matter of Example 277 includes, wherein the one or more non-transitory computer-readable storage media store additional computer-readable instructions that, when executed by the one or more hardware processors, cause the one or more hardware processors to perform additional operations comprising determining, using a LR model, the methylation LR model cancer or non-cancer classification.

In Example 279, the subject matter of Examples 276-278 includes, wherein the one or more non-transitory computer-readable storage media store additional computer-readable instructions that, when executed by the one or more hardware processors, cause the one or more hardware processors to perform additional operations comprising determining the epigenetic factors of each of the plurality of cell-free nucleic acid samples based on a cancer signal from cell-free nucleic acid fragmentation patterns associated with a plurality of sequence fragments from the plurality of cell-free nucleic acid samples.

In Example 280, the subject matter of Example 279 includes, wherein the one or more non-transitory computer-readable storage media store additional computer-readable instructions that, when executed by the one or more hardware processors, cause the one or more hardware processors to perform additional operations comprising determining, using a fragmentomics model, the cancer signal from the cell-free nucleic acid fragmentation patterns associated with the plurality of sequence fragments from the plurality of cell-free nucleic acid samples.

In Example 281, the subject matter of Examples 276-280 includes, wherein the one or more non-transitory computer-readable storage media store additional computer-readable instructions that, when executed by the one or more hardware processors, cause the one or more hardware processors to perform additional operations comprising determining the epigenetic factors of each of the plurality of cell-free nucleic acid samples based on a methylation logistic regression (LR) model cancer or non-cancer classification and based on a cancer signal from cell-free nucleic acid fragmentation patterns associated with a plurality of sequence fragments from the plurality of cell-free nucleic acid samples.

In Example 282, the subject matter of Example 281 includes, wherein determining the cell-free nucleic acid score is further based on a tumor fraction regression (TFR) score, wherein the TFR score is indicative of a fraction of molecules of the plurality of cell-free nucleic acid samples that indicate a tumor.

In Example 283, the subject matter of Examples 270-282 includes, wherein the one or more non-transitory computer-readable storage media store additional computer-readable instructions that, when executed by the one or more hardware processors, cause the one or more hardware processors to perform additional operations comprising determining the genomic alterations of each of the plurality of cell-free nucleic acid samples.

In Example 284, the subject matter of Example 283 includes, wherein the computer-readable instructions that cause the one or more hardware processors to perform operations comprising determining the genomic alterations of each of the plurality of cell-free nucleic acid samples includes computer-readable instructions that cause the one or more hardware processors to perform operations comprising determining somatic variants observed in molecules from each of a plurality of sequence fragments from the plurality of cell-free nucleic acid samples.

In Example 285, the subject matter of Examples 270-284 includes, wherein the plurality of cell-free nucleic acid samples are from a plurality of genomic regions, wherein the plurality of genomic regions includes at least one of: a genomic region known to be associated with a cancer type, a genomic region associated with a known methylation status, a genomic region known to be associated with hypomethylation, or a genomic region known to be associated with therapy response.

In Example 286, the subject matter of Examples 270-285 includes, wherein the plurality of cell-free nucleic acid samples are from a plurality of genomic regions, wherein the plurality of genomic regions includes at least one genomic region known to be associated with colorectal cancer.

In Example 287, the subject matter of Examples 270-286 includes, wherein determining that the plurality of cell-free nucleic acid samples is tumor-derived or non-tumor derived is further based on a methylation value satisfying a threshold, wherein the methylation score is indicative of a quantity of molecules of the plurality of cell-free nucleic acid samples that indicate a tumor.

In Example 288, the subject matter of Examples 270-287 includes, wherein the plurality of cell-free nucleic acid samples includes cell-free deoxyribonucleic (cfDNA) samples.

In Example 289, the subject matter of Examples 270-288 includes, wherein the plurality of cell-free nucleic acid samples includes ribonucleic acid (RNA) samples.

In Example 290, the subject matter of Examples 270-289 includes, wherein the plurality of cell-free nucleic acid samples includes cell-free ribonucleic acid (cfRNA) samples.

In Example 291, the subject matter of Examples 270-290 includes, wherein the plurality of cell-free nucleic acid samples includes mitochondrial deoxyribonucleic (mtDNA) samples.

In Example 292, the subject matter of Examples 270-291 includes, wherein the plurality of cell-free nucleic acid samples includes mitochondrial ribonucleic (mtRNA) samples.

In Example 293, the subject matter of Examples 270-292 includes, wherein the plurality of cell-free nucleic acid samples includes extracellular vesicle-bound deoxyribonucleic (evDNA) samples.

Example 294 is a computing system comprising: one or more hardware processors; and one or more non-transitory computer-readable storage media including computer-readable instructions that, when executed by the one or more hardware processors, cause the one or more hardware processors to perform operations comprising: detecting one or more biomarkers in a biological sample; determining, based on a quantification of an observed tumor-associated aberrant methylation of each of a plurality of cell-free nucleic acid samples, using a tumor fraction regression (TFR) model, a TFR score, wherein the TFR score includes, a fraction of molecules of the plurality of cell-free nucleic acid samples that indicate a tumor; determining, based on at least one of epigenetic factors or genomic alterations of each of the plurality of cell-free nucleic acid samples, a cell-free nucleic acid score indicative of presence of a tumor; and determining, based on at least one of the detected biomarkers, the cell-free nucleic acid score, or the TFR score satisfying a respective threshold, that the biological sample is tumor-derived or non-tumor derived.

In Example 295, the subject matter of Example 294 includes, wherein the one or more non-transitory computer-readable storage media store additional computer-readable instructions that, when executed by the one or more hardware processors, cause the one or more hardware processors to perform additional operations comprising determining the quantification of the observed tumor-associated aberrant methylation of each of the plurality of cell-free nucleic acid samples.

In Example 296, the subject matter of Example 295 includes, wherein the computer-readable instructions that cause the one or more hardware processors to perform operations comprising determining the quantification of the observed tumor-associated aberrant methylation of each of the plurality of cell-free nucleic acid samples includes computer-readable instructions that cause the one or more hardware processors to perform operations comprising quantifying a number of unique methylated molecules mapping to each of a plurality of genomic regions.

In Example 297, the subject matter of Examples 294-296 includes, wherein the one or more non-transitory computer-readable storage media store additional computer-readable instructions that, when executed by the one or more hardware processors, cause the one or more hardware processors to perform additional operations comprising determining the epigenetic factors of each of the plurality of cell-free nucleic acid samples.

In Example 298, the subject matter of Example 297 includes, wherein the one or more non-transitory computer-readable storage media store additional computer-readable instructions that, when executed by the one or more hardware processors, cause the one or more hardware processors to perform additional operations comprising determining the epigenetic factors of each of the plurality of cell-free nucleic acid samples based on a methylation logistic regression (LR) model cancer or non-cancer classification.

In Example 299, the subject matter of Example 298 includes, wherein the one or more non-transitory computer-readable storage media store additional computer-readable instructions that, when executed by the one or more hardware processors, cause the one or more hardware processors to perform additional operations comprising determining, using a LR model, the methylation LR model cancer or non-cancer classification.

In Example 300, the subject matter of Examples 297-299 includes, wherein the one or more non-transitory computer-readable storage media store additional computer-readable instructions that, when executed by the one or more hardware processors, cause the one or more hardware processors to perform additional operations comprising determining the epigenetic factors of each of the plurality of cell-free nucleic acid samples based on a cancer signal from cell-free nucleic acid fragmentation patterns associated with a plurality of sequence fragments from the plurality of cell-free nucleic acid samples.

In Example 301, the subject matter of Example 300 includes, wherein the one or more non-transitory computer-readable storage media store additional computer-readable instructions that, when executed by the one or more hardware processors, cause the one or more hardware processors to perform additional operations comprising determining, using a fragmentomics model, the cancer signal from the cell-free nucleic acid fragmentation patterns associated with the plurality of sequence fragments from the plurality of cell-free nucleic acid samples.

In Example 302, the subject matter of Examples 297-301 includes, wherein the one or more non-transitory computer-readable storage media store additional computer-readable instructions that, when executed by the one or more hardware processors, cause the one or more hardware processors to perform additional operations comprising determining the epigenetic factors of each of the plurality of cell-free nucleic acid samples based on a methylation logistic regression (LR) model cancer or non-cancer classification and based on a cancer signal from cell-free nucleic acid fragmentation patterns associated with a plurality of sequence fragments from the plurality of cell-free nucleic acid samples.

In Example 303, the subject matter of Example 302 includes, wherein determining the cell-free nucleic acid score is further based on the TFR score.

In Example 304, the subject matter of Examples 294-303 includes, wherein the one or more non-transitory computer-readable storage media store additional computer-readable instructions that, when executed by the one or more hardware processors, cause the one or more hardware processors to perform additional operations comprising determining the genomic alterations of each of the plurality of cell-free nucleic acid samples.

In Example 305, the subject matter of Example 304 includes, wherein the computer-readable instructions that cause the one or more hardware processors to perform operations comprising determining the genomic alterations of each of the plurality of cell-free nucleic acid samples includes computer-readable instructions that cause the one or more hardware processors to perform operations comprising determining somatic variants observed in molecules from each of a plurality of sequence fragments from the plurality of cell-free nucleic acid samples.

In Example 306, the subject matter of Examples 294-305 includes, wherein the plurality of cell-free nucleic acid samples are from a plurality of genomic regions, wherein the plurality of genomic regions includes at least one of: a genomic region known to be associated with a cancer type, a genomic region associated with a known methylation status, a genomic region known to be associated with hypomethylation, or a genomic region known to be associated with therapy response.

In Example 307, the subject matter of Examples 294-306 includes, wherein the plurality of cell-free nucleic acid samples are from a plurality of genomic regions, wherein the plurality of genomic regions includes at least one genomic region known to be associated with colorectal cancer.

In Example 308, the subject matter of Examples 294-307 includes, wherein determining the cell-free nucleic acid score is further based on the TFR score.

In Example 309, the subject matter of Examples 294-308 includes, wherein the plurality of cell-free nucleic acid samples includes cell-free deoxyribonucleic (cfDNA) samples.

In Example 310, the subject matter of Examples 294-309 includes, wherein the plurality of cell-free nucleic acid samples includes ribonucleic acid (RNA) samples.

In Example 311, the subject matter of Examples 294-310 includes, wherein the plurality of cell-free nucleic acid samples includes cell-free ribonucleic acid (cfRNA) samples.

In Example 312, the subject matter of Examples 294-311 includes, wherein the plurality of cell-free nucleic acid samples includes mitochondrial deoxyribonucleic (mtDNA) samples.

In Example 313, the subject matter of Examples 294-312 includes, wherein the plurality of cell-free nucleic acid samples includes mitochondrial ribonucleic (mtRNA) samples.

In Example 314, the subject matter of Examples 294-313 includes, wherein the plurality of cell-free nucleic acid samples includes extracellular vesicle-bound deoxyribonucleic (evDNA) samples.

In Example 315, the subject matter of Examples 294-314 includes, wherein the biomarker is one or more of those selected from: proteins, exosomes, exomeres, microvesicles, apoptotic bodies, neutrophil extracellular traps (NETs), immune cells, tumor-educated platelets (TEPs), microbiome, virome, toll-like receptors (TLRs), and mitochondrial DNA (mtDNA).

In Example 316, the subject matter of Examples 294-315 includes, wherein the computer-readable instructions that cause the one or more hardware processors to perform operations comprising detecting one ore more biomarkers includes computer-readable instructions that cause the one or more hardware processors to perform operations comprising detecting the presence or levels of the one or more biomarkers.

In Example 317, the subject matter of Examples 294-316 includes, wherein the computer-readable instructions that cause the one or more hardware processors to perform operations comprising determining that the biological sample is tumor-derived or non-tumor derived includes computer-readable instructions that cause the one or more hardware processors to perform operations comprising comparing the levels of the one or more biomarkers in the biological sample to a control.

In Example 318, the subject matter of Example 317 includes, wherein the control is a reference level or a level present in a healthy, non-cancer subject.

Example 319 is one or more non-transitory computer readable media storing processor-executable instructions that, when executed by at least one processor, cause the at least one processor to: determining, based on a quantification of an observed tumor-associated aberrant methylation of each of a plurality of cell-free nucleic acid samples, using a tumor fraction regression (TFR) model, a TFR score, wherein the TFR score includes, a fraction of molecules of the plurality of cell-free nucleic acid samples that indicate a tumor; determining, based on at least one of epigenetic factors or genomic alterations of each of the plurality of cell-free nucleic acid samples, a cell-free nucleic acid score indicative of presence of a tumor; and determining, based on at least one of the cell-free nucleic acid score or the TFR score satisfying a respective threshold, using a predictive model, that the plurality of cell-free nucleic acid samples is tumor-derived or non-tumor derived.

In Example 320, the subject matter of Example 319 includes, wherein the one or more non-transitory computer-readable storage media store additional computer-readable instructions that, when executed by the at least one processor, cause the at least one processor to perform additional operations comprising determining the quantification of the observed tumor-associated aberrant methylation of each of the plurality of cell-free nucleic acid samples.

In Example 321, the subject matter of Example 320 includes, wherein the computer-readable instructions that cause the at least one processor to perform operations comprising determining the quantification of the observed tumor-associated aberrant methylation of each of the plurality of cell-free nucleic acid samples includes computer-readable instructions that cause the at least one processor to perform operations comprising quantifying a number of unique methylated molecules mapping to each of a plurality of genomic regions.

In Example 322, the subject matter of Examples 319-321 includes, wherein the one or more non-transitory computer-readable storage media store additional computer-readable instructions that, when executed by the at least one processor, cause the at least one processor to perform additional operations comprising determining the epigenetic factors of each of the plurality of cell-free nucleic acid samples.

In Example 323, the subject matter of Example 322 includes, wherein the one or more non-transitory computer-readable storage media store additional computer-readable instructions that, when executed by the at least one processor, cause the at least one processor to perform additional operations comprising determining the epigenetic factors of each of the plurality of cell-free nucleic acid samples based on a methylation logistic regression (LR) model cancer or non-cancer classification.

In Example 324, the subject matter of Example 323 includes, wherein the one or more non-transitory computer-readable storage media store additional computer-readable instructions that, when executed by the at least one processor, cause the at least one processor to perform additional operations comprising determining, using a LR model, the methylation LR model cancer or non-cancer classification.

In Example 325, the subject matter of Examples 322-324 includes, wherein the one or more non-transitory computer-readable storage media store additional computer-readable instructions that, when executed by the at least one processor, cause the at least one processor to perform additional operations comprising determining the epigenetic factors of each of the plurality of cell-free nucleic acid samples based on a cancer signal from cell-free nucleic acid fragmentation patterns associated with a plurality of sequence fragments from the plurality of cell-free nucleic acid samples.

In Example 326, the subject matter of Example 325 includes, wherein the one or more non-transitory computer-readable storage media store additional computer-readable instructions that, when executed by the at least one processor, cause the at least one processor to perform additional operations comprising determining, using a fragmentomics model, the cancer signal from the cell-free nucleic acid fragmentation patterns associated with the plurality of sequence fragments from the plurality of cell-free nucleic acid samples.

In Example 327, the subject matter of Examples 322-326 includes, wherein the one or more non-transitory computer-readable storage media store additional computer-readable instructions that, when executed by the at least one processor, cause the at least one processor to perform additional operations comprising determining the epigenetic factors of each of the plurality of cell-free nucleic acid samples based on a methylation logistic regression (LR) model cancer or non-cancer classification and based on a cancer signal from cell-free nucleic acid fragmentation patterns associated with a plurality of sequence fragments from the plurality of cell-free nucleic acid samples.

In Example 328, the subject matter of Example 327 includes, wherein determining the cell-free nucleic acid score is further based on the TFR score.

In Example 329, the subject matter of Examples 319-328 includes, wherein the one or more non-transitory computer-readable storage media store additional computer-readable instructions that, when executed by the at least one processor, cause the at least one processor to perform additional operations comprising determining the genomic alterations of each of the plurality of cell-free nucleic acid samples.

In Example 330, the subject matter of Example 329 includes, wherein the computer-readable instructions that cause the at least one processor to perform operations comprising determining the genomic alterations of each of the plurality of cell-free nucleic acid samples includes computer-readable instructions that cause the at least one processor to perform operations comprising determining somatic variants observed in molecules from each of a plurality of sequence fragments from the plurality of cell-free nucleic acid samples.

In Example 331, the subject matter of Examples 319-330 includes, wherein the plurality of cell-free nucleic acid samples are from a plurality of genomic regions, wherein the plurality of genomic regions includes at least one of: a genomic region known to be associated with a cancer type, a genomic region associated with a known methylation status, a genomic region known to be associated with hypomethylation, or a genomic region known to be associated with therapy response.

In Example 332, the subject matter of Examples 319-331 includes, wherein the plurality of cell-free nucleic acid samples are from a plurality of genomic regions, wherein the plurality of genomic regions includes at least one genomic region known to be associated with colorectal cancer.

In Example 333, the subject matter of Examples 319-332 includes, wherein determining the cell-free nucleic acid score is further based on the TFR score.

In Example 334, the subject matter of Examples 319-333 includes, wherein the plurality of cell-free nucleic acid samples includes cell-free deoxyribonucleic (cfDNA) samples.

In Example 335, the subject matter of Examples 319-334 includes, wherein the plurality of cell-free nucleic acid samples includes ribonucleic acid (RNA) samples.

In Example 336, the subject matter of Examples 319-335 includes, wherein the plurality of cell-free nucleic acid samples includes cell-free ribonucleic acid (cfRNA) samples.

In Example 337, the subject matter of Examples 319-336 includes, wherein the plurality of cell-free nucleic acid samples includes mitochondrial deoxyribonucleic (mtDNA) samples.

In Example 338, the subject matter of Examples 319-337 includes, wherein the plurality of cell-free nucleic acid samples includes mitochondrial ribonucleic (mtRNA) samples.

In Example 339, the subject matter of Examples 319-338 includes, wherein the plurality of cell-free nucleic acid samples includes extracellular vesicle-bound deoxyribonucleic (evDNA) samples.

Example 340 is one or more non-transitory computer readable media storing processor-executable instructions that, when executed by at least one processor, cause the at least one processor to: determining, based on a quantification of an observed tumor-associated aberrant methylation of each of the plurality of cell-free nucleic acid samples, using a tumor fraction regression (TFR) model, a TFR score, wherein the TFR score includes, a fraction of molecules of the plurality of cell-free nucleic acid samples that indicate a tumor; and determining, based on the TFR score satisfying a respective threshold, using a predictive model, that the plurality of cell-free nucleic acid samples is tumor-derived or non-tumor derived.

In Example 341, the subject matter of Example 340 includes, wherein the one or more non-transitory computer-readable storage media store additional computer-readable instructions that, when executed by the at least one processor, cause the at least one processor to perform additional operations comprising determining the quantification of the observed tumor-associated aberrant methylation of each of the plurality of cell-free nucleic acid samples.

In Example 342, the subject matter of Example 341 includes, wherein the computer-readable instructions that cause the at least one processor to perform operations comprising determining the quantification of the observed tumor-associated aberrant methylation of each of the plurality of cell-free nucleic acid samples includes computer-readable instructions that cause the at least one processor to perform operations comprising quantifying a number of unique methylated molecules mapping to each of a plurality of genomic regions.

In Example 343, the subject matter of Examples 340-342 includes, wherein determining that the plurality of cell-free nucleic acid samples is tumor-derived or non-tumor derived is further based on a cell-free nucleic acid score indicative of presence of a tumor.

In Example 344, the subject matter of Example 343 includes, wherein the one or more non-transitory computer-readable storage media store additional computer-readable instructions that, when executed by the at least one processor, cause the at least one processor to perform additional operations comprising determining, based on at least one of epigenetic factors or genomic alterations of each of the plurality of cell-free nucleic acid samples, the cell-free nucleic acid score indicative of presence of a tumor.

In Example 345, the subject matter of Example 344 includes, wherein the one or more non-transitory computer-readable storage media store additional computer-readable instructions that, when executed by the at least one processor, cause the at least one processor to perform additional operations comprising determining the epigenetic factors of each of the plurality of cell-free nucleic acid samples.

In Example 346, the subject matter of Example 345 includes, wherein the one or more non-transitory computer-readable storage media store additional computer-readable instructions that, when executed by the at least one processor, cause the at least one processor to perform additional operations comprising determining the epigenetic factors of each of the plurality of cell-free nucleic acid samples based on a methylation logistic regression (LR) model cancer or non-cancer classification.

In Example 347, the subject matter of Example 346 includes, wherein the one or more non-transitory computer-readable storage media store additional computer-readable instructions that, when executed by the at least one processor, cause the at least one processor to perform additional operations comprising determining, using a LR model, the methylation LR model cancer or non-cancer classification.

In Example 348, the subject matter of Examples 345-347 includes, wherein the one or more non-transitory computer-readable storage media store additional computer-readable instructions that, when executed by the at least one processor, cause the at least one processor to perform additional operations comprising determining the epigenetic factors of each of the plurality of cell-free nucleic acid samples based on a cancer signal from cell-free nucleic acid fragmentation patterns associated with a plurality of sequence fragments from the plurality of cell-free nucleic acid samples.

In Example 349, the subject matter of Example 348 includes, wherein the one or more non-transitory computer-readable storage media store additional computer-readable instructions that, when executed by the at least one processor, cause the at least one processor to perform additional operations comprising determining, using a fragmentomics model, the cancer signal from the cell-free nucleic acid fragmentation patterns associated with the plurality of sequence fragments from the plurality of cell-free nucleic acid samples.

In Example 350, the subject matter of Examples 344-349 includes, wherein the one or more non-transitory computer-readable storage media store additional computer-readable instructions that, when executed by the at least one processor, cause the at least one processor to perform additional operations comprising determining the genomic alterations of each of the plurality of cell-free nucleic acid samples.

In Example 351, the subject matter of Example 350 includes, wherein the computer-readable instructions that cause the at least one processor to perform operations comprising determining the genomic alterations of each of the plurality of cell-free nucleic acid samples includes computer-readable instructions that cause the at least one processor to perform operations comprising determining somatic variants observed in molecules from each of a plurality of sequence fragments from the plurality of cell-free nucleic acid samples.

In Example 352, the subject matter of Examples 344-351 includes, wherein the one or more non-transitory computer-readable storage media store additional computer-readable instructions that, when executed by the at least one processor, cause the at least one processor to perform additional operations comprising determining the epigenetic factors of each of the plurality of cell-free nucleic acid samples based on a methylation logistic regression (LR) model cancer or non-cancer classification and based on a cancer signal from cell-free nucleic acid fragmentation patterns associated with a plurality of sequence fragments from the plurality of cell-free nucleic acid samples.

In Example 353, the subject matter of Example 352 includes, wherein determining the cell-free nucleic acid score is further based on the TFR score.

In Example 354, the subject matter of Examples 340-353 includes, wherein the plurality of genomic regions includes at least one of: a genomic region known to be associated with a cancer type, a genomic region associated with a known methylation status, a genomic region known to be associated with hypomethylation, or a genomic region known to be associated with therapy response.

In Example 355, the subject matter of Examples 340-354 includes, wherein the plurality of genomic regions includes at least one genomic region known to be associated with colorectal cancer.

In Example 356, the subject matter of Examples 340-355 includes, wherein the plurality of cell-free nucleic acid samples includes cell-free deoxyribonucleic (cfDNA) samples.

In Example 357, the subject matter of Examples 340-356 includes, wherein the plurality of cell-free nucleic acid samples includes ribonucleic acid (RNA) samples.

In Example 358, the subject matter of Examples 340-357 includes, wherein the plurality of cell-free nucleic acid samples includes cell-free ribonucleic acid (cfRNA) samples.

In Example 359, the subject matter of Examples 340-358 includes, wherein the plurality of cell-free nucleic acid samples includes mitochondrial deoxyribonucleic (mtDNA) samples.

In Example 360, the subject matter of Examples 340-359 includes, wherein the plurality of cell-free nucleic acid samples includes mitochondrial ribonucleic (mtRNA) samples.

In Example 361, the subject matter of Examples 340-360 includes, wherein the plurality of cell-free nucleic acid samples includes extracellular vesicle-bound deoxyribonucleic (evDNA) samples.

Example 362 is one or more non-transitory computer readable media storing processor-executable instructions that, when executed by at least one processor, cause the at least one processor to: determining, based on at least one of epigenetic factors or genomic alterations of each of the plurality of cell-free nucleic acid samples, a cell-free nucleic acid score indicative of presence of a tumor; and determining, based on the cell-free nucleic acid score satisfying a respective threshold, using a predictive model, that the plurality of cell-free nucleic acid samples is tumor-derived or non-tumor derived.

In Example 363, the subject matter of Example 362 includes, wherein determining that the plurality of cell-free nucleic acid samples is tumor-derived or non-tumor derived is further based on a tumor fraction regression (TFR) score satisfying a threshold, wherein the TFR score is indicative of a fraction of molecules of the plurality of cell-free nucleic acid samples that indicate a tumor.

In Example 364, the subject matter of Example 363 includes, wherein the one or more non-transitory computer-readable storage media store additional computer-readable instructions that, when executed by the at least one processor, cause the at least one processor to perform additional operations comprising determining, based on a quantification of an observed tumor-associated aberrant methylation of each of the plurality of cell-free nucleic acid samples, using a TFR model, the TFR score.

In Example 365, the subject matter of Example 364 includes, wherein the one or more non-transitory computer-readable storage media store additional computer-readable instructions that, when executed by the at least one processor, cause the at least one processor to perform additional operations comprising determining the quantification of the observed tumor-associated aberrant methylation of each of the plurality of cell-free nucleic acid samples.

In Example 366, the subject matter of Example 365 includes, wherein the computer-readable instructions that cause the at least one processor to perform operations comprising determining the quantification of the observed tumor-associated aberrant methylation of each of the plurality of cell-free nucleic acid samples includes computer-readable instructions that cause the at least one processor to perform operations comprising quantifying a number of unique methylated molecules mapping to each of a plurality of genomic regions.

In Example 367, the subject matter of Examples 363-366 includes, wherein determining the cell-free nucleic acid score is further based on the TFR score.

In Example 368, the subject matter of Examples 362-367 includes, wherein the one or more non-transitory computer-readable storage media store additional computer-readable instructions that, when executed by the at least one processor, cause the at least one processor to perform additional operations comprising determining the epigenetic factors of each of the plurality of cell-free nucleic acid samples.

In Example 369, the subject matter of Example 368 includes, wherein the one or more non-transitory computer-readable storage media store additional computer-readable instructions that, when executed by the at least one processor, cause the at least one processor to perform additional operations comprising determining the epigenetic factors of each of the plurality of cell-free nucleic acid samples based on a methylation logistic regression (LR) model cancer or non-cancer classification.

In Example 370, the subject matter of Example 369 includes, wherein the one or more non-transitory computer-readable storage media store additional computer-readable instructions that, when executed by the at least one processor, cause the at least one processor to perform additional operations comprising determining, using a LR model, the methylation LR model cancer or non-cancer classification.

In Example 371, the subject matter of Examples 368-370 includes, wherein the one or more non-transitory computer-readable storage media store additional computer-readable instructions that, when executed by the at least one processor, cause the at least one processor to perform additional operations comprising determining the epigenetic factors of each of the plurality of cell-free nucleic acid samples based on a cancer signal from cell-free nucleic acid fragmentation patterns associated with a plurality of sequence fragments from the plurality of cell-free nucleic acid samples.

In Example 372, the subject matter of Example 371 includes, wherein the one or more non-transitory computer-readable storage media store additional computer-readable instructions that, when executed by the at least one processor, cause the at least one processor to perform additional operations comprising determining, using a fragmentomics model, the cancer signal from the cell-free nucleic acid fragmentation patterns associated with the plurality of sequence fragments from the plurality of cell-free nucleic acid samples.

In Example 373, the subject matter of Examples 368-372 includes, wherein the one or more non-transitory computer-readable storage media store additional computer-readable instructions that, when executed by the at least one processor, cause the at least one processor to perform additional operations comprising determining the epigenetic factors of each of the plurality of cell-free nucleic acid samples based on a methylation logistic regression (LR) model cancer or non-cancer classification and based on a cancer signal from cell-free nucleic acid fragmentation patterns associated with a plurality of sequence fragments from the plurality of cell-free nucleic acid samples.

In Example 374, the subject matter of Example 373 includes, wherein determining the cell-free nucleic acid score is further based on a tumor fraction regression (TFR) score, wherein the TFR score is indicative of a fraction of molecules of the plurality of cell-free nucleic acid samples that indicate a tumor.

In Example 375, the subject matter of Examples 362-374 includes, wherein the one or more non-transitory computer-readable storage media store additional computer-readable instructions that, when executed by the at least one processor, cause the at least one processor to perform additional operations comprising determining the genomic alterations of each of the plurality of cell-free nucleic acid samples.

In Example 376, the subject matter of Example 375 includes, wherein the computer-readable instructions that cause the at least one processor to perform operations comprising determining the genomic alterations of each of the plurality of cell-free nucleic acid samples includes computer-readable instructions that cause the at least one processor to perform operations comprising determining somatic variants observed in molecules from each of a plurality of sequence fragments from the plurality of cell-free nucleic acid samples.

In Example 377, the subject matter of Examples 362-376 includes, wherein the plurality of cell-free nucleic acid samples are from a plurality of genomic regions, wherein the plurality of genomic regions includes at least one of: a genomic region known to be associated with a cancer type, a genomic region associated with a known methylation status, a genomic region known to be associated with hypomethylation, or a genomic region known to be associated with therapy response.

In Example 378, the subject matter of Examples 362-377 includes, wherein the plurality of cell-free nucleic acid samples are from a plurality of genomic regions, wherein the plurality of genomic regions includes at least one genomic region known to be associated with colorectal cancer.

In Example 379, the subject matter of Examples 362-378 includes, wherein determining that the plurality of cell-free nucleic acid samples is tumor-derived or non-tumor derived is further based on a methylation value satisfying a threshold, wherein the methylation score is indicative of a quantity of molecules of the plurality of cell-free nucleic acid samples that indicate a tumor.

In Example 380, the subject matter of Examples 362-379 includes, wherein the plurality of cell-free nucleic acid samples includes cell-free deoxyribonucleic (cfDNA) samples.

In Example 381, the subject matter of Examples 362-380 includes, wherein the plurality of cell-free nucleic acid samples includes ribonucleic acid (RNA) samples.

In Example 382, the subject matter of Examples 362-381 includes, wherein the plurality of cell-free nucleic acid samples includes cell-free ribonucleic acid (cfRNA) samples.

In Example 383, the subject matter of Examples 362-382 includes, wherein the plurality of cell-free nucleic acid samples includes mitochondrial deoxyribonucleic (mtDNA) samples.

In Example 384, the subject matter of Examples 362-383 includes, wherein the plurality of cell-free nucleic acid samples includes mitochondrial ribonucleic (mtRNA) samples.

In Example 385, the subject matter of Examples 362-384 includes, wherein the plurality of cell-free nucleic acid samples includes extracellular vesicle-bound deoxyribonucleic (evDNA) samples.

Example 386 is one or more non-transitory computer readable media storing processor-executable instructions that, when executed by at least one processor, cause the at least one processor to: determining, based on a quantification of an observed tumor-associated aberrant methylation of each of a plurality of cell-free nucleic acid samples, using a tumor fraction regression (TFR) model, a TFR score, wherein the TFR score includes, a fraction of molecules of the plurality of cell-free nucleic acid samples that indicate a tumor, wherein each of the plurality of cell-free nucleic acid samples is labeled with a tumor-derived label or a non-tumor-derived label; determining, based on at least one of epigenetic factors or genomic alterations of each of the plurality of cell-free nucleic acid samples, a cell-free nucleic acid score indicative of presence of a tumor; determining, based on at least one of the cell-free nucleic acid score or the TFR score satisfying a respective threshold, a tumor prediction for each of the plurality of cell-free nucleic acid samples; generating, based on the tumor-derived label or the non-tumor-derived label and the tumor prediction for each of the plurality of cell-free nucleic acid samples, a predictive model to predict a tumor in the plurality of cell-free nucleic acid samples; and outputting the predictive model.

In Example 387, the subject matter of Example 386 includes, wherein the one or more non-transitory computer-readable storage media store additional computer-readable instructions that, when executed by the at least one processor, cause the at least one processor to perform additional operations comprising determining the quantification of the observed tumor-associated aberrant methylation of each of the plurality of cell-free nucleic acid samples.

In Example 388, the subject matter of Example 387 includes, wherein the computer-readable instructions that cause the at least one processor to perform operations comprising determining the quantification of the observed tumor-associated aberrant methylation of each of the plurality of cell-free nucleic acid samples includes computer-readable instructions that cause the at least one processor to perform operations comprising quantifying a number of unique methylated molecules mapping to each of a plurality of genomic regions.

In Example 389, the subject matter of Examples 386-388 includes, wherein the one or more non-transitory computer-readable storage media store additional computer-readable instructions that, when executed by the at least one processor, cause the at least one processor to perform additional operations comprising determining the epigenetic factors of each of the plurality of cell-free nucleic acid samples.

In Example 390, the subject matter of Example 389 includes, wherein the one or more non-transitory computer-readable storage media store additional computer-readable instructions that, when executed by the at least one processor, cause the at least one processor to perform additional operations comprising determining the epigenetic factors of each of the plurality of cell-free nucleic acid samples based on a methylation logistic regression (LR) model cancer or non-cancer classification.

In Example 391, the subject matter of Example 390 includes, wherein the one or more non-transitory computer-readable storage media store additional computer-readable instructions that, when executed by the at least one processor, cause the at least one processor to perform additional operations comprising determining, using a LR model, the methylation LR model cancer or non-cancer classification.

In Example 392, the subject matter of Examples 389-391 includes, wherein determining the epigenetic factors of each of the plurality of cell-free nucleic acid samples is based on a cancer signal from cell-free nucleic acid fragmentation patterns associated with a plurality of sequence fragments from the plurality of cell-free nucleic acid samples.

In Example 393, the subject matter of Example 392 includes, wherein the one or more non-transitory computer-readable storage media store additional computer-readable instructions that, when executed by the at least one processor, cause the at least one processor to perform additional operations comprising determining, using a fragmentomics model, the cancer signal from the cell-free nucleic acid fragmentation patterns associated with the plurality of sequence fragments from the plurality of cell-free nucleic acid samples.

In Example 394, the subject matter of Examples 389-393 includes, wherein the one or more non-transitory computer-readable storage media store additional computer-readable instructions that, when executed by the at least one processor, cause the at least one processor to perform additional operations comprising determining the epigenetic factors of each of the plurality of cell-free nucleic acid samples based on a methylation logistic regression (LR) model cancer or non-cancer classification and based on a cancer signal from cell-free nucleic acid fragmentation patterns associated with a plurality of sequence fragments from the plurality of cell-free nucleic acid samples.

In Example 395, the subject matter of Example 394 includes, wherein determining the cell-free nucleic acid score is further based on the TFR score.

In Example 396, the subject matter of Examples 386-395 includes, wherein the one or more non-transitory computer-readable storage media store additional computer-readable instructions that, when executed by the at least one processor, cause the at least one processor to perform additional operations comprising determining the genomic alterations of each of the plurality of cell-free nucleic acid samples.

In Example 397, the subject matter of Example 396 includes, wherein the computer-readable instructions that cause the at least one processor to perform operations comprising determining the genomic alterations of each of the plurality of cell-free nucleic acid samples includes computer-readable instructions that cause the at least one processor to perform operations comprising determining somatic variants observed in molecules from the plurality of cell-free nucleic acid samples.

In Example 398, the subject matter of Examples 386-397 includes, wherein the plurality of cell-free nucleic acid samples are from a plurality of genomic regions, wherein the plurality of genomic regions includes at least one of: a genomic region known to be associated with a cancer type, a genomic region associated with a known methylation status, a genomic region known to be associated with hypomethylation, or a genomic region known to be associated with therapy response.

In Example 399, the subject matter of Examples 386-398 includes, wherein the plurality of cell-free nucleic acid samples are from a plurality of genomic regions, wherein the plurality of genomic regions includes at least one genomic region known to be associated with colorectal cancer.

In Example 400, the subject matter of Examples 386-399 includes, wherein determining the cell-free nucleic acid score is further based on the TFR score.

In Example 401, the subject matter of Examples 386-400 includes, wherein the plurality of cell-free nucleic acid samples includes cell-free deoxyribonucleic (cfDNA) samples.

In Example 402, the subject matter of Examples 386-401 includes, wherein the plurality of cell-free nucleic acid samples includes ribonucleic acid (RNA) samples.

In Example 403, the subject matter of Examples 386-402 includes, wherein the plurality of cell-free nucleic acid samples includes cell-free ribonucleic acid (cfRNA) samples.

In Example 404, the subject matter of Examples 386-403 includes, wherein the plurality of cell-free nucleic acid samples includes mitochondrial deoxyribonucleic (mtDNA) samples.

In Example 405, the subject matter of Examples 386-404 includes, wherein the plurality of cell-free nucleic acid samples includes mitochondrial ribonucleic (mtRNA) samples.

In Example 406, the subject matter of Examples 386-405 includes, wherein the plurality of cell-free nucleic acid samples includes extracellular vesicle-bound deoxyribonucleic (evDNA) samples.

Example 407 is one or more non-transitory computer readable media storing processor-executable instructions that, when executed by at least one processor, cause the at least one processor to: determining, based on a quantification of an observed tumor-associated aberrant methylation of each of a plurality of cell-free nucleic acid samples, using a tumor fraction regression (TFR) model, a TFR score, wherein the TFR score includes, a fraction of molecules of the plurality of cell-free nucleic acid samples that indicate a tumor, wherein each of the plurality of cell-free nucleic acid samples is labeled with a tumor-derived label or a non-tumor-derived label; determining, based on the TFR score satisfying a respective threshold, a tumor prediction for each of the plurality of cell-free nucleic acid samples; generating, based on the tumor-derived label or the non-tumor-derived label and the tumor prediction of the plurality of cell-free nucleic acid samples, a predictive model to predict a tumor in the plurality of cell-free nucleic acid samples; and outputting the predictive model.

In Example 408, the subject matter of Example 407 includes, wherein the one or more non-transitory computer-readable storage media store additional computer-readable instructions that, when executed by the at least one processor, cause the at least one processor to perform additional operations comprising determining the quantification of the observed tumor-associated aberrant methylation of each of the plurality of cell-free nucleic acid samples.

In Example 409, the subject matter of Example 408 includes, wherein the computer-readable instructions that cause the at least one processor to perform operations comprising determining the quantification of the observed tumor-associated aberrant methylation of each of the plurality of cell-free nucleic acid samples includes computer-readable instructions that cause the at least one processor to perform operations comprising quantifying a number of unique methylated molecules mapping to each of a plurality of genomic regions.

In Example 410, the subject matter of Examples 407-409 includes, wherein determining the tumor prediction for each of the plurality of cell-free nucleic acid samples is further based on a cell-free nucleic acid score indicative of presence of a tumor.

In Example 411, the subject matter of Example 410 includes, wherein the one or more non-transitory computer-readable storage media store additional computer-readable instructions that, when executed by the at least one processor, cause the at least one processor to perform additional operations comprising determining, based on at least one of epigenetic factors or genomic alterations of each of the plurality of cell-free nucleic acid samples, the cell-free nucleic acid score indicative of presence of a tumor.

In Example 412, the subject matter of Example 411 includes, wherein the one or more non-transitory computer-readable storage media store additional computer-readable instructions that, when executed by the at least one processor, cause the at least one processor to perform additional operations comprising determining the epigenetic factors of each of the plurality of cell-free nucleic acid samples.

In Example 413, the subject matter of Example 412 includes, wherein the one or more non-transitory computer-readable storage media store additional computer-readable instructions that, when executed by the at least one processor, cause the at least one processor to perform additional operations comprising determining the epigenetic factors of each of the plurality of cell-free nucleic acid samples based on a methylation logistic regression (LR) model cancer or non-cancer classification.

In Example 414, the subject matter of Example 413 includes, wherein the one or more non-transitory computer-readable storage media store additional computer-readable instructions that, when executed by the at least one processor, cause the at least one processor to perform additional operations comprising determining, using a LR model, the methylation LR model cancer or non-cancer classification.

In Example 415, the subject matter of Examples 412-414 includes, wherein the one or more non-transitory computer-readable storage media store additional computer-readable instructions that, when executed by the at least one processor, cause the at least one processor to perform additional operations comprising determining the epigenetic factors of each of the plurality of cell-free nucleic acid samples based on a cancer signal from cell-free nucleic acid fragmentation patterns associated with a plurality of sequence fragments from the plurality of cell-free nucleic acid samples.

In Example 416, the subject matter of Example 415 includes, wherein the one or more non-transitory computer-readable storage media store additional computer-readable instructions that, when executed by the at least one processor, cause the at least one processor to perform additional operations comprising determining, using a fragmentomics model, the cancer signal from the cell-free nucleic acid fragmentation patterns associated with a plurality of sequence fragments from the plurality of cell-free nucleic acid samples.

In Example 417, the subject matter of Examples 411-416 includes, wherein the one or more non-transitory computer-readable storage media store additional computer-readable instructions that, when executed by the at least one processor, cause the at least one processor to perform additional operations comprising determining the genomic alterations of each of the plurality of cell-free nucleic acid samples.

In Example 418, the subject matter of Example 417 includes, wherein the computer-readable instructions that cause the at least one processor to perform operations comprising determining the genomic alterations of each of the plurality of cell-free nucleic acid samples includes computer-readable instructions that cause the at least one processor to perform operations comprising determining somatic variants observed in molecules from each of a plurality of sequence fragments from the plurality of cell-free nucleic acid samples.

In Example 419, the subject matter of Examples 411-418 includes, wherein the one or more non-transitory computer-readable storage media store additional computer-readable instructions that, when executed by the at least one processor, cause the at least one processor to perform additional operations comprising determining the epigenetic factors of each of the plurality of cell-free nucleic acid samples based on a methylation logistic regression (LR) model cancer or non-cancer classification and based on a cancer signal from cell-free nucleic acid fragmentation patterns associated with a plurality of sequence fragments from the plurality of cell-free nucleic acid samples.

In Example 420, the subject matter of Example 419 includes, wherein determining the cell-free nucleic acid score is further based on the TFR score.

In Example 421, the subject matter of Examples 407-420 includes, wherein the plurality of cell-free nucleic acid samples are from a plurality of genomic regions, wherein the plurality of genomic regions includes at least one of: a genomic region known to be associated with a cancer type, a genomic region associated with a known methylation status, a genomic region known to be associated with hypomethylation, or a genomic region known to be associated with therapy response.

In Example 422, the subject matter of Examples 407-421 includes, wherein the plurality of cell-free nucleic acid samples are from a plurality of genomic regions, wherein the plurality of genomic regions includes at least one genomic region known to be associated with colorectal cancer.

In Example 423, the subject matter of Examples 407-422 includes, wherein the plurality of cell-free nucleic acid samples includes cell-free deoxyribonucleic (cfDNA) samples.

In Example 424, the subject matter of Examples 406-423 includes, wherein the plurality of cell-free nucleic acid samples includes ribonucleic acid (RNA) samples.

In Example 425, the subject matter of Examples 407-424 includes, wherein the plurality of cell-free nucleic acid samples includes cell-free ribonucleic acid (cfRNA) samples.

In Example 426, the subject matter of Examples 407-425 includes, wherein the plurality of cell-free nucleic acid samples includes mitochondrial deoxyribonucleic (mtDNA) samples.

In Example 427, the subject matter of Examples 407-426 includes, wherein the plurality of cell-free nucleic acid samples includes mitochondrial ribonucleic (mtRNA) samples.

In Example 428, the subject matter of Examples 407-427 includes, wherein the plurality of cell-free nucleic acid samples includes extracellular vesicle-bound deoxyribonucleic (evDNA) samples.

Example 429 is one or more non-transitory computer readable media storing processor-executable instructions that, when executed by at least one processor, cause the at least one processor to: determining, based on at least one of epigenetic factors or genomic alterations of each of a plurality of cell-free nucleic acid samples, a cell-free nucleic acid score indicative of presence of a tumor, wherein each of the plurality of cell-free nucleic acid samples is labeled with a tumor-derived label or a non-tumor-derived label; determining, based on the cell-free nucleic acid score satisfying a respective threshold, a tumor prediction for each of the plurality of cell-free nucleic acid samples; generating, based on the tumor-derived label or the non-tumor-derived label and the tumor prediction for each of the plurality of cell-free nucleic acid samples, a predictive model to predict a tumor in the plurality of cell-free nucleic acid samples; and outputting the predictive model.

In Example 430, the subject matter of Example 429 includes, wherein determining the tumor prediction for each of the plurality of cell-free nucleic acid is further based on a tumor fraction regression (TFR) score satisfying a threshold, wherein the TFR score is indicative of a fraction of molecules of the plurality of cell-free nucleic acid samples that indicate a tumor.

In Example 431, the subject matter of Example 430 includes, wherein the one or more non-transitory computer-readable storage media store additional computer-readable instructions that, when executed by the at least one processor, cause the at least one processor to perform additional operations comprising determining, based on a quantification of an observed tumor-associated aberrant methylation of each of the plurality of cell-free nucleic acid samples, using a TFR model, the TFR score.

In Example 432, the subject matter of Example 431 includes, wherein the one or more non-transitory computer-readable storage media store additional computer-readable instructions that, when executed by the at least one processor, cause the at least one processor to perform additional operations comprising determining the quantification of the observed tumor-associated aberrant methylation of each of the plurality of cell-free nucleic acid samples.

In Example 433, the subject matter of Example 432 includes, wherein the computer-readable instructions that cause the at least one processor to perform operations comprising determining the quantification of the observed tumor-associated aberrant methylation of each of the plurality of cell-free nucleic acid samples includes computer-readable instructions that cause the at least one processor to perform operations comprising quantifying a number of unique methylated molecules mapping to each of a plurality of genomic regions.

In Example 434, the subject matter of Examples 430-433 includes, wherein determining the cell-free nucleic acid score is further based on the TFR score.

In Example 435, the subject matter of Examples 429-434 includes, wherein the one or more non-transitory computer-readable storage media store additional computer-readable instructions that, when executed by the at least one processor, cause the at least one processor to perform additional operations comprising determining the epigenetic factors of each of the plurality of cell-free nucleic acid samples.

In Example 436, the subject matter of Example 435 includes, wherein the one or more non-transitory computer-readable storage media store additional computer-readable instructions that, when executed by the at least one processor, cause the at least one processor to perform additional operations comprising determining the epigenetic factors of each of the plurality of cell-free nucleic acid samples based on a methylation logistic regression (LR) model cancer or non-cancer classification.

In Example 437, the subject matter of Example 436 includes, wherein the one or more non-transitory computer-readable storage media store additional computer-readable instructions that, when executed by the at least one processor, cause the at least one processor to perform additional operations comprising determining, using a LR model, the methylation LR model cancer or non-cancer classification.

In Example 438, the subject matter of Examples 435-437 includes, wherein the one or more non-transitory computer-readable storage media store additional computer-readable instructions that, when executed by the at least one processor, cause the at least one processor to perform additional operations comprising determining the epigenetic factors of each of the plurality of cell-free nucleic acid samples based on a cancer signal from cell-free nucleic acid fragmentation patterns associated with a plurality of sequence fragments from the plurality of cell-free nucleic acid samples.

In Example 439, the subject matter of Example 438 includes, wherein the one or more non-transitory computer-readable storage media store additional computer-readable instructions that, when executed by the at least one processor, cause the at least one processor to perform additional operations comprising determining, using a fragmentomics model, the cancer signal from the cell-free nucleic acid fragmentation patterns associated with the plurality of sequence fragments from the plurality of cell-free nucleic acid samples.

In Example 440, the subject matter of Examples 435-439 includes, wherein the one or more non-transitory computer-readable storage media store additional computer-readable instructions that, when executed by the at least one processor, cause the at least one processor to perform additional operations comprising determining the epigenetic factors of each of the plurality of cell-free nucleic acid samples based on a methylation logistic regression (LR) model cancer or non-cancer classification and based on a cancer signal from cell-free nucleic acid fragmentation patterns associated with a plurality of sequence fragments from the plurality of cell-free nucleic acid samples.

In Example 441, the subject matter of Example 440 includes, wherein determining the cell-free nucleic acid score is further based on a tumor fraction regression (TFR) score, wherein the TFR score is indicative of a fraction of molecules of the plurality of cell-free nucleic acid samples that indicate a tumor.

In Example 442, the subject matter of Examples 429-441 includes, wherein the one or more non-transitory computer-readable storage media store additional computer-readable instructions that, when executed by the at least one processor, cause the at least one processor to perform additional operations comprising determining the genomic alterations of each of the plurality of cell-free nucleic acid samples.

In Example 443, the subject matter of Example 442 includes, wherein the computer-readable instructions that cause the at least one processor to perform operations comprising determining the genomic alterations of each of the plurality of cell-free nucleic acid samples includes computer-readable instructions that cause the at least one processor to perform operations comprising determining somatic variants observed in molecules from each of a plurality of sequence fragments from the plurality of cell-free nucleic acid samples.

In Example 444, the subject matter of Examples 429-443 includes, wherein the plurality of cell-free nucleic acid samples are from a plurality of genomic regions, wherein the plurality of genomic regions includes at least one of: a genomic region known to be associated with a cancer type, a genomic region associated with a known methylation status, a genomic region known to be associated with hypomethylation, or a genomic region known to be associated with therapy response.

In Example 445, the subject matter of Examples 429-444 includes, wherein the plurality of cell-free nucleic acid samples are from a plurality of genomic regions, wherein the plurality of genomic regions includes at least one genomic region known to be associated with colorectal cancer.

In Example 446, the subject matter of Examples 429-445 includes, wherein determining that the plurality of cell-free nucleic acid samples is tumor-derived or non-tumor derived is further based on a methylation value satisfying a threshold, wherein the methylation score is indicative of a quantity of molecules of the plurality of cell-free nucleic acid samples that indicate a tumor.

In Example 447, the subject matter of Examples 429-446 includes, wherein the plurality of cell-free nucleic acid samples includes cell-free deoxyribonucleic (cfDNA) samples.

In Example 448, the subject matter of Examples 429-447 includes, wherein the plurality of cell-free nucleic acid samples includes ribonucleic acid (RNA) samples.

In Example 449, the subject matter of Examples 429-448 includes, wherein the plurality of cell-free nucleic acid samples includes cell-free ribonucleic acid (cfRNA) samples.

In Example 450, the subject matter of Examples 429-449 includes, wherein the plurality of cell-free nucleic acid samples includes mitochondrial deoxyribonucleic (mtDNA) samples.

In Example 451, the subject matter of Examples 429-450 includes, wherein the plurality of cell-free nucleic acid samples includes mitochondrial ribonucleic (mtRNA) samples.

In Example 452, the subject matter of Examples 429-451 includes, wherein the plurality of cell-free nucleic acid samples includes extracellular vesicle-bound deoxyribonucleic (evDNA) samples.

Example 453 is one or more non-transitory computer readable media storing processor-executable instructions that, when executed by at least one processor, cause the at least one processor to: detecting one or more biomarkers in a biological sample; determining, based on a quantification of an observed tumor-associated aberrant methylation of each of a plurality of cell-free nucleic acid samples, using a tumor fraction regression (TFR) model, a TFR score, wherein the TFR score includes, a fraction of molecules of the plurality of cell-free nucleic acid samples that indicate a tumor; determining, based on at least one of epigenetic factors or genomic alterations of each of the plurality of cell-free nucleic acid samples, a cell-free nucleic acid score indicative of presence of a tumor; and determining, based on at least one of the detected biomarkers, the cell-free nucleic acid score, or the TFR score satisfying a respective threshold, that the biological sample is tumor-derived or non-tumor derived.

In Example 454, the subject matter of Example 453 includes, wherein the one or more non-transitory computer-readable storage media store additional computer-readable instructions that, when executed by the at least one processor, cause the at least one processor to perform additional operations comprising determining the quantification of the observed tumor-associated aberrant methylation of each of the plurality of cell-free nucleic acid samples.

In Example 455, the subject matter of Example 454 includes, wherein the computer-readable instructions that cause the at least one processor to perform operations comprising determining the quantification of the observed tumor-associated aberrant methylation of each of the plurality of cell-free nucleic acid samples includes computer-readable instructions that cause the at least one processor to perform operations comprising quantifying a number of unique methylated molecules mapping to each of a plurality of genomic regions.

In Example 456, the subject matter of Examples 453-455 includes, wherein the one or more non-transitory computer-readable storage media store additional computer-readable instructions that, when executed by the at least one processor, cause the at least one processor to perform additional operations comprising determining the epigenetic factors of each of the plurality of cell-free nucleic acid samples.

In Example 457, the subject matter of Example 456 includes, wherein the one or more non-transitory computer-readable storage media store additional computer-readable instructions that, when executed by the at least one processor, cause the at least one processor to perform additional operations comprising determining the epigenetic factors of each of the plurality of cell-free nucleic acid samples based on a methylation logistic regression (LR) model cancer or non-cancer classification.

In Example 458, the subject matter of Example 457 includes, wherein the one or more non-transitory computer-readable storage media store additional computer-readable instructions that, when executed by the at least one processor, cause the at least one processor to perform additional operations comprising determining, using a LR model, the methylation LR model cancer or non-cancer classification.

In Example 459, the subject matter of Examples 456-458 includes, wherein the one or more non-transitory computer-readable storage media store additional computer-readable instructions that, when executed by the at least one processor, cause the at least one processor to perform additional operations comprising determining the epigenetic factors of each of the plurality of cell-free nucleic acid samples based on a cancer signal from cell-free nucleic acid fragmentation patterns associated with a plurality of sequence fragments from the plurality of cell-free nucleic acid samples.

In Example 460, the subject matter of Example 459 includes, wherein the one or more non-transitory computer-readable storage media store additional computer-readable instructions that, when executed by the at least one processor, cause the at least one processor to perform additional operations comprising determining, using a fragmentomics model, the cancer signal from the cell-free nucleic acid fragmentation patterns associated with the plurality of sequence fragments from the plurality of cell-free nucleic acid samples.

In Example 461, the subject matter of Examples 456-460 includes, wherein the one or more non-transitory computer-readable storage media store additional computer-readable instructions that, when executed by the at least one processor, cause the at least one processor to perform additional operations comprising determining the epigenetic factors of each of the plurality of cell-free nucleic acid samples based on a methylation logistic regression (LR) model cancer or non-cancer classification and based on a cancer signal from cell-free nucleic acid fragmentation patterns associated with a plurality of sequence fragments from the plurality of cell-free nucleic acid samples.

In Example 462, the subject matter of Example 461 includes, wherein determining the cell-free nucleic acid score is further based on the TFR score.

In Example 463, the subject matter of Examples 453-462 includes, wherein the one or more non-transitory computer-readable storage media store additional computer-readable instructions that, when executed by the at least one processor, cause the at least one processor to perform additional operations comprising determining the genomic alterations of each of the plurality of cell-free nucleic acid samples.

In Example 464, the subject matter of Example 463 includes, wherein the computer-readable instructions that cause the at least one processor to perform operations comprising determining the genomic alterations of each of the plurality of cell-free nucleic acid samples includes computer-readable instructions that cause the at least one processor to perform operations comprising determining somatic variants observed in molecules from each of a plurality of sequence fragments from the plurality of cell-free nucleic acid samples.

In Example 465, the subject matter of Examples 453-464 includes, wherein the plurality of cell-free nucleic acid samples are from a plurality of genomic regions, wherein the plurality of genomic regions includes at least one of: a genomic region known to be associated with a cancer type, a genomic region associated with a known methylation status, a genomic region known to be associated with hypomethylation, or a genomic region known to be associated with therapy response.

In Example 466, the subject matter of Examples 453-465 includes, wherein the plurality of cell-free nucleic acid samples are from a plurality of genomic regions, wherein the plurality of genomic regions includes at least one genomic region known to be associated with colorectal cancer.

In Example 467, the subject matter of Examples 453-466 includes, wherein determining the cell-free nucleic acid score is further based on the TFR score.

In Example 468, the subject matter of Examples 453-467 includes, wherein the plurality of cell-free nucleic acid samples includes cell-free deoxyribonucleic (cfDNA) samples.

In Example 469, the subject matter of Examples 453-468 includes, wherein the plurality of cell-free nucleic acid samples includes ribonucleic acid (RNA) samples.

In Example 470, the subject matter of Examples 453-469 includes, wherein the plurality of cell-free nucleic acid samples includes cell-free ribonucleic acid (cfRNA) samples.

In Example 471, the subject matter of Examples 453-470 includes, wherein the plurality of cell-free nucleic acid samples includes mitochondrial deoxyribonucleic (mtDNA) samples.

In Example 472, the subject matter of Examples 453-471 includes, wherein the plurality of cell-free nucleic acid samples includes mitochondrial ribonucleic (mtRNA) samples.

In Example 473, the subject matter of Examples 453-472 includes, wherein the plurality of cell-free nucleic acid samples includes extracellular vesicle-bound deoxyribonucleic (evDNA) samples.

In Example 474, the subject matter of Examples 453-473 includes, wherein the biomarker is one or more of those selected from: proteins, exosomes, exomeres, microvesicles, apoptotic bodies, neutrophil extracellular traps (NETs), immune cells, tumor-educated platelets (TEPs), microbiome, virome, toll-like receptors (TLRs), and mitochondrial DNA (mtDNA).

In Example 475, the subject matter of Examples 453-474 includes, wherein the computer-readable instructions that cause the at least one processor to perform operations comprising detecting one ore more biomarkers includes computer-readable instructions that cause the at least one processor to perform operations comprising detecting the presence or levels of the one or more biomarkers.

In Example 476, the subject matter of Examples 453-475 includes, wherein the computer-readable instructions that cause the at least one processor to perform operations comprising determining that the biological sample is tumor-derived or non-tumor derived includes computer-readable instructions that cause the at least one processor to perform operations comprising comparing the levels of the one or more biomarkers in the biological sample to a control.

In Example 477, the subject matter of Example 476 includes, wherein the control is a reference level or a level present in a healthy, non-cancer subject.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise if different versions of a publication, website or the like are published at different times, the version most recently published at the effective filing date of the application is meant unless otherwise indicated. Any feature, step, element, embodiment, or aspect of the disclosure can be used in combination with any other unless specifically indicated otherwise. Although the present disclosure has been described in some detail by way of illustration and example for purposes of clarity and understanding, it will be apparent that certain changes and modifications may be practiced within the scope of the appended claims.

1-67. (canceled)

68. A method comprising:
determining, based on a quantification of an observed tumor-associated aberrant methylation of each of a plurality of cell-free nucleic acid samples, using a tumor fraction regression (TFR) model, a TFR score, wherein the TFR score includes a fraction of molecules of the plurality of cell-free nucleic acid samples that indicate a tumor, wherein each of the plurality of cell-free nucleic acid samples is labeled with a tumor-derived label or a non-tumor-derived label;
determining, based on at least one of epigenetic factors or genomic alterations of each of the plurality of cell-free nucleic acid samples, a cell-free nucleic acid score indicative of presence of a tumor;
determining, based on at least one of the cell-free nucleic acid score or the TFR score satisfying a respective threshold, a tumor prediction for each of the plurality of cell-free nucleic acid samples;
generating, based on the tumor-derived label or the non-tumor-derived label and the tumor prediction for each of the plurality of cell-free nucleic acid samples, a predictive model to predict a tumor in the plurality of cell-free nucleic acid samples; and
outputting the predictive model.

69. The method of claim 68, further comprising determining the quantification of the observed tumor-associated aberrant methylation of each of the plurality of cell-free nucleic acid samples.

70. The method of claim 69, wherein determining the quantification of the observed tumor-associated aberrant methylation of each of the plurality of cell-free nucleic acid samples includes quantifying a number of unique methylated molecules mapping to each of a plurality of genomic regions.

71. The method of claim 68, further comprising determining the epigenetic factors of each of the plurality of cell-free nucleic acid samples.

72. The method of claim 71, further comprising determining the epigenetic factors of each of the plurality of cell-free nucleic acid samples based on a methylation logistic regression (LR) model cancer or non-cancer classification.

73. The method of claim 72, further comprising determining, using a LR model, the methylation LR model cancer or non-cancer classification.

74. The method of claim 71, wherein determining the epigenetic factors of each of the plurality of cell-free nucleic acid samples is based on a cancer signal from cell-free nucleic acid fragmentation patterns associated with a plurality of sequence fragments from the plurality of cell-free nucleic acid samples.

75. The method of claim 74, further comprising determining, using a fragmentomics model, the cancer signal from the cell-free nucleic acid fragmentation patterns associated with the plurality of sequence fragments from the plurality of cell-free nucleic acid samples.

76-77. (canceled)

78. The method of claim 68, further comprising determining the genomic alterations of each of the plurality of cell-free nucleic acid samples.

79. The method of claim 78, wherein determining the genomic alterations of each of the plurality of cell-free nucleic acid samples includes determining somatic variants observed in molecules from the plurality of cell-free nucleic acid samples.

80-134. (canceled)

135. A method comprising:
detecting one or more biomarkers in a biological sample;
determining, based on a quantification of an observed tumor-associated aberrant methylation of each of a plurality of cell-free nucleic acid samples, using a tumor fraction regression (TFR) model, a TFR score, wherein the TFR score includes a fraction of molecules of the plurality of cell-free nucleic acid samples that indicate a tumor;
determining, based on at least one of epigenetic factors or genomic alterations of each of the plurality of cell-free nucleic acid samples, a cell-free nucleic acid score indicative of presence of a tumor; and
determining, based on at least one of the detected biomarkers, the cell-free nucleic acid score, or the TFR score satisfying a respective threshold, that the biological sample is tumor-derived or non-tumor derived.

136. The method of claim 135, further comprising determining the quantification of the observed tumor-associated aberrant methylation of each of the plurality of cell-free nucleic acid samples.

137. The method of claim 136, wherein determining the quantification of the observed tumor-associated aberrant methylation of each of the plurality of cell-free nucleic acid samples includes quantifying a number of unique methylated molecules mapping to each of a plurality of genomic regions.

138. The method of claim 135, further comprising determining the epigenetic factors of each of the plurality of cell-free nucleic acid samples.

139. The method of claim 138, further comprising determining the epigenetic factors of each of the plurality of cell-free nucleic acid samples based on a methylation logistic regression (LR) model cancer or non-cancer classification.

140. The method of claim 139, further comprising determining, using a LR model, the methylation LR model cancer or non-cancer classification.

141. The method of claim 138, further comprising determining the epigenetic factors of each of the plurality of cell-free nucleic acid samples based on a cancer signal from cell-free nucleic acid fragmentation patterns associated with a plurality of sequence fragments from the plurality of cell-free nucleic acid samples.

142. The method of claim 141, further comprising determining, using a fragmentomics model, the cancer signal from the cell-free nucleic acid fragmentation patterns associated with the plurality of sequence fragments from the plurality of cell-free nucleic acid samples.

143-144. (canceled)

145. The method of claim 135, further comprising determining the genomic alterations of each of the plurality of cell-free nucleic acid samples.

146. The method of claim 145, wherein determining the genomic alterations of each of the plurality of cell-free nucleic acid samples includes determining somatic variants observed in molecules from each of a plurality of sequence fragments from the plurality of cell-free nucleic acid samples.

147-159. (canceled)
